(19) United States
(12) Patent Application Publication
(10) Pub. No.: US 2019/0343908 A1
(43) Pub. Date: Nov. 14, 2019

(54) Therapeutic Combination for the Treatment of Cancer

(71) Applicant: Nerium Biotechnology Inc., San Antonio, TX (US)

(72) Inventors: Dennis R. Knocke, Shavano Park, TX (US); Joseph B. Nester, San Antonio, TX (US); Ioannis Papasotiriou, Filotas (GR)

(73) Assignee: Nerium Biotechnology Inc., San Antonio, TX (US)

(21) Appl. No.: 16/419,226

(22) Filed: May 22, 2019

Related U.S. Application Data
(63) Continuation of application No. 13/838,739, filed on Mar. 15, 2013, now abandoned.
Application No. 13/838,739 is a continuation-in-part of application No. PCT/US2012/061226, filed on Oct. 20, 2012.
(60) Provisional application No. 61/549,386, filed on Oct. 20, 2011.

(51) Int. Cl. A61K 36/24; A61K 36/886; A61K 31/665; A61K 31/7048; A61K 33/24; A61K 31/282; A61K 31/555

(52) U.S. Cl. CPC A61K 36/24; A61K 36/886; A61K 31/665; A61K 31/555; A61K 33/24; A61K 31/282; A61K 31/7048

(57) ABSTRACT

This invention relates to therapeutic combinations comprising a platinum-based anti-neoplastic agent, such as cisplatin, and an extract from a species of the genus Nerium, such as Nerium oleander, as well as methods of using such combinations to treat subjects, including humans, suffering from certain cancers, such as prostate cancer, melanoma, pancreatic cancer, lung cancer, breast cancer, or colorectal cancer.

### FIELD OF THE INVENTION

This invention relates to therapeutic combinations comprising a platinum-based anti-neoplastic agent and an extract from a species of the genus Nerium, as well as methods of using such combinations to treat patients suffering from certain types of cancer.

### BACKGROUND OF THE INVENTION

Because of the continuing need for more effective ways to treat cancer, great effort continues to be exerted to develop new drugs that are less toxic to the patient being treated. However, another approach is to try to lower the toxicity of existing anti-cancer drugs by combining them with other drugs.

Platinum-based anti-cancer drugs, such as cisplatin, find use in chemotherapy of various types of cancer, but their toxicity remains a serious problem. Furthermore, the emergence of cisplatin resistance in some tumors vitiates its utility at the toxicity-limited dosage. To mitigate these limitations, cisplatin is commonly used in combination with other drugs, such as 5-fluorouracil, that exert their toxic effects on different organs than those affected by cisplatin.

Extracts of Nerium oleander comprise various polysaccharides and proteins as well as two toxic cardiac glycosides, oleandrin and oleandrigenin, that are known to exhibit anti-cancer activity.

### EMBODIMENTS OF THE INVENTION

An embodiment of the invention is a therapeutic combination comprising (I) an anti-neoplastic agent comprising platinum and (II) an extract from a species of the genus Nerium.

Another embodiment of the invention is the above therapeutic combination, wherein (I) is present in an amount that, in the absence of (II), is effective to produce an increase in anti-neoplastic activity.

Another embodiment of the invention is the above therapeutic combination, wherein (I) is present in an amount that, in the absence of (II), is ineffective to produce an increase in anti-neoplastic activity.

Another embodiment of the invention is the above therapeutic combination, wherein (I) is selected from the group consisting of cisplatin, carboplatin, oxaliplatin, satraplatin, picoplatin, nedaplatin, triplatin, and phosphaplatins.

Another embodiment of the invention is the above therapeutic combination, wherein (I) is cisplatin.

Another embodiment of the invention is the above therapeutic combination, wherein (II) is an extract from a species selected from the group consisting of Nerium indicum, Nerium oleander, and Nerium odorum.

Another embodiment of the invention is the above therapeutic combination, wherein (II) is an extract from the species Nerium oleander.

Another embodiment of the invention is the above therapeutic combination, wherein (II) is a water-based extract.

Another embodiment of the invention is the above therapeutic combination, wherein (II) is an aloe-based extract.

Another embodiment of the invention is the above therapeutic combination, wherein (I) is cisplatin and (II) is an extract from the species Nerium oleander.

Another embodiment of the invention is the above therapeutic combination, wherein (I) and (II) are present as separate formulations.

Yet another embodiment of the invention is a method for treating cancer comprising administering to a patient in need thereof a therapeutic combination comprising (I) an anti-neoplastic agent comprising platinum and (II) an extract from a species of the genus Nerium.

Another embodiment of the invention is the above method, wherein (I) is present in an amount that, in the absence of (II), is effective to produce an increase in anti-neoplastic activity.

Another embodiment of the invention is the above method, wherein (I) is present in an amount that, in the absence of (II), is ineffective to produce an increase in anti-neoplastic activity.

Another embodiment of the invention is the above method, wherein (I) is selected from the group consisting of cisplatin, carboplatin, oxaliplatin, satraplatin, picoplatin, nedaplatin, triplatin, and phosphaplatins.

Another embodiment of the invention is the above method, wherein (I) is cisplatin.

Another embodiment of the invention is the above method, wherein (II) is an extract from a species selected from the group consisting of Nerium indicum, Nerium oleander, and Nerium odorum.

Another embodiment of the invention is the above method, wherein (II) is an extract from the species Nerium oleander.

Another embodiment of the invention is the above method, wherein (II) is a water-based extract.

Another embodiment of the invention is the above method, wherein (II) is an aloe-based extract.

Another embodiment of the invention is the above method, wherein (I) is cisplatin and (II) is an extract from the species Nerium oleander.

Another embodiment of the invention is the above method, wherein (I) and (II) of said therapeutic combination are administered to the patient simultaneously, separately, or sequentially.

Another embodiment of the invention is the above method, wherein (II) is administered to the patient intramuscularly, sublingually, or intramuscularly and sublingually.

Another embodiment of the invention is the above method, wherein (II) is administered to the patient sublingually in two or more doses.

Another embodiment of the invention is the above method, wherein the cancer treated is prostate cancer, melanoma, pancreatic cancer, lung cancer, breast cancer, or colorectal cancer.

### BRIEF DESCRIPTION OF THE DRAWINGS

FIG. 1 depicts a graph of the relative reduction in cells of the LNCaP prostate cancer cell line in the presence of Anvirzel™, cisplatin, and a combination of the two.

FIG. 2 depicts a graph of the relative reduction in cells of the A375 melanoma cancer cell line in the presence of Anvirzel™, cisplatin, and a combination of the two.

FIG. 3 depicts a graph of the relative reduction in cells of the PANC-1 pancreatic cancer cell line in the presence of Anvirzel™, cisplatin, and a combination of the two.

FIG. 4 depicts a graph of the relative reduction in cells of the COLO699N lung cancer cell line in the presence of Anvirzel™, cisplatin, and a combination of the two.

### DETAILED DESCRIPTION OF THE INVENTION

It has surprisingly been found that combinations of an anti-neoplastic agent comprising platinum and an extract from a species of the genus Nerium synergistically exhibit increased anti-neoplastic activity. As a result, otherwise ineffective but well-tolerated doses of such platinum-based anti-neoplastic agents, when combined with dosages of the extract that lack efficacy themselves, exert clinically useful anti-neoplastic efficacy.

The therapeutic combinations of this invention comprise an anti-neoplastic agent comprising platinum and an extract from a species of the genus Nerium.

The anti-neoplastic agent used in the therapeutic combinations of this invention can be any known anti-cancer or anti-tumor agent or drug that contains platinum. Non-limiting examples include cisplatin, carboplatin, oxaliplatin, satraplatin, picoplatin, nedaplatin, triplatin, and phosphaplatins, such as those disclosed in U.S. Pat. No. 8,034,964 and which are incorporated herein by reference. These compounds can be prepared by methods known in the art. Preferably, the anti-neoplastic agent is cisplatin. Cisplatin is a divalent inorganic water-soluble, platinum containing complex widely used to treat testicular, bladder, and ovarian cancers.

The platinum-based anti-neoplastic agents used in the therapeutic combinations of this invention can be combined with any pharmaceutically acceptable excipient, carrier, adjunct, or diluent known in the art.

The extract used in the therapeutic combinations of this invention can be derived from any species of the genus Nerium. Non-limiting examples include extracts from the species Nerium indicum, Nerium oleander, and Nerium odorum. Preferably, the extract is derived from Nerium oleander.

Extracts of Nerium oleander can be prepared by methods known in the art. Such methods include extraction with hot water (U.S. Pat. Nos. 6,565,897 and 5,135,745), room temperature water and aqueous alcohol, particularly aqueous methanol and ethanol (U.S. Patent App. Pub. No. 2007/0154573 A1), supercritical carbon dioxide (U.S. Pat. No. 7,402,325), and aloe (U.S. Patent App. Pub. No. 2010/0092585 A1). These aforementioned extraction methods disclosed in U.S. Pat. Nos. 6,565,897, 5,135,745, and 7,402,325 and U.S. Patent App. Pub. Nos. 2007/0154573 A1 and 2010/0092585 A1 are incorporated herein by reference. Anvirzel™ is a hot water extract of Nerium oleander that contains oleandrin and oleandrigenin as its main constituents.

The extracts used in the combinations of the invention can contain any pharmaceutically acceptable excipient, carrier, adjunct, or diluent known in the art. Examples include mannitol, ascorbic acid, sodium ascorbate, methyl paraben, propyl paraben, and mixtures thereof.

Preferably, the therapeutic combination of the invention comprises cisplatin and an extract from the species Nerium oleander.

The anti-neoplastic agent comprising platinum and the extract from a species of the genus Nerium can be present in the therapeutic combinations of the invention as either separate or combined formulations.

The method of this invention for treating cancer comprises administering to a patient in need thereof a therapeutic combination comprising an anti-neoplastic agent comprising platinum and an extract from a species of the genus Nerium. As used herein, the term “patient” refers to a mammal afflicted with cancer. The preferred patient is a human.

The anti-neoplastic agent comprising platinum can be present in the therapeutic combinations of the invention and used in the method of this invention in an amount that, in the absence of the extract from a species of the genus Nerium, is either effective or ineffective to produce an increase in anti-neoplastic activity. An amount or dose that is “effective” is that which produces a particular cancer cell growth- or proliferation-inhibiting effect, tumor growth inhibiting effect, tumor volume increase-inhibiting effect, or cancer treatment effect in a cancer cell or tumor.

The anti-neoplastic agent comprising platinum can be administered to the patient via any mode known in the art, such as intravenously in the case of cisplatin, carboplatin, and oxalplatin, and orally in the case of satraplatin. A typical dosage of cisplatin for an adult is 70-100 mg/m² a day for three days, followed by two weeks off, constituting one cycle. Patients can receive a maximum of six cycles of therapy, but owing to toxicity problems most patients can only endure four cycles of such therapy. A typical adult (170 cm tall and weighing 70 kg), has 1.83 m² (The Journal of Pediatrics 1978 93:1:62-66; Gehan E A, George S L), corresponding to a dosage of about 125-185 mg.

The extract used in this method can be administered intramuscularly, sublingually, or intramuscularly and sublingually, either in a single dose or in multiple doses. The normal dosage for Anvirzel™ is 0.5 to 1.0 mL intramuscularly, 1.0 to 2.0 mL sublingually, or a combination of intramuscular and sublingual administration so that the total amount in a 24 hour period is no greater that 2 mL. Anvirzel™ administered sublingually only is divided into a series of smaller sub-doses, typically two to four doses of 0.5 mL or less per 24 hours.

As is common with pharmaceutical agents, the respective therapeutic doses of the components of the therapeutic combinations of the invention may vary with the condition of the patient and the route by which the drug is administered. The dose, and perhaps the dose frequency, will also vary according to the age, body weight, and response of the individual patient. It may be necessary to use dosages outside these ranges in some cases, as will be apparent to those skilled in the art. Further, it is noted that the clinician or treating physician will know how and when to interrupt, adjust, or terminate therapy in conjunction with individual patient response. The terms “therapeutic amount” and “therapeutically effective amount” are encompassed by the above-described dosage amounts and dose frequency schedules.

The therapeutic combinations of the invention can be administered to the patient simultaneously, separately, or sequentially.

The therapeutic combinations of the invention can be used according to this method to treat any type of cancer. Examples include prostate cancer, melanoma, pancreatic cancer, lung cancer, breast cancer, and colorectal cancer.

The following non-limiting examples demonstrate the synergistic cytostatic effect of co-administration of a Nerium oleander extract with cisplatin.

While these examples show certain specific embodiments of the invention, it will be manifest to those skilled in the art that various modifications and rearrangements of the parts may be made without departing from the spirit and scope of the underlying inventive concept and that the same is not limited to the particular forms herein shown and described.

### EXAMPLES

Viability Assays

Three different viability assays were chosen to complement each other and yielded results dependent on concentration and cell line: the Methyl Tetrazolium dye assay, the Sulforhodamine B fluorescent dye assay, and the Crystal Violet dye assay.

The Methyl-Tetrazolium dye assay measures the activity of enzymes in mitochondria. The dye precursor is taken up by cells, where it undergoes reduction to the purple formazan. It measures only the mitochondrial activity and, thus, it is not always a reliable indication of whether the cells themselves are alive or dead, since even a recently dead cell has lower enzyme activity in mitochondria. Intracellular glucose concentration, pH, and time before assay all affect the measurements.

The Sulforhodamine B and Crystal Violet assays provide information about protein and were used to complement the Methyl-Tetrazolium dye assay. The Sulforhodamine B assay is more sensitive for the detection of small number of cells and shows a linear relationship between cell number and its staining intensity. It is used for the quantification of cellular proteins of cultured cells. The assay using Crystal Violet, a dye that binds electrostatically to proteins and stains DNA, provides a reliable, simple assay for measuring viability of cells.

The Methyl Tetrazolium, Sulforhodamine B, and Crystal Violet assays were used to assess cell viabilities. The incubation times were 24 hours, 48 hours, and 72 hours. The Nerium oleander extract Anvirzel™ (a hot water extract of oleander obtained from Salud Integral, Honduras) was tested in concentrations ranging from 0.01 ng/mL to 10 ng/mL. Cisplatin (Sigma, P4394) was tested in concentrations ranging from 0.1 μg/mL to 100 μg/mL.

Cells were detached by trypsinization (Trypsin-0.25% EDTA, Invitrogen, 25200-072) during the logarithmic phase of culture growth and plated in 96-well plates (18.000 cells/well) (Corning, Costar 359) in a final volume of 200 μl of medium per well. After 70 to 80% confluence of the culture, the medium was removed and the Anvirzel™ diluted in water and cisplatin diluted in N,N-dimethylformamide (Fluka, 40255) were added to the cells in graduated densities. The absorbance was measured after 24 hours, 48 hours, and 72 hours of incubation.

For the Sulforhodamine B assay, 96-well plates were fixed by 10% trichloroacetic acid (Fluka, 91228) and were incubated at 4° C. for 1 hour. Afterwards, plates were rinsed with water and cells were stained with 0.4% Sulforhodamine B (Sigma, 341738), dissolved in 1% acetic acid (Carlo Ebra, 401422) for 15 minutes at room temperature (RT). The unbound stain was washed twice with 1% acetic acid. Finally, 10 mM Tris Buffer pH 10.5 (Sigma, T6791) was added to dissolve the dye.

For the Crystal Violet assay, the medium was removed from the 96-well plates, the plates rinsed with PBS (Sigma, P3813) and then the cells were rinsed by the addition of 10% formalin (MERCK, 1.04003.2500) for 20 minutes at RT. Formalin was removed and 0.25% aqueous crystal violet (Sigma, HT901), dissolved in water, was added for 10 minutes at RT. Unbound Crystal Violet was rinsed by washing with water and finally 33% acetic acid was added to dissolve the dye.

For the Methyl-Tetrazolium assay, the dye (Sigma, M2128; 5 mg/mL, diluted in phosphate-buffered saline) was added to each well and plates were incubated for 3 hours at 37° C. After the end of the incubation period, the medium was discarded and the cells were rinsed with phosphate-buffered saline. Finally, the formazan crystals were dissolved in dimethylsulphoxide (Sigma, D4540).

The optical density of each plate was measured on a μQuant spectrophotometer and the data were analyzed with Gen5 software (μQuant Biomolecular Spectrophotometer MQX200 and Gen5™ Microplate Data Collection & Analysis software, BioTek® Instruments. Inc, April 2008). Absorbance was measured at 570 nm for all assays and a second wavelength was measured in order to subtract noise. For the Methyl-Tetrazolium assay this second wavelength was 630 nm and for the Sulforhodamine B and Crystal Violet assays it was 690 nm.

Cell Lines

Human carcinoma cell lines were obtained by the European Collection of Cell Cultures from Health Protective Agency, UK, and included lines derived from human prostate cancer (PC3, LNCaP and 22Rv1), human breast cancer (MDA-MB 231, T47D, MCF-7), non-small cell lung carcinoma (CALU-1, COLO699N, COR-L 105), colorectal cancer (HCT-116, HT55, HCT-15), melanoma (A375), and pancreatic cancer (PANC-1).

Cells were cultured in 75 cm² flasks (Orange Scientific, 5520200, Belgium) in the medium indicated for each line with the appropriate amount of heat inactivated Fetal Bovine Serum (FBS, Invitrogen, 10106-169, California) and 2 mM L-Glutamine (Sigma, G5792, Germany) for each cell line and incubated at 37° C. in a 5% CO₂ atmosphere.

Statistical Analysis

All treatments for each cell line were performed in triplicate. The statistical significance of all effects was evaluated by “difference of the means” test (p<0.05).

Anvirzel™ concentrations ranged from 0.01 ng/mL to 10 ng/mL, while those for cisplatin ranged from 0.1 μg/mL to 100 μg/mL. Surprisingly, lower concentrations of both the Anvirzel™ (0.01 to 0.1 ng/mL) and the cisplatin (0.1 μg/mL) gave better results than higher concentrations, which yielded unreliable and irreproducible results. The results were also time-dependent, as was observed after 48 and 72 hours of incubation.

### Example 1: LNCaP Cell Line

Table 1 below shows results for LNCaP cells, a prostatic cancer-derived cell line, with cell population densities estimated from optical absorbance data.

By Crystal Violet assay, after 48 hours of incubation, unstimulated cells showed an apparent absorbance of 1.307 (a dimensionless quantity), with essentially the same value (1.249) obtained in the presence of 0.01 ng/mL of Anvirzel™. The presence of 0.1 μg/mL of cisplatin reduced the apparent absorbance to 0.408, but the presence of 0.01 of ng/mL Anvirzel™ and 0.1 μg/mL of cisplatin further reduced the value to 0.178 (p=0.00008<0.05). A similar diminution in apparent absorbance was found after 72 hours incubation as well.

By Sulforhodamine B assay, after 48 hours of incubation, unstimulated cells showed an apparent absorbance of 2.616, with essentially the same value (2.667) obtained in the presence of 0.01 ng/mL of Anvirzel™. The presence of 0.1 μg/mL of cisplatin reduced the apparent absorbance to 1.681, but the presence of 0.01 ng/mL of Anvirzel™ and 0.1 μg/mL of cisplatin further reduced the value to 1.033 (p=0.007<0.05). A similar diminution in apparent absorbance was found after 72 hours incubation as well.

By Methyl-Tetrazolium assay, after 48 hours of incubation, unstimulated cells showed an apparent absorbance of 0.429, with a somewhat lower value (0.292) obtained in the presence of 0.01 ng/mL of Anvirzel™. The presence of 0.1 μg/mL of cisplatin reduced the apparent absorbance to 0.161, but the presence of 0.01 ng/mL of Anvirzel™ and 0.1 μg/mL of cisplatin further reduced the value to 0.033 (p=0.005<0.05). A similar diminution in apparent absorbance was found after 72 hours incubation as well.

TABLE 1
Statistical evaluation of absorbance in LNCaP prostate cancer cell line.
LNCaP Cell Line ABSORBANCE

| | 48 h | 72 h |
|---|---|---|
| Crystal Violet Assay | | |
| Unstimulated cells | 1.307 | 2.08 |
| 0.01 ng/mL Anvirzel | 1.249 | 2.416 |
| 0.1 μg/mL cisplatin | 0.408 | 0.448 |
| 0.01 ng/mL Anvirzel + | 0.178 | 0.187 |
| 0.1 μg/mL cisplatin | (p = 0.00008 < | (p = 0.002 < |
| | 0.05) | 0.05) |
| Sulforhodamine B Assay | | |
| Unstimulated cells | 2.616 | 2.906 |
| 0.01 ng/mL Anvirzel | 2.667 | 2.927 |
| 0.1 μg/mL cisplatin | 1.681 | 1.766 |
| 0.01 ng/mL Anvirzel + | 1.033 | 1.095 |
| 0.1 μg/mL cisplatin | (p = 0.007 < | (p = 0.001 < |
| | 0.05) | 0.05) |
| Methyl-Tetrazolium Assay | | |
| Unstimulated cells | 0.429 | 0.553 |
| 0.01 ng/mL Anvirzel | 0.292 | 0.373 |
| 0.1 μg/mL cisplatin | 0.161 | 0.17 |
| 0.01 ng/mL Anvirzel + | 0.033 | 0.035 |
| 0.1 μg/mL cisplatin | (p = 0.005 < | (p = 0.0001 < |
| | 0.05) | 0.05) |

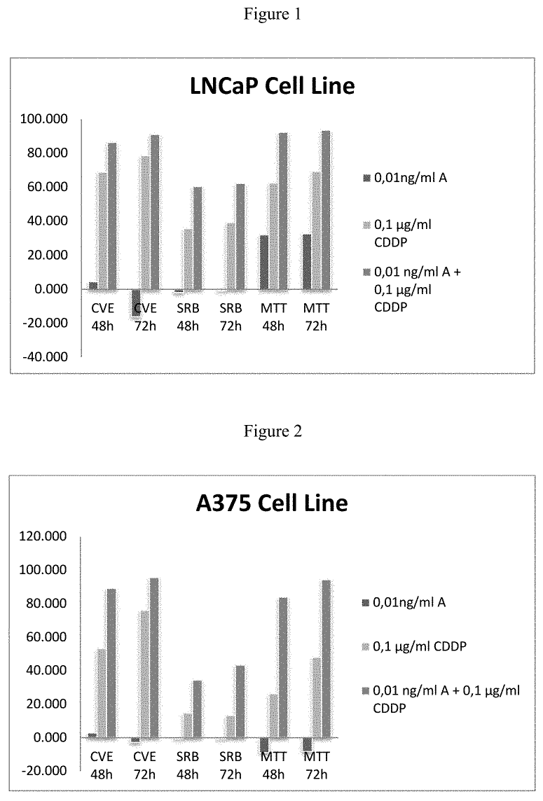

These results in Table 1 are graphically depicted in FIG. 1.

### Example 2: A375 Cell Line

Table 2 below shows results for A375 cells, a melanoma-derived cell line, with cell population densities estimated from optical absorbance data.

By Crystal Violet assay, after 48 hours of incubation, unstimulated cells showed an apparent absorbance of 2.791, with essentially the same value (2.71) obtained in the presence of 0.01 ng/mL of Anvirzel™. The presence of 0.1 μg/mL of cisplatin reduced the apparent absorbance to 1.304, but the presence of 0.01 ng/mL of Anvirzel™ and 0.1 μg/mL of cisplatin reduced the value to 0.178 (p=0.00008<0.05). A similar diminution in apparent absorbance was found after 72 hours incubation as well.

By Sulforhodamine B assay, after 48 hours of incubation, unstimulated cells showed an apparent absorbance of 2.836, with essentially the same value (2.854) obtained in the presence of 0.01 ng/mL of Anvirzel™. The presence of 0.1 μg/mL of cisplatin reduced the apparent absorbance to 2.421, but the presence of 0.01 ng/mL of Anvirzel™ of and 0.1 μg/mL of cisplatin further reduced the value to 1.89 (p=0.01<0.05). A similar diminution in apparent absorbance was found after 72 hours incubation as well.

By Methyl-Tetrazolium assay, after 48 hours of incubation, unstimulated cells showed an apparent absorbance of 0.45, with essentially the same value (0.49) obtained in the presence of 0.01 ng/mL of Anvirzel™. The presence of 0.1 μg/mL of cisplatin reduced the apparent absorbance to 0.332, but the presence of 0.01 ng/mL of Anvirzel™ and 0.1 μg/mL of cisplatin reduced the value to 0.072 (p=0.002<0.05). A similar diminution in apparent absorbance was found after 72 hours incubation as well.

TABLE 2
Statistical evaluation of absorbance in A375 melanoma cancer cell line.
A375 Cell Line ABSORBANCE

| | 48 h | 72 h |
|---|---|---|
| Crystal Violet Assay | | |
| Unstimulated cells | 2.791 | 2.767 |
| 0.01 ng/mL Anvirzel | 2.71 | 2.846 |
| 0.1 μg/mL cisplatin | 1.304 | 0.663 |
| 0.01 ng/mL Anvirzel + | 0.306 | 0.123 |
| 0.1 μg/mL cisplatin | (p = 0.002 < 0.05) | (p = 0.0009 < 0.05) |
| Sulforhodamine B Assay | | |
| Unstimulated cells | 2.836 | 2.867 |
| 0.01 ng/mL Anvirzel | 2.854 | 2.884 |
| 0.1 μg/mL cisplatin | 2.421 | 2.487 |
| 0.01 ng/mL Anvirzel + | 1.89 | 1.624 |
| 0.1 μg/mL cisplatin | (p = 0.01 < 0.05) | (p = 0.006 < 0.05) |
| Methyl-Tetrazolium Assay | | |
| Unstimulated cells | 0.45 | 0.922 |
| 0.01 ng/mL Anvirzel | 0.49 | 0.999 |
| 0.1 μg/mL cisplatin | 0.332 | 0.479 |
| 0.01 ng/mL Anvirzel + | 0.072 | 0.053 |
| 0.1 μg/mL cisplatin | (p = 0.002 < 0.05) | (p = 0.005 < 0.05) |

These results in Table 2 are graphically depicted in FIG. 2.

### Example 3: PANC-1 Cell Line

Table 3 below shows results for PANC-1 cells, a pancreatic cancer-derived cell line, with cell population densities estimated from optical absorbance data.

By Crystal Violet assay, after 48 hours of incubation, unstimulated cells showed an apparent absorbance of 0.964, with essentially the same value (1.04) obtained in the presence of 0.01 ng/mL of Anvirzel™. The presence of 0.1 μg/mL of cisplatin reduced the apparent absorbance to 0.753, but the presence of 0.01 ng/mL of Anvirzel™ and 0.1 μg/mL of cisplatin reduced the value to 0.283 (p=0.01<0.05). A similar diminution in apparent absorbance was found after 72 hours incubation as well.

By Sulforhodamine B assay, after 48 hours of incubation, unstimulated cells showed an apparent absorbance of 2.332, with essentially the same value (2.328) obtained in the presence of 0.01 ng/mL of Anvirzel™. The presence of 0.1 μg/mL of cisplatin reduced the apparent absorbance to 1.861, but the presence of 0.01 ng/mL of Anvirzel™ and 0.1 μg/mL of cisplatin further reduced the value to 1.25 (p=0.002<0.05). A similar diminution in apparent absorbance was found after 72 hours incubation as well.

By Methyl-Tetrazolium assay, after 48 hours of incubation, unstimulated cells showed an apparent absorbance of 0.546, with a similar value (0.518) obtained in the presence of 0.01 ng/mL of Anvirzel™. The presence of 0.1 μg/mL of cisplatin reduced the apparent absorbance to 0.286, but the presence of 0.01 ng/mL of Anvirzel™ and 0.1 μg/mL of cisplatin reduced the value to 0.143 (p=0.0007<0.05). A similar diminution in apparent absorbance was found after 72 hours incubation as well.

TABLE 3
Statistical evaluation of absorbance in PANC-1 pancreatic cancer cell line.
PANC-1 Cell Line ABSORBANCE

| | 48 h | 72 h |
|---|---|---|
| Crystal Violet Assay | | |
| Unstimulated cells | 0.964 | 2.862 |
| 0.01 ng/mL Anvirzel | 1.04 | 2.711 |
| 0.1 μg/mL cisplatin | 0.753 | 0.872 |
| 0.01 ng/mL Anvirzel + | 0.283 | 0.336 |
| 0.1 μg/mL cisplatin | (p = 0.01 < 0.05) | (p = 0.001 < 0.05) |
| Sulforhodamine B Assay | | |
| Unstimulated cells | 2.332 | 2.924 |
| 0.01 ng/mL Anvirzel | 2.328 | 2.882 |
| 0.1 μg/mL cisplatin | 1.861 | 2.536 |
| 0.01 ng/mL Anvirzel + | 1.25 | 1.837 |
| 0.1 μg/mL cisplatin | (p = 0.002 < 0.05) | (p = 0.004 < 0.05) |
| Methyl-Tetrazolium Assay | | |
| Unstimulated cells | 0.546 | 1.012 |
| 0.01 ng/mL Anvirzel | 0.518 | 0.954 |
| 0.1 μg/mL cisplatin | 0.286 | 0.344 |
| 0.01 ng/mL Anvirzel + | 0.143 | 0.115 |
| 0.1 μg/mL cisplatin | (p = 0.0007 < 0.05) | (p = 0.002 < 0.05) |

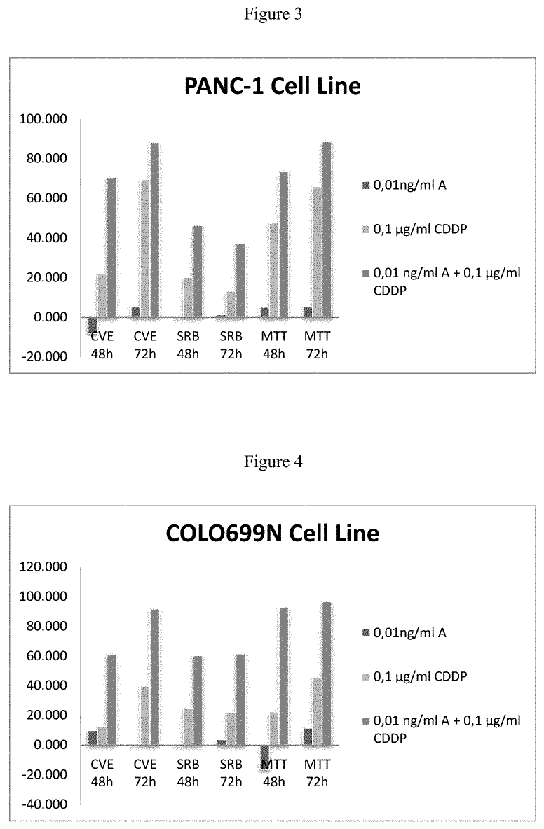

These results in Table 3 are graphically depicted in FIG. 3.

### Example 4: COLO699N Cell Line

Table 4 below shows results for COLO699N cells, a lung cancer-derived cell line, with cell population densities estimated from optical absorbance data.

By Crystal Violet assay, after 48 hours of incubation, unstimulated cells showed an apparent absorbance of 0.918, with a similar value (0.826) obtained in the presence of 0.01 ng/mL of Anvirzel™. The presence of 0.1 μg/mL of cisplatin reduced the apparent absorbance slightly to 0.801, but the presence of 0.01 ng/mL of Anvirzel™ and 0.1 μg/mL of cisplatin reduced the value to 0.361 (p=0.01<0.05). A similar diminution in apparent absorbance was found after 72 hours incubation as well.

By Sulforhodamine B assay, after 48 hours of incubation, unstimulated cells showed an apparent absorbance of 2.636, with essentially the same value (2.633) obtained in the presence of 0.01 ng/mL of Anvirzel™. The presence of 0.1 μg/mL of cisplatin reduced the apparent absorbance to 1.975, but the presence of 0.01 ng/mL of Anvirzel™ and 0.1 μg/mL of cisplatin further reduced the value to 1.046 (p=0.00001<0.05). A similar diminution in apparent absorbance was found after 72 hours incubation as well.

By Methyl-Tetrazolium assay, after 48 hours of incubation, unstimulated cells showed an apparent absorbance of 0.82, with a somewhat higher value (0.953) obtained in the presence of 0.01 ng/mL of Anvirzel™. The presence of 0.1 μg/mL of cisplatin reduced the apparent absorbance to 0.636, but the presence of 0.01 ng/mL of Anvirzel™ and 0.1 μg/mL of cisplatin reduced the value to 0.058 (p=0.001<0.05). A similar diminution in apparent absorbance was found after 72 hours incubation as well.

TABLE 4
Statistical evaluation of absorbance in COLO699N lung cancer cell line.
COLO699N Cell Line ABSORBANCE

| | 48 h | 72 h |
|---|---|---|
| Crystal Violet Assay | | |
| Unstimulated cells | 0.918 | 1.659 |
| 0.01 ng/mL Anvirzel | 0.826 | 1.654 |
| 0.1 μg/mL cisplatin | 0.801 | 1 |
| 0.01 ng/mL Anvirzel + | 0.361 | 0.138 |
| 0.1 μg/mL cisplatin | (p = 0.01 < | (p = 0.005 < |
| | 0.05) | 0.05) |
| Sulforhodamine B Assay | | |
| Unstimulated cells | 2.636 | 2.758 |
| 0.01 ng/mL Anvirzel | 2.633 | 2.655 |
| 0.1 μg/mL cisplatin | 1.975 | 2.145 |
| 0.01 ng/mL Anvirzel + | 1.046 | 1.062 |
| 0.1 μg/mL cisplatin | (p = 0.00001 < | (p = 0.00009 < |
| | 0.05) | 0.05) |
| Methyl-Tetrazolium Assay | | |
| Unstimulated cells | 0.82 | 1.34 |
| 0.01 ng/mL Anvirzel | 0.953 | 1.184 |
| 0.1 μg/mL cisplatin | 0.636 | 0.73 |
| 0.01 ng/mL Anvirzel + | 0.058 | 0.047 |
| 0.1 μg/mL cisplatin | (p = 0.001 < | (p = 0.001 < |
| | 0.05) | 0.05) |

These results in Table 4 are graphically depicted in FIG. 4.

TABLE 5

| | 22Rv1 | Run 1 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated cells | 0.302 | 0.658 | 1.307 | 1.669 | 2.125 | 2.735 | 0.113 | 0.248 | 0.45 |
| 0.01 ng/ml A | 0.345 | 0.709 | 1.226 | 1.706 | 2.276 | 2.709 | 0.072 | 0.246 | 0.452 |
| 0.1 μg/ml CDDP | 0.27 | 0.501 | 0.803 | 1.847 | 1.685 | 2.018 | 0.042 | 0.141 | 0.168 |
| 0.01 ng/ml A + 0.1 | 0.262 | 0.499 | 0.379 | 1.699 | 1.636 | 1.866 | 0.054 | 0.135 | 0.117 |
| μg/ml CDDP | | | | | | | | | |
| Unstimulated cells | 0.302 | 0.658 | 1.307 | 1.669 | 2.125 | 2.735 | 0.113 | 0.248 | 0.45 |
| 0.01 ng/ml A | 0.345 | 0.709 | 1.226 | 1.706 | 2.276 | 2.709 | 0.072 | 0.246 | 0.452 |
| 1 μg/ml CDDP | 0.152 | 0.437 | 0.606 | 1.943 | 1.549 | 1.56 | 0.013 | 0.013 | 0.015 |
| 0.01 ng/ml A + 1 | 0.126 | 0.384 | 0.202 | 1.823 | 1.548 | 1.656 | 0.011 | 0.009 | 0.01 |
| μg/ml CDDP | | | | | | | | | |
| Unstimulated cells | 0.302 | 0.658 | 1.307 | 1.669 | 2.125 | 2.735 | 0.113 | 0.248 | 0.45 |
| 0.01 ng/ml A | 0.345 | 0.709 | 1.226 | 1.706 | 2.276 | 2.709 | 0.072 | 0.246 | 0.452 |
| 10 μg/ml CDDP | 0.158 | 0.351 | 0.439 | 1.744 | 1.217 | 1.141 | 0.033 | 0.013 | 0.01 |
| 0.01 ng/ml A + 10 | 0.18 | 0.216 | 0.197 | 1.795 | 1.179 | 1.461 | 0.023 | 0.012 | 0.01 |
| μg/ml CDDP | | | | | | | | | |
| Unstimulated cells | 0.302 | 0.658 | 1.307 | 1.669 | 2.125 | 2.735 | 0.113 | 0.248 | 0.45 |
| 0.01 ng/ml A | 0.345 | 0.709 | 1.226 | 1.706 | 2.276 | 2.709 | 0.072 | 0.246 | 0.452 |
| 100 μg/ml CDDP | 0.124 | 0.273 | 0.233 | 1.878 | 1.121 | 1.204 | 0.011 | 0.008 | 0.009 |
| 0.01 ng/ml A + | 0.311 | 0.295 | 0.284 | 1.93 | 1.292 | 1.519 | 0.008 | 0.007 | 0.008 |
| 100 μg/ml CDDP | | | | | | | | | |
| Average | | | | | | | | | |
| Unstimulated cells | 0.302 | 0.658 | 1.307 | 1.669 | 2.125 | 2.735 | 0.113 | 0.248 | 0.45 |
| 0.1 ng/ml A | 0.318 | 0.855 | 1.336 | 1.789 | 2.198 | 2.654 | 0.114 | 0.266 | 0.484 |
| 0.1 μg/ml CDDP | 0.27 | 0.501 | 0.803 | 1.847 | 1.685 | 2.018 | 0.042 | 0.141 | 0.168 |
| 0.1 ng/ml A + 0.1 | 0.255 | 0.624 | 0.51 | 1.901 | 1.694 | 1.723 | 0.066 | 0.125 | 0.085 |
| μg/ml CDDP | | | | | | | | | |
| Unstimulated cells | 0.302 | 0.658 | 1.307 | 1.669 | 2.125 | 2.735 | 0.113 | 0.248 | 0.45 |
| 0.1 ng/ml A | 0.318 | 0.855 | 1.336 | 1.789 | 2.198 | 2.654 | 0.114 | 0.266 | 0.484 |
| 1 μg/ml CDDP | 0.152 | 0.437 | 0.606 | 1.943 | 1.549 | 1.56 | 0.013 | 0.013 | 0.015 |
| 0.1 ng/ml A + 1 | 0.115 | 0.318 | 0.297 | 1.968 | 1.6 | 1.418 | 0.012 | 0.01 | 0.01 |
| μg/ml CDDP | | | | | | | | | |
| Unstimulated cells | 0.302 | 0.658 | 1.307 | 1.669 | 2.125 | 2.735 | 0.113 | 0.248 | 0.45 |
| 0.1 ng/ml A | 0.318 | 0.855 | 1.336 | 1.789 | 2.198 | 2.654 | 0.114 | 0.266 | 0.484 |
| 10 μg/ml CDDP | 0.158 | 0.351 | 0.439 | 1.744 | 1.217 | 1.141 | 0.033 | 0.013 | 0.01 |
| 0.1 ng/ml A + 10 | 0.208 | 0.213 | 0.333 | 1.809 | 1.421 | 1.073 | 0.026 | 0.011 | 0.01 |
| μg/ml CDDP | | | | | | | | | |
| Unstimulated cells | 0.302 | 0.658 | 1.307 | 1.669 | 2.125 | 2.735 | 0.113 | 0.248 | 0.45 |
| 0.1 ng/ml A | 0.318 | 0.855 | 1.336 | 1.789 | 2.198 | 2.654 | 0.114 | 0.266 | 0.484 |
| 100 μg/ml CDDP | 0.124 | 0.273 | 0.233 | 1.878 | 1.121 | 1.204 | 0.011 | 0.008 | 0.009 |
| 0.1 ng/ml A + 100 | 0.284 | 0.519 | 0.368 | 2.396 | 1.254 | 1.506 | 0.009 | 0.007 | 0.007 |
| μg/ml CDDP | | | | | | | | | |
| Average | | | | | | | | | |
| Unstimulated cells | 0.302 | 0.658 | 1.307 | 1.669 | 2.125 | 2.735 | 0.113 | 0.248 | 0.45 |
| 1 ng/ml A | 0.393 | 0.934 | 1.486 | 1.823 | 2.098 | 2.684 | 0.144 | 0.187 | 0.344 |
| 0.1 μg/ml CDDP | 0.27 | 0.501 | 0.803 | 1.847 | 1.685 | 2.018 | 0.042 | 0.141 | 0.168 |
| 1 ng/ml A + 0.1 | 0.305 | 0.65 | 0.51 | 1.936 | 1.542 | 1.76 | 0.055 | 0.083 | 0.08 |
| μg/ml CDDP | | | | | | | | | |
| Unstimulated cells | 0.302 | 0.658 | 1.307 | 1.669 | 2.125 | 2.735 | 0.113 | 0.248 | 0.45 |
| 1 ng/ml A | 0.393 | 0.934 | 1.486 | 1.823 | 2.098 | 2.684 | 0.144 | 0.187 | 0.344 |
| 1 μg/ml CDDP | 0.152 | 0.437 | 0.606 | 1.943 | 1.549 | 1.56 | 0.013 | 0.013 | 0.015 |
| 1 ng/ml A + 1 | 0.161 | 0.314 | 0.269 | 2.076 | 2.165 | 1.56 | 0.009 | 0.009 | 0.007 |
| μg/ml CDDP | | | | | | | | | |
| Unstimulated cells | 0.302 | 0.658 | 1.307 | 1.669 | 2.125 | 2.735 | 0.113 | 0.248 | 0.45 |
| 1 ng/ml A | 0.393 | 0.934 | 1.486 | 1.823 | 2.098 | 2.684 | 0.144 | 0.187 | 0.344 |
| 10 μg/ml CDDP | 0.158 | 0.351 | 0.439 | 1.744 | 1.217 | 1.141 | 0.033 | 0.013 | 0.01 |
| 1 ng/ml A + 10 | 0.182 | 0.4 | 0.438 | 2.318 | 1.503 | 1.252 | 0.023 | 0.013 | 0.01 |
| μg/ml CDDP | | | | | | | | | |
| Unstimulated cells | 0.302 | 0.658 | 1.307 | 1.669 | 2.125 | 2.735 | 0.113 | 0.248 | 0.45 |
| 1 ng/ml A | 0.393 | 0.934 | 1.486 | 1.823 | 2.098 | 2.684 | 0.144 | 0.187 | 0.344 |
| 100 μg/ml CDDP | 0.124 | 0.273 | 0.233 | 1.878 | 1.121 | 1.204 | 0.011 | 0.008 | 0.009 |
| 1 ng/ml A + 100 | 0.243 | 0.602 | 0.741 | 2.476 | 1.636 | 1.601 | 0.011 | 0.01 | 0.009 |
| μg/ml CDDP | | | | | | | | | |
| Average | | | | | | | | | |
| Unstimulated cells | 0.302 | 0.658 | 1.307 | 1.669 | 2.125 | 2.735 | 0.113 | 0.248 | 0.45 |
| 10 ng/ml A | 0.457 | 0.811 | 1.116 | 1.946 | 1.919 | 2.5 | 0.187 | 0.204 | 0.377 |
| 0.1 μg/ml CDDP | 0.27 | 0.501 | 0.803 | 1.847 | 1.685 | 2.018 | 0.042 | 0.141 | 0.168 |
| 10 ng/ml A + 0.1 | 0.324 | 0.511 | 0.531 | 1.801 | 1.376 | 1.521 | 0.084 | 0.094 | 0.105 |
| μg/ml CDDP | | | | | | | | | |
| Unstimulated cells | 0.302 | 0.658 | 1.307 | 1.669 | 2.125 | 2.735 | 0.113 | 0.248 | 0.45 |
| 10 ng/ml A | 0.457 | 0.811 | 1.116 | 1.946 | 1.919 | 2.5 | 0.187 | 0.204 | 0.377 |
| 1 μg/ml CDDP | 0.152 | 0.437 | 0.606 | 1.943 | 1.549 | 1.56 | 0.013 | 0.013 | 0.015 |
| 10 ng/ml A + 1 | 0.187 | 0.468 | 0.552 | 2.225 | 1.458 | 1.548 | 0.009 | 0.01 | 0.008 |
| μg/ml CDDP | | | | | | | | | |
| Unstimulated cells | 0.302 | 0.658 | 1.307 | 1.669 | 2.125 | 2.735 | 0.113 | 0.248 | 0.45 |
| 10 ng/ml A | 0.457 | 0.811 | 1.116 | 1.946 | 1.919 | 2.5 | 0.187 | 0.204 | 0.377 |
| 10 μg/ml CDDP | 0.158 | 0.351 | 0.439 | 1.744 | 1.217 | 1.141 | 0.033 | 0.013 | 0.01 |
| 10 ng/ml A + 10 | 0.258 | 0.487 | 0.312 | 2.108 | 1.261 | 1.672 | 0.026 | 0.013 | 0.01 |
| μg/ml CDDP | | | | | | | | | |
| Unstimulated cells | 0.302 | 0.658 | 1.307 | 1.669 | 2.125 | 2.735 | 0.113 | 0.248 | 0.45 |
| 10 ng/ml A | 0.457 | 0.811 | 1.116 | 1.946 | 1.919 | 2.5 | 0.187 | 0.204 | 0.377 |
| 100 μg/ml CDDP | 0.124 | 0.273 | 0.233 | 1.878 | 1.121 | 1.204 | 0.011 | 0.008 | 0.009 |
| 10 ng/ml A + 100 | 0.488 | 0.857 | 0.535 | 2.485 | 1.785 | 1.775 | 0.008 | 0.008 | 0.007 |
| μg/ml CDDP | | | | | | | | | |

TABLE 6

| | 22Rv1 | Run 2 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated cells | 0.485 | 1.147 | 1.613 | 1.647 | 2.485 | 2.799 | 0.084 | 0.377 | 0.575 |
| 0.01 ng/ml A | 0.468 | 1.433 | 1.684 | 1.633 | 2.459 | 2.678 | 0.077 | 0.252 | 0.522 |
| 0.1 μg/ml CDDP | 0.541 | 0.776 | 0.784 | 1.727 | 2.139 | 2.392 | 0.077 | 0.172 | 0.29 |
| 0.01 ng/ml A + 0.1 | 0.309 | 0.344 | 0.421 | 1.41 | 1.549 | 1.556 | 0.041 | 0.057 | 0.053 |
| μg/ml CDDP | | | | | | | | | |
| Unstimulated cells | 0.485 | 1.147 | 1.613 | 1.647 | 2.485 | 2.799 | 0.084 | 0.377 | 0.575 |
| 0.01 ng/ml A | 0.468 | 1.433 | 1.684 | 1.633 | 2.459 | 2.678 | 0.077 | 0.252 | 0.522 |
| 1 μg/ml CDDP | 0.305 | 0.517 | 0.286 | 1.647 | 1.824 | 1.395 | 0.011 | 0.014 | 0.016 |
| 0.01 ng/ml A + 1 | 0.294 | 0.235 | 0.216 | 1.696 | 1.631 | 1.553 | 0.008 | 0.008 | 0.006 |
| μg/ml CDDP | | | | | | | | | |
| Unstimulated cells | 0.485 | 1.147 | 1.613 | 1.647 | 2.485 | 2.799 | 0.084 | 0.377 | 0.575 |
| 0.01 ng/ml A | 0.468 | 1.433 | 1.684 | 1.633 | 2.459 | 2.678 | 0.077 | 0.252 | 0.522 |
| 10 μg/ml CDDP | 0.376 | 0.434 | 0.206 | 1.49 | 1.46 | 1.606 | 0.019 | 0.016 | 0.013 |
| 0.01 ng/ml A + 10 | 0.246 | 0.415 | 0.252 | 1.596 | 1.35 | 1.14 | 0.029 | 0.02 | 0.01 |
| μg/ml CDDP | | | | | | | | | |
| Unstimulated cells | 0.485 | 1.147 | 1.613 | 1.647 | 2.485 | 2.799 | 0.084 | 0.377 | 0.575 |
| 0.01 ng/ml A | 0.468 | 1.433 | 1.684 | 1.633 | 2.459 | 2.678 | 0.077 | 0.252 | 0.522 |
| 100 μg/ml CDDP | 0.228 | 0.228 | 0.256 | 1.495 | 1.621 | 1.247 | 0.009 | 0.008 | 0.008 |
| 0.01 ng/ml A + | 0.27 | 0.487 | 0.289 | 1.76 | 1.661 | 1.56 | 0.008 | 0.008 | 0.005 |
| 100 μg/ml CDDP | | | | | | | | | |
| Average | | | | | | | | | |
| Unstimulated cells | 0.485 | 1.147 | 1.613 | 1.647 | 2.485 | 2.799 | 0.084 | 0.377 | 0.575 |
| 0.1 ng/ml A | 0.479 | 1.211 | 1.504 | 1.642 | 2.492 | 2.784 | 0.07 | 0.226 | 0.494 |
| 0.1 μg/ml CDDP | 0.541 | 0.776 | 0.784 | 1.727 | 2.139 | 2.392 | 0.077 | 0.172 | 0.29 |
| 0.1 ng/ml A + 0.1 | 0.52 | 0.561 | 0.55 | 1.688 | 1.783 | 1.826 | 0.055 | 0.113 | 0.115 |
| μg/ml CDDP | | | | | | | | | |
| Unstimulated cells | 0.485 | 1.147 | 1.613 | 1.647 | 2.485 | 2.799 | 0.084 | 0.377 | 0.575 |
| 0.1 ng/ml A | 0.479 | 1.211 | 1.504 | 1.642 | 2.492 | 2.784 | 0.07 | 0.226 | 0.494 |
| 1 μg/ml CDDP | 0.305 | 0.517 | 0.286 | 1.647 | 1.824 | 1.395 | 0.011 | 0.014 | 0.016 |
| 0.1 ng/ml A + 1 | 0.194 | 0.441 | 0.51 | 1.818 | 1.73 | 1.455 | 0.008 | 0.008 | 0.007 |
| μg/ml CDDP | | | | | | | | | |
| Unstimulated cells | 0.485 | 1.147 | 1.613 | 1.647 | 2.485 | 2.799 | 0.084 | 0.377 | 0.575 |
| 0.1 ng/ml A | 0.479 | 1.211 | 1.504 | 1.642 | 2.492 | 2.784 | 0.07 | 0.226 | 0.494 |
| 10 μg/ml CDDP | 0.376 | 0.434 | 0.206 | 1.49 | 1.46 | 1.606 | 0.019 | 0.016 | 0.013 |
| 0.1 ng/ml A + 10 | 0.378 | 0.596 | 0.524 | 1.443 | 1.518 | 1.281 | 0.022 | 0.015 | 0.007 |
| μg/ml CDDP | | | | | | | | | |
| Unstimulated cells | 0.485 | 1.147 | 1.613 | 1.647 | 2.485 | 2.799 | 0.084 | 0.377 | 0.575 |
| 0.1 ng/ml A | 0.479 | 1.211 | 1.504 | 1.642 | 2.492 | 2.784 | 0.07 | 0.226 | 0.494 |
| 100 μg/ml CDDP | 0.228 | 0.228 | 0.256 | 1.495 | 1.621 | 1.247 | 0.009 | 0.008 | 0.008 |
| 0.1 ng/ml A + 100 | 0.517 | 0.561 | 0.317 | 1.665 | 1.988 | 1.401 | 0.008 | 0.008 | 0.006 |
| μg/ml CDDP | | | | | | | | | |
| Average | | | | | | | | | |
| Unstimulated cells | 0.485 | 1.147 | 1.613 | 1.647 | 2.485 | 2.799 | 0.084 | 0.377 | 0.575 |
| 1 ng/ml A | 0.561 | 1.25 | 2.043 | 1.578 | 2.665 | 2.857 | 0.088 | 0.236 | 0.381 |
| 0.1 μg/ml CDDP | 0.541 | 0.776 | 0.784 | 1.727 | 2.139 | 2.392 | 0.077 | 0.172 | 0.29 |
| 1 ng/ml A + 0.1 | 0.519 | 0.711 | 0.642 | 1.843 | 1.897 | 1.967 | 0.07 | 0.094 | 0.193 |
| μg/ml CDDP | | | | | | | | | |
| Unstimulated cells | 0.485 | 1.147 | 1.613 | 1.647 | 2.485 | 2.799 | 0.084 | 0.377 | 0.575 |
| 1 ng/ml A | 0.561 | 1.25 | 2.043 | 1.578 | 2.665 | 2.857 | 0.088 | 0.236 | 0.381 |
| 1 μg/ml CDDP | 0.305 | 0.517 | 0.286 | 1.647 | 1.824 | 1.395 | 0.011 | 0.014 | 0.016 |
| 1 ng/ml A + 1 | 0.3 | 0.402 | 0.324 | 1.985 | 1.634 | 1.532 | 0.007 | 0.01 | 0.007 |
| μg/ml CDDP | | | | | | | | | |
| Unstimulated cells | 0.485 | 1.147 | 1.613 | 1.647 | 2.485 | 2.799 | 0.084 | 0.377 | 0.575 |
| 1 ng/ml A | 0.561 | 1.25 | 2.043 | 1.578 | 2.665 | 2.857 | 0.088 | 0.236 | 0.381 |
| 10 μg/ml CDDP | 0.376 | 0.434 | 0.206 | 1.49 | 1.46 | 1.606 | 0.019 | 0.016 | 0.013 |
| 1 ng/ml A + 10 | 0.438 | 0.511 | 0.34 | 1.872 | 1.672 | 1.535 | 0.024 | 0.018 | 0.01 |
| μg/ml CDDP | | | | | | | | | |
| Unstimulated cells | 0.485 | 1.147 | 1.613 | 1.647 | 2.485 | 2.799 | 0.084 | 0.377 | 0.575 |
| 1 ng/ml A | 0.561 | 1.25 | 2.043 | 1.578 | 2.665 | 2.857 | 0.088 | 0.236 | 0.381 |
| 100 μg/ml CDDP | 0.228 | 0.228 | 0.256 | 1.495 | 1.621 | 1.247 | 0.009 | 0.008 | 0.008 |
| 1 ng/ml A + 100 | 0.55 | 0.517 | 0.358 | 1.89 | 1.929 | 1.809 | 0.009 | 0.009 | 0.006 |
| μg/ml CDDP | | | | | | | | | |
| Average | | | | | | | | | |
| Unstimulated cells | 0.485 | 1.147 | 1.613 | 1.647 | 2.485 | 2.799 | 0.084 | 0.377 | 0.575 |
| 10 ng/ml A | 0.683 | 1.222 | 2.193 | 1.496 | 2.492 | 2.817 | 0.106 | 0.206 | 0.427 |
| 0.1 μg/ml CDDP | 0.541 | 0.776 | 0.784 | 1.727 | 2.139 | 2.392 | 0.077 | 0.172 | 0.29 |
| 10 ng/ml A + 0.1 | 0.519 | 0.673 | 0.637 | 1.666 | 1.78 | 1.83 | 0.083 | 0.154 | 0.165 |
| μg/ml CDDP | | | | | | | | | |
| Unstimulated cells | 0.485 | 1.147 | 1.613 | 1.647 | 2.485 | 2.799 | 0.084 | 0.377 | 0.575 |
| 10 ng/ml A | 0.683 | 1.222 | 2.193 | 1.496 | 2.492 | 2.817 | 0.106 | 0.206 | 0.427 |
| 1 μg/ml CDDP | 0.305 | 0.517 | 0.286 | 1.647 | 1.824 | 1.395 | 0.011 | 0.014 | 0.016 |
| 10 ng/ml A + 1 | 0.518 | 0.263 | 0.271 | 1.729 | 1.452 | 1.383 | 0.009 | 0.008 | 0.008 |
| μg/ml CDDP | | | | | | | | | |
| Unstimulated cells | 0.485 | 1.147 | 1.613 | 1.647 | 2.485 | 2.799 | 0.084 | 0.377 | 0.575 |
| 10 ng/ml A | 0.683 | 1.222 | 2.193 | 1.496 | 2.492 | 2.817 | 0.106 | 0.206 | 0.427 |
| 10 μg/ml CDDP | 0.376 | 0.434 | 0.206 | 1.49 | 1.46 | 1.606 | 0.019 | 0.016 | 0.013 |
| 10 ng/ml A + 10 | 0.65 | 0.414 | 0.333 | 1.774 | 1.486 | 1.601 | 0.03 | 0.016 | 0.01 |
| μg/ml CDDP | | | | | | | | | |
| Unstimulated cells | 0.485 | 1.147 | 1.613 | 1.647 | 2.485 | 2.799 | 0.084 | 0.377 | 0.575 |
| 10 ng/ml A | 0.683 | 1.222 | 2.193 | 1.496 | 2.492 | 2.817 | 0.106 | 0.206 | 0.427 |
| 100 μg/ml CDDP | 0.228 | 0.228 | 0.256 | 1.495 | 1.621 | 1.247 | 0.009 | 0.008 | 0.008 |
| 10 ng/ml A + 100 | 1.568 | 0.623 | 0.637 | 2.112 | 1.769 | 1.781 | 0.013 | 0.033 | 0.005 |
| μg/ml CDDP | | | | | | | | | |

TABLE 7

| | 22Rv1 | Run 3 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated cells | 0.28 | 0.583 | 1.391 | 0.28 | 0.583 | 1.391 | 0.105 | 0.239 | 0.362 |
| 0.01 ng/ml A | 0.496 | 0.637 | 1.231 | 0.504 | 1.02 | 1.619 | 0.093 | 0.233 | 0.288 |
| 0.1 μg/ml CDDP | 0.85 | 0.644 | 0.45 | 0.85 | 0.644 | 0.45 | 0.08 | 0.135 | 0.128 |
| 0.01 ng/ml A + 0.1 | 0.226 | 0.391 | 0.392 | 0.381 | 0.494 | 0.514 | 0.073 | 0.11 | 0.082 |
| μg/ml CDDP | | | | | | | | | |
| Unstimulated cells | 0.28 | 0.583 | 1.391 | 0.28 | 0.583 | 1.391 | 0.105 | 0.239 | 0.362 |
| 0.01 ng/ml A | 0.496 | 0.637 | 1.231 | 0.504 | 1.02 | 1.619 | 0.093 | 0.233 | 0.288 |
| 1 μg/ml CDDP | 0.434 | 0.404 | 0.284 | 0.434 | 0.404 | 0.284 | 0.015 | 0.016 | 0.015 |
| 0.01 ng/ml A + 1 | 0.217 | 0.182 | 0.18 | 0.349 | 0.38 | 0.269 | 0.01 | 0.011 | 0.011 |
| μg/ml CDDP | | | | | | | | | |
| Unstimulated cells | 0.28 | 0.583 | 1.391 | 0.28 | 0.583 | 1.391 | 0.105 | 0.239 | 0.362 |
| 0.01 ng/ml A | 0.496 | 0.637 | 1.231 | 0.504 | 1.02 | 1.619 | 0.093 | 0.233 | 0.288 |
| 10 μg/ml CDDP | 0.295 | 0.289 | 0.283 | 0.295 | 0.289 | 0.283 | 0.023 | 0.017 | 0.013 |
| 0.01 ng/ml A + 10 | 0.495 | 0.137 | 0.227 | 0.515 | 0.635 | 0.66 | 0.024 | 0.013 | 0.013 |
| μg/ml CDDP | | | | | | | | | |
| Unstimulated cells | 0.28 | 0.583 | 1.391 | 0.28 | 0.583 | 1.391 | 0.105 | 0.239 | 0.362 |
| 0.01 ng/ml A | 0.496 | 0.637 | 1.231 | 0.504 | 1.02 | 1.619 | 0.093 | 0.233 | 0.288 |
| 100 μg/ml CDDP | 0.215 | 0.212 | 0.234 | 0.215 | 0.212 | 0.234 | 0.011 | 0.011 | 0.011 |
| 0.01 ng/ml A + | 0.313 | 0.274 | 0.505 | 0.624 | 0.54 | 0.494 | 0.01 | 0.01 | 0.01 |
| 100 ng/ml CDDP | | | | | | | | | |
| Average | | | | | | | | | |
| Unstimulated cells | 0.28 | 0.583 | 1.391 | 1.281 | 2.096 | 2.722 | 0.105 | 0.239 | 0.362 |
| 0.1 ng/ml A | 0.726 | 0.675 | 1.414 | 1.38 | 2.262 | 2.737 | 0.102 | 0.206 | 0.484 |
| 0.1 μg/ml CDDP | 0.85 | 0.644 | 0.45 | 1.352 | 2.114 | 1.867 | 0.08 | 0.135 | 0.128 |
| 0.1 ng/ml A + 0.1 | 0.341 | 0.6 | 0.323 | 1.395 | 1.951 | 1.577 | 0.091 | 0.073 | 0.068 |
| μg/ml CDDP | | | | | | | | | |
| Unstimulated cells | 0.28 | 0.583 | 1.391 | 1.281 | 2.096 | 2.722 | 0.105 | 0.239 | 0.362 |
| 0.1 ng/ml A | 0.726 | 0.675 | 1.414 | 1.38 | 2.262 | 2.737 | 0.102 | 0.206 | 0.484 |
| 1 μg/ml CDDP | 0.434 | 0.404 | 0.284 | 1.383 | 1.868 | 1.718 | 0.015 | 0.016 | 0.015 |
| 0.1 ng/ml A + 1 | 0.333 | 0.206 | 0.236 | 1.295 | 1.497 | 1.475 | 0.012 | 0.013 | 0.012 |
| μg/ml CDDP | | | | | | | | | |
| Unstimulated cells | 0.28 | 0.583 | 1.391 | 1.281 | 2.096 | 2.722 | 0.105 | 0.239 | 0.362 |
| 0.1 ng/ml A | 0.726 | 0.675 | 1.414 | 1.38 | 2.262 | 2.737 | 0.102 | 0.206 | 0.484 |
| 10 μg/ml CDDP | 0.295 | 0.289 | 0.283 | 1.135 | 1.734 | 1.444 | 0.023 | 0.017 | 0.013 |
| 0.1 ng/ml A + 10 | 0.555 | 0.183 | 0.35 | 1.023 | 1.483 | 1.447 | 0.023 | 0.013 | 0.011 |
| μg/ml CDDP | | | | | | | | | |
| Unstimulated cells | 0.28 | 0.583 | 1.391 | 1.281 | 2.096 | 2.722 | 0.105 | 0.239 | 0.362 |
| 0.1 ng/ml A | 0.726 | 0.675 | 1.414 | 1.38 | 2.262 | 2.737 | 0.102 | 0.206 | 0.484 |
| 100 μg/ml CDDP | 0.215 | 0.212 | 0.234 | 1.456 | 1.538 | 1.204 | 0.011 | 0.011 | 0.011 |
| 0.1 ng/ml A + 100 | 0.613 | 0.562 | 0.85 | 1.617 | 1.916 | 1.632 | 0.011 | 0.01 | 0.01 |
| μg/ml CDDP | | | | | | | | | |
| Average | | | | | | | | | |
| Unstimulated cells | 0.28 | 0.583 | 1.391 | 1.281 | 2.096 | 2.722 | 0.105 | 0.239 | 0.362 |
| 1 ng/ml A | 0.807 | 0.84 | 1.743 | 1.458 | 2.527 | 2.803 | 0.08 | 0.161 | 0.815 |
| 0.1 μg/ml CDDP | 0.85 | 0.644 | 0.45 | 1.352 | 2.114 | 1.867 | 0.08 | 0.135 | 0.128 |
| 1 ng/ml A + 0.1 | 0.368 | 0.597 | 0.404 | 1.447 | 1.945 | 1.616 | 0.038 | 0.073 | 0.088 |
| μg/ml CDDP | | | | | | | | | |
| Unstimulated cells | 0.28 | 0.583 | 1.391 | 1.281 | 2.096 | 2.722 | 0.105 | 0.239 | 0.362 |
| 1 ng/ml A | 0.807 | 0.84 | 1.743 | 1.458 | 2.527 | 2.803 | 0.08 | 0.161 | 0.815 |
| 1 μg/ml CDDP | 0.434 | 0.404 | 0.284 | 1.383 | 1.868 | 1.718 | 0.015 | 0.016 | 0.015 |
| 1 ng/ml A + 1 | 0.437 | 0.227 | 0.243 | 1.444 | 1.797 | 1.633 | 0.012 | 0.011 | 0.01 |
| μg/ml CDDP | | | | | | | | | |
| Unstimulated cells | 0.28 | 0.583 | 1.391 | 1.281 | 2.096 | 2.722 | 0.105 | 0.239 | 0.362 |
| 1 ng/ml A | 0.807 | 0.84 | 1.743 | 1.458 | 2.527 | 2.803 | 0.08 | 0.161 | 0.815 |
| 10 μg/ml CDDP | 0.295 | 0.289 | 0.283 | 1.135 | 1.734 | 1.444 | 0.023 | 0.017 | 0.013 |
| 1 ng/ml A + 10 | 0.743 | 0.283 | 0.511 | 1.466 | 1.972 | 1.958 | 0.027 | 0.017 | 0.01 |
| μg/ml CDDP | | | | | | | | | |
| Unstimulated cells | 0.28 | 0.583 | 1.391 | 1.281 | 2.096 | 2.722 | 0.105 | 0.239 | 0.362 |
| 1 ng/ml A | 0.807 | 0.84 | 1.743 | 1.458 | 2.527 | 2.803 | 0.08 | 0.161 | 0.815 |
| 100 μg/ml CDDP | 0.215 | 0.212 | 0.234 | 1.456 | 1.538 | 1.204 | 0.011 | 0.011 | 0.011 |
| 1 ng/ml A + 100 | 0.508 | 0.652 | 0.556 | 1.866 | 2.396 | 1.89 | 0.011 | 0.011 | 0.01 |
| μg/ml CDDP | | | | | | | | | |
| Average | | | | | | | | | |
| Unstimulated cells | 0.28 | 0.583 | 1.391 | 1.281 | 2.096 | 2.722 | 0.105 | 0.239 | 0.362 |
| 10 ng/ml A | 0.504 | 1.02 | 1.619 | 1.373 | 2.518 | 2.673 | 0.207 | 0.213 | 0.473 |
| 0.1 μg/ml CDDP | 0.85 | 0.644 | 0.45 | 1.352 | 2.114 | 1.867 | 0.08 | 0.135 | 0.128 |
| 10 ng/ml A + 0.1 | 0.381 | 0.494 | 0.514 | 1.295 | 1.712 | 1.803 | 0.082 | 0.088 | 0.095 |
| μg/ml CDDP | | | | | | | | | |
| Unstimulated cells | 0.28 | 0.583 | 1.391 | 1.281 | 2.096 | 2.722 | 0.105 | 0.239 | 0.362 |
| 10 ng/ml A | 0.504 | 1.02 | 1.619 | 1.373 | 2.518 | 2.673 | 0.207 | 0.213 | 0.473 |
| 1 μg/ml CDDP | 0.434 | 0.404 | 0.284 | 1.383 | 1.868 | 1.718 | 0.015 | 0.016 | 0.015 |
| 10 ng/ml A + 1 | 0.349 | 0.38 | 0.269 | 1.459 | 1.544 | 1.909 | 0.011 | 0.012 | 0.01 |
| μg/ml CDDP | | | | | | | | | |
| Unstimulated cells | 0.28 | 0.583 | 1.391 | 1.281 | 2.096 | 2.722 | 0.105 | 0.239 | 0.362 |
| 10 ng/ml A | 0.504 | 1.02 | 1.619 | 1.373 | 2.518 | 2.673 | 0.207 | 0.213 | 0.473 |
| 10 μg/ml CDDP | 0.295 | 0.289 | 0.283 | 1.135 | 1.734 | 1.444 | 0.023 | 0.017 | 0.013 |
| 10 ng/ml A + 10 | 0.515 | 0.635 | 0.66 | 1.462 | 2.532 | 1.821 | 0.022 | 0.015 | 0.012 |
| μg/ml CDDP | | | | | | | | | |
| Unstimulated cells | 0.28 | 0.583 | 1.391 | 1.281 | 2.096 | 2.722 | 0.105 | 0.239 | 0.362 |
| 10 ng/ml A | 0.504 | 1.02 | 1.619 | 1.373 | 2.518 | 2.673 | 0.207 | 0.213 | 0.473 |
| 100 μg/ml CDDP | 0.215 | 0.212 | 0.234 | 1.456 | 1.538 | 1.204 | 0.011 | 0.011 | 0.011 |
| 10 ng/ml A + 100 | 0.624 | 0.54 | 0.494 | 1.949 | 2.346 | 1.905 | 0.01 | 0.01 | 0.01 |
| μg/ml CDDP | | | | | | | | | |

TABLE 8

| | A375 | Run 1 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated cells | 0.765 | 2.791 | 2.767 | 2.086 | 2.836 | 2.867 | 0.412 | 0.45 | 0.922 |
| 0.01 ng/ml A | 0.708 | 2.71 | 2.846 | 2.074 | 2.854 | 2.884 | 0.4 | 0.49 | 0.999 |
| 0.1 μg/ml CDDP | 0.607 | 1.304 | 0.663 | 2.072 | 2.421 | 2.487 | 0.34 | 0.332 | 0.479 |
| 0.01 ng/ml A + 0.1 | 0.469 | 0.306 | 0.123 | 1.653 | 1.86 | 1.624 | 0.168 | 0.072 | 0.053 |
| μg/ml CDDP | | | | | | | | | |
| Unstimulated cells | 0.765 | 2.791 | 2.767 | 2.086 | 2.836 | 2.867 | 0.412 | 0.45 | 0.922 |
| 0.01 ng/ml A | 0.708 | 2.71 | 2.846 | 2.074 | 2.854 | 2.884 | 0.4 | 0.49 | 0.999 |
| 1 μg/ml CDDP | 0.277 | 0.396 | 0.178 | 1.579 | 1.987 | 2.256 | 0.068 | 0.048 | 0.042 |
| 0.01 ng/ml A + 1 | 0.289 | 0.192 | 0.185 | 1.604 | 1.852 | 1.389 | 0.032 | 0.031 | 0.022 |
| μg/ml CDDP | | | | | | | | | |
| Unstimulated cells | 0.765 | 2.791 | 2.767 | 2.086 | 2.836 | 2.867 | 0.412 | 0.45 | 0.922 |
| 0.01 ng/ml A | 0.708 | 2.71 | 2.846 | 2.074 | 2.854 | 2.884 | 0.4 | 0.49 | 0.999 |
| 10 μg/ml CDDP | 0.32 | 0.405 | 0.282 | 1.117 | 1.678 | 1.913 | 0.066 | 0.044 | 0.04 |
| 0.01 ng/ml A + 10 | 0.276 | 0.304 | 0.647 | 1.388 | 1.657 | 1.227 | 0.046 | 0.034 | 0.027 |
| μg/ml CDDP | | | | | | | | | |
| Unstimulated cells | 0.765 | 2.791 | 2.767 | 2.086 | 2.836 | 2.867 | 0.412 | 0.45 | 0.922 |
| 0.01 ng/ml A | 0.708 | 2.71 | 2.846 | 2.074 | 2.854 | 2.884 | 0.4 | 0.49 | 0.999 |
| 100 μg/ml CDDP | 0.329 | 0.288 | 0.311 | 1.283 | 1.782 | 1.319 | 0.049 | 0.038 | 0.036 |
| 0.01 ng/ml A + | 0.472 | 0.311 | 0.361 | 1.654 | 1.914 | 1.456 | 0.031 | 0.028 | 0.022 |
| 100 μg/ml CDDP | | | | | | | | | |
| Average | | | | | | | | | |
| Unstimulated cells | 0.765 | 2.791 | 2.767 | 2.086 | 2.836 | 2.867 | 0.412 | 0.45 | 0.922 |
| 0.1 ng/ml A | 0.807 | 2.829 | 3.301 | 2.074 | 2.887 | 2.875 | 0.357 | 0.555 | 0.912 |
| 0.1 μg/ml CDDP | 0.607 | 1.304 | 0.663 | 2.072 | 2.421 | 2.487 | 0.34 | 0.332 | 0.479 |
| 0.1 ng/ml A + 0.1 | 0.815 | 0.868 | 0.202 | 2.015 | 2.205 | 1.999 | 0.255 | 0.187 | 0.077 |
| μg/ml CDDP | | | | | | | | | |
| Unstimulated cells | 0.765 | 2.791 | 2.767 | 2.086 | 2.836 | 2.867 | 0.412 | 0.45 | 0.922 |
| 0.1 ng/ml A | 0.807 | 2.829 | 3.301 | 2.074 | 2.887 | 2.875 | 0.357 | 0.555 | 0.912 |
| 1 μg/ml CDDP | 0.277 | 0.396 | 0.178 | 1.579 | 1.987 | 2.256 | 0.068 | 0.048 | 0.042 |
| 0.1 ng/ml A + 1 | 0.203 | 0.218 | 0.299 | 1.662 | 1.889 | 1.641 | 0.031 | 0.031 | 0.024 |
| μg/ml CDDP | | | | | | | | | |
| Unstimulated cells | 0.765 | 2.791 | 2.767 | 2.086 | 2.836 | 2.867 | 0.412 | 0.45 | 0.922 |
| 0.1 ng/ml A | 0.807 | 2.829 | 3.301 | 2.074 | 2.887 | 2.875 | 0.357 | 0.555 | 0.912 |
| 10 μg/ml CDDP | 0.32 | 0.405 | 0.282 | 1.117 | 1.678 | 1.913 | 0.066 | 0.044 | 0.04 |
| 0.1 ng/ml A + 10 | 0.238 | 0.271 | 0.285 | 1.294 | 1.508 | 1.482 | 0.045 | 0.039 | 0.031 |
| μg/ml CDDP | | | | | | | | | |
| Unstimulated cells | 0.765 | 2.791 | 2.767 | 2.086 | 2.836 | 2.867 | 0.412 | 0.45 | 0.922 |
| 0.1 ng/ml A | 0.807 | 2.829 | 3.301 | 2.074 | 2.887 | 2.875 | 0.357 | 0.555 | 0.912 |
| 100 μg/ml CDDP | 0.329 | 0.288 | 0.311 | 1.283 | 1.782 | 1.319 | 0.049 | 0.038 | 0.036 |
| 0.1 ng/ml A + 100 | 0.662 | 0.525 | 0.249 | 1.69 | 1.821 | 1.687 | 0.026 | 0.029 | 0.023 |
| μg/ml CDDP | | | | | | | | | |
| Average | | | | | | | | | |
| Unstimulated cells | 0.765 | 2.791 | 2.767 | 2.086 | 2.836 | 2.867 | 0.412 | 0.45 | 0.922 |
| 1 ng/ml A | 1.04 | 2.998 | 3.69 | 1.987 | 2.879 | 2.871 | 0.331 | 0.452 | 0.807 |
| 0.1 μg/ml CDDP | 0.607 | 1.304 | 0.663 | 2.072 | 2.421 | 2.487 | 0.34 | 0.332 | 0.479 |
| 1 ng/ml A + 0.1 | 0.847 | 1.279 | 0.523 | 1.891 | 2.368 | 2.029 | 0.25 | 0.224 | 0.169 |
| μg/ml CDDP | | | | | | | | | |
| Unstimulated cells | 0.765 | 2.791 | 2.767 | 2.086 | 2.836 | 2.867 | 0.412 | 0.45 | 0.922 |
| 1 ng/ml A | 1.04 | 2.998 | 3.69 | 1.987 | 2.879 | 2.871 | 0.331 | 0.452 | 0.807 |
| 1 μg/ml CDDP | 0.277 | 0.396 | 0.178 | 1.579 | 1.987 | 2.256 | 0.068 | 0.048 | 0.042 |
| 1 ng/ml A + 1 | 0.232 | 0.363 | 0.19 | 1.574 | 2.053 | 1.997 | 0.037 | 0.033 | 0.026 |
| μg/ml CDDP | | | | | | | | | |
| Unstimulated cells | 0.765 | 2.791 | 2.767 | 2.086 | 2.836 | 2.867 | 0.412 | 0.45 | 0.922 |
| 1 ng/ml A | 1.04 | 2.998 | 3.69 | 1.987 | 2.879 | 2.871 | 0.331 | 0.452 | 0.807 |
| 10 μg/ml CDDP | 0.32 | 0.405 | 0.282 | 1.117 | 1.678 | 1.913 | 0.066 | 0.044 | 0.04 |
| 1 ng/ml A + 10 | 0.47 | 0.397 | 0.333 | 1.641 | 1.844 | 1.668 | 0.056 | 0.048 | 0.037 |
| μg/ml CDDP | | | | | | | | | |
| Unstimulated cells | 0.765 | 2.791 | 2.767 | 2.086 | 2.836 | 2.867 | 0.412 | 0.45 | 0.922 |
| 1 ng/ml A | 1.04 | 2.998 | 3.69 | 1.987 | 2.879 | 2.871 | 0.331 | 0.452 | 0.807 |
| 100 μg/ml CDDP | 0.329 | 0.288 | 0.311 | 1.283 | 1.782 | 1.319 | 0.049 | 0.038 | 0.036 |
| 1 ng/ml A + 100 | 0.564 | 0.554 | 0.407 | 1.458 | 2.273 | 1.972 | 0.037 | 0.041 | 0.029 |
| μg/ml CDDP | | | | | | | | | |
| Average | | | | | | | | | |
| Unstimulated cells | 0.765 | 2.791 | 2.767 | 2.086 | 2.836 | 2.867 | 0.412 | 0.45 | 0.922 |
| 10 ng/ml A | 1.39 | 3.059 | 3.793 | 1.97 | 2.866 | 2.857 | 0.333 | 0.495 | 0.776 |
| 0.1 μg/ml CDDP | 0.607 | 1.304 | 0.663 | 2.072 | 2.421 | 2.487 | 0.34 | 0.332 | 0.479 |
| 10 ng/ml A + 0.1 | 0.925 | 1.017 | 0.568 | 1.931 | 2.083 | 1.779 | 0.249 | 0.218 | 0.105 |
| μg/ml CDDP | | | | | | | | | |
| Unstimulated cells | 0.765 | 2.791 | 2.767 | 2.086 | 2.836 | 2.867 | 0.412 | 0.45 | 0.922 |
| 10 ng/ml A | 1.39 | 3.059 | 3.793 | 1.97 | 2.866 | 2.857 | 0.333 | 0.495 | 0.776 |
| 1 μg/ml CDDP | 0.277 | 0.396 | 0.178 | 1.579 | 1.987 | 2.256 | 0.068 | 0.048 | 0.042 |
| 10 ng/ml A + 1 | 0.314 | 0.479 | 0.315 | 1.476 | 1.936 | 1.79 | 0.033 | 0.034 | 0.027 |
| μg/ml CDDP | | | | | | | | | |
| Unstimulated cells | 0.765 | 2.791 | 2.767 | 2.086 | 2.836 | 2.867 | 0.412 | 0.45 | 0.922 |
| 10 ng/ml A | 1.39 | 3.059 | 3.793 | 1.97 | 2.866 | 2.857 | 0.333 | 0.495 | 0.776 |
| 10 μg/ml CDDP | 0.32 | 0.405 | 0.282 | 1.117 | 1.678 | 1.913 | 0.066 | 0.044 | 0.04 |
| 10 ng/ml A + 10 | 0.679 | 0.494 | 0.373 | 1.589 | 1.874 | 1.737 | 0.05 | 0.043 | 0.037 |
| μg/ml CDDP | | | | | | | | | |
| Unstimulated cells | 0.765 | 2.791 | 2.767 | 2.086 | 2.836 | 2.867 | 0.412 | 0.45 | 0.922 |
| 10 ng/ml A | 1.39 | 3.059 | 3.793 | 1.97 | 2.866 | 2.857 | 0.333 | 0.495 | 0.776 |
| 100 μg/ml CDDP | 0.329 | 0.288 | 0.311 | 1.283 | 1.782 | 1.319 | 0.049 | 0.038 | 0.036 |
| 10 ng/ml A + 100 | 0.766 | 0.594 | 0.379 | 1.747 | 2.081 | 1.948 | 0.047 | 0.036 | 0.028 |
| μg/ml CDDP | | | | | | | | | |

TABLE 9

| | A375 | Run 2 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated cells | 0.859 | 2.118 | 2.778 | 2.433 | 2.842 | 2.894 | 0.406 | 0.688 | 0.996 |
| 0.01 ng/ml A | 0.884 | 2.077 | 3.008 | 2.472 | 2.812 | 2.892 | 0.401 | 0.887 | 0.926 |
| 0.1 μg/ml CDDP | 0.603 | 1.204 | 1.312 | 2.189 | 2.571 | 2.634 | 0.367 | 0.598 | 0.696 |
| 0.01 ng/ml A + 0.1 | 0.561 | 0.671 | 0.555 | 2.154 | 2.356 | 1.713 | 0.319 | 0.31 | 0.115 |
| μg/ml CDDP | | | | | | | | | |
| Unstimulated cells | 0.859 | 2.118 | 2.778 | 2.433 | 2.842 | 2.894 | 0.406 | 0.688 | 0.996 |
| 0.01 ng/ml A | 0.884 | 2.077 | 3.008 | 2.472 | 2.812 | 2.892 | 0.401 | 0.887 | 0.926 |
| 1 μg/ml CDDP | 0.197 | 0.605 | 1.001 | 1.916 | 1.366 | 2.085 | 0.047 | 0.046 | 0.049 |
| 0.01 ng/ml A + 1 | 0.161 | 0.317 | 0.202 | 1.833 | 1.539 | 1.646 | 0.029 | 0.026 | 0.032 |
| μg/ml CDDP | | | | | | | | | |
| Unstimulated cells | 0.859 | 2.118 | 2.778 | 2.433 | 2.842 | 2.894 | 0.406 | 0.688 | 0.996 |
| 0.01 ng/ml A | 0.884 | 2.077 | 3.008 | 2.472 | 2.812 | 2.892 | 0.401 | 0.887 | 0.926 |
| 10 μg/ml CDDP | 0.246 | 0.514 | 0.729 | 1.827 | 1.265 | 1.617 | 0.049 | 0.045 | 0.042 |
| 0.01 ng/ml A + 10 | 0.181 | 0.16 | 0.448 | 1.633 | 1.135 | 1.237 | 0.056 | 0.032 | 0.044 |
| μg/ml CDDP | | | | | | | | | |
| Unstimulated cells | 0.859 | 2.118 | 2.778 | 2.433 | 2.842 | 2.894 | 0.406 | 0.688 | 0.996 |
| 0.01 ng/ml A | 0.884 | 2.077 | 3.008 | 2.472 | 2.812 | 2.892 | 0.401 | 0.887 | 0.926 |
| 100 μg/ml CDDP | 0.241 | 0.238 | 0.293 | 1.939 | 1.293 | 1.491 | 0.031 | 0.048 | 0.038 |
| 0.01 ng/ml A + | 0.237 | 0.12 | 0.69 | 1.717 | 1.706 | 1.372 | 0.027 | 0.026 | 0.033 |
| 100 μg/ml CDDP | | | | | | | | | |
| Average | | | | | | | | | |
| Unstimulated cells | 0.859 | 2.118 | 2.778 | 2.433 | 2.842 | 2.894 | 0.406 | 0.688 | 0.996 |
| 0.1 ng/ml A | 0.961 | 2.232 | 3.112 | 2.508 | 2.861 | 2.918 | 0.454 | 0.765 | 0.89 |
| 0.1 μg/ml CDDP | 0.603 | 1.204 | 1.312 | 2.189 | 2.571 | 2.634 | 0.367 | 0.598 | 0.696 |
| 0.1 ng/ml A + 0.1 | 0.681 | 0.726 | 0.349 | 2.416 | 1.928 | 1.928 | 0.307 | 0.264 | 0.093 |
| μg/ml CDDP | | | | | | | | | |
| Unstimulated cells | 0.859 | 2.118 | 2.778 | 2.433 | 2.842 | 2.894 | 0.406 | 0.688 | 0.996 |
| 0.1 ng/ml A | 0.961 | 2.232 | 3.112 | 2.508 | 2.861 | 2.918 | 0.454 | 0.765 | 0.89 |
| 1 μg/ml CDDP | 0.197 | 0.605 | 1.001 | 1.916 | 1.366 | 2.085 | 0.047 | 0.046 | 0.049 |
| 0.1 ng/ml A + 1 | 0.221 | 0.196 | 0.329 | 2.043 | 1.612 | 1.835 | 0.04 | 0.027 | 0.032 |
| μg/ml CDDP | | | | | | | | | |
| Unstimulated cells | 0.859 | 2.118 | 2.778 | 2.433 | 2.842 | 2.894 | 0.406 | 0.688 | 0.996 |
| 0.1 ng/ml A | 0.961 | 2.232 | 3.112 | 2.508 | 2.861 | 2.918 | 0.454 | 0.765 | 0.89 |
| 10 μg/ml CDDP | 0.246 | 0.514 | 0.729 | 1.827 | 1.265 | 1.617 | 0.049 | 0.045 | 0.042 |
| 0.1 ng/ml A + 10 | 0.217 | 0.168 | 0.793 | 1.578 | 1.349 | 1.551 | 0.05 | 0.036 | 0.042 |
| μg/ml CDDP | | | | | | | | | |
| Unstimulated cells | 0.859 | 2.118 | 2.778 | 2.433 | 2.842 | 2.894 | 0.406 | 0.688 | 0.996 |
| 0.1 ng/ml A | 0.961 | 2.232 | 3.112 | 2.508 | 2.861 | 2.918 | 0.454 | 0.765 | 0.89 |
| 100 μg/ml CDDP | 0.241 | 0.238 | 0.293 | 1.939 | 1.293 | 1.491 | 0.031 | 0.048 | 0.038 |
| 0.1 ng/ml A + 100 | 0.256 | 0.168 | 1.08 | 1.956 | 1.831 | 1.746 | 0.023 | 0.024 | 0.031 |
| μg/ml CDDP | | | | | | | | | |
| Average | | | | | | | | | |
| Unstimulated cells | 0.859 | 2.118 | 2.778 | 2.433 | 2.842 | 2.894 | 0.406 | 0.688 | 0.996 |
| 1 ng/ml A | 1.044 | 2.638 | 3.639 | 2.403 | 2.853 | 2.925 | 0.405 | 0.684 | 0.767 |
| 0.1 μg/ml CDDP | 0.603 | 1.204 | 1.312 | 2.189 | 2.571 | 2.634 | 0.367 | 0.598 | 0.696 |
| 1 ng/ml A + 0.1 | 0.838 | 1.236 | 0.634 | 2.382 | 2.182 | 2.119 | 0.32 | 0.337 | 0.196 |
| μg/ml CDDP | | | | | | | | | |
| Unstimulated cells | 0.859 | 2.118 | 2.778 | 2.433 | 2.842 | 2.894 | 0.406 | 0.688 | 0.996 |
| 1 ng/ml A | 1.044 | 2.638 | 3.639 | 2.403 | 2.853 | 2.925 | 0.405 | 0.684 | 0.767 |
| 1 μg/ml CDDP | 0.197 | 0.605 | 1.001 | 1.916 | 1.366 | 2.085 | 0.047 | 0.046 | 0.049 |
| 1 ng/ml A + 1 | 0.331 | 0.392 | 0.286 | 2.28 | 1.631 | 2.037 | 0.05 | 0.043 | 0.033 |
| μg/ml CDDP | | | | | | | | | |
| Unstimulated cells | 0.859 | 2.118 | 2.778 | 2.433 | 2.842 | 2.894 | 0.406 | 0.688 | 0.996 |
| 1 ng/ml A | 1.044 | 2.638 | 3.639 | 2.403 | 2.853 | 2.925 | 0.405 | 0.684 | 0.767 |
| 10 μg/ml CDDP | 0.246 | 0.514 | 0.729 | 1.827 | 1.265 | 1.617 | 0.049 | 0.045 | 0.042 |
| 1 ng/ml A + 10 | 0.367 | 0.319 | 0.65 | 1.919 | 1.567 | 1.788 | 0.068 | 0.046 | 0.041 |
| μg/ml CDDP | | | | | | | | | |
| Unstimulated cells | 0.859 | 2.118 | 2.778 | 2.433 | 2.842 | 2.894 | 0.406 | 0.688 | 0.996 |
| 1 ng/ml A | 1.044 | 2.638 | 3.639 | 2.403 | 2.853 | 2.925 | 0.405 | 0.684 | 0.767 |
| 100 μg/ml CDDP | 0.241 | 0.238 | 0.293 | 1.939 | 1.293 | 1.491 | 0.031 | 0.048 | 0.038 |
| 1 ng/ml A + 100 | 0.506 | 0.412 | 0.958 | 2.694 | 2.245 | 1.835 | 0.062 | 0.034 | 0.038 |
| μg/ml CDDP | | | | | | | | | |
| Average | | | | | | | | | |
| Unstimulated cells | 0.859 | 2.118 | 2.778 | 2.433 | 2.842 | 2.894 | 0.406 | 0.688 | 0.996 |
| 10 ng/ml A | 1.249 | 2.709 | 3.515 | 2.13 | 2.854 | 2.9 | 0.462 | 0.677 | 0.87 |
| 0.1 μg/ml CDDP | 0.603 | 1.204 | 1.312 | 2.189 | 2.571 | 2.634 | 0.367 | 0.598 | 0.696 |
| 10 ng/ml A + 0.1 | 1.024 | 0.997 | 0.493 | 2.198 | 2.354 | 2.096 | 0.307 | 0.324 | 0.195 |
| μg/ml CDDP | | | | | | | | | |
| Unstimulated cells | 0.859 | 2.118 | 2.778 | 2.433 | 2.842 | 2.894 | 0.406 | 0.688 | 0.996 |
| 10 ng/ml A | 1.249 | 2.709 | 3.515 | 2.13 | 2.854 | 2.9 | 0.462 | 0.677 | 0.87 |
| 1 μg/ml CDDP | 0.197 | 0.605 | 1.001 | 1.916 | 1.366 | 2.085 | 0.047 | 0.046 | 0.049 |
| 10 ng/ml A + 1 | 0.387 | 0.273 | 0.308 | 2.141 | 1.701 | 1.757 | 0.029 | 0.035 | 0.035 |
| μg/ml CDDP | | | | | | | | | |
| Unstimulated cells | 0.859 | 2.118 | 2.778 | 2.433 | 2.842 | 2.894 | 0.406 | 0.688 | 0.996 |
| 10 ng/ml A | 1.249 | 2.709 | 3.515 | 2.13 | 2.854 | 2.9 | 0.462 | 0.677 | 0.87 |
| 10 μg/ml CDDP | 0.246 | 0.514 | 0.729 | 1.827 | 1.265 | 1.617 | 0.049 | 0.045 | 0.042 |
| 10 ng/ml A + 10 | 0.3 | 0.453 | 0.621 | 1.67 | 1.707 | 1.697 | 0.052 | 0.042 | 0.046 |
| μg/ml CDDP | | | | | | | | | |
| Unstimulated cells | 0.859 | 2.118 | 2.778 | 2.433 | 2.842 | 2.894 | 0.406 | 0.688 | 0.996 |
| 10 ng/ml A | 1.249 | 2.709 | 3.515 | 2.13 | 2.854 | 2.9 | 0.462 | 0.677 | 0.87 |
| 100 μg/ml CDDP | 0.241 | 0.238 | 0.293 | 1.939 | 1.293 | 1.491 | 0.031 | 0.048 | 0.038 |
| 10 ng/ml A + 100 | 0.581 | 0.562 | 0.703 | 2.476 | 1.701 | 1.641 | 0.029 | 0.03 | 0.037 |
| μg/ml CDDP | | | | | | | | | |

TABLE 10

| | A375 | Run 3 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated cells | 0.939 | 2.457 | 2.724 | 1.861 | 2.702 | 2.857 | 0.299 | 0.646 | 0.766 |
| 0.01 ng/ml A | 1.019 | 2.144 | 2.958 | 1.792 | 2.765 | 2.874 | 0.311 | 0.663 | 0.725 |
| 0.1 μg/ml CDDP | 0.856 | 0.92 | 0.961 | 1.597 | 2.175 | 2.184 | 0.25 | 0.422 | 0.413 |
| 0.01 ng/ml A + 0.1 | 0.492 | 0.31 | 0.235 | 1.6 | 1.379 | 1.224 | 0.127 | 0.09 | 0.041 |
| μg/ml CDDP | | | | | | | | | |
| Unstimulated cells | 0.939 | 2.457 | 2.724 | 1.861 | 2.702 | 2.857 | 0.299 | 0.646 | 0.766 |
| 0.01 ng/ml A | 1.019 | 2.144 | 2.958 | 1.792 | 2.765 | 2.874 | 0.311 | 0.663 | 0.725 |
| 1 μg/ml CDDP | 0.655 | 0.375 | 0.388 | 1.126 | 1.83 | 1.491 | 0.052 | 0.041 | 0.026 |
| 0.01 ng/ml A + 1 | 0.318 | 0.188 | 0.205 | 1.263 | 1.284 | 1.176 | 0.043 | 0.028 | 0.017 |
| μg/ml CDDP | | | | | | | | | |
| Unstimulated cells | 0.939 | 2.457 | 2.724 | 1.861 | 2.702 | 2.857 | 0.299 | 0.646 | 0.766 |
| 0.01 ng/ml A | 1.019 | 2.144 | 2.958 | 1.792 | 2.765 | 2.874 | 0.311 | 0.663 | 0.725 |
| 10 μg/ml CDDP | 0.529 | 0.309 | 0.344 | 1.294 | 1.586 | 1.571 | 0.054 | 0.037 | 0.033 |
| 0.01 ng/ml A + 10 | 0.336 | 0.151 | 0.302 | 1.036 | 1.237 | 1.409 | 0.043 | 0.039 | 0.025 |
| μg/ml CDDP | | | | | | | | | |
| Unstimulated cells | 0.939 | 2.457 | 2.724 | 1.861 | 2.702 | 2.857 | 0.299 | 0.646 | 0.766 |
| 0.01 ng/ml A | 1.019 | 2.144 | 2.958 | 1.792 | 2.765 | 2.874 | 0.311 | 0.663 | 0.725 |
| 100 μg/ml CDDP | 0.224 | 0.252 | 0.24 | 0.982 | 1.572 | 1.271 | 0.01 | 0.036 | 0.031 |
| 0.01 ng/ml A + | 0.493 | 0.236 | 0.319 | 1.361 | 1.577 | 1.532 | 0.035 | 0.029 | 0.019 |
| 100 μg/ml CDDP | | | | | | | | | |
| Average | | | | | | | | | |
| Unstimulated cells | 0.939 | 2.457 | 2.724 | 1.861 | 2.702 | 2.857 | 0.299 | 0.646 | 0.766 |
| 0.1 ng/ml A | 1.297 | 2.245 | 2.755 | 1.685 | 2.803 | 2.834 | 0.347 | 0.728 | 0.779 |
| 0.1 μg/ml CDDP | 0.856 | 0.92 | 0.961 | 1.597 | 2.175 | 2.184 | 0.25 | 0.422 | 0.413 |
| 0.1 ng/ml A + 0.1 | 1.004 | 0.535 | 0.348 | 1.878 | 1.596 | 1.425 | 0.215 | 0.179 | 0.054 |
| μg/ml CDDP | | | | | | | | | |
| Unstimulated cells | 0.939 | 2.457 | 2.724 | 1.861 | 2.702 | 2.857 | 0.299 | 0.646 | 0.766 |
| 0.1 ng/ml A | 1.297 | 2.245 | 2.755 | 1.685 | 2.803 | 2.834 | 0.347 | 0.728 | 0.779 |
| 1 μg/ml CDDP | 0.655 | 0.375 | 0.388 | 1.126 | 1.83 | 1.491 | 0.052 | 0.041 | 0.026 |
| 0.1 ng/ml A + 1 | 0.299 | 0.238 | 0.184 | 1.034 | 1.551 | 1.654 | 0.04 | 0.029 | 0.019 |
| μg/ml CDDP | | | | | | | | | |
| Unstimulated cells | 0.939 | 2.457 | 2.724 | 1.861 | 2.702 | 2.857 | 0.299 | 0.646 | 0.766 |
| 0.1 ng/ml A | 1.297 | 2.245 | 2.755 | 1.685 | 2.803 | 2.834 | 0.347 | 0.728 | 0.779 |
| 10 μg/ml CDDP | 0.529 | 0.309 | 0.344 | 1.294 | 1.586 | 1.571 | 0.054 | 0.037 | 0.033 |
| 0.1 ng/ml A + 10 | 0.552 | 0.246 | 0.356 | 0.978 | 1.29 | 1.928 | 0.051 | 0.036 | 0.024 |
| μg/ml CDDP | | | | | | | | | |
| Unstimulated cells | 0.939 | 2.457 | 2.724 | 1.861 | 2.702 | 2.857 | 0.299 | 0.646 | 0.766 |
| 0.1 ng/ml A | 1.297 | 2.245 | 2.755 | 1.685 | 2.803 | 2.834 | 0.347 | 0.728 | 0.779 |
| 100 μg/ml CDDP | 0.224 | 0.252 | 0.24 | 0.982 | 1.572 | 1.271 | 0.01 | 0.036 | 0.031 |
| 0.1 ng/ml A + 100 | 0.666 | 0.349 | 0.345 | 1.191 | 1.72 | 1.832 | 0.036 | 0.033 | 0.02 |
| μg/ml CDDP | | | | | | | | | |
| Average | | | | | | | | | |
| Unstimulated cells | 0.939 | 2.457 | 2.724 | 1.861 | 2.702 | 2.857 | 0.299 | 0.646 | 0.766 |
| 1 ng/ml A | 1.224 | 2.443 | 3.246 | 1.684 | 2.776 | 2.9 | 0.334 | 0.645 | 0.762 |
| 0.1 μg/ml CDDP | 0.856 | 0.92 | 0.961 | 1.597 | 2.175 | 2.184 | 0.25 | 0.422 | 0.413 |
| 1 ng/ml A + 0.1 | 0.865 | 0.765 | 0.435 | 1.745 | 1.979 | 1.921 | 0.24 | 0.244 | 0.112 |
| μg/ml CDDP | | | | | | | | | |
| Unstimulated cells | 0.939 | 2.457 | 2.724 | 1.861 | 2.702 | 2.857 | 0.299 | 0.646 | 0.766 |
| 1 ng/ml A | 1.224 | 2.443 | 3.246 | 1.684 | 2.776 | 2.9 | 0.334 | 0.645 | 0.762 |
| 1 μg/ml CDDP | 0.655 | 0.375 | 0.388 | 1.126 | 1.83 | 1.491 | 0.052 | 0.041 | 0.026 |
| 1 ng/ml A + 1 | 0.41 | 0.243 | 0.26 | 0.979 | 1.832 | 1.849 | 0.041 | 0.034 | 0.025 |
| μg/ml CDDP | | | | | | | | | |
| Unstimulated cells | 0.939 | 2.457 | 2.724 | 1.861 | 2.702 | 2.857 | 0.299 | 0.646 | 0.766 |
| 1 ng/ml A | 1.224 | 2.443 | 3.246 | 1.684 | 2.776 | 2.9 | 0.334 | 0.645 | 0.762 |
| 10 μg/ml CDDP | 0.529 | 0.309 | 0.344 | 1.294 | 1.586 | 1.571 | 0.054 | 0.037 | 0.033 |
| 1 ng/ml A + 10 | 0.65 | 0.322 | 0.444 | 1.395 | 1.445 | 1.85 | 0.063 | 0.045 | 0.034 |
| μg/ml CDDP | | | | | | | | | |
| Unstimulated cells | 0.939 | 2.457 | 2.724 | 1.861 | 2.702 | 2.857 | 0.299 | 0.646 | 0.766 |
| 1 ng/ml A | 1.224 | 2.443 | 3.246 | 1.684 | 2.776 | 2.9 | 0.334 | 0.645 | 0.762 |
| 100 μg/ml CDDP | 0.224 | 0.252 | 0.24 | 0.982 | 1.572 | 1.271 | 0.01 | 0.036 | 0.031 |
| 1 ng/ml A + 100 | 0.903 | 0.711 | 0.304 | 1.498 | 1.967 | 1.94 | 0.041 | 0.035 | 0.023 |
| μg/ml CDDP | | | | | | | | | |
| Average | | | | | | | | | |
| Unstimulated cells | 0.939 | 2.457 | 2.724 | 1.861 | 2.702 | 2.857 | 0.299 | 0.646 | 0.766 |
| 10 ng/ml A | 1.24 | 2.459 | 3.406 | 1.6 | 2.784 | 2.888 | 0.361 | 0.701 | 0.689 |
| 0.1 μg/ml CDDP | 0.856 | 0.92 | 0.961 | 1.597 | 2.175 | 2.184 | 0.25 | 0.422 | 0.413 |
| 10 ng/ml A + 0.1 | 0.66 | 0.724 | 0.316 | 1.634 | 1.919 | 1.7 | 0.216 | 0.228 | 0.083 |
| μg/ml CDDP | | | | | | | | | |
| Unstimulated cells | 0.939 | 2.457 | 2.724 | 1.861 | 2.702 | 2.857 | 0.299 | 0.646 | 0.766 |
| 10 ng/ml A | 1.24 | 2.459 | 3.406 | 1.6 | 2.784 | 2.888 | 0.361 | 0.701 | 0.689 |
| 1 μg/ml CDDP | 0.655 | 0.375 | 0.388 | 1.126 | 1.83 | 1.491 | 0.052 | 0.041 | 0.026 |
| 10 ng/ml A + 1 | 0.341 | 0.304 | 0.375 | 1.219 | 1.617 | 1.455 | 0.043 | 0.033 | 0.026 |
| μg/ml CDDP | | | | | | | | | |
| Unstimulated cells | 0.939 | 2.457 | 2.724 | 1.861 | 2.702 | 2.857 | 0.299 | 0.646 | 0.766 |
| 10 ng/ml A | 1.24 | 2.459 | 3.406 | 1.6 | 2.784 | 2.888 | 0.361 | 0.701 | 0.689 |
| 10 μg/ml CDDP | 0.529 | 0.309 | 0.344 | 1.294 | 1.586 | 1.571 | 0.054 | 0.037 | 0.033 |
| 10 ng/ml A + 10 | 0.809 | 0.482 | 0.392 | 1.66 | 1.439 | 1.755 | 0.061 | 0.04 | 0.035 |
| μg/ml CDDP | | | | | | | | | |
| Unstimulated cells | 0.939 | 2.457 | 2.724 | 1.861 | 2.702 | 2.857 | 0.299 | 0.646 | 0.766 |
| 10 ng/ml A | 1.24 | 2.459 | 3.406 | 1.6 | 2.784 | 2.888 | 0.361 | 0.701 | 0.689 |
| 100 μg/ml CDDP | 0.224 | 0.252 | 0.24 | 0.982 | 1.572 | 1.271 | 0.01 | 0.036 | 0.031 |
| 10 ng/ml A + 100 | 0.751 | 0.565 | 0.352 | 1.678 | 1.71 | 1.563 | 0.041 | 0.03 | 0.028 |
| μg/ml CDDP | | | | | | | | | |

TABLE 11

| | CALU- | | | | | | | | |
|---|---|---|---|---|---|---|---|---|---|
| | 1 | Run 1 | | | | | | | |
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated cells | 0.444 | 0.683 | 1.163 | 1.955 | 1.901 | 2.612 | 0.292 | 0.31 | 0.757 |
| 0.01 ng/ml A | 0.433 | 1.014 | 1.001 | 1.63 | 2.111 | 2.627 | 0.257 | 0.373 | 0.543 |
| 0.1 μg/ml CDDP | 0.554 | 0.652 | 0.441 | 1.457 | 1.939 | 1.598 | 0.178 | 0.272 | 0.272 |
| 0.01 ng/ml A + 0.1 | 0.661 | 0.373 | 0.248 | 1.68 | 1.568 | 2.089 | 0.17 | 0.166 | 0.168 |
| 1 μg/ml CDDP | | | | | | | | | |
| Unstimulated cells | 0.444 | 0.683 | 1.163 | 1.955 | 1.901 | 2.612 | 0.292 | 0.31 | 0.757 |
| 0.01 ng/ml A | 0.433 | 1.014 | 1.001 | 1.63 | 2.111 | 2.627 | 0.257 | 0.373 | 0.543 |
| 1 μg/ml CDDP | 0.231 | 0.661 | 0.135 | 1.438 | 1.991 | 1.5 | 0.024 | 0.024 | 0.032 |
| 0.01 ng/ml A + 1 | 0.459 | 0.224 | 0.072 | 1.254 | 1.399 | 1.687 | 0.02 | 0.012 | 0.017 |
| μg/ml CDDP | | | | | | | | | |
| Unstimulated cells | 0.444 | 0.683 | 1.163 | 1.955 | 1.901 | 2.612 | 0.292 | 0.31 | 0.757 |
| 0.01 ng/ml A | 0.433 | 1.014 | 1.001 | 1.63 | 2.111 | 2.627 | 0.257 | 0.373 | 0.543 |
| 10 μg/ml CDDP | 0.421 | 0.503 | 0.157 | 1.401 | 1.563 | 1.108 | 0.043 | 0.031 | 0.024 |
| 0.01 ng/ml A + 10 | 0.511 | 0.268 | 0.114 | 1.356 | 1.182 | 1.305 | 0.027 | 0.013 | 0.018 |
| μg/ml CDDP | | | | | | | | | |
| Unstimulated cells | 0.444 | 0.683 | 1.163 | 1.955 | 1.901 | 2.612 | 0.292 | 0.31 | 0.757 |
| 0.01 ng/ml A | 0.433 | 1.014 | 1.001 | 1.63 | 2.111 | 2.627 | 0.257 | 0.373 | 0.543 |
| 100 μg/ml CDDP | 0.306 | 0.409 | 0.125 | 1.348 | 1.521 | 1.299 | 0.033 | 0.033 | 0.019 |
| 0.01 ng/ml A + | 0.343 | 0.521 | 0.255 | 1.652 | 1.714 | 1.396 | 0.019 | 0.014 | 0.016 |
| 100 μg/ml CDDP | | | | | | | | | |
| Unstimulated cells | 0.444 | 0.683 | 1.163 | 1.955 | 1.901 | 2.612 | 0.292 | 0.31 | 0.757 |
| 0.1 ng/ml A | 0.474 | 0.967 | 0.976 | 1.371 | 2.276 | 2.466 | 0.261 | 0.315 | 0.387 |
| 0.1 μg/ml CDDP | 0.554 | 0.652 | 0.441 | 1.457 | 1.939 | 1.598 | 0.178 | 0.272 | 0.272 |
| 0.1 ng/ml A + 0.1 | 0.715 | 0.441 | 0.299 | 1.583 | 1.67 | 1.88 | 0.164 | 0.187 | 0.218 |
| μg/ml CDDP | | | | | | | | | |
| Unstimulated cells | 0.444 | 0.683 | 1.163 | 1.955 | 1.901 | 2.612 | 0.292 | 0.31 | 0.757 |
| 0.1 ng/ml A | 0.474 | 0.967 | 0.976 | 1.371 | 2.276 | 2.466 | 0.261 | 0.315 | 0.387 |
| 1 μg/ml CDDP | 0.231 | 0.661 | 0.135 | 1.438 | 1.991 | 1.5 | 0.024 | 0.024 | 0.032 |
| 0.1 ng/ml A + 1 | 0.899 | 0.222 | 0.151 | 1.588 | 2.032 | 1.541 | 0.018 | 0.015 | 0.015 |
| μg/ml CDDP | | | | | | | | | |
| Unstimulated cells | 0.444 | 0.683 | 1.163 | 1.955 | 1.901 | 2.612 | 0.292 | 0.31 | 0.757 |
| 0.1 ng/ml A | 0.474 | 0.967 | 0.976 | 1.371 | 2.276 | 2.466 | 0.261 | 0.315 | 0.387 |
| 10 ng/ml CDDP | 0.421 | 0.503 | 0.157 | 1.401 | 1.563 | 1.108 | 0.043 | 0.031 | 0.024 |
| 0.1 ng/ml A + 10 | 0.58 | 0.408 | 0.122 | 1.639 | 1.603 | 1.555 | 0.033 | 0.011 | 0.017 |
| μg/ml CDDP | | | | | | | | | |
| Unstimulated cells | 0.444 | 0.683 | 1.163 | 1.955 | 1.901 | 2.612 | 0.292 | 0.31 | 0.757 |
| 0.1 ng/ml A | 0.474 | 0.967 | 0.976 | 1.371 | 2.276 | 2.466 | 0.261 | 0.315 | 0.387 |
| 100 CDDP | 0.306 | 0.409 | 0.125 | 1.348 | 1.521 | 1.299 | 0.033 | 0.033 | 0.019 |
| 0.1 ng/ml A + 100 | 0.646 | 0.814 | 0.397 | 2.196 | 1.569 | 1.35 | 0.017 | 0.013 | 0.02 |
| μg/ml CDDP | | | | | | | | | |
| Unstimulated cells | 0.444 | 0.683 | 1.163 | 1.955 | 1.901 | 2.612 | 0.292 | 0.31 | 0.757 |
| 1 ng/ml A | 0.544 | 1.116 | 1.302 | 1.421 | 2.288 | 2.624 | 0.253 | 0.327 | 0.345 |
| 0.1 μg/ml CDDP | 0.554 | 0.652 | 0.441 | 1.457 | 1.939 | 1.598 | 0.178 | 0.272 | 0.272 |
| 1 ng/ml A + 0.1 | 0.547 | 0.599 | 0.429 | 1.661 | 2.085 | 1.855 | 0.16 | 0.183 | 0.219 |
| μg/ml CDDP | | | | | | | | | |
| Unstimulated cells | 0.444 | 0.683 | 1.163 | 1.955 | 1.901 | 2.612 | 0.292 | 0.3 | 0.757 |
| 1 ng/ml A | 0.544 | 1.116 | 1.302 | 1.421 | 2.288 | 2.624 | 0.253 | 0.327 | 0.345 |
| 1 μg/ml CDDP | 0.231 | 0.661 | 0.135 | 1.438 | 1.991 | 1.5 | 0.024 | 0.024 | 0.032 |
| 1 ng/ml A + 1 | 0.363 | 0.297 | 0.117 | 1.483 | 2.558 | 1.633 | 0.02 | 0.026 | 0.02 |
| μg/ml CDDP | | | | | | | | | |
| Unstimulated cells | 0.444 | 0.683 | 1.163 | 1.955 | 1.901 | 2.612 | 0.292 | 0.31 | 0.757 |
| 1 ng/ml A | 0.544 | 1.116 | 1.302 | 1.421 | 2.288 | 2.624 | 0.253 | 0.327 | 0.345 |
| 10 ng/ml CDDP | 0.421 | 0.503 | 0.157 | 1.401 | 1.563 | 1.108 | 0.043 | 0.031 | 0.024 |
| 1 ng/ml A + 10 | 0.647 | 0.485 | 0.141 | 1.585 | 1.703 | 1.608 | 0.068 | 0.015 | 0.021 |
| μg/ml CDDP | | | | | | | | | |
| Unstimulated cells | 0.444 | 0.683 | 1.163 | 1.955 | 1.901 | 2.612 | 0.292 | 0.31 | 0.757 |
| 1 ng/ml A | 0.544 | 1.116 | 1.302 | 1.421 | 2.288 | 2.624 | 0.253 | 0.327 | 0.345 |
| 100 μg/ml CDDP | 0.306 | 0.409 | 0.125 | 1.348 | 1.521 | 1.299 | 0.033 | 0.033 | 0.019 |
| 1 ng/ml A+ 100 | 0.609 | 0.487 | 0.23 | 2.394 | 1.79 | 1.479 | 0.023 | 0.017 | 0.019 |
| μg/ml CDDP | | | | | | | | | |
| Unstimulated cells | 0.444 | 0.683 | 1.163 | 1.955 | 1.901 | 2.612 | 0.292 | 0.31 | 0.757 |
| 10 ng/ml A | 0.669 | 0.906 | 1.34 | 1.417 | 1.841 | 2.25 | 0.245 | 0.395 | 0.33 |
| 0.1 μg/ml CDDP | 0.554 | 0.652 | 0.441 | 1.457 | 1.939 | 1.598 | 0.178 | 0.272 | 0.272 |
| 10 ng/ml A + 0.1 | 0.603 | 0.888 | 0.526 | 1.56 | 2.243 | 1.5 | 0.198 | 0.255 | 0.186 |
| μg/ml CDDP | | | | | | | | | |
| Unstimulated cells | 0.444 | 0.683 | 1.163 | 1.955 | 1.901 | 2.612 | 0.292 | 0.3 | 0.757 |
| 10 ng/ml A | 0.669 | 0.906 | 1.34 | 1.417 | 1.841 | 2.25 | 0.245 | 0.395 | 0.33 |
| 1 μg/ml CDDP | 0.231 | 0.661 | 0.135 | 1.438 | 1.991 | 1.5 | 0.024 | 0.024 | 0.032 |
| 10 ng/ml A + 1 | 0.275 | 0.434 | 0.134 | 1.522 | 2.211 | 1.544 | 0.026 | 0.013 | 0.019 |
| μg/ml CDDP | | | | | | | | | |
| Unstimulated cells | 0.444 | 0.683 | 1.163 | 1.955 | 1.901 | 2.612 | 0.292 | 0.31 | 0.757 |
| 10 ng/ml A | 0.669 | 0.906 | 1.34 | 1.417 | 1.841 | 2.25 | 0.245 | 0.395 | 0.33 |
| 10 m/ml CDDP | 0.421 | 0.503 | 0.157 | 1.401 | 1.563 | 1.108 | 0.043 | 0.031 | 0.024 |
| 10 ng/ml A + 10 | 0.439 | 0.67 | 0.249 | 1.407 | 1.355 | 1.41 | 0.083 | 0.014 | 0.021 |
| μg/ml CDDP | | | | | | | | | |
| Unstimulated cells | 0.444 | 0.683 | 1.163 | 1.955 | 1.901 | 2.612 | 0.292 | 0.31 | 0.757 |
| 10 ng/ml A | 0.669 | 0.906 | 1.34 | 1.417 | 1.841 | 2.25 | 0.245 | 0.395 | 0.33 |
| 100 CDDP | 0.306 | 0.409 | 0.125 | 1.348 | 1.521 | 1.299 | 0.033 | 0.033 | 0.019 |
| 10 ng/ml A + 100 | 0.454 | 0.409 | 0.235 | 1.652 | 1.685 | 2.046 | 0.022 | 0.014 | 0.02 |
| μg/ml CDDP | | | | | | | | | |

TABLE 12

| | CALU- | | | | | | | | |
|---|---|---|---|---|---|---|---|---|---|
| | 1 | Run 2 | | | | | | | |
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated cells | 0.542 | 0.867 | 1.625 | 1.538 | 1.944 | 2.859 | 0.3 | 0.414 | 0.454 |
| 0.01 ng/ml A | 0.528 | 0.858 | 1.824 | 1.793 | 2.352 | 2.923 | 0.32 | 0.517 | 0.376 |
| 0.1 μg/ml CDDP | 0.484 | 0.886 | 0.786 | 1.437 | 1.79 | 2.674 | 0.244 | 0.322 | 0.29 |
| 0.01 ng/ml A + 0.1 | 0.41 | 0.489 | 0.255 | 1.393 | 1.504 | 1.897 | 0.145 | 0.088 | 0.054 |
| μg/ml CDDP | | | | | | | | | |
| Unstimulated cells | 0.542 | 0.867 | 1.625 | 1.538 | 1.944 | 2.859 | 0.3 | 0.414 | 0.454 |
| 0.01 ng/ml A | 0.528 | 0.858 | 1.824 | 1.793 | 2.352 | 2.923 | 0.32 | 0.517 | 0.376 |
| 1 μg/ml CDDP | 0.576 | 0.563 | 0.214 | 1.725 | 1.837 | 2.563 | 0.055 | 0.024 | 0.021 |
| 0.01 ng/ml A + 1 | 0.295 | 0.247 | 0.21 | 1.522 | 1.55 | 1.684 | 0.026 | 0.012 | 0.017 |
| μg/ml CDDP | | | | | | | | | |
| Unstimulated cells | 0.542 | 0.867 | 1.625 | 1.538 | 1.944 | 2.859 | 0.3 | 0.414 | 0.454 |
| 0.01 ng/ml A | 0.528 | 0.858 | 1.824 | 1.793 | 2.352 | 2.923 | 0.32 | 0.517 | 0.376 |
| 10 μg/ml CDDP | 0.64 | 0.46 | 0.254 | 1.48 | 1.702 | 2.434 | 0.056 | 0.019 | 0.015 |
| 0.01 ng/ml A + 10 | 0.341 | 0.298 | 0.561 | 1.169 | 1.284 | 2.313 | 0.049 | 0.014 | 0.016 |
| μg/ml CDDP | | | | | | | | | |
| Unstimulated cells | 0.542 | 0.867 | 1.625 | 1.538 | 1.944 | 2.859 | 0.3 | 0.414 | 0.454 |
| 0.01 ng/ml A | 0.528 | 0.858 | 1.824 | 1.793 | 2.352 | 2.923 | 0.32 | 0.517 | 0.376 |
| 100 μg/ml CDDP | 0.416 | 0.352 | 0.344 | 1.318 | 1.541 | 1.575 | 0.035 | 0.016 | 0.018 |
| 0.01 ng/ml A + | 0.6 | 0.379 | 0.531 | 1.689 | 1.608 | 2.684 | 0.025 | 0.013 | 0.014 |
| 100 μg/ml CDDP | | | | | | | | | |
| Unstimulated cells | 0.542 | 0.867 | 1.625 | 1.538 | 1.944 | 2.859 | 0.3 | 0.414 | 0.454 |
| 0.1 ng/ml A | 0.659 | 1.501 | 1.962 | 1.915 | 2.346 | 2.838 | 0.337 | 0.408 | 0.366 |
| 0.1 μg/ml CDDP | 0.484 | 0.886 | 0.786 | 1.437 | 1.79 | 2.674 | 0.244 | 0.322 | 0.29 |
| 0.1 ng/ml A + 0.1 | 0.651 | 0.714 | 0.48 | 1.887 | 1.961 | 2.406 | 0.194 | 0.249 | 0.192 |
| μg/ml CDDP | | | | | | | | | |
| Unstimulated cells | 0.542 | 0.867 | 1.625 | 1.538 | 1.944 | 2.859 | 0.3 | 0.414 | 0.454 |
| 0.1 ng/ml A | 0.659 | 1.501 | 1.962 | 1.915 | 2.346 | 2.838 | 0.337 | 0.408 | 0.366 |
| 1 μg/ml CDDP | 0.576 | 0.563 | 0.214 | 1.725 | 1.837 | 2.563 | 0.055 | 0.024 | 0.021 |
| 0.1 ng/ml A + 1 | 0.424 | 0.339 | 0.188 | 2.079 | 1.636 | 1.723 | 0.027 | 0.011 | 0.01 |
| μg/ml CDDP | | | | | | | | | |
| Unstimulated cells | 0.542 | 0.867 | 1.625 | 1.538 | 1.944 | 2.859 | 0.3 | 0.414 | 0.454 |
| 0.1 ng/ml A | 0.659 | 1.501 | 1.962 | 1.915 | 2.346 | 2.838 | 0.337 | 0.408 | 0.366 |
| 10 μg/ml CDDP | 0.64 | 0.46 | 0.254 | 1.48 | 1.702 | 2.434 | 0.056 | 0.019 | 0.015 |
| 0.1 ng/ml A + 10 | 0.397 | 0.596 | 0.373 | 1.286 | 1.449 | 2.55 | 0.036 | 0.011 | 0.016 |
| μg/ml CDDP | | | | | | | | | |
| Unstimulated cells | 0.542 | 0.867 | 1.625 | 1.538 | 1.944 | 2.859 | 0.3 | 0.414 | 0.454 |
| 0.1 ng/ml A | 0.659 | 1.501 | 1.962 | 1.915 | 2.346 | 2.838 | 0.337 | 0.408 | 0.366 |
| 100 μg/ml CDDP | 0.416 | 0.352 | 0.344 | 1.318 | 1.541 | 1.575 | 0.035 | 0.016 | 0.018 |
| 0.1 ng/ml A + 100 | 0.515 | 0.68 | 0.466 | 1.966 | 1.699 | 2.87 | 0.029 | 0.013 | 0.013 |
| μg/ml CDDP | | | | | | | | | |
| Unstimulated cells | 0.542 | 0.867 | 1.625 | 1.538 | 1.944 | 2.859 | 0.3 | 0.414 | 0.454 |
| 1 ng/ml A | 0.86 | 1.258 | 2.426 | 1.925 | 2.484 | 2.834 | 0.344 | 0.394 | 0.651 |
| 0.1 μg/ml CDDP | 0.484 | 0.886 | 0.786 | 1.437 | 1.79 | 2.674 | 0.244 | 0.322 | 0.29 |
| 1 ng/ml A + 0.1 | 0.787 | 0.922 | 0.824 | 2.169 | 2.061 | 2.701 | 0.207 | 0.259 | 0.328 |
| μg/ml CDDP | | | | | | | | | |
| Unstimulated cells | 0.542 | 0.867 | 1.625 | 1.538 | 1.944 | 2.859 | 0.3 | 0.414 | 0.454 |
| 1 ng/ml A | 0.86 | 1.258 | 2.426 | 1.925 | 2.484 | 2.834 | 0.344 | 0.394 | 0.651 |
| 1 μg/ml CDDP | 0.576 | 0.563 | 0.214 | 1.725 | 1.837 | 2.563 | 0.055 | 0.024 | 0.021 |
| 1 ng/ml A + 1 | 0.544 | 0.867 | 0.292 | 1.599 | 1.891 | 2.039 | 0.027 | 0.016 | 0.017 |
| μg/ml CDDP | | | | | | | | | |
| Unstimulated cells | 0.542 | 0.867 | 1.625 | 1.538 | 1.944 | 2.859 | 0.3 | 0.414 | 0.454 |
| 1 ng/ml A | 0.86 | 1.258 | 2.426 | 1.925 | 2.484 | 2.834 | 0.344 | 0.394 | 0.651 |
| 10 μg/ml CDDP | 0.64 | 0.46 | 0.254 | 1.48 | 1.702 | 2.434 | 0.056 | 0.019 | 0.015 |
| 1 ng/ml A + 10 | 0.815 | 0.359 | 0.372 | 2.07 | 1.75 | 2.822 | 0.05 | 0.016 | 0.014 |
| μg/ml CDDP | | | | | | | | | |
| Unstimulated cells | 0.542 | 0.867 | 1.625 | 1.538 | 1.944 | 2.859 | 0.3 | 0.414 | 0.454 |
| 1 ng/ml A | 0.86 | 1.258 | 2.426 | 1.925 | 2.484 | 2.834 | 0.344 | 0.394 | 0.651 |
| 100 μg/ml CDDP | 0.416 | 0.352 | 0.344 | 1.318 | 1.541 | 1.575 | 0.035 | 0.016 | 0.018 |
| 1 ng/ml A + 100 | 0.642 | 0.701 | 0.441 | 2.266 | 1.878 | 2.897 | 0.026 | 0.014 | 0.012 |
| μg/ml CDDP | | | | | | | | | |
| Unstimulated cells | 0.542 | 0.867 | 1.625 | 1.538 | 1.944 | 2.859 | 0.3 | 0.414 | 0.454 |
| 10 ng/ml A | 0.925 | 1.187 | 2.24 | 1.692 | 2.299 | 2.739 | 0.368 | 0.367 | 0.536 |
| 0.1 μg/ml CDDP | 0.484 | 0.886 | 0.786 | 1.437 | 1.79 | 2.674 | 0.244 | 0.322 | 0.29 |
| 10 ng/ml A + 0.1 | 0.936 | 0.989 | 0.886 | 1.742 | 1.891 | 2.143 | 0.212 | 0.22 | 0.281 |
| μg/ml CDDP | | | | | | | | | |
| Unstimulated cells | 0.542 | 0.867 | 1.625 | 1.538 | 1.944 | 2.859 | 0.3 | 0.414 | 0.454 |
| 10 ng/ml A | 0.925 | 1.187 | 2.24 | 1.692 | 2.299 | 2.739 | 0.368 | 0.367 | 0.536 |
| 1 μg/ml CDDP | 0.576 | 0.563 | 0.214 | 1.725 | 1.837 | 2.563 | 0.055 | 0.024 | 0.021 |
| 10 ng/ml A + 1 | 0.557 | 0.463 | 0.343 | 1.833 | 1.749 | 1.863 | 0.027 | 0.016 | 0.025 |
| μg/ml CDDP | | | | | | | | | |
| Unstimulated cells | 0.542 | 0.867 | 1.625 | 1.538 | 1.944 | 2.859 | 0.3 | 0.414 | 0.454 |
| 10 ng/ml A | 0.925 | 1.187 | 2.24 | 1.692 | 2.299 | 2.739 | 0.368 | 0.367 | 0.536 |
| 10 μg/ml CDDP | 0.64 | 0.46 | 0.254 | 1.48 | 1.702 | 2.434 | 0.056 | 0.019 | 0.015 |
| 10 ng/ml A + 10 | 0.67 | 0.344 | 0.324 | 1.753 | 1.929 | 2.775 | 0.056 | 0.013 | 0.016 |
| μg/ml CDDP | | | | | | | | | |
| Unstimulated cells | 0.542 | 0.867 | 1.625 | 1.538 | 1.944 | 2.859 | 0.3 | 0.414 | 0.454 |
| 10 ng/ml A | 0.925 | 1.187 | 2.24 | 1.692 | 2.299 | 2.739 | 0.368 | 0.367 | 0.536 |
| 100 μg/ml CDDP | 0.416 | 0.352 | 0.344 | 1.318 | 1.541 | 1.575 | 0.035 | 0.016 | 0.018 |
| 10 ng/ml A + 100 | 0.715 | 0.406 | 0.535 | 1.755 | 1.757 | 2.622 | 0.025 | 0.012 | 0.011 |
| μg/ml CDDP | | | | | | | | | |

TABLE 13

| | CALU- | | | | | | | | |
|---|---|---|---|---|---|---|---|---|---|
| | 1 | Run 3 | | | | | | | |
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated cells | 0.536 | 0.618 | 1.37 | 1.333 | 2.124 | 2.535 | 0.218 | 0.355 | 0.474 |
| 0.01 ng/ml A | 0.623 | 0.593 | 1.368 | 1.601 | 2.066 | 2.606 | 0.239 | 0.44 | 0.509 |
| 0.1 μg/ml CDDP | 0.492 | 0.389 | 0.505 | 1.492 | 1.491 | 1.835 | 0.178 | 0.281 | 0.295 |
| 0.01 ng/ml A + 0.1 | 0.453 | 0.33 | 0.405 | 1.448 | 1.45 | 1.528 | 0.144 | 0.189 | 0.214 |
| μg/ml CDDP | | | | | | | | | |
| Unstimulated cells | 0.536 | 0.618 | 1.37 | 1.333 | 2.124 | 2.535 | 0.218 | 0.355 | 0.474 |
| 0.01 ng/ml A | 0.623 | 0.593 | 1.368 | 1.601 | 2.066 | 2.606 | 0.239 | 0.44 | 0.509 |
| 1 μg/ml CDDP | 0.255 | 0.158 | 0.184 | 1.295 | 1.463 | 1.452 | 0.029 | 0.033 | 0.031 |
| 0.01 ng/ml A + 1 | 0.219 | 0.171 | 0.221 | 1.084 | 1.067 | 1.177 | 0.019 | 0.025 | 0.022 |
| μg/ml CDDP | | | | | | | | | |
| Unstimulated cells | 0.536 | 0.618 | 1.37 | 1.333 | 2.124 | 2.535 | 0.218 | 0.355 | 0.474 |
| 0.01 ng/ml A | 0.623 | 0.593 | 1.368 | 1.601 | 2.066 | 2.606 | 0.239 | 0.44 | 0.509 |
| 10 μg/ml CDDP | 0.558 | 0.175 | 0.189 | 1.139 | 1.152 | 1.193 | 0.057 | 0.025 | 0.022 |
| 0.01 ng/ml A + 10 | 0.51 | 0.119 | 0.294 | 1.387 | 0.956 | 1.238 | 0.093 | 0.028 | 0.022 |
| μg/ml CDDP | | | | | | | | | |
| Unstimulated cells | 0.536 | 0.618 | 1.37 | 1.333 | 2.124 | 2.535 | 0.218 | 0.355 | 0.474 |
| 0.01 ng/ml A | 0.623 | 0.593 | 1.368 | 1.601 | 2.066 | 2.606 | 0.239 | 0.44 | 0.509 |
| 100 μg/ml CDDP | 0.375 | 0.159 | 0.197 | 0.98 | 1.449 | 1.264 | 0.028 | 0.026 | 0.002 |
| 0.01 ng/ml A + | 0.365 | 0.267 | 0.256 | 1.594 | 1.192 | 1.58 | 0.018 | 0.026 | 0.021 |
| 100 μg/ml CDDP | | | | | | | | | |
| Unstimulated cells | 0.536 | 0.618 | 1.37 | 1.333 | 2.124 | 2.535 | 0.218 | 0.355 | 0.474 |
| 0.1 ng/ml A | 0.68 | 0.571 | 1.346 | 1.531 | 2.086 | 2.536 | 0.235 | 0.394 | 0.492 |
| 0.1 μg/ml CDDP | 0.492 | 0.389 | 0.505 | 1.492 | 1.491 | 1.835 | 0.178 | 0.281 | 0.295 |
| 0.1 ng/ml A + 0.1 | 0.64 | 0.364 | 0.437 | 1.435 | 1.415 | 1.552 | 0.167 | 0.222 | 0.161 |
| μg/ml CDDP | | | | | | | | | |
| Unstimulated cells | 0.536 | 0.618 | 1.37 | 1.333 | 2.124 | 2.535 | 0.218 | 0.355 | 0.474 |
| 0.1 ng/ml A | 0.68 | 0.571 | 1.346 | 1.531 | 2.086 | 2.536 | 0.235 | 0.394 | 0.492 |
| 1 μg/ml CDDP | 0.255 | 0.158 | 0.184 | 1.295 | 1.463 | 1.452 | 0.029 | 0.033 | 0.031 |
| 0.1 ng/ml A + 1 | 0.237 | 0.218 | 0.267 | 1.605 | 1.197 | 1.339 | 0.02 | 0.024 | 0.02 |
| μg/ml CDDP | | | | | | | | | |
| Unstimulated cells | 0.536 | 0.618 | 1.37 | 1.333 | 2.124 | 2.535 | 0.218 | 0.355 | 0.474 |
| 0.1 ng/ml A | 0.68 | 0.571 | 1.346 | 1.531 | 2.086 | 2.536 | 0.235 | 0.394 | 0.492 |
| 10 μg/ml CDDP | 0.558 | 0.175 | 0.189 | 1.139 | 1.152 | 1.193 | 0.057 | 0.025 | 0.022 |
| 0.1 ng/ml A + 10 | 0.476 | 0.239 | 0.71 | 1.514 | 1.103 | 1.362 | 0.081 | 0.025 | 0.019 |
| μg/ml CDDP | | | | | | | | | |
| Unstimulated cells | 0.536 | 0.618 | 1.37 | 1.333 | 2.124 | 2.535 | 0.218 | 0.355 | 0.474 |
| 0.1 ng/ml A | 0.68 | 0.571 | 1.346 | 1.531 | 2.086 | 2.536 | 0.235 | 0.394 | 0.492 |
| 100 μg/ml CDDP | 0.375 | 0.159 | 0.197 | 0.98 | 1.449 | 1.264 | 0.028 | 0.026 | 0.002 |
| 0.1 ng/ml A + 100 | 0.439 | 0.334 | 0.557 | 1.674 | 1.574 | 1.329 | 0.018 | 0.024 | 0.021 |
| μg/ml CDDP | | | | | | | | | |
| Unstimulated cells | 0.536 | 0.618 | 1.37 | 1.333 | 2.124 | 2.535 | 0.218 | 0.355 | 0.474 |
| 1 ng/ml A | 0.902 | 0.73 | 1.694 | 1.463 | 2.039 | 2.642 | 0.249 | 0.426 | 0.45 |
| 0.1 μg/ml CDDP | 0.492 | 0.389 | 0.505 | 1.492 | 1.491 | 1.835 | 0.178 | 0.281 | 0.295 |
| 1 ng/ml A + 0.1 | 0.749 | 0.452 | 0.575 | 1.523 | 1.491 | 1.798 | 0.169 | 0.26 | 0.214 |
| μg/ml CDDP | | | | | | | | | |
| Unstimulated cells | 0.536 | 0.618 | 1.37 | 1.333 | 2.124 | 2.535 | 0.218 | 0.355 | 0.474 |
| 1 ng/ml A | 0.902 | 0.73 | 1.694 | 1.463 | 2.039 | 2.642 | 0.249 | 0.426 | 0.45 |
| 1 μg/ml CDDP | 0.255 | 0.158 | 0.184 | 1.295 | 1.463 | 1.452 | 0.029 | 0.033 | 0.031 |
| 1 ng/ml A + 1 | 0.325 | 0.213 | 0.271 | 1.965 | 1.416 | 1.496 | 0.021 | 0.031 | 0.02 |
| μg/ml CDDP | | | | | | | | | |
| Unstimulated cells | 0.536 | 0.618 | 1.37 | 1.333 | 2.124 | 2.535 | 0.218 | 0.355 | 0.474 |
| 1 ng/ml A | 0.902 | 0.73 | 1.694 | 1.463 | 2.039 | 2.642 | 0.249 | 0.426 | 0.45 |
| 10 μg/ml CDDP | 0.558 | 0.175 | 0.189 | 1.139 | 1.152 | 1.193 | 0.057 | 0.025 | 0.022 |
| 1 ng/ml A + 10 | 0.589 | 0.385 | 0.399 | 1.983 | 1.39 | 1.412 | 0.11 | 0.032 | 0.021 |
| μg/ml CDDP | | | | | | | | | |
| Unstimulated cells | 0.536 | 0.618 | 1.37 | 1.333 | 2.124 | 2.535 | 0.218 | 0.355 | 0.474 |
| 1 ng/ml A | 0.902 | 0.73 | 1.694 | 1.463 | 2.039 | 2.642 | 0.249 | 0.426 | 0.45 |
| 100 μg/ml CDDP | 0.375 | 0.159 | 0.197 | 0.98 | 1.449 | 1.264 | 0.028 | 0.026 | 0.002 |
| 1 ng/ml A + 100 | 0.58 | 0.301 | 0.523 | 1.665 | 1.727 | 1.809 | 0.026 | 0.024 | 0.023 |
| μg/ml CDDP | | | | | | | | | |
| Unstimulated cells | 0.536 | 0.618 | 1.37 | 1.333 | 2.124 | 2.535 | 0.218 | 0.355 | 0.474 |
| 10 ng/ml A | 0.926 | 0.88 | 1.495 | 1.457 | 2.088 | 2.578 | 0.279 | 0.417 | 0.49 |
| 0.1 μg/ml CDDP | 0.492 | 0.389 | 0.505 | 1.492 | 1.491 | 1.835 | 0.178 | 0.281 | 0.295 |
| 10 ng/ml A + 0.1 | 1.02 | 0.598 | 0.617 | 1.54 | 1.775 | 1.607 | 0.188 | 0.253 | 0.153 |
| μg/ml CDDP | | | | | | | | | |
| Unstimulated cells | 0.536 | 0.618 | 1.37 | 1.333 | 2.124 | 2.535 | 0.218 | 0.355 | 0.474 |
| 10 ng/ml A | 0.926 | 0.88 | 1.495 | 1.457 | 2.088 | 2.578 | 0.279 | 0.417 | 0.49 |
| 1 μg/ml CDDP | 0.255 | 0.158 | 0.184 | 1.295 | 1.463 | 1.452 | 0.029 | 0.033 | 0.031 |
| 10 ng/ml A + 1 | 0.402 | 0.213 | 0.207 | 1.493 | 1.551 | 1.32 | 0.024 | 0.03 | 0.019 |
| μg/ml CDDP | | | | | | | | | |
| Unstimulated cells | 0.536 | 0.618 | 1.37 | 1.333 | 2.124 | 2.535 | 0.218 | 0.355 | 0.474 |
| 10 ng/ml A | 0.926 | 0.88 | 1.495 | 1.457 | 2.088 | 2.578 | 0.279 | 0.417 | 0.49 |
| 10 μg/ml CDDP | 0.558 | 0.175 | 0.189 | 1.139 | 1.152 | 1.193 | 0.057 | 0.025 | 0.022 |
| 10 ng/ml A + 10 | 0.692 | 0.567 | 0.382 | 1.391 | 1.123 | 1.109 | 0.083 | 0.026 | 0.02 |
| μg/ml CDDP | | | | | | | | | |
| Unstimulated cells | 0.536 | 0.618 | 1.37 | 1.333 | 2.124 | 2.535 | 0.218 | 0.355 | 0.474 |
| 10 ng/ml A | 0.926 | 0.88 | 1.495 | 1.457 | 2.088 | 2.578 | 0.279 | 0.417 | 0.49 |
| 100 μg/ml CDDP | 0.375 | 0.159 | 0.197 | 0.98 | 1.449 | 1.264 | 0.028 | 0.026 | 0.002 |
| 10 ng/ml A + 100 | 0.724 | 0.39 | 0.385 | 1.776 | 1.989 | 1.629 | 0.021 | 0.021 | 0.021 |
| μg/ml CDDP | | | | | | | | | |

TABLE 14

| | COLO699N | Run 1 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.361 | 0.407 | 0.508 | 1.325 | 1.419 | 1.669 | 0.172 | 0.222 | 0.393 |
| cells | | | | | | | | | |
| 0.01 ng/ml A | 0.368 | 0.392 | 0.476 | 1.635 | 1.586 | 1.671 | 0.147 | 0.251 | 0.38 |
| 0.1 μg/ml CDDP | 0.303 | 0.393 | 0.418 | 1.552 | 1.317 | 1.658 | 0.122 | 0.179 | 0.237 |
| 0.01 ng/ml A + | 0.266 | 0.266 | 0.362 | 1.472 | 1.167 | 1.325 | 0.116 | 0.139 | 0.12 |
| 0.1 μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 0.01 ng/ml A | 0.368 | 0.392 | 0.476 | 1.635 | 1.586 | 1.671 | 0.147 | 0.251 | 0.38 |
| 1 μg/ml CDDP | 0.222 | 0.136 | 0.313 | 1.526 | 1.388 | 1.152 | 0.023 | 0.016 | 0.019 |
| 0.01 ng/ml A + 1 | 0.201 | 0.1 | 0.243 | 1.629 | 1.243 | 1.006 | 0.019 | 0.009 | 0.013 |
| μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 0.01 ng/ml A | 0.368 | 0.392 | 0.476 | 1.635 | 1.586 | 1.671 | 0.147 | 0.251 | 0.38 |
| 10 μg/ml CDDP | 0.313 | 0.47 | 0.256 | 1.384 | 1.524 | 0.922 | 0.384 | 0.012 | 0.016 |
| 0.01 ng/ml A + | 0.263 | 0.127 | 0.166 | 1.183 | 1.194 | 1.053 | 0.057 | 0.01 | 0.014 |
| 10 μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 0.01 ng/ml A | 0.368 | 0.392 | 0.476 | 1.635 | 1.586 | 1.671 | 0.147 | 0.251 | 0.38 |
| 100 μg/ml CDDP | 0.223 | 0.189 | 0.172 | 1.085 | 1.452 | 1.114 | 0.033 | 0.011 | 0.014 |
| 0.01 ng/ml A + | 0.373 | 0.258 | 0.27 | 1.399 | 1.317 | 1.125 | 0.014 | 0.009 | 0.012 |
| 100 μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 0.1 ng/ml A | 0.453 | 0.388 | 0.493 | 1.677 | 1.56 | 1.522 | 0.217 | 0.243 | 0.311 |
| 0.1 μg/ml CDDP | 0.303 | 0.393 | 0.418 | 1.552 | 1.317 | 1.658 | 0.122 | 0.179 | 0.237 |
| 0.1 ng/ml A + 0.1 | 0.307 | 0.238 | 0.261 | 1.39 | 1.352 | 1.293 | 0.114 | 0.122 | 0.118 |
| μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 0.1 ng/ml A | 0.453 | 0.388 | 0.493 | 1.677 | 1.56 | 1.522 | 0.217 | 0.243 | 0.311 |
| 1 μg/ml CDDP | 0.222 | 0.136 | 0.313 | 1.526 | 1.388 | 1.152 | 0.023 | 0.016 | 0.019 |
| 0.1 ng/ml A + 1 | 0.178 | 0.121 | 0.183 | 1.466 | 1.245 | 1.115 | 0.019 | 0.01 | 0.014 |
| μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 0.1 ng/ml A | 0.453 | 0.388 | 0.493 | 1.677 | 1.56 | 1.522 | 0.217 | 0.243 | 0.311 |
| 10 μg/ml CDDP | 0.313 | 0.47 | 0.256 | 1.384 | 1.524 | 0.922 | 0.384 | 0.012 | 0.016 |
| 0.1 ng/ml A + 10 | 0.234 | 0.147 | 0.293 | 1.561 | 1.259 | 1.039 | 0.08 | 0.01 | 0.012 |
| μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 0.1 ng/ml A | 0.453 | 0.388 | 0.493 | 1.677 | 1.56 | 1.522 | 0.217 | 0.243 | 0.311 |
| 100 g/ml CDDP | 0.223 | 0.189 | 0.172 | 1.085 | 1.452 | 1.114 | 0.033 | 0.011 | 0.014 |
| 0.1 ng/ml A + | 0.396 | 0.351 | 0.406 | 1.603 | 1.255 | 1.346 | 0.012 | 0.008 | 0.012 |
| 100 μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 1 ng/ml A | 0.481 | 0.489 | 0.618 | 1.423 | 1.627 | 1.668 | 0.172 | 0.242 | 0.313 |
| 0.1 μg/ml CDDP | 0.303 | 0.393 | 0.418 | 1.552 | 1.317 | 1.658 | 0.122 | 0.179 | 0.237 |
| 1 ng/ml A + 0.1 | 0.399 | 0.356 | 0.395 | 1.433 | 1.531 | 1.261 | 0.099 | 0.117 | 0.12 |
| μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 1 ng/ml A | 0.481 | 0.489 | 0.618 | 1.423 | 1.627 | 1.668 | 0.172 | 0.242 | 0.313 |
| 1 μg/ml CDDP | 0.222 | 0.136 | 0.313 | 1.526 | 1.388 | 1.152 | 0.023 | 0.016 | 0.019 |
| 1 ng/ml A + 1 | 0.229 | 0.184 | 0.152 | 1.209 | 1.727 | 1.117 | 0.019 | 0.009 | 0.014 |
| μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 1 ng/ml A | 0.481 | 0.489 | 0.618 | 1.423 | 1.627 | 1.668 | 0.172 | 0.242 | 0.313 |
| 10 μg/ml CDDP | 0.313 | 0.47 | 0.256 | 1.384 | 1.524 | 0.922 | 0.384 | 0.012 | 0.016 |
| 1 ng/ml A + 10 | 0.273 | 0.161 | 0.352 | 1.498 | 1.252 | 1.087 | 0.065 | 0.012 | 0.016 |
| μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 1 ng/ml A | 0.481 | 0.489 | 0.618 | 1.423 | 1.627 | 1.668 | 0.172 | 0.242 | 0.313 |
| 100 μg/ml CDDP | 0.223 | 0.189 | 0.172 | 1.085 | 1.452 | 1.114 | 0.033 | 0.011 | 0.014 |
| 1 ng/ml A + 100 | 0.335 | 0.262 | 0.392 | 1.572 | 1.658 | 1.464 | 0.026 | 0.011 | 0.013 |
| μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 10 ng/ml A | 0.465 | 0.545 | 0.631 | 1.176 | 1.674 | 1.576 | 0.183 | 0.231 | 0.261 |
| 0.1 μg/ml CDDP | 0.303 | 0.393 | 0.418 | 1.552 | 1.317 | 1.658 | 0.122 | 0.179 | 0.237 |
| 10 ng/ml A + 0.1 | 0.471 | 0.422 | 0.39 | 1.177 | 1.664 | 1.259 | 0.462 | 0.127 | 0.122 |
| μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 10 ng/ml A | 0.465 | 0.545 | 0.631 | 1.176 | 1.674 | 1.576 | 0.183 | 0.231 | 0.261 |
| 1 μg/ml CDDP | 0.222 | 0.136 | 0.313 | 1.526 | 1.388 | 1.152 | 0.023 | 0.016 | 0.019 |
| 10 ng/ml A + 1 | 0.264 | 0.121 | 0.23 | 1.382 | 1.458 | 1.268 | 0.025 | 0.011 | 0.012 |
| μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 10 ng/ml A | 0.465 | 0.545 | 0.631 | 1.176 | 1.674 | 1.576 | 0.183 | 0.231 | 0.261 |
| 10 μg/ml CDDP | 0.313 | 0.47 | 0.256 | 1.384 | 1.524 | 0.922 | 0.384 | 0.012 | 0.016 |
| 10 ng/ml A + 10 | 0.313 | 0.207 | 0.61 | 1.501 | 1.381 | 1.158 | 0.423 | 0.013 | 0.014 |
| μg/ml CDDP | | | | | | | | | |
| Unstimulated | 0.361 | 0.407 | 0.508 | 1.325 | 1.419 | 1.669 | 0.172 | 0.222 | 0.393 |
| cells | | | | | | | | | |
| 10 ng/ml A | 0.465 | 0.545 | 0.631 | 1.176 | 1.674 | 1.576 | 0.183 | 0.231 | 0.261 |
| 100 μg/ml CDDP | 0.223 | 0.189 | 0.172 | 1.085 | 1.452 | 1.114 | 0.033 | 0.011 | 0.014 |
| 10 ng/ml A + 100 | 0.394 | 0.26 | 0.544 | 1.483 | 1.35 | 1.292 | 0.016 | 0.01 | 0.012 |
| μg/ml CDDP | | | | | | | | | |

TABLE 15

| | COLO699N | Run 2 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.672 | 0.918 | 1.659 | 2.018 | 2.636 | 2.758 | 0.54 | 0.82 | 1.34 |
| cells | | | | | | | | | |
| 0.01 ng/ml A | 0.673 | 0.826 | 1.654 | 1.854 | 2.633 | 2.655 | 0.621 | 0.953 | 1.184 |
| 0.1 μg/ml | 0.551 | 0.801 | 1 | 1.709 | 1.975 | 2.145 | 0.362 | 0.636 | 0.73 |
| CDDP | | | | | | | | | |
| 0.01 ng/ml A + | 0.241 | 0.361 | 0.138 | 1.442 | 1.046 | 1.062 | 0.08 | 0.058 | 0.047 |
| 0.1 μg/ml | | | | | | | | | |
| CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 0.01 ng/ml A | 0.673 | 0.826 | 1.654 | 1.854 | 2.633 | 2.655 | 0.621 | 0.953 | 1.184 |
| 1 μg/ml CDDP | 0.232 | 0.311 | 0.337 | 1.61 | 1.253 | 1.256 | 0.055 | 0.04 | 0.044 |
| 0.01 ng/ml A + | 0.31 | 0.605 | 0.335 | 1.545 | 1.26 | 1.04 | 0.041 | 0.031 | 0.015 |
| 1 μg/ml CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 0.01 ng/ml A | 0.673 | 0.826 | 1.654 | 1.854 | 2.633 | 2.655 | 0.621 | 0.953 | 1.184 |
| 10 μg/ml CDDP | 0.504 | 0.205 | 0.127 | 1.523 | 1.27 | 1.524 | 0.178 | 0.049 | 0.057 |
| 0.01 ng/ml A + | 0.5 | 0.575 | 0.177 | 1.347 | 1.386 | 1.137 | 0.117 | 0.027 | 0.015 |
| 10 μg/ml CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 0.01 ng/ml A | 0.673 | 0.826 | 1.654 | 1.854 | 2.633 | 2.655 | 0.621 | 0.953 | 1.184 |
| 100 μg/ml | 0.266 | 0.287 | 0.268 | 1.354 | 1.256 | 1.312 | 0.04 | 0.018 | 0.068 |
| CDDP | | | | | | | | | |
| 0.01 ng/ml A + | 0.44 | 0.474 | 0.409 | 1.575 | 1.536 | 1.381 | 0.037 | 0.022 | 0.01 |
| 100 μg/ml | | | | | | | | | |
| CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 0.1 ng/ml A | 0.679 | 1.005 | 1.369 | 1.823 | 2.562 | 2.607 | 0.537 | 0.825 | 1.187 |
| 0.1 μg/ml | 0.551 | 0.801 | 1 | 1.709 | 1.975 | 2.145 | 0.362 | 0.636 | 0.73 |
| CDDP | | | | | | | | | |
| 0.1 ng/ml A + | 0.583 | 0.527 | 0.529 | 1.768 | 1.851 | 1.894 | 0.32 | 0.339 | 0.359 |
| 0.1 μg/ml | | | | | | | | | |
| CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 0.1 ng/ml A | 0.679 | 1.005 | 1.369 | 1.823 | 2.562 | 2.607 | 0.537 | 0.825 | 1.187 |
| 1 μg/ml CDDP | 0.232 | 0.311 | 0.337 | 1.61 | 1.253 | 1.256 | 0.055 | 0.04 | 0.044 |
| 0.1 ng/ml A + 1 | 0.237 | 0.241 | 0.247 | 1.63 | 1.561 | 1.369 | 0.037 | 0.019 | 0.018 |
| μg/ml CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 0.1 ng/ml A | 0.679 | 1.005 | 1.369 | 1.823 | 2.562 | 2.607 | 0.537 | 0.825 | 1.187 |
| 10 μg/ml CDDP | 0.504 | 0.205 | 0.127 | 1.523 | 1.27 | 1.524 | 0.178 | 0.049 | 0.057 |
| 0.1 ng/ml A + | 0.333 | 0.419 | 0.173 | 1.377 | 1.435 | 1.542 | 0.089 | 0.021 | 0.014 |
| 10 μg/ml CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 0.1 ng/ml A | 0.679 | 1.005 | 1.369 | 1.823 | 2.562 | 2.607 | 0.537 | 0.825 | 1.187 |
| 100 μg/ml | 0.266 | 0.287 | 0.268 | 1.354 | 1.256 | 1.312 | 0.04 | 0.018 | 0.068 |
| CDDP | | | | | | | | | |
| 0.1 ng/ml A + | 0.417 | 0.538 | 0.354 | 1.711 | 1.224 | 1.637 | 0.035 | 0.014 | 0.016 |
| 100 μg/ml | | | | | | | | | |
| CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 1 ng/ml A | 0.728 | 0.992 | 1.456 | 1.999 | 2.7 | 2.694 | 0.52 | 0.789 | 1.271 |
| 0.1 μg/ml | 0.551 | 0.801 | 1 | 1.709 | 1.975 | 2.145 | 0.362 | 0.636 | 0.73 |
| CDDP | | | | | | | | | |
| 1 ng/ml A + 0.1 | 0.56 | 0.556 | 0.519 | 1.853 | 1.941 | 1.846 | 0.285 | 0.459 | 0.47 |
| μg/ml CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 1 ng/ml A | 0.728 | 0.992 | 1.456 | 1.999 | 2.7 | 2.694 | 0.52 | 0.789 | 1.271 |
| 1 μg/ml CDDP | 0.232 | 0.311 | 0.337 | 1.61 | 1.253 | 1.256 | 0.055 | 0.04 | 0.044 |
| 1 ng/ml A + 1 | 0.292 | 0.308 | 0.31 | 1.638 | 1.705 | 1.599 | 0.033 | 0.022 | 0.021 |
| μg/ml CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 1 ng/ml A | 0.728 | 0.992 | 1.456 | 1.999 | 2.7 | 2.694 | 0.52 | 0.789 | 1.271 |
| 10 μg/ml CDDP | 0.504 | 0.205 | 0.127 | 1.523 | 1.27 | 1.524 | 0.178 | 0.049 | 0.057 |
| 1 ng/ml A+ 10 | 0.431 | 0.558 | 0.205 | 1.589 | 1.899 | 1.029 | 0.147 | 0.021 | 0.018 |
| μg/ml CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 1 ng/ml A | 0.728 | 0.992 | 1.456 | 1.999 | 2.7 | 2.694 | 0.52 | 0.789 | 1.271 |
| 100 μg/ml | 0.266 | 0.287 | 0.268 | 1.354 | 1.256 | 1.312 | 0.04 | 0.018 | 0.068 |
| CDDP | | | | | | | | | |
| 1 ng/ml A+ | 0.361 | 0.594 | 0.77 | 1.721 | 1.457 | 1.387 | 0.035 | 0.019 | 0.012 |
| 100 μg/ml | | | | | | | | | |
| CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 10 ng/ml A | 0.939 | 1.15 | 1.622 | 1.852 | 2.733 | 2.693 | 0.57 | 0.703 | 1.27 |
| 0.1 μg/ml | 0.551 | 0.801 | 1 | 1.709 | 1.975 | 2.145 | 0.362 | 0.636 | 0.73 |
| CDDP | | | | | | | | | |
| 10 ng/ml A + | 0.483 | 0.534 | 0.599 | 1.584 | 1.72 | 1.842 | 0.329 | 0.241 | 0.374 |
| 0.1 μg/ml | | | | | | | | | |
| CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 10 ng/ml A | 0.939 | 1.15 | 1.622 | 1.852 | 2.733 | 2.693 | 0.57 | 0.703 | 1.27 |
| 1 μg/ml CDDP | 0.232 | 0.311 | 0.337 | 1.61 | 1.253 | 1.256 | 0.055 | 0.04 | 0.044 |
| 10 ng/ml A + 1 | 0.177 | 0.24 | 0.551 | 1.58 | 1.192 | 1.26 | 0.034 | 0.017 | 0.026 |
| μg/ml CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 10 ng/ml A | 0.939 | 1.15 | 1.622 | 1.852 | 2.733 | 2.693 | 0.57 | 0.703 | 1.27 |
| 10 μg/ml CDDP | 0.504 | 0.205 | 0.127 | 1.523 | 1.27 | 1.524 | 0.178 | 0.049 | 0.057 |
| 10 ng/ml A + | 0.326 | 0.355 | 0.658 | 1.596 | 1.695 | 1.301 | 0.098 | 0.014 | 0.019 |
| 10 μg/ml CDDP | | | | | | | | | |
| Unstimulated | 0.672 | 0.918 | 1.659 | 2.018 | 2.636 | 2.758 | 0.54 | 0.82 | 1.34 |
| cells | | | | | | | | | |
| 10 ng/ml A | 0.939 | 1.15 | 1.622 | 1.852 | 2.733 | 2.693 | 0.57 | 0.703 | 1.27 |
| 100 μg/ml | 0.266 | 0.287 | 0.268 | 1.354 | 1.256 | 1.312 | 0.04 | 0.018 | 0.068 |
| CDDP | | | | | | | | | |
| 10 ng/ml A + | 0.396 | 0.486 | 0.602 | 1.565 | 1.394 | 1.574 | 0.028 | 0.014 | 0.011 |
| 100 μg/ml | | | | | | | | | |
| CDDP | | | | | | | | | |

TABLE 16

| | COLO699N | Run 3 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.695 | 1.4 | 1.705 | 2.251 | 2.62 | 2.719 | 0.551 | 0.704 | 1.143 |
| cells | | | | | | | | | |
| 0.01 ng/ml A | 0.738 | 1.079 | 1.52 | 2.043 | 2.541 | 2.694 | 0.526 | 0.722 | 1.267 |
| 0.1 μg/ml | 0.675 | 0.624 | 0.759 | 1.99 | 1.978 | 1.946 | 0.365 | 0.442 | 0.638 |
| CDDP | | | | | | | | | |
| 0.01 ng/ml A + | 0.275 | 0.152 | 0.119 | 1.383 | 0.962 | 1.041 | 0.073 | 0.053 | 0.057 |
| 0.1 μg/ml | | | | | | | | | |
| CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 0.01 ng/ml A | 0.738 | 1.079 | 1.52 | 2.043 | 2.541 | 2.694 | 0.526 | 0.722 | 1.267 |
| 1 μg/ml CDDP | 0.194 | 0.815 | 0.295 | 1.839 | 1.092 | 1.249 | 0.04 | 0.063 | 0.078 |
| 0.01 ng/ml A + | 0.29 | 0.137 | 0.305 | 1.921 | 1.067 | 1.621 | 0.033 | 0.023 | 0.021 |
| 1 μg/ml CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 0.01 ng/ml A | 0.738 | 1.079 | 1.52 | 2.043 | 2.541 | 2.694 | 0.526 | 0.722 | 1.267 |
| 10 μg/ml | 0.398 | 0.199 | 0.155 | 1.527 | 1.259 | 1.014 | 0.148 | 0.041 | 0.026 |
| CDDP | | | | | | | | | |
| 0.01 ng/ml A + | 0.473 | 0.191 | 0.161 | 1.772 | 1.269 | 1.633 | 0.11 | 0.025 | 0.025 |
| 10 μg/ml | | | | | | | | | |
| CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 0.01 ng/ml A | 0.738 | 1.079 | 1.52 | 2.043 | 2.541 | 2.694 | 0.526 | 0.722 | 1.267 |
| 100 μg/ml | 0.224 | 0.185 | 0.393 | 1.387 | 1.218 | 1.033 | 0.041 | 0.025 | 0.027 |
| CDDP | | | | | | | | | |
| 0.01 ng/ml A + | 0.506 | 0.365 | 0.283 | 1.864 | 1.17 | 1.577 | 0.026 | 0.02 | 0.018 |
| 100 μg/ml | | | | | | | | | |
| CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 0.1 ng/ml A | 0.563 | 0.954 | 1.511 | 2.128 | 2.429 | 2.641 | 0.553 | 0.574 | 1.296 |
| 0.1 μg/ml | 0.675 | 0.624 | 0.759 | 1.99 | 1.978 | 1.946 | 0.365 | 0.442 | 0.638 |
| CDDP | | | | | | | | | |
| 0.1 ng/ml A + | 0.453 | 0.67 | 0.375 | 1.771 | 1.659 | 2.036 | 0.341 | 0.337 | 0.347 |
| 0.1 μg/ml | | | | | | | | | |
| CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 0.1 ng/ml A | 0.563 | 0.954 | 1.511 | 2.128 | 2.429 | 2.641 | 0.553 | 0.574 | 1.296 |
| 1 μg/ml CDDP | 0.194 | 0.815 | 0.295 | 1.839 | 1.092 | 1.249 | 0.04 | 0.063 | 0.078 |
| 0.1 ng/ml A + | 0.305 | 0.351 | 0.224 | 2.038 | 1.155 | 1.671 | 0.032 | 0.021 | 0.024 |
| 1 μg/ml CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 0.1 ng/ml A | 0.563 | 0.954 | 1.511 | 2.128 | 2.429 | 2.641 | 0.553 | 0.574 | 1.296 |
| 10 μg/ml | 0.398 | 0.199 | 0.155 | 1.527 | 1.259 | 1.014 | 0.148 | 0.041 | 0.026 |
| CDDP | | | | | | | | | |
| 0.1 ng/ml A + | 0.82 | 0.269 | 0.156 | 1.712 | 1.258 | 1.382 | 0.065 | 0.023 | 0.023 |
| 10 μg/ml | | | | | | | | | |
| CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 0.1 ng/ml A | 0.563 | 0.954 | 1.511 | 2.128 | 2.429 | 2.641 | 0.553 | 0.574 | 1.296 |
| 100 μg/ml | 0.224 | 0.185 | 0.393 | 1.387 | 1.218 | 1.033 | 0.041 | 0.025 | 0.027 |
| CDDP | | | | | | | | | |
| 0.1 ng/ml A + | 0.546 | 0.572 | 0.499 | 2.004 | 1.275 | 1.512 | 0.027 | 0.017 | 0.022 |
| 100 μg/ml | | | | | | | | | |
| CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 1 ng/ml A | 0.608 | 0.945 | 1.68 | 2.104 | 2.577 | 2.696 | 0.546 | 0.658 | 1.442 |
| 0.1 μg/ml | 0.675 | 0.624 | 0.759 | 1.99 | 1.978 | 1.946 | 0.365 | 0.442 | 0.638 |
| CDDP | | | | | | | | | |
| 1 ng/ml A + | 0.51 | 1.087 | 0.53 | 1.821 | 1.9 | 2.085 | 0.308 | 0.39 | 0.488 |
| 0.1 μg/ml | | | | | | | | | |
| CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 1 ng/ml A | 0.608 | 0.945 | 1.68 | 2.104 | 2.577 | 2.696 | 0.546 | 0.658 | 1.442 |
| 1 CDDP | 0.194 | 0.815 | 0.295 | 1.839 | 1.092 | 1.249 | 0.04 | 0.063 | 0.078 |
| 1 ng/ml A + 1 | 0.326 | 0.336 | 0.357 | 1.828 | 1.242 | 1.634 | 0.033 | 0.02 | 0.02 |
| μg/ml CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 1 ng/ml A | 0.608 | 0.945 | 1.68 | 2.104 | 2.577 | 2.696 | 0.546 | 0.658 | 1.442 |
| 10 μg/ml | 0.398 | 0.199 | 0.155 | 1.527 | 1.259 | 1.014 | 0.148 | 0.041 | 0.026 |
| CDP | | | | | | | | | |
| 1 ng/ml A + 10 | 0.628 | 0.475 | 0.317 | 1.79 | 1.286 | 1.318 | 0.147 | 0.024 | 0.021 |
| μg/ml CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 1 ng/ml A | 0.608 | 0.945 | 1.68 | 2.104 | 2.577 | 2.696 | 0.546 | 0.658 | 1.442 |
| 100 μg/ml | 0.224 | 0.185 | 0.393 | 1.387 | 1.218 | 1.033 | 0.041 | 0.025 | 0.027 |
| CDDP | | | | | | | | | |
| 1 ng/ml A + | 0.383 | 1.451 | 0.87 | 1.933 | 1.389 | 1.515 | 0.028 | 0.019 | 0.019 |
| 100 μg/ml | | | | | | | | | |
| CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 10 ng/ml A | 0.602 | 0.937 | 1.76 | 1.915 | 2.591 | 2.668 | 0.583 | 0.645 | 1.341 |
| 0.1 μg/ml | 0.675 | 0.624 | 0.759 | 1.99 | 1.978 | 1.946 | 0.365 | 0.442 | 0.638 |
| CDDP | | | | | | | | | |
| 10 ng/ml A + | 0.368 | 0.428 | 0.546 | 1.669 | 2.003 | 1.826 | 0.333 | 0.316 | 0.316 |
| 0.1 μg/ml | | | | | | | | | |
| CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 10 ng/ml A | 0.602 | 0.937 | 1.76 | 1.915 | 2.591 | 2.668 | 0.583 | 0.645 | 1.341 |
| 1 μg/ml CDDP | 0.194 | 0.815 | 0.295 | 1.839 | 1.092 | 1.249 | 0.04 | 0.063 | 0.078 |
| 10 ng/ml A + 1 | 0.284 | 0.38 | 0.387 | 1.556 | 1.042 | 1.721 | 0.034 | 0.024 | 0.02 |
| μg/ml CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 10 ng/ml A | 0.602 | 0.937 | 1.76 | 1.915 | 2.591 | 2.668 | 0.583 | 0.645 | 1.341 |
| 10 μg/ml | 0.398 | 0.199 | 0.155 | 1.527 | 1.259 | 1.014 | 0.148 | 0.041 | 0.026 |
| CDDP | | | | | | | | | |
| 10 ng/ml A + | 0.437 | 0.305 | 0.382 | 1.583 | 1.198 | 1.989 | 0.089 | 0.019 | 0.021 |
| 10 μg/ml | | | | | | | | | |
| CDDP | | | | | | | | | |
| Unstimulated | 0.695 | 1.4 | 1.705 | 2.251 | 2.62 | 2.719 | 0.551 | 0.704 | 1.143 |
| cells | | | | | | | | | |
| 10 ng/ml A | 0.602 | 0.937 | 1.76 | 1.915 | 2.591 | 2.668 | 0.583 | 0.645 | 1.341 |
| 100 μg/ml | 0.224 | 0.185 | 0.393 | 1.387 | 1.218 | 1.033 | 0.041 | 0.025 | 0.027 |
| CDDP | | | | | | | | | |
| 10 ng/ml A + | 0.337 | 0.758 | 0.818 | 1.58 | 1.507 | 1.719 | 0.029 | 0.018 | 0.016 |
| 100 μg/ml | | | | | | | | | |
| CDDP | | | | | | | | | |

TABLE 17

| | COR-L | | | | | | | | |
|---|---|---|---|---|---|---|---|---|---|
| | 105 | Run 1 | | | | | | | |
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.387 | 0.327 | 0.541 | 1.184 | 1.586 | 1.872 | 0.108 | 0.157 | 0.091 |
| cells | | | | | | | | | |
| 0.01 ng/ml A | 0.611 | 0.356 | 0.488 | 1.471 | 1.806 | 1.826 | 0.078 | 0.137 | 0.107 |
| 0.1 μg/ml CDDP | 0.413 | 0.599 | 0.298 | 1.526 | 1.705 | 1.491 | 0.083 | 0.099 | 0.056 |
| 0.01 ng/ml A + | 0.464 | 0.386 | 0.228 | 1.543 | 1.549 | 1.571 | 0.066 | 0.116 | 0.054 |
| 0.1 μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 0.01 ng/ml A | 0.611 | 0.356 | 0.488 | 1.471 | 1.806 | 1.826 | 0.078 | 0.137 | 0.107 |
| 1 μg/ml CDDP | 0.431 | 0.643 | 0.23 | 1.714 | 1.716 | 1.356 | 0.023 | 0.018 | 0.017 |
| 0.01 ng/ml A + 1 | 0.295 | 0.17 | 0.115 | 1.56 | 1.468 | 1.596 | 0.014 | 0.017 | 0.016 |
| μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 0.01 ng/ml A | 0.611 | 0.356 | 0.488 | 1.471 | 1.806 | 1.826 | 0.078 | 0.137 | 0.107 |
| 10 μg/ml CDDP | 0.484 | 0.632 | 0.216 | 1.386 | 1.465 | 1.544 | 0.057 | 0.023 | 0.021 |
| 0.01 ng/ml A + 10 | 0.62 | 0.342 | 0.207 | 1.466 | 1.432 | 1.411 | 0.032 | 0.021 | 0.019 |
| μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 0.01 ng/ml A | 0.611 | 0.356 | 0.488 | 1.471 | 1.806 | 1.826 | 0.078 | 0.137 | 0.107 |
| 100 μg/ml CDDP | 0.307 | 0.379 | 0.244 | 1.523 | 1.415 | 1.456 | 0.015 | 0.016 | 0.018 |
| 0.01 ng/ml A + | 0.84 | 0.415 | 0.289 | 1.682 | 1.77 | 1.549 | 0.012 | 0.015 | 0.016 |
| 100 μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 0.1 ng/ml A | 0.621 | 0.406 | 0.502 | 2.037 | 1.981 | 1.779 | 0.164 | 0.094 | 0.086 |
| 0.1 μg/ml CDDP | 0.413 | 0.599 | 0.298 | 1.526 | 1.705 | 1.491 | 0.083 | 0.099 | 0.056 |
| 0.1 ng/ml A + 0.1 | 0.623 | 0.273 | 0.257 | 1.57 | 1.59 | 1.606 | 0.091 | 0.069 | 0.044 |
| μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 0.1 ng/ml A | 0.621 | 0.406 | 0.502 | 2.037 | 1.981 | 1.779 | 0.164 | 0.094 | 0.086 |
| 1 μg/ml CDDP | 0.431 | 0.643 | 0.23 | 1.714 | 1.716 | 1.356 | 0.023 | 0.018 | 0.017 |
| 0.1 ng/ml A + 1 | 0.527 | 0.157 | 0.189 | 1.729 | 1.658 | 1.693 | 0.013 | 0.012 | 0.014 |
| μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 0.1 ng/ml A | 0.621 | 0.406 | 0.502 | 2.037 | 1.981 | 1.779 | 0.164 | 0.094 | 0.086 |
| 10 CDDP | 0.484 | 0.632 | 0.216 | 1.386 | 1.465 | 1.544 | 0.057 | 0.023 | 0.021 |
| 0.1 ng/ml A + 10 | 0.373 | 0.345 | 0.195 | 1.47 | 1.68 | 1.571 | 0.035 | 0.018 | 0.019 |
| μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 0.1 ng/ml A | 0.621 | 0.406 | 0.502 | 2.037 | 1.981 | 1.779 | 0.164 | 0.094 | 0.086 |
| 100 μg/ml CDDP | 0.307 | 0.379 | 0.244 | 1.523 | 1.415 | 1.456 | 0.015 | 0.016 | 0.018 |
| 0.1 ng/ml A + 100 | 0.505 | 0.662 | 0.288 | 1.925 | 2.094 | 2.05 | 0.014 | 0.014 | 0.016 |
| μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 1 ng/ml A | 0.556 | 0.418 | 0.591 | 1.6 | 2.089 | 2.015 | 0.096 | 0.089 | 0.151 |
| 0.1 μg/ml CDDP | 0.413 | 0.599 | 0.298 | 1.526 | 1.705 | 1.491 | 0.083 | 0.099 | 0.056 |
| 1 ng/ml A + 0.1 | 0.583 | 0.31 | 0.317 | 2.192 | 1.795 | 2.018 | 0.112 | 0.064 | 0.05 |
| μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 1 ng/ml A | 0.556 | 0.418 | 0.591 | 1.6 | 2.089 | 2.015 | 0.096 | 0.089 | 0.151 |
| 1 μg/ml CDDP | 0.431 | 0.643 | 0.23 | 1.714 | 1.716 | 1.356 | 0.023 | 0.018 | 0.017 |
| 1 ng/ml A + 1 | 0.595 | 0.234 | 0.292 | 1.716 | 1.562 | 2.202 | 0.015 | 0.017 | 0.017 |
| μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 1 ng/ml A | 0.556 | 0.418 | 0.591 | 1.6 | 2.089 | 2.015 | 0.096 | 0.089 | 0.151 |
| 10 μg/ml CDDP | 0.484 | 0.632 | 0.216 | 1.386 | 1.465 | 1.544 | 0.057 | 0.023 | 0.021 |
| 1 ng/ml A + 10 | 0.527 | 0.335 | 0.353 | 1.987 | 1.548 | 1.985 | 0.047 | 0.021 | 0.018 |
| μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 1 ng/ml A | 0.556 | 0.418 | 0.591 | 1.6 | 2.089 | 2.015 | 0.096 | 0.089 | 0.151 |
| 100 μg/ml CDDP | 0.307 | 0.379 | 0.244 | 1.523 | 1.415 | 1.456 | 0.015 | 0.016 | 0.018 |
| 1 ng/ml A + 100 | 0.338 | 0.394 | 0.445 | 2.317 | 2.012 | 2.093 | 0.011 | 0.016 | 0.014 |
| μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 10 ng/ml A | 0.61 | 0.397 | 0.602 | 1.338 | 1.774 | 1.878 | 0.097 | 0.117 | 0.145 |
| 0.1 μg/ml CDDP | 0.413 | 0.599 | 0.298 | 1.526 | 1.705 | 1.491 | 0.083 | 0.099 | 0.056 |
| 10 ng/ml A + 0.1 | 0.537 | 0.37 | 0.753 | 1.517 | 1.498 | 1.779 | 0.082 | 0.095 | 0.058 |
| μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 10 ng/ml A | 0.61 | 0.397 | 0.602 | 1.338 | 1.774 | 1.878 | 0.097 | 0.117 | 0.145 |
| 1 μg/ml CDDP | 0.431 | 0.643 | 0.23 | 1.714 | 1.716 | 1.356 | 0.023 | 0.018 | 0.017 |
| 10 ng/ml A + 1 | 0.461 | 0.348 | 0.369 | 1.449 | 1.485 | 1.497 | 0.012 | 0.016 | 0.015 |
| μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 10 ng/ml A | 0.61 | 0.397 | 0.602 | 1.338 | 1.774 | 1.878 | 0.097 | 0.117 | 0.145 |
| 10 μg/ml CDDP | 0.484 | 0.632 | 0.216 | 1.386 | 1.465 | 1.544 | 0.057 | 0.023 | 0.021 |
| 10 ng/ml A + 10 | 0.492 | 0.331 | 0.485 | 1.372 | 1.478 | 1.688 | 0.039 | 0.021 | 0.017 |
| μg/ml CDDP | | | | | | | | | |
| Unstimulated | 0.387 | 0.327 | 0.541 | 1.184 | 1.586 | 1.872 | 0.108 | 0.157 | 0.091 |
| cells | | | | | | | | | |
| 10 ng/ml A | 0.61 | 0.397 | 0.602 | 1.338 | 1.774 | 1.878 | 0.097 | 0.117 | 0.145 |
| 100 μg/ml CDDP | 0.307 | 0.379 | 0.244 | 1.523 | 1.415 | 1.456 | 0.015 | 0.016 | 0.018 |
| 10 ng/ml A + 100 | 0.292 | 1.052 | 0.64 | 1.841 | 1.602 | 2.574 | 0.012 | 0.014 | 0.013 |
| μg/ml CDDP | | | | | | | | | |

TABLE 18

| | COR-L | | | | | | | | |
|---|---|---|---|---|---|---|---|---|---|
| | 105 | Run 2 | | | | | | | |
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.36 | 0.649 | 0.773 | 1.269 | 2.865 | 2.89 | 0.114 | 0.126 | 0.192 |
| cells | | | | | | | | | |
| 0.01 ng/ml A | 0.36 | 0.63 | 0.624 | 1.365 | 2.81 | 2.917 | 0.062 | 0.108 | 0.143 |
| 0.1 μg/ml CDDP | 0.388 | 0.709 | 0.457 | 1.731 | 2.759 | 2.681 | 0.056 | 0.069 | 0.088 |
| 0.01 ng/ml A + | 0.393 | 0.535 | 0.402 | 1.762 | 2.825 | 2.792 | 0.055 | 0.082 | 0.06 |
| 0.1 μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 0.01 ng/ml A | 0.36 | 0.63 | 0.624 | 1.365 | 2.81 | 2.917 | 0.062 | 0.108 | 0.143 |
| 1 μg/ml CDDP | 0.363 | 0.517 | 0.263 | 1.862 | 2.832 | 2.654 | 0.02 | 0.018 | 0.012 |
| 0.01 ng/ml A + 1 | 0.294 | 0.39 | 0.169 | 1.416 | 2.441 | 2.782 | 0.021 | 0.019 | 0.011 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 0.01 ng/ml A | 0.36 | 0.63 | 0.624 | 1.365 | 2.81 | 2.917 | 0.062 | 0.108 | 0.143 |
| 10 μg/ml CDDP | 0.358 | 0.653 | 0.387 | 1.565 | 2.85 | 2.737 | 0.039 | 0.023 | 0.01 |
| 0.01 ng/ml A + | 0.485 | 0.362 | 0.116 | 1.406 | 2.157 | 2.882 | 0.029 | 0.022 | 0.011 |
| 10 μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 0.01 ng/ml A | 0.36 | 0.63 | 0.624 | 1.365 | 2.81 | 2.917 | 0.062 | 0.108 | 0.143 |
| 100 μg/ml CDDP | 0.282 | 0.341 | 0.389 | 1.113 | 2.596 | 2.819 | 0.019 | 0.018 | 0.009 |
| 0.01 ng/ml A + | 0.397 | 0.163 | 0.092 | 1.324 | 2.083 | 2.169 | 0.017 | 0.011 | 0.009 |
| 100 μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 0.1 ng/ml A | 0.483 | 0.759 | 0.632 | 1.35 | 2.785 | 2.817 | 0.082 | 0.119 | 0.145 |
| 0.1 μg/ml CDDP | 0.388 | 0.709 | 0.457 | 1.731 | 2.759 | 2.681 | 0.056 | 0.069 | 0.088 |
| 0.1 ng/ml A + 0.1 | 0.518 | 0.625 | 0.44 | 1.698 | 2.899 | 2.755 | 0.046 | 0.07 | 0.046 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 0.1 ng/ml A | 0.483 | 0.759 | 0.632 | 1.35 | 2.785 | 2.817 | 0.082 | 0.119 | 0.145 |
| 1 μg/ml CDDP | 0.363 | 0.517 | 0.263 | 1.862 | 2.832 | 2.654 | 0.02 | 0.018 | 0.012 |
| 0.1 ng/ml A + 1 | 0.334 | 0.561 | 0.251 | 1.391 | 2.782 | 2.88 | 0.018 | 0.016 | 0.012 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 0.1 ng/ml A | 0.483 | 0.759 | 0.632 | 1.35 | 2.785 | 2.817 | 0.082 | 0.119 | 0.145 |
| 10 μg/ml CDDP | 0.358 | 0.653 | 0.387 | 1.565 | 2.85 | 2.737 | 0.039 | 0.023 | 0.01 |
| 0.1 ng/ml A + 10 | 0.465 | 0.305 | 0.148 | 1.787 | 2.727 | 2.775 | 0.04 | 0.023 | 0.011 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 0.1 ng/ml A | 0.483 | 0.759 | 0.632 | 1.35 | 2.785 | 2.817 | 0.082 | 0.119 | 0.145 |
| 100 μg/ml CDDP | 0.282 | 0.341 | 0.389 | 1.113 | 2.596 | 2.819 | 0.019 | 0.018 | 0.009 |
| 0.1 ng/ml A + | 0.335 | 0.215 | 0.126 | 2.09 | 2.347 | 2.674 | 0.016 | 0.004 | 0.008 |
| 100 μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 1 ng/ml A | 0.469 | 0.944 | 0.792 | 1.433 | 2.855 | 2.803 | 0.061 | 0.104 | 0.154 |
| 0.1 μg/ml CDDP | 0.388 | 0.709 | 0.457 | 1.731 | 2.759 | 2.681 | 0.056 | 0.069 | 0.088 |
| 1 ng/ml A + 0.1 | 0.475 | 0.542 | 0.451 | 1.357 | 2.877 | 2.689 | 0.045 | 0.055 | 0.073 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 1 ng/ml A | 0.469 | 0.944 | 0.792 | 1.433 | 2.855 | 2.803 | 0.061 | 0.104 | 0.154 |
| 1 μg/ml CDDP | 0.363 | 0.517 | 0.263 | 1.862 | 2.832 | 2.654 | 0.02 | 0.018 | 0.012 |
| 1 ng/ml A + 1 | 0.308 | 0.452 | 0.336 | 1.571 | 2.669 | 2.643 | 0.021 | 0.013 | 0.007 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 1 ng/ml A | 0.469 | 0.944 | 0.792 | 1.433 | 2.855 | 2.803 | 0.061 | 0.104 | 0.154 |
| 10 μg/ml CDDP | 0.358 | 0.653 | 0.387 | 1.565 | 2.85 | 2.737 | 0.039 | 0.023 | 0.01 |
| 1 ng/ml A + 10 | 0.591 | 0.422 | 0.159 | 1.982 | 2.837 | 2.751 | 0.039 | 0.025 | 0.011 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 1 ng/ml A | 0.469 | 0.944 | 0.792 | 1.433 | 2.855 | 2.803 | 0.061 | 0.104 | 0.154 |
| 100 μg/ml CDDP | 0.282 | 0.341 | 0.389 | 1.113 | 2.596 | 2.819 | 0.019 | 0.018 | 0.009 |
| 1 ng/ml A + 100 | 0.563 | 0.173 | 0.159 | 2.198 | 2.565 | 2.389 | 0.021 | 0.01 | 0.01 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 10 ng/ml A | 0.554 | 0.722 | 0.618 | 1.511 | 2.786 | 2.805 | 0.063 | 0.102 | 0.099 |
| 0.1 μg/ml CDDP | 0.388 | 0.709 | 0.457 | 1.731 | 2.759 | 2.681 | 0.056 | 0.069 | 0.088 |
| 10 ng/ml A + 0.1 | 0.574 | 0.613 | 0.511 | 1.134 | 2.818 | 2.721 | 0.06 | 0.082 | 0.056 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 10 ng/ml A | 0.554 | 0.722 | 0.618 | 1.511 | 2.786 | 2.805 | 0.063 | 0.102 | 0.099 |
| 1 μg/ml CDDP | 0.363 | 0.517 | 0.263 | 1.862 | 2.832 | 2.654 | 0.02 | 0.018 | 0.012 |
| 10 ng/ml A + 1 | 0.302 | 0.457 | 0.273 | 1.8 | 2.655 | 2.873 | 0.019 | 0.014 | 0.008 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 10 ng/ml A | 0.554 | 0.722 | 0.618 | 1.511 | 2.786 | 2.805 | 0.063 | 0.102 | 0.099 |
| 10 μg/ml CDDP | 0.358 | 0.653 | 0.387 | 1.565 | 2.85 | 2.737 | 0.039 | 0.023 | 0.01 |
| 10 ng/ml A + 10 | 0.496 | 0.224 | 0.158 | 1.879 | 2.645 | 2.771 | 0.045 | 0.019 | 0.012 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 10 ng/ml A | 0.554 | 0.722 | 0.618 | 1.511 | 2.786 | 2.805 | 0.063 | 0.102 | 0.099 |
| 100 μg/ml CDDP | 0.282 | 0.341 | 0.389 | 1.113 | 2.596 | 2.819 | 0.019 | 0.018 | 0.009 |
| 10 ng/ml A + 100 | 0.535 | 0.123 | 0.151 | 1.887 | 2.301 | 2.19 | 0.018 | 0.015 | 0.009 |
| μg/ml CDDP | | | | | | | | | |

TABLE 19

| | COR-L | | | | | | | | |
|---|---|---|---|---|---|---|---|---|---|
| | 105 | Run 3 | | | | | | | |
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.36 | 0.649 | 0.773 | 1.269 | 2.865 | 2.89 | 0.114 | 0.126 | 0.192 |
| cells | | | | | | | | | |
| 0.01 ng/ml A | 0.36 | 0.63 | 0.624 | 1.365 | 2.81 | 2.917 | 0.062 | 0.108 | 0.143 |
| 0.1 μg/ml CDDP | 0.388 | 0.709 | 0.457 | 1.731 | 2.759 | 2.681 | 0.056 | 0.069 | 0.088 |
| 0.01 ng/ml A + | 0.393 | 0.535 | 0.402 | 1.762 | 2.825 | 2.792 | 0.055 | 0.082 | 0.06 |
| 0.1 μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 0.01 ng/ml A | 0.36 | 0.63 | 0.624 | 1.365 | 2.81 | 2.917 | 0.062 | 0.108 | 0.143 |
| 1 μg/ml CDDP | 0.363 | 0.517 | 0.263 | 1.862 | 2.832 | 2.654 | 0.02 | 0.018 | 0.012 |
| 0.01 ng/ml A + 1 | 0.294 | 0.39 | 0.169 | 1.416 | 2.441 | 2.782 | 0.021 | 0.019 | 0.011 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 0.01 ng/ml A | 0.36 | 0.63 | 0.624 | 1.365 | 2.81 | 2.917 | 0.062 | 0.108 | 0.143 |
| 10 μg/ml CDDP | 0.358 | 0.653 | 0.387 | 1.565 | 2.85 | 2.737 | 0.039 | 0.023 | 0.01 |
| 0.01 ng/ml A + | 0.485 | 0.362 | 0.116 | 1.406 | 2.157 | 2.882 | 0.029 | 0.022 | 0.011 |
| 10 μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 0.01 ng/ml A | 0.36 | 0.63 | 0.624 | 1.365 | 2.81 | 2.917 | 0.062 | 0.108 | 0.143 |
| 100 μg/ml CDDP | 0.282 | 0.341 | 0.389 | 1.113 | 2.596 | 2.819 | 0.019 | 0.018 | 0.009 |
| 0.01 ng/ml A + | 0.397 | 0.163 | 0.092 | 1.324 | 2.083 | 2.169 | 0.017 | 0.011 | 0.009 |
| 100 μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 0.1 ng/ml A | 0.483 | 0.759 | 0.632 | 1.35 | 2.785 | 2.817 | 0.082 | 0.119 | 0.145 |
| 0.1 μg/ml CDDP | 0.388 | 0.709 | 0.457 | 1.731 | 2.759 | 2.681 | 0.056 | 0.069 | 0.088 |
| 0.1 ng/ml A + 0.1 | 0.518 | 0.625 | 0.44 | 1.698 | 2.899 | 2.755 | 0.046 | 0.07 | 0.046 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 0.1 ng/ml A | 0.483 | 0.759 | 0.632 | 1.35 | 2.785 | 2.817 | 0.082 | 0.119 | 0.145 |
| 1 μg/ml CDDP | 0.363 | 0.517 | 0.263 | 1.862 | 2.832 | 2.654 | 0.02 | 0.018 | 0.012 |
| 0.1 ng/ml A + 1 | 0.334 | 0.561 | 0.251 | 1.391 | 2.782 | 2.88 | 0.018 | 0.016 | 0.012 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 0.1 ng/ml A | 0.483 | 0.759 | 0.632 | 1.35 | 2.785 | 2.817 | 0.082 | 0.119 | 0.145 |
| 10 μg/ml CDDP | 0.358 | 0.653 | 0.387 | 1.565 | 2.85 | 2.737 | 0.039 | 0.023 | 0.01 |
| 0.1 ng/ml A + 10 | 0.465 | 0.305 | 0.148 | 1.787 | 2.727 | 2.775 | 0.04 | 0.023 | 0.011 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 0.1 ng/ml A | 0.483 | 0.759 | 0.632 | 1.35 | 2.785 | 2.817 | 0.082 | 0.119 | 0.145 |
| 100 μg/ml CDDP | 0.282 | 0.341 | 0.389 | 1.113 | 2.596 | 2.819 | 0.019 | 0.018 | 0.009 |
| 0.1 ng/ml A + | 0.335 | 0.215 | 0.126 | 2.09 | 2.347 | 2.674 | 0.016 | 0.004 | 0.008 |
| 100 μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 1 ng/ml A | 0.469 | 0.944 | 0.792 | 1.433 | 2.855 | 2.803 | 0.061 | 0.104 | 0.154 |
| 0.1 μg/ml CDDP | 0.388 | 0.709 | 0.457 | 1.731 | 2.759 | 2.681 | 0.056 | 0.069 | 0.088 |
| 1 ng/ml A + 0.1 | 0.475 | 0.542 | 0.451 | 1.357 | 2.877 | 2.689 | 0.045 | 0.055 | 0.073 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 1 ng/ml A | 0.469 | 0.944 | 0.792 | 1.433 | 2.855 | 2.803 | 0.061 | 0.104 | 0.154 |
| 1 μg/ml CDDP | 0.363 | 0.517 | 0.263 | 1.862 | 2.832 | 2.654 | 0.02 | 0.018 | 0.012 |
| 1 ng/ml A + 1 | 0.308 | 0.452 | 0.336 | 1.571 | 2.669 | 2.643 | 0.021 | 0.013 | 0.007 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 1 ng/ml A | 0.469 | 0.944 | 0.792 | 1.433 | 2.855 | 2.803 | 0.061 | 0.104 | 0.154 |
| 10 μg/ml CDDP | 0.358 | 0.653 | 0.387 | 1.565 | 2.85 | 2.737 | 0.039 | 0.023 | 0.01 |
| 1 ng/ml A + 10 | 0.591 | 0.422 | 0.159 | 1.982 | 2.837 | 2.751 | 0.039 | 0.025 | 0.011 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 1 ng/ml A | 0.469 | 0.944 | 0.792 | 1.433 | 2.855 | 2.803 | 0.061 | 0.104 | 0.154 |
| 100 μg/ml CDDP | 0.282 | 0.341 | 0.389 | 1.113 | 2.596 | 2.819 | 0.019 | 0.018 | 0.009 |
| 1 ng/ml A + 100 | 0.563 | 0.173 | 0.159 | 2.198 | 2.565 | 2.389 | 0.021 | 0.01 | 0.01 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 10 ng/ml A | 0.554 | 0.722 | 0.618 | 1.511 | 2.786 | 2.805 | 0.063 | 0.102 | 0.099 |
| 0.1 μg/ml CDDP | 0.388 | 0.709 | 0.457 | 1.731 | 2.759 | 2.681 | 0.056 | 0.069 | 0.088 |
| 10 ng/ml A + 0.1 | 0.574 | 0.613 | 0.511 | 1.134 | 2.818 | 2.721 | 0.06 | 0.082 | 0.056 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 10 ng/ml A | 0.554 | 0.722 | 0.618 | 1.511 | 2.786 | 2.805 | 0.063 | 0.102 | 0.099 |
| 1 μg/ml CDDP | 0.363 | 0.517 | 0.263 | 1.862 | 2.832 | 2.654 | 0.02 | 0.018 | 0.012 |
| 10 ng/ml A + 1 | 0.302 | 0.457 | 0.273 | 1.8 | 2.655 | 2.873 | 0.019 | 0.014 | 0.008 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 10 ng/ml A | 0.554 | 0.722 | 0.618 | 1.511 | 2.786 | 2.805 | 0.063 | 0.102 | 0.099 |
| 10 μg/ml CDDP | 0.358 | 0.653 | 0.387 | 1.565 | 2.85 | 2.737 | 0.039 | 0.023 | 0.01 |
| 10 ng/ml A + 10 | 0.496 | 0.224 | 0.158 | 1.879 | 2.645 | 2.771 | 0.045 | 0.019 | 0.012 |
| μg/ml CDDP | | | | | | | | | |
| Unstimulated | 0.36 | 0.649 | 0.773 | 1.269 | 2.865 | 2.89 | 0.114 | 0.126 | 0.192 |
| cells | | | | | | | | | |
| 10 ng/ml A | 0.554 | 0.722 | 0.618 | 1.511 | 2.786 | 2.805 | 0.063 | 0.102 | 0.099 |
| 100 μg/ml CDDP | 0.282 | 0.341 | 0.389 | 1.113 | 2.596 | 2.819 | 0.019 | 0.018 | 0.009 |
| 10 ng/ml A + 100 | 0.535 | 0.123 | 0.151 | 1.887 | 2.301 | 2.19 | 0.018 | 0.015 | 0.009 |
| μg/ml CDDP | | | | | | | | | |

TABLE 20

| | HCT-15 | Run 1 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.946 | 1.555 | 2.958 | 2.219 | 2.766 | 2.832 | 0.274 | 0.429 | 0.659 |
| cells | | | | | | | | | |
| 0.01 ng/ml A | 0.938 | 0.716 | 3.796 | 2.357 | 2.747 | 2.87 | 0.237 | 0.392 | 0.574 |
| 0.1 μg/ml CDDP | 0.787 | 0.99 | 2.312 | 2.592 | 2.747 | 2.792 | 0.218 | 0.324 | 0.464 |
| 0.01 ng/ml A + | 0.667 | 0.791 | 0.917 | 2.376 | 2.506 | 2.55 | 0.207 | 0.239 | 0.285 |
| 0.1 μg/ml CDDP | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 0.01 ng/ml A | 0.938 | 0.716 | 3.796 | 2.357 | 2.747 | 2.87 | 0.237 | 0.392 | 0.574 |
| 1 μg/ml CDDP | 0.451 | 0.187 | 0.425 | 1.974 | 2.397 | 1.697 | 0.032 | 0.032 | 0.025 |
| 0.01 ng/ml A + 1 | 0.301 | 0.274 | 0.181 | 1.948 | 1.724 | 1.511 | 0.023 | 0.025 | 0.015 |
| μg/ml CDDP | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 0.01 ng/ml A | 0.938 | 0.716 | 3.796 | 2.357 | 2.747 | 2.87 | 0.237 | 0.392 | 0.574 |
| 10 μg/ml CDDP | 0.59 | 0.277 | 0.282 | 1.657 | 2.449 | 1.71 | 0.1 | 0.046 | 0.03 |
| 0.01 ng/ml A + 10 | 0.536 | 0.371 | 0.381 | 1.968 | 1.73 | 1.648 | 0.118 | 0.075 | 0.032 |
| μg/ml CDDP | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 0.01 ng/ml A | 0.938 | 0.716 | 3.796 | 2.357 | 2.747 | 2.87 | 0.237 | 0.392 | 0.574 |
| 100 μg/ml CDDP | 0.456 | 0.214 | 0.231 | 1.854 | 1.937 | 1.634 | 0.034 | 0.023 | 0.017 |
| 0.01 ng/ml A + | 0.442 | 0.401 | 0.4 | 1.876 | 1.927 | 1.771 | 0.021 | 0.02 | 0.015 |
| 100 μg/ml CDDP | | | | | | | | | |
| Average | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 0.1 ng/ml A | 1.033 | 1.406 | 3.196 | 2.341 | 2.78 | 2.85 | 0.295 | 0.362 | 0.594 |
| 0.1 μg/ml CDDP | 0.787 | 0.99 | 2.312 | 2.592 | 2.747 | 2.792 | 0.218 | 0.324 | 0.464 |
| 0.1 ng/ml A + 0.1 | 0.775 | 0.824 | 1.399 | 2.391 | 2.78 | 2.718 | 0.251 | 0.252 | 0.314 |
| μg/ml CDDP | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 0.1 ng/ml A | 1.033 | 1.406 | 3.196 | 2.341 | 2.78 | 2.85 | 0.295 | 0.362 | 0.594 |
| 1 μg/ml CDDP | 0.451 | 0.187 | 0.425 | 1.974 | 2.397 | 1.697 | 0.032 | 0.032 | 0.025 |
| 0.1 ng/ml A +1 | 0.363 | 0.211 | 0.238 | 1.763 | 1.706 | 1.63 | 0.022 | 0.02 | 0.012 |
| μg/ml CDDP | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 0.1 ng/ml A | 1.033 | 1.406 | 3.196 | 2.341 | 2.78 | 2.85 | 0.295 | 0.362 | 0.594 |
| 10 μg/ml CDDP | 0.59 | 0.277 | 0.282 | 1.657 | 2.449 | 1.71 | 0.1 | 0.046 | 0.03 |
| 0.1 ng/ml A + 10 | 0.657 | 0.234 | 0.607 | 1.998 | 1.963 | 1.934 | 0.13 | 0.056 | 0.025 |
| μg/ml CDDP | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 0.1 ng/ml A | 1.033 | 1.406 | 3.196 | 2.341 | 2.78 | 2.85 | 0.295 | 0.362 | 0.594 |
| 100 μg/ml CDDP | 0.456 | 0.214 | 0.231 | 1.854 | 1.937 | 1.634 | 0.034 | 0.023 | 0.017 |
| 0.1 ng/ml A + 100 | 0.514 | 0.36 | 0.487 | 2.266 | 2.291 | 2.378 | 0.024 | 0.019 | 0.016 |
| μg/ml CDDP | | | | | | | | | |
| Average | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 1 ng/ml A | 1.126 | 1.567 | 3.673 | 2.446 | 2.805 | 2.87 | 0.295 | 0.324 | 0.457 |
| 0.1 μg/ml CDDP | 0.787 | 0.99 | 2.312 | 2.592 | 2.747 | 2.792 | 0.218 | 0.324 | 0.464 |
| 1 ng/ml A + 0.1 | 0.981 | 0.934 | 1.7 | 2.177 | 2.678 | 2.743 | 0.254 | 0.243 | 0.307 |
| μg/ml CDDP | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 1 ng/ml A | 1.126 | 1.567 | 3.673 | 2.446 | 2.805 | 2.87 | 0.295 | 0.324 | 0.457 |
| 1 μg/ml CDDP | 0.451 | 0.187 | 0.425 | 1.974 | 2.397 | 1.697 | 0.032 | 0.032 | 0.025 |
| 1 ng/ml A + 1 | 0.367 | 0.237 | 0.302 | 1.708 | 2.168 | 1.961 | 0.042 | 0.025 | 0.017 |
| μg/ml CDDP | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 1 ng/ml A | 1.126 | 1.567 | 3.673 | 2.446 | 2.805 | 2.87 | 0.295 | 0.324 | 0.457 |
| 10 μg/ml CDDP | 0.59 | 0.277 | 0.282 | 1.657 | 2.449 | 1.71 | 0.1 | 0.046 | 0.03 |
| 1 ng/ml A + 10 | 0.712 | 0.459 | 0.557 | 2.264 | 2.449 | 2.341 | 0.132 | 0.061 | 0.026 |
| μg/ml CDDP | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 1 ng/ml A | 1.126 | 1.567 | 3.673 | 2.446 | 2.805 | 2.87 | 0.295 | 0.324 | 0.457 |
| 100 μg/ml CDDP | 0.456 | 0.214 | 0.231 | 1.854 | 1.937 | 1.634 | 0.034 | 0.023 | 0.017 |
| 1 ng/ml A + 100 | 0.857 | 0.544 | 0.438 | 2.337 | 2.522 | 2.179 | 0.027 | 0.02 | 0.016 |
| μg/ml CDDP | | | | | | | | | |
| Average | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 10 ng/ml A | 1.183 | 1.677 | 3.433 | 2.178 | 2.794 | 2.856 | 0.22 | 0.421 | 0.429 |
| 0.1 μg/ml CDDP | 0.787 | 0.99 | 2.312 | 2.592 | 2.747 | 2.792 | 0.218 | 0.324 | 0.464 |
| 10 ng/ml A + 0.1 | 1.124 | 0.908 | 1.188 | 1.987 | 2.646 | 2.814 | 0.2 | 0.284 | 0.246 |
| μg/ml CDDP | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 10 ng/ml A | 1.183 | 1.677 | 3.433 | 2.178 | 2.794 | 2.856 | 0.22 | 0.421 | 0.429 |
| 1 μg/ml CDDP | 0.451 | 0.187 | 0.425 | 1.974 | 2.397 | 1.697 | 0.032 | 0.032 | 0.025 |
| l0 ng/ml A + 1 | 0.58 | 0.18 | 0.425 | 1.637 | 2.106 | 2.25 | 0.038 | 0.023 | 0.018 |
| μg/ml CDDP | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 10 ng/ml A | 1.183 | 1.677 | 3.433 | 2.178 | 2.794 | 2.856 | 0.22 | 0.421 | 0.429 |
| 10 μg/ml CDDP | 0.59 | 0.277 | 0.282 | 1.657 | 2.449 | 1.71 | 0.1 | 0.046 | 0.03 |
| 10 ng/ml A + 10 | 0.939 | 0.69 | 0.369 | 2.217 | 2.389 | 2.331 | 0.121 | 0.059 | 0.025 |
| μg/ml CDDP | | | | | | | | | |
| Unstimulated | 0.946 | 1.555 | 2.958 | 2.219 | 2.766 | 2.832 | 0.274 | 0.429 | 0.659 |
| cells | | | | | | | | | |
| 10 ng/ml A | 1.183 | 1.677 | 3.433 | 2.178 | 2.794 | 2.856 | 0.22 | 0.421 | 0.429 |
| 100 μg/ml CDDP | 0.456 | 0.214 | 0.231 | 1.854 | 1.937 | 1.634 | 0.034 | 0.023 | 0.017 |
| 10 ng/ml A + 100 | 0.74 | 0.723 | 0.342 | 1.686 | 2.332 | 2.303 | 0.024 | 0.02 | 0.017 |
| μg/ml CDDP | | | | | | | | | |

TABLE 21

| | HCT-15 | Run 2 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.887 | 1.93 | 3.734 | 2.263 | 2.675 | 2.894 | 0.224 | 0.301 | 0.407 |
| cells | | | | | | | | | |
| 0.01 ng/ml A | 0.857 | 1.906 | NA | 2.468 | 2.716 | 2.891 | 0.181 | 0.32 | 0.334 |
| 0.1 μg/ml CDDP | 0.766 | 1.542 | 2.449 | 2.271 | 2.54 | 2.797 | 0.141 | 0.233 | 0.214 |
| 0.01 ng/ml A + | 0.791 | 0.949 | 0.797 | 2.147 | 2.492 | 2.593 | 0.124 | 0.19 | 0.131 |
| 0.1 μg/ml CDDP | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 0.01 ng/ml A | 0.857 | 1.906 | NA | 2.468 | 2.716 | 2.891 | 0.181 | 0.32 | 0.334 |
| 1 μg/ml CDDP | 0.387 | 0.533 | 0.533 | 1.762 | 2.198 | 2.068 | 0.03 | 0.031 | 0.016 |
| 0.01 ng/ml A + 1 | 0.27 | 0.47 | 0.243 | 1.471 | 1.967 | 1.722 | 0.024 | 0.018 | 0.011 |
| μg/ml CDDP | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 0.01 ng/ml A | 0.857 | 1.906 | NA | 2.468 | 2.716 | 2.891 | 0.181 | 0.32 | 0.334 |
| 10 μg/ml CDDP | 0.824 | 0.742 | 0.46 | 2.09 | 2.456 | 1.85 | 0.094 | 0.067 | 0.041 |
| 0.01 ng/ml A + | 0.387 | 0.293 | 0.381 | 1.841 | 1.552 | 1.55 | 0.048 | 0.036 | 0.017 |
| 10 μg/ml CDDP | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 0.01 ng/ml A | 0.857 | 1.906 | NA | 2.468 | 2.716 | 2.891 | 0.181 | 0.32 | 0.334 |
| 100 μg/ml CDDP | 0.41 | 0.378 | 0.327 | 1.78 | 2.169 | 1.585 | 0.025 | 0.014 | 0.01 |
| 0.01 ng/ml A + | 0.346 | 0.37 | 0.316 | 1.745 | 1.692 | 1.777 | 0.023 | 0.012 | 0.007 |
| 100 μg/ml CDDP | | | | | | | | | |
| Average | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 0.1 ng/ml A | 0.925 | 1.887 | 3.769 | 2.53 | 2.772 | 2.897 | 0.182 | 0.329 | 0.368 |
| 0.1 μg/ml CDDP | 0.766 | 1.542 | 2.449 | 2.271 | 2.54 | 2.797 | 0.141 | 0.233 | 0.214 |
| 0.1 ng/ml A + 0.1 | 0.846 | 1.305 | 1.118 | 2.131 | 2.526 | 2.688 | 0.139 | 0.236 | 0.204 |
| μg/ml CDDP | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 0.1 ng/ml A | 0.925 | 1.887 | 3.769 | 2.53 | 2.772 | 2.897 | 0.182 | 0.329 | 0.368 |
| 1 μg/ml CDDP | 0.387 | 0.533 | 0.533 | 1.762 | 2.198 | 2.068 | 0.03 | 0.031 | 0.016 |
| 0.1 ng/ml A + 1 | 0.262 | 0.426 | 0.248 | 1.7 | 1.532 | 1.819 | 0.028 | 0.016 | 0.009 |
| μg/ml CDDP | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 0.1 ng/ml A | 0.925 | 1.887 | 3.769 | 2.53 | 2.772 | 2.897 | 0.182 | 0.329 | 0.368 |
| 10 μg/ml CDDP | 0.824 | 0.742 | 0.46 | 2.09 | 2.456 | 1.85 | 0.094 | 0.067 | 0.041 |
| 0.1 ng/ml A + 10 | 0.506 | 0.297 | 0.261 | 1.826 | 1.915 | 1.49 | 0.094 | 0.037 | 0.02 |
| μg/ml CDDP | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 0.1 ng/ml A | 0.925 | 1.887 | 3.769 | 2.53 | 2.772 | 2.897 | 0.182 | 0.329 | 0.368 |
| 100 μg/ml CDDP | 0.41 | 0.378 | 0.327 | 1.78 | 2.169 | 1.585 | 0.025 | 0.014 | 0.01 |
| 0.1 ng/ml A + | 0.451 | 0.323 | 0.609 | 1.956 | 2.12 | 2.063 | 0.025 | 0.014 | 0.009 |
| 100 μg/ml CDDP | | | | | | | | | |
| Average | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 1 ng/ml A | 1.146 | 2.336 | NA | 2.477 | 2.717 | 2.898 | 0.164 | 0.272 | 0.335 |
| 0.1 μg/ml CDDP | 0.766 | 1.542 | 2.449 | 2.271 | 2.54 | 2.797 | 0.141 | 0.233 | 0.214 |
| 1 ng/ml A + 0.1 | 0.893 | 1.972 | 1.837 | 2.283 | 2.566 | 2.617 | 0.138 | 0.204 | 0.229 |
| μg/ml CDDP | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 1 ng/ml A | 1.146 | 2.336 | NA | 2.477 | 2.717 | 2.898 | 0.164 | 0.272 | 0.335 |
| 1 μg/ml CDDP | 0.387 | 0.533 | 0.533 | 1.762 | 2.198 | 2.068 | 0.03 | 0.031 | 0.016 |
| 1 ng/ml A + 1 | 0.34 | 0.494 | 0.334 | 1.829 | 1.757 | 1.682 | 0.03 | 0.02 | 0.013 |
| μg/ml CDDP | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 1 ng/ml A | 1.146 | 2.336 | NA | 2.477 | 2.717 | 2.898 | 0.164 | 0.272 | 0.335 |
| 10 μg/ml CDDP | 0.824 | 0.742 | 0.46 | 2.09 | 2.456 | 1.85 | 0.094 | 0.067 | 0.041 |
| 1 ng/ml A + 10 | 0.779 | 0.488 | 0.499 | 2.307 | 1.988 | 1.579 | 0.094 | 0.062 | 0.026 |
| μg/ml CDDP | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 1 ng/ml A | 1.146 | 2.336 | NA | 2.477 | 2.717 | 2.898 | 0.164 | 0.272 | 0.335 |
| 100 μg/ml CDDP | 0.41 | 0.378 | 0.327 | 1.78 | 2.169 | 1.585 | 0.025 | 0.014 | 0.01 |
| 1 ng/ml A + 100 | 0.482 | 0.558 | 0.556 | 2.063 | 2.331 | 2.549 | 0.022 | 0.01 | 0.01 |
| μg/ml CDDP | | | | | | | | | |
| Average | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 10 ng/ml A | 1.186 | 2.601 | NA | 2.43 | 2.743 | 2.907 | 0.171 | 0.298 | 0.249 |
| 0.1 μg/ml CDDP | 0.766 | 1.542 | 2.449 | 2.271 | 2.54 | 2.797 | 0.141 | 0.233 | 0.214 |
| 10 ng/ml A + 0.1 | 1.079 | 1.764 | 1.666 | 2.302 | 2.712 | 2.516 | 0.142 | 0.164 | 0.106 |
| μg/ml CDDP | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 10 ng/ml A | 1.186 | 2.601 | NA | 2.43 | 2.743 | 2.907 | 0.171 | 0.298 | 0.249 |
| 1 μg/ml CDDP | 0.387 | 0.533 | 0.533 | 1.762 | 2.198 | 2.068 | 0.03 | 0.031 | 0.016 |
| 10 ng/ml A + 1 | 0.429 | 0.729 | 0.365 | 1.594 | 1.796 | 1.774 | 0.029 | 0.016 | 0.009 |
| μg/ml CDDP | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 10 ng/ml A | 1.186 | 2.601 | NA | 2.43 | 2.743 | 2.907 | 0.171 | 0.298 | 0.249 |
| 10 μg/ml CDDP | 0.824 | 0.742 | 0.46 | 2.09 | 2.456 | 1.85 | 0.094 | 0.067 | 0.041 |
| 10 ng/ml A + 10 | 0.91 | 0.651 | 0.358 | 1.965 | 2.391 | 1.84 | 0.082 | 0.041 | 0.016 |
| μg/ml CDDP | | | | | | | | | |
| Unstimulated | 0.887 | 1.93 | 3.734 | 2.263 | 2.675 | 2.894 | 0.224 | 0.301 | 0.407 |
| cells | | | | | | | | | |
| 10 ng/ml A | 1.186 | 2.601 | NA | 2.43 | 2.743 | 2.907 | 0.171 | 0.298 | 0.249 |
| 100 μg/ml CDDP | 0.41 | 0.378 | 0.327 | 1.78 | 2.169 | 1.585 | 0.025 | 0.014 | 0.01 |
| 10 ng/ml A + 100 | 0.62 | 0.643 | 0.551 | 2.515 | 2.241 | 1.86 | 0.029 | 0.011 | 0.008 |
| μg/ml CDDP | | | | | | | | | |

TABLE 22

| | HCT-15 | Run 3 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 2.04 | 1.874 | 3.914 | 2.405 | 2.823 | 2.921 | 0.315 | 0.495 | 0.428 |
| cells | | | | | | | | | |
| 0.01 ng/ml A | 2.063 | 2.446 | NA | 2.598 | 2.838 | 3.032 | 0.361 | 0.463 | 0.465 |
| 0.1 μg/ml CDDP | 2.043 | 1.335 | 2.814 | 2.578 | 2.768 | 2.96 | 0.294 | 0.391 | 0.457 |
| 0.01 ng/ml A + | 1.693 | 0.82 | 1.416 | 2.523 | 2.629 | 2.822 | 0.223 | 0.312 | 0.309 |
| 0.1 μg/ml CDDP | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 0.01 ng/ml A | 2.063 | 2.446 | NA | 2.598 | 2.838 | 3.032 | 0.361 | 0.463 | 0.465 |
| 1 μg/ml CDDP | 0.459 | 0.126 | 0.623 | 1.713 | 1.845 | 1.752 | 0.028 | 0.035 | 0.039 |
| 0.01 ng/ml A + 1 | 0.291 | 0.063 | 0.517 | 1.851 | 1.372 | 1.748 | 0.023 | 0.02 | 0.021 |
| μg/ml CDDP | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 0.01 ng/ml A | 2.063 | 2.446 | NA | 2.598 | 2.838 | 3.032 | 0.361 | 0.463 | 0.465 |
| 10 μg/ml CDDP | 1.477 | 0.363 | 0.669 | 1.941 | 2.104 | 2.063 | 0.137 | 0.104 | 0.043 |
| 0.01 ng/ml A + 10 | 1.389 | 0.23 | 0.415 | 2.173 | 1.726 | 1.688 | 0.154 | 0.052 | 0.032 |
| μg/ml CDDP | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 0.01 ng/ml A | 2.063 | 2.446 | NA | 2.598 | 2.838 | 3.032 | 0.361 | 0.463 | 0.465 |
| 100 μg/ml CDDP | 0.473 | 0.114 | 0.499 | 1.254 | 1.77 | 1.935 | 0.039 | 0.026 | 0.017 |
| 0.01 ng/ml A + | 0.9 | 0.314 | 0.466 | 2.069 | 1.903 | 2.003 | 0.02 | 0.018 | 0.018 |
| 100 μg CDDP | | | | | | | | | |
| Average | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 0.1 ng/ml A | 1.956 | 2.506 | 3.635 | 2.563 | 2.867 | 3.012 | 0.38 | 0.451 | 0.433 |
| 0.1 μg/ml CDDP | 2.043 | 1.335 | 2.814 | 2.578 | 2.768 | 2.96 | 0.294 | 0.391 | 0.457 |
| 0.1 ng/ml A + 0.1 | 1.597 | 0.82 | 1.667 | 2.347 | 2.62 | 2.806 | 0.235 | 0.288 | 0.256 |
| μg/ml CDDP | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 0.1 ng/ml A | 1.956 | 2.506 | 3.635 | 2.563 | 2.867 | 3.012 | 0.38 | 0.451 | 0.433 |
| 1 μg/ml CDDP | 0.459 | 0.126 | 0.623 | 1.713 | 1.845 | 1.752 | 0.028 | 0.035 | 0.039 |
| 0.1 ng/ml A + 1 | 0.526 | 0.098 | 0.602 | 1.472 | 1.352 | 1.956 | 0.021 | 0.024 | 0.023 |
| μg/ml CDDP | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 0.1 ng/ml A | 1.956 | 2.506 | 3.635 | 2.563 | 2.867 | 3.012 | 0.38 | 0.451 | 0.433 |
| 10 μg/ml CDDP | 1.477 | 0.363 | 0.669 | 1.941 | 2.104 | 2.063 | 0.137 | 0.104 | 0.043 |
| 0.1 ng/ml A + 10 | 1.765 | 0.325 | 0.575 | 1.909 | 1.788 | 2.007 | 0.137 | 0.051 | 0.032 |
| μg/ml CDDP | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 0.1 ng/ml A | 1.956 | 2.506 | 3.635 | 2.563 | 2.867 | 3.012 | 0.38 | 0.451 | 0.433 |
| 100 μg/ml CDDP | 0.473 | 0.114 | 0.499 | 1.254 | 1.77 | 1.935 | 0.039 | 0.026 | 0.017 |
| 0.1 ng/ml A + 100 | 0.542 | 0.298 | 0.542 | 2.057 | 2.147 | 2.098 | 0.021 | 0.018 | 0.017 |
| μg/ml CDDP | | | | | | | | | |
| Average | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 1 ng/ml A | 2.275 | 2.635 | 3.911 | 2.495 | 2.867 | 3.019 | 0.282 | 0.442 | 0.347 |
| 0.1 μg/ml CDDP | 2.043 | 1.335 | 2.814 | 2.578 | 2.768 | 2.96 | 0.294 | 0.391 | 0.457 |
| 1 ng/ml A + 0.1 | 1.75 | 1.132 | 2.449 | 2.188 | 2.709 | 2.888 | 0.182 | 0.308 | 0.244 |
| μg/ml CDDP | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 1 ng/ml A | 2.275 | 2.635 | 3.911 | 2.495 | 2.867 | 3.019 | 0.282 | 0.442 | 0.347 |
| 1 μg/ml CDDP | 0.459 | 0.126 | 0.623 | 1.713 | 1.845 | 1.752 | 0.028 | 0.035 | 0.039 |
| 1 ng/ml A + 1 | 0.518 | 0.119 | 0.591 | 1.471 | 1.537 | 1.91 | 0.033 | 0.027 | 0.016 |
| μg/ml CDDP | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 1 ng/ml A | 2.275 | 2.635 | 3.911 | 2.495 | 2.867 | 3.019 | 0.282 | 0.442 | 0.347 |
| 10 μg/ml CDDP | 1.477 | 0.363 | 0.669 | 1.941 | 2.104 | 2.063 | 0.137 | 0.104 | 0.043 |
| 1 ng/ml A + 10 | 1.476 | 0.396 | 0.762 | 2.2 | 2.128 | 1.931 | 0.131 | 0.095 | 0.039 |
| μg/ml CDDP | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 1 ng/ml A | 2.275 | 2.635 | 3.911 | 2.495 | 2.867 | 3.019 | 0.282 | 0.442 | 0.347 |
| 100 μg/ml CDDP | 0.473 | 0.114 | 0.499 | 1.254 | 1.77 | 1.935 | 0.039 | 0.026 | 0.017 |
| 1 ng/ml A + 100 | 0.64 | 0.232 | 0.58 | 2.23 | 2.496 | 1.961 | 0.035 | 0.023 | 0.02 |
| μg/ml CDDP | | | | | | | | | |
| Average | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 10 ng/ml A | 2.302 | 2.571 | NA | 2.262 | 2.853 | 2.966 | 0.287 | 0.414 | 0.411 |
| 0.1 μg/ml CDDP | 2.043 | 1.335 | 2.814 | 2.578 | 2.768 | 2.96 | 0.294 | 0.391 | 0.457 |
| 10 ng/ml A + 0.1 | 1.91 | 1.21 | 1.922 | 2.069 | 2.599 | 2.746 | 0.23 | 0.299 | 0.261 |
| μg/ml CDDP | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 10 ng/ml A | 2.302 | 2.571 | NA | 2.262 | 2.853 | 2.966 | 0.287 | 0.414 | 0.411 |
| 1 μg/ml CDDP | 0.459 | 0.126 | 0.623 | 1.713 | 1.845 | 1.752 | 0.028 | 0.035 | 0.039 |
| 10 ng/ml A + 1 | 0.387 | 0.135 | 0.688 | 1.721 | 1.712 | 1.704 | 0.029 | 0.026 | 0.011 |
| μg/ml CDDP | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 10 ng/ml A | 2.302 | 2.571 | NA | 2.262 | 2.853 | 2.966 | 0.287 | 0.414 | 0.411 |
| 10 μg/ml CDDP | 1.477 | 0.363 | 0.669 | 1.941 | 2.104 | 2.063 | 0.137 | 0.104 | 0.043 |
| 10 ng/ml A + 10 | 1.277 | 0.419 | 0.87 | 1.899 | 1.81 | 1.791 | 0.161 | 0.064 | 0.033 |
| μg/ml CDDP | | | | | | | | | |
| Unstimulated | 2.04 | 1.874 | 3.914 | 2.405 | 2.823 | 2.921 | 0.315 | 0.495 | 0.428 |
| cells | | | | | | | | | |
| 10 ng/ml A | 2.302 | 2.571 | NA | 2.262 | 2.853 | 2.966 | 0.287 | 0.414 | 0.411 |
| 100 μg/ml CDDP | 0.473 | 0.114 | 0.499 | 1.254 | 1.77 | 1.935 | 0.039 | 0.026 | 0.017 |
| 10 ng/ml A + 100 | 0.778 | 0.372 | 0.731 | 1.958 | 2.251 | 2.23 | 0.029 | 0.02 | 0.15 |
| μg/ml CDDP | | | | | | | | | |

TABLE 23

| | HCT-116 | Run 1 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.694 | 1.446 | 2.944 | 1.547 | 2.253 | 2.722 | 0.128 | 0.307 | 0.452 |
| cells | | | | | | | | | |
| 0.01 ng/ml A | 0.689 | 1.324 | 2.947 | 1.638 | 2.376 | 2.8 | 0.118 | 0.295 | 0.412 |
| 0.1 μg/ml CDDP | 0.569 | 0.984 | 1.352 | 1.56 | 2.278 | 2.449 | 0.215 | 0.184 | 0.332 |
| 0.01 ng/ml A + | 0.412 | 0.744 | 0.854 | 1.755 | 1.924 | 2.136 | 0.22 | 0.154 | 0.202 |
| 0.1 μg/ml CDDP | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 0.01 ng/ml A | 0.689 | 1.324 | 2.947 | 1.638 | 2.376 | 2.8 | 0.118 | 0.295 | 0.412 |
| 1 μg/ml CDDP | 0.563 | 0.624 | 0.308 | 1.606 | 2.16 | 1.569 | 0.037 | 0.027 | 0.03 |
| 0.01 ng/ml A + 1 | 0.152 | 0.354 | 0.248 | 1.525 | 1.233 | 1.516 | 0.028 | 0.021 | 0.027 |
| μg/ml CDDP | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 0.01 ng/ml A | 0.689 | 1.324 | 2.947 | 1.638 | 2.376 | 2.8 | 0.118 | 0.295 | 0.412 |
| 10 μg/ml CDDP | 1.173 | 0.488 | 0.501 | 1.527 | 2.055 | 1.67 | 0.072 | 0.054 | 0.033 |
| 0.01 ng/ml A + | 0.33 | 0.339 | 0.392 | 1.808 | 1.406 | 1.839 | 0.131 | 0.083 | 0.05 |
| 10 μg/ml CDDP | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 0.01 ng/ml A | 0.689 | 1.324 | 2.947 | 1.638 | 2.376 | 2.8 | 0.118 | 0.295 | 0.412 |
| 100 μg/ml CDDP | 0.344 | 0.356 | 0.51 | 1.736 | 1.169 | 1.616 | 0.038 | 0.031 | 0.031 |
| 0.01 ng/ml A + | 0.415 | 0.368 | 0.261 | 1.83 | 1.619 | 1.895 | 0.029 | 0.021 | 0.026 |
| 100 μg/ml CDDP | | | | | | | | | |
| Average | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 0.1 ng/ml A | 0.909 | 1.576 | 2.612 | 1.791 | 2.526 | 2.783 | 0.224 | 0.365 | 0.449 |
| 0.1 μg/ml CDDP | 0.569 | 0.984 | 1.352 | 1.56 | 2.278 | 2.449 | 0.215 | 0.184 | 0.332 |
| 0.1 ng/ml A + 0.1 | 0.629 | 1.025 | 0.976 | 1.568 | 2.018 | 2.456 | 0.182 | 0.169 | 0.234 |
| μg/ml CDDP | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 0.1 ng/ml A | 0.909 | 1.576 | 2.612 | 1.791 | 2.526 | 2.783 | 0.224 | 0.365 | 0.449 |
| 1 μg/ml CDDP | 0.563 | 0.624 | 0.308 | 1.606 | 2.16 | 1.569 | 0.037 | 0.027 | 0.03 |
| 0.1 ng/ml A + 1 | 0.277 | 0.404 | 0.339 | 1.532 | 1.308 | 2.055 | 0.026 | 0.018 | 0.024 |
| μg/ml CDDP | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 0.1 ng/ml A | 0.909 | 1.576 | 2.612 | 1.791 | 2.526 | 2.783 | 0.224 | 0.365 | 0.449 |
| 10 μg/ml CDDP | 1.173 | 0.488 | 0.501 | 1.527 | 2.055 | 1.67 | 0.072 | 0.054 | 0.033 |
| 0.1 ng/ml A + 10 | 0.653 | 0.405 | 0.403 | 1.742 | 1.39 | 1.678 | 0.152 | 0.079 | 0.041 |
| μg/ml CDDP | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 0.1 ng/ml A | 0.909 | 1.576 | 2.612 | 1.791 | 2.526 | 2.783 | 0.224 | 0.365 | 0.449 |
| 100 μg/ml CDDP | 0.344 | 0.356 | 0.51 | 1.736 | 1.169 | 1.616 | 0.038 | 0.031 | 0.031 |
| 0.1 ng/ml A + | 0.736 | 0.425 | 0.525 | 1.679 | 1.748 | 1.956 | 0.03 | 0.023 | 0.028 |
| 100 μg/ml CDDP | | | | | | | | | |
| Average | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 1 ng/ml A | 0.891 | 1.975 | 3.013 | 1.536 | 2.344 | 2.788 | 0.132 | 0.274 | 0.333 |
| 0.1 μg/ml CDDP | 0.569 | 0.984 | 1.352 | 1.56 | 2.278 | 2.449 | 0.215 | 0.184 | 0.332 |
| 1 ng/ml A + 0.1 | 0.657 | 1.063 | 1.47 | 1.652 | 2.08 | 2.57 | 0.088 | 0.133 | 0.158 |
| μg/ml CDDP | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 1 ng/ml A | 0.891 | 1.975 | 3.013 | 1.536 | 2.344 | 2.788 | 0.132 | 0.274 | 0.333 |
| 1 μg/ml CDDP | 0.563 | 0.624 | 0.308 | 1.606 | 2.16 | 1.569 | 0.037 | 0.027 | 0.03 |
| 1 ng/ml A + 1 | 0.473 | 0.401 | 0.653 | 1.695 | 1.833 | 1.758 | 0.033 | 0.026 | 0.028 |
| μg/ml CDDP | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 1 ng/ml A | 0.891 | 1.975 | 3.013 | 1.536 | 2.344 | 2.788 | 0.132 | 0.274 | 0.333 |
| 10 μg/ml CDDP | 1.173 | 0.488 | 0.501 | 1.527 | 2.055 | 1.67 | 0.072 | 0.054 | 0.033 |
| 1 ng/ml A + 10 | 1.142 | 0.85 | 0.53 | 1.691 | 2.065 | 2.143 | 0.081 | 0.086 | 0.064 |
| μg/ml CDDP | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 1 ng/ml A | 0.891 | 1.975 | 3.013 | 1.536 | 2.344 | 2.788 | 0.132 | 0.274 | 0.333 |
| 100 μg/ml CDDP | 0.344 | 0.356 | 0.51 | 1.736 | 1.169 | 1.616 | 0.038 | 0.031 | 0.031 |
| 1 ng/ml A + 100 | 0.725 | 0.464 | 0.678 | 1.721 | 2.034 | 2.11 | 0.032 | 0.027 | 0.028 |
| μg/ml CDDP | | | | | | | | | |
| Average | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 10 ng/ml A | 0.828 | 1.634 | 3.032 | 1.682 | 2.223 | 2.716 | 0.129 | 0.291 | 0.355 |
| 0.1 μg/ml CDDP | 0.569 | 0.984 | 1.352 | 1.56 | 2.278 | 2.449 | 0.215 | 0.184 | 0.332 |
| 10 ng/ml A + 0.1 | 0.726 | 1.191 | 1.394 | 1.888 | 1.803 | 2.239 | 0.082 | 0.126 | 0.131 |
| μg/ml CDDP | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 10 ng/ml A | 0.828 | 1.634 | 3.032 | 1.682 | 2.223 | 2.716 | 0.129 | 0.291 | 0.355 |
| 1 μg/ml CDDP | 0.563 | 0.624 | 0.308 | 1.606 | 2.16 | 1.569 | 0.037 | 0.027 | 0.03 |
| 10 ng/ml A + 1 | 0.461 | 0.44 | 0.565 | 1.578 | 1.893 | 1.744 | 0.031 | 0.075 | 0.023 |
| μg/ml CDDP | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 10 ng/ml A | 0.828 | 1.634 | 3.032 | 1.682 | 2.223 | 2.716 | 0.129 | 0.291 | 0.355 |
| 10 μg/ml CDDP | 1.173 | 0.488 | 0.501 | 1.527 | 2.055 | 1.67 | 0.072 | 0.054 | 0.033 |
| 10 ng/ml A + 10 | 1.1 | 0.73 | 0.552 | 2.112 | 1.914 | 1.679 | 0.086 | 0.069 | 0.044 |
| μg/ml CDDP | | | | | | | | | |
| Unstimulated | 0.694 | 1.446 | 2.944 | 1.547 | 2.253 | 2.722 | 0.128 | 0.307 | 0.452 |
| cells | | | | | | | | | |
| 10 ng/ml A | 0.828 | 1.634 | 3.032 | 1.682 | 2.223 | 2.716 | 0.129 | 0.291 | 0.355 |
| 100 μg/ml CDDP | 0.344 | 0.356 | 0.51 | 1.736 | 1.169 | 1.616 | 0.038 | 0.031 | 0.031 |
| 10 ng/ml A + 100 | 1.048 | 0.454 | 0.627 | 2.059 | 1.991 | 1.814 | 0.032 | 0.027 | 0.029 |
| μg/ml CDDP | | | | | | | | | |

TABLE 24

| | HCT-116 | Run 2 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.61 | 1.45 | 2.306 | 1.297 | 2.941 | 2.944 | 0.137 | 0.328 | 0.512 |
| cells | | | | | | | | | |
| 0.01 ng/ml A | 0.619 | 1.252 | 2.198 | 1.45 | 2.962 | 2.97 | 0.115 | 0.275 | 0.483 |
| 0.1 μg/ml CDDP | 0.52 | 0.971 | 1.187 | 1.357 | 2.886 | 2.909 | 0.111 | 0.179 | 0.296 |
| 0.01 ng/ml A + | 0.532 | 0.613 | 0.869 | 1.205 | 2.811 | 2.803 | 0.109 | 0.163 | 0.216 |
| 0.1 μg/ml CDDP | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 0.01 ng/ml A | 0.619 | 1.252 | 2.198 | 1.45 | 2.962 | 2.97 | 0.115 | 0.275 | 0.483 |
| 1 μg/ml CDDP | 0.302 | 0.425 | 0.551 | 1.199 | 2.878 | 2.703 | 0.053 | 0.044 | 0.054 |
| 0.01 ng/ml A + 1 | 0.234 | 0.294 | 0.369 | 1.201 | 2.683 | 2.519 | 0.044 | 0.032 | 0.027 |
| μg/ml CDDP | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 0.01 ng/ml A | 0.619 | 1.252 | 2.198 | 1.45 | 2.962 | 2.97 | 0.115 | 0.275 | 0.483 |
| 10 μg/ml CDDP | 0.477 | 0.608 | 0.435 | 1.056 | 2.746 | 2.728 | 0.081 | 0.045 | 0.045 |
| 0.01 ng/ml A + 10 | 0.449 | 0.261 | 0.26 | 1.414 | 2.456 | 2.761 | 0.09 | 0.071 | 0.041 |
| μg/ml CDDP | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 0.01 ng/ml A | 0.619 | 1.252 | 2.198 | 1.45 | 2.962 | 2.97 | 0.115 | 0.275 | 0.483 |
| 100 μg/ml CDDP | 0.303 | 0.529 | 0.492 | 1.173 | 2.664 | 2.514 | 0.072 | 0.04 | 0.037 |
| 0.01 ng/ml A + | 0.274 | 0.147 | 0.219 | 1.649 | 2.14 | 2.648 | 0.049 | 0.033 | 0.029 |
| 100 μg/ml CDDP | | | | | | | | | |
| Average | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 0.1 ng/ml A | 0.623 | 1.436 | 2.361 | 1.476 | 2.99 | 2.982 | 0.138 | 0.261 | 0.488 |
| 0.1 μg/ml CDDP | 0.52 | 0.971 | 1.187 | 1.357 | 2.886 | 2.909 | 0.111 | 0.179 | 0.296 |
| 0.1 ng/ml A + 0.1 | 0.56 | 0.976 | 1.036 | 1.353 | 2.971 | 2.75 | 0.097 | 0.149 | 0.229 |
| μg/ml CDDP | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 0.1 ng/ml A | 0.623 | 1.436 | 2.361 | 1.476 | 2.99 | 2.982 | 0.138 | 0.261 | 0.488 |
| 1 μg/ml CDDP | 0.302 | 0.425 | 0.551 | 1.199 | 2.878 | 2.703 | 0.053 | 0.044 | 0.054 |
| 0.1 ng/ml A + 1 | 0.292 | 0.288 | 0.416 | 1.546 | 2.807 | 2.679 | 0.042 | 0.033 | 0.03 |
| μg/ml CDDP | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 0.1 ng/ml A | 0.623 | 1.436 | 2.361 | 1.476 | 2.99 | 2.982 | 0.138 | 0.261 | 0.488 |
| 10 μg/ml CDDP | 0.477 | 0.608 | 0.435 | 1.056 | 2.746 | 2.728 | 0.081 | 0.045 | 0.045 |
| 0.1 ng/ml A + 10 | 0.465 | 0.296 | 0.308 | 1.311 | 2.858 | 2.801 | 0.097 | 0.052 | 0.037 |
| μg/ml CDDP | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 0.1 ng/ml A | 0.623 | 1.436 | 2.361 | 1.476 | 2.99 | 2.982 | 0.138 | 0.261 | 0.488 |
| 100 μg/ml CDDP | 0.303 | 0.529 | 0.492 | 1.173 | 2.664 | 2.514 | 0.072 | 0.04 | 0.037 |
| 0.1 ng/ml A + 100 | 0.25 | 0.171 | 0.254 | 1.573 | 2.554 | 2.813 | 0.04 | 0.034 | 0.03 |
| μg/ml CDDP | | | | | | | | | |
| Average | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 1 ng/ml A | 0.778 | 1.976 | 2.507 | 1.589 | 2.961 | 2.959 | 0.118 | 0.299 | 0.466 |
| 0.1 μg/ml CDDP | 0.52 | 0.971 | 1.187 | 1.357 | 2.886 | 2.909 | 0.111 | 0.179 | 0.296 |
| 1 ng/ml A + 0.1 | 0.608 | 1.144 | 1.057 | 1.511 | 2.846 | 2.86 | 0.09 | 0.17 | 0.253 |
| μg/ml CDDP | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 1 ng/ml A | 0.778 | 1.976 | 2.507 | 1.589 | 2.961 | 2.959 | 0.118 | 0.299 | 0.466 |
| 1 μg/ml CDDP | 0.302 | 0.425 | 0.551 | 1.199 | 2.878 | 2.703 | 0.053 | 0.044 | 0.054 |
| 1 ng/ml A + 1 | 0.297 | 0.412 | 0.365 | 1.8 | 2.872 | 2.579 | 0.051 | 0.029 | 0.031 |
| μg/ml CDDP | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 1 ng/ml A | 0.778 | 1.976 | 2.507 | 1.589 | 2.961 | 2.959 | 0.118 | 0.299 | 0.466 |
| 10 μg/ml CDDP | 0.477 | 0.608 | 0.435 | 1.056 | 2.746 | 2.728 | 0.081 | 0.045 | 0.045 |
| 1 ng/ml A + 10 | 0.577 | 0.507 | 0.346 | 1.551 | 2.881 | 2.64 | 0.096 | 0.073 | 0.043 |
| μg/ml CDDP | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 1 ng/ml A | 0.778 | 1.976 | 2.507 | 1.589 | 2.961 | 2.959 | 0.118 | 0.299 | 0.466 |
| 100 μg/ml CDDP | 0.303 | 0.529 | 0.492 | 1.173 | 2.664 | 2.514 | 0.072 | 0.04 | 0.037 |
| 1 ng/ml A + 100 | 0.429 | 0.179 | 0.337 | 1.719 | 2.436 | 2.408 | 0.099 | 0.035 | 0.033 |
| μg/ml CDDP | | | | | | | | | |
| Average | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 10 ng/ml A | 0.741 | 2.09 | 2.758 | 1.41 | 2.926 | 2.957 | 0.147 | 0.363 | 0.552 |
| 0.1 μg/ml CDDP | 0.52 | 0.971 | 1.187 | 1.357 | 2.886 | 2.909 | 0.111 | 0.179 | 0.296 |
| 10 ng/ml A + 0.1 | 0.607 | 1.186 | 1.172 | 1.41 | 2.781 | 2.736 | 0.125 | 0.2 | 0.201 |
| μg/ml CDDP | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 10 ng/ml A | 0.741 | 2.09 | 2.758 | 1.41 | 2.926 | 2.957 | 0.147 | 0.363 | 0.552 |
| 1 μg/ml CDDP | 0.302 | 0.425 | 0.551 | 1.199 | 2.878 | 2.703 | 0.053 | 0.044 | 0.054 |
| 10 ng/m1 A + 1 | 0.375 | 0.378 | 0.418 | 1.486 | 2.642 | 2.607 | 0.043 | 0.032 | 0.028 |
| μg/ml CDDP | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 10 ng/ml A | 0.741 | 2.09 | 2.758 | 1.41 | 2.926 | 2.957 | 0.147 | 0.363 | 0.552 |
| 10 μg/ml CDDP | 0.477 | 0.608 | 0.435 | 1.056 | 2.746 | 2.728 | 0.081 | 0.045 | 0.045 |
| 10 ng/ml A + 10 | 0.771 | 0.418 | 0.355 | 1.339 | 2.652 | 2.694 | 0.105 | 0.081 | 0.047 |
| μg/ml CDDP | | | | | | | | | |
| Unstimulated | 0.61 | 1.45 | 2.306 | 1.297 | 2.941 | 2.944 | 0.137 | 0.328 | 0.512 |
| cells | | | | | | | | | |
| 10 ng/ml A | 0.741 | 2.09 | 2.758 | 1.41 | 2.926 | 2.957 | 0.147 | 0.363 | 0.552 |
| 100 μg/ml CDDP | 0.303 | 0.529 | 0.492 | 1.173 | 2.664 | 2.514 | 0.072 | 0.04 | 0.037 |
| 10 ng/ml A + 100 | 0.497 | 0.166 | 0.3 | 1.435 | 1.977 | 2.279 | 0.042 | 0.035 | 0.028 |
| μg/ml CDDP | | | | | | | | | |

TABLE 25

| | HCT-116 | Run 3 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.434 | 1.093 | 2.36 | 1.813 | 2.359 | 2.707 | 0.175 | 0.447 | 0.553 |
| cells | | | | | | | | | |
| 0.01 ng/ml A | 0.504 | 1.175 | 2.387 | 1.916 | 2.205 | 2.802 | 0.163 | 0.459 | 0.591 |
| 0.1 μg/ml CDDP | 0.371 | 1.153 | 1.078 | 2.01 | 1.933 | 2.357 | 0.16 | 0.302 | 0.385 |
| 0.01 ng/ml A + | 0.325 | 0.844 | 0.676 | 1.912 | 1.851 | 1.912 | 0.147 | 0.148 | 0.271 |
| 0.1 μg/ml CDDP | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 0.01 ng/ml A | 0.504 | 1.175 | 2.387 | 1.916 | 2.205 | 2.802 | 0.163 | 0.459 | 0.591 |
| 1 μg/ml CDDP | 0.267 | 0.687 | 0.235 | 2.129 | 1.394 | 1.78 | 0.041 | 0.039 | 0.041 |
| 0.01 ng/ml A + 1 | 0.245 | 0.25 | 0.18 | 1.911 | 1.241 | 1.177 | 0.032 | 0.028 | 0.035 |
| μg/ml CDDP | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 0.01 ng/ml A | 0.504 | 1.175 | 2.387 | 1.916 | 2.205 | 2.802 | 0.163 | 0.459 | 0.591 |
| 10 μg/ml CDDP | 0.393 | 0.993 | 0.476 | 2.05 | 1.353 | 1.876 | 0.09 | 0.108 | 0.147 |
| 0.01 ng/ml A + | 0.486 | 0.296 | 0.232 | 1.933 | 1.532 | 1.11 | 0.133 | 0.101 | 0.075 |
| 10 μg CDDP | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 0.01 ng/ml A | 0.504 | 1.175 | 2.387 | 1.916 | 2.205 | 2.802 | 0.163 | 0.459 | 0.591 |
| 100 μg/ml CDDP | 0.256 | 0.359 | 0.214 | 2.084 | 1.136 | 1.404 | 0.044 | 0.037 | 0.043 |
| 0.01 ng/ml A + | 0.538 | 0.415 | 0.306 | 1.942 | 1.271 | 1.316 | 0.028 | 0.028 | 0.033 |
| 100 μg/ml CDDP | | | | | | | | | |
| Average | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 0.1 ng/ml A | 0.572 | 1.327 | 2.23 | 2.081 | 2.06 | 2.785 | 0.194 | 0.31 | 0.61 |
| 0.1 μg/ml CDDP | 0.371 | 1.153 | 1.078 | 2.01 | 1.933 | 2.357 | 0.16 | 0.302 | 0.385 |
| 0.1 ng/ml A + 0.1 | 0.44 | 0.509 | 0.647 | 1.929 | 1.799 | 2.017 | 0.19 | 0.186 | 0.264 |
| μg/ml CDDP | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 0.1 ng/ml A | 0.572 | 1.327 | 2.23 | 2.081 | 2.06 | 2.785 | 0.194 | 0.31 | 0.61 |
| 1 μg/ml CDDP | 0.267 | 0.687 | 0.235 | 2.129 | 1.394 | 1.78 | 0.041 | 0.039 | 0.041 |
| 0.1 ng/ml A + 1 | 0.288 | 0.228 | 0.184 | 2.31 | 1.282 | 1.59 | 0.031 | 0.03 | 0.034 |
| μg/ml CDDP | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 0.1 ng/ml A | 0.572 | 1.327 | 2.23 | 2.081 | 2.06 | 2.785 | 0.194 | 0.31 | 0.61 |
| 10 μg/ml CDDP | 0.393 | 0.993 | 0.476 | 2.05 | 1.353 | 1.876 | 0.09 | 0.108 | 0.147 |
| 0.1 ng/ml A + 10 | 0.294 | 0.367 | 0.274 | 1.944 | 1.303 | 1.368 | 0.117 | 0.081 | 0.053 |
| μg/ml CDDP | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 0.1 ng/ml A | 0.572 | 1.327 | 2.23 | 2.081 | 2.06 | 2.785 | 0.194 | 0.31 | 0.61 |
| 100 μg/ml CDDP | 0.256 | 0.359 | 0.214 | 2.084 | 1.136 | 1.404 | 0.044 | 0.037 | 0.043 |
| 0.1 ng/ml A + | 0.414 | 0.833 | 0.376 | 1.932 | 1.167 | 1.631 | 0.03 | 0.028 | 0.037 |
| 100 μg/ml CDDP | | | | | | | | | |
| Average | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 1 ng/ml A | 0.611 | 1.473 | 2.671 | 2.2 | 2.179 | 2.76 | 0.124 | 0.289 | 0.422 |
| 0.1 μg/ml CDDP | 0.371 | 1.153 | 1.078 | 2.01 | 1.933 | 2.357 | 0.16 | 0.302 | 0.385 |
| 1 ng/ml A + 0.1 | 0.586 | 1.164 | 0.891 | 2.127 | 1.473 | 1.973 | 0.092 | 0.182 | 0.268 |
| μg/ml CDDP | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 1 ng/ml A | 0.611 | 1.473 | 2.671 | 2.2 | 2.179 | 2.76 | 0.124 | 0.289 | 0.422 |
| 1 μg/ml CDDP | 0.267 | 0.687 | 0.235 | 2.129 | 1.394 | 1.78 | 0.041 | 0.039 | 0.041 |
| 1 ng/ml A + 1 | 0.33 | 0.357 | 0.285 | 2.183 | 1.137 | 1.437 | 0.037 | 0.032 | 0.038 |
| μg/ml CDDP | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 1 ng/ml A | 0.611 | 1.473 | 2.671 | 2.2 | 2.179 | 2.76 | 0.124 | 0.289 | 0.422 |
| 10 μg/ml CDDP | 0.393 | 0.993 | 0.476 | 2.05 | 1.353 | 1.876 | 0.09 | 0.108 | 0.147 |
| 1 ng/ml A + 10 | 0.55 | 0.803 | 0.468 | 2.03 | 1.412 | 1.657 | 0.097 | 0.115 | 0.08 |
| μg/ml CDDP | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 1 ng/ml A | 0.611 | 1.473 | 2.671 | 2.2 | 2.179 | 2.76 | 0.124 | 0.289 | 0.422 |
| 100 μg/ml CDDP | 0.256 | 0.359 | 0.214 | 2.084 | 1.136 | 1.404 | 0.044 | 0.037 | 0.043 |
| 1 ng/ml A + 100 | 0.544 | 1.093 | 0.533 | 2.418 | 1.646 | 2.065 | 0.039 | 0.035 | 0.043 |
| μg/ml CDDP | | | | | | | | | |
| Average | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 10 ng/ml A | 0.627 | 1.561 | 2.566 | 2.135 | 1.963 | 2.644 | 0.113 | 0.318 | 0.522 |
| 0.1 μg/ml CDDP | 0.371 | 1.153 | 1.078 | 2.01 | 1.933 | 2.357 | 0.16 | 0.302 | 0.385 |
| 10 ng/ml A + 0.1 | 0.585 | 1.11 | 0.811 | 1.977 | 1.593 | 1.915 | 0.098 | 0.185 | 0.236 |
| μg/ml CDDP | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 10 ng/ml A | 0.627 | 1.561 | 2.566 | 2.135 | 1.963 | 2.644 | 0.113 | 0.318 | 0.522 |
| 1 μg/ml DDP | 0.267 | 0.687 | 0.235 | 2.129 | 1.394 | 1.78 | 0.041 | 0.039 | 0.041 |
| 10 ng/ml A + 1 | 0.331 | 0.529 | 0.278 | 1.95 | 1.239 | 1.629 | 0.033 | 0.03 | 0.035 |
| μg/ml CDDP | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 10 ng/ml A | 0.627 | 1.561 | 2.566 | 2.135 | 1.963 | 2.644 | 0.113 | 0.318 | 0.522 |
| 10 μg/ml CDDP | 0.393 | 0.993 | 0.476 | 2.05 | 1.353 | 1.876 | 0.09 | 0.108 | 0.147 |
| 10 ng/ml A + 10 | 0.696 | 0.787 | 0.408 | 2.105 | 1.255 | 1.596 | 0.096 | 0.092 | 0.061 |
| μg/ml CDDP | | | | | | | | | |
| Unstimulated | 0.434 | 1.093 | 2.36 | 1.813 | 2.359 | 2.707 | 0.175 | 0.447 | 0.553 |
| cells | | | | | | | | | |
| 10 ng/ml A | 0.627 | 1.561 | 2.566 | 2.135 | 1.963 | 2.644 | 0.113 | 0.318 | 0.522 |
| 100 μg/ml CDDP | 0.256 | 0.359 | 0.214 | 2.084 | 1.136 | 1.404 | 0.044 | 0.037 | 0.043 |
| 10 ng/ml A + 100 | 0.515 | 0.768 | 0.446 | 2.219 | 1.213 | 1.997 | 0.037 | 0.029 | 0.04 |
| μg/ml CDDP | | | | | | | | | |

TABLE 26

| | HT55 | Run 1 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.558 | 0.661 | 1.223 | 1.751 | 2.348 | 2.403 | 0.119 | 0.242 | 0.153 |
| cells | | | | | | | | | |
| 0.01 ng/ml A | 1.064 | 0.729 | 1.252 | 2.089 | 2.387 | 2.506 | 0.11 | 0.229 | 0.177 |
| 0.1 μg/ml CDDP | 0.838 | 0.588 | 0.901 | 2.17 | 2.437 | 2.413 | 0.096 | 0.177 | 0.315 |
| 0.01 ng/ml A + | 0.579 | 0.446 | 0.43 | 2.101 | 2.413 | 2.242 | 0.081 | 0.111 | 0.24 |
| 0.1 μg/ml CDDP | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 0.01 ng/ml A | 1.064 | 0.729 | 1.252 | 2.089 | 2.387 | 2.506 | 0.11 | 0.229 | 0.177 |
| 1 μg/ml CDDP | 0.557 | 0.314 | 0.437 | 2.229 | 2.238 | 2.434 | 0.02 | 0.015 | 0.025 |
| 0.01 ng/ml A + 1 | 0.364 | 0.376 | 0.422 | 2.041 | 2.3 | 2.132 | 0.016 | 0.013 | 0.016 |
| μg/ml CDDP | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 0.01 ng/ml A | 1.064 | 0.729 | 1.252 | 2.089 | 2.387 | 2.506 | 0.11 | 0.229 | 0.177 |
| 10 μg/ml CDDP | 0.728 | 0.405 | 0.436 | 2.246 | 2 | 2.222 | 0.054 | 0.03 | 0.026 |
| 0.01 ng/ml A + | 0.832 | 0.574 | 0.428 | 1.794 | 2.025 | 1.737 | 0.08 | 0.033 | 0.031 |
| 10 μg/ml CDDP | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 0.01 ng/ml A | 1.064 | 0.729 | 1.252 | 2.089 | 2.387 | 2.506 | 0.11 | 0.229 | 0.177 |
| 100 μg/ml CDDP | 0.383 | 0.269 | 0.243 | 2.052 | 2.009 | 1.875 | 0.019 | 0.012 | 0.017 |
| 0.01 ng/ml A + | 1.089 | 0.163 | 0.339 | 1.939 | 2.131 | 2.027 | 0.017 | 0.01 | 0.016 |
| 100 μg/ml CDDP | | | | | | | | | |
| Average | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 0.1 ng/ml A | 1.904 | 0.797 | 1.268 | 2.015 | 2.24 | 2.466 | 0.134 | 0.294 | 0.454 |
| 0.1 μg/ml CDDP | 0.838 | 0.588 | 0.901 | 2.17 | 2.437 | 2.413 | 0.096 | 0.177 | 0.315 |
| 0.1 ng/ml A + 0.1 | 0.88 | 0.544 | 0.822 | 2.196 | 2.295 | 2.669 | 0.098 | 0.175 | 0.199 |
| μg/ml CDDP | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 0.1 ng/ml A | 1.904 | 0.797 | 1.268 | 2.015 | 2.24 | 2.466 | 0.134 | 0.294 | 0.454 |
| 1 μg/ml CDDP | 0.557 | 0.314 | 0.437 | 2.229 | 2.238 | 2.434 | 0.02 | 0.015 | 0.025 |
| 0.1 ng/ml A + 1 | 0.846 | 0.428 | 0.751 | 2.108 | 2.339 | 2.176 | 0.014 | 0.007 | 0.012 |
| μg/ml CDDP | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 0.1 ng/ml A | 1.904 | 0.797 | 1.268 | 2.015 | 2.24 | 2.466 | 0.134 | 0.294 | 0.454 |
| 10 μg/ml CDDP | 0.728 | 0.405 | 0.436 | 2.246 | 2 | 2.222 | 0.054 | 0.03 | 0.026 |
| 0.1 ng/ml A + 10 | 0.93 | 0.292 | 0.915 | 2.248 | 2.199 | 1.924 | 0.062 | 0.042 | 0.028 |
| μg/ml CDDP | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 0.1 ng/ml A | 1.904 | 0.797 | 1.268 | 2.015 | 2.24 | 2.466 | 0.134 | 0.294 | 0.454 |
| 100 μg/ml CDDP | 0.383 | 0.269 | 0.243 | 2.052 | 2.009 | 1.875 | 0.019 | 0.012 | 0.017 |
| 0.1 ng/ml A + | 0.577 | 0.363 | 0.556 | 2.403 | 2.398 | 2.174 | 0.015 | 0.012 | 0.013 |
| 100 μg/ml CDDP | | | | | | | | | |
| Average | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 1 ng/ml A | 0.753 | 0.958 | 1.352 | 2.222 | 2.115 | 2.676 | 0.072 | 0.218 | 0.169 |
| 0.1 μg/ml CDDP | 0.838 | 0.588 | 0.901 | 2.17 | 2.437 | 2.413 | 0.096 | 0.177 | 0.315 |
| 1 ng/ml A + 0.1 | 0.877 | 0.681 | 0.875 | 2.244 | 2.224 | 2.275 | 0.062 | 0.1 | 0.094 |
| μg/ml CDDP | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 1 ng/ml A | 0.753 | 0.958 | 1.352 | 2.222 | 2.115 | 2.676 | 0.072 | 0.218 | 0.169 |
| 1 μg/ml CDDP | 0.557 | 0.314 | 0.437 | 2.229 | 2.238 | 2.434 | 0.02 | 0.015 | 0.025 |
| 1 ng/ml A + 1 | 0.564 | 0.229 | 0.727 | 2.364 | 2.026 | 2.055 | 0.019 | 0.015 | 0.017 |
| μg/ml CDDP | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 1 ng/ml A | 0.753 | 0.958 | 1.352 | 2.222 | 2.115 | 2.676 | 0.072 | 0.218 | 0.169 |
| 10 μg/ml CDDP | 0.728 | 0.405 | 0.436 | 2.246 | 2 | 2.222 | 0.054 | 0.03 | 0.026 |
| 1 ng/ml A + 10 | 0.925 | 0.477 | 0.474 | 2.462 | 2.086 | 2.249 | 0.057 | 0.037 | 0.023 |
| μg/ml CDDP | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 1 ng/ml A | 0.753 | 0.958 | 1.352 | 2.222 | 2.115 | 2.676 | 0.072 | 0.218 | 0.169 |
| 100 μg/ml CDDP | 0.383 | 0.269 | 0.243 | 2.052 | 2.009 | 1.875 | 0.019 | 0.012 | 0.017 |
| 1 ng/ml A + 100 | 0.83 | 0.636 | 0.618 | 2.539 | 2.375 | 2.726 | 0.017 | 0.012 | 0.015 |
| μg/ml CDDP | | | | | | | | | |
| Average | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 10 ng/ml A | 0.72 | 0.983 | 1.438 | 2.118 | 2.188 | 2.484 | 0.079 | 0.151 | 0.162 |
| 0.1 μg/ml CDDP | 0.838 | 0.588 | 0.901 | 2.17 | 2.437 | 2.413 | 0.096 | 0.177 | 0.315 |
| 10 ng/ml A + 0.1 | 0.724 | 0.747 | 0.738 | 2.046 | 1.938 | 2.189 | 0.074 | 0.106 | 0.104 |
| μg/ml CDDP | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 10 ng/ml A | 0.72 | 0.983 | 1.438 | 2.118 | 2.188 | 2.484 | 0.079 | 0.151 | 0.162 |
| 1 μg/ml CDDP | 0.557 | 0.314 | 0.437 | 2.229 | 2.238 | 2.434 | 0.02 | 0.015 | 0.025 |
| 10 ng/m1 A + 1 | 0.633 | 0.424 | 0.469 | 2.163 | 2.047 | 2.225 | 0.049 | 0.039 | 0.028 |
| μg/ml CDDP | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 10 ng/ml A | 0.72 | 0.983 | 1.438 | 2.118 | 2.188 | 2.484 | 0.079 | 0.151 | 0.162 |
| 10 μg/ml CDDP | 0.728 | 0.405 | 0.436 | 2.246 | 2 | 2.222 | 0.054 | 0.03 | 0.026 |
| 10 ng/ml A + 10 | 0.645 | 0.604 | 0.418 | 2.171 | 2.218 | 2.186 | 0.07 | 0.03 | 0.023 |
| μg/ml CDDP | | | | | | | | | |
| Unstimulated | 0.558 | 0.661 | 1.223 | 1.751 | 2.348 | 2.403 | 0.119 | 0.242 | 0.153 |
| cells | | | | | | | | | |
| 10 ng/ml A | 0.72 | 0.983 | 1.438 | 2.118 | 2.188 | 2.484 | 0.079 | 0.151 | 0.162 |
| 100 μg/ml CDDP | 0.383 | 0.269 | 0.243 | 2.052 | 2.009 | 1.875 | 0.019 | 0.012 | 0.017 |
| 10 ng/ml A + 100 | 0.54 | 0.42 | 0.423 | 2.542 | 2.288 | 2.413 | 0.016 | 0.02 | 0.01 |
| μg/ml CDDP | | | | | | | | | |

TABLE 27

| | HT55 | Run 2 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 1.041 | 1.25 | 1.915 | 2.662 | 2.621 | 2.809 | 0.224 | 0.469 | 0.619 |
| cells | | | | | | | | | |
| 0.01 ng/ml A | 1.161 | 1.323 | 2.002 | 2.647 | 2.675 | 2.844 | 0.234 | 0.473 | 0.61 |
| 0.1 μg/ml CDDP | 1.745 | 0.793 | 0.937 | 2.445 | 2.408 | 2.564 | 0.215 | 0.381 | 0.488 |
| 0.01 ng/ml A + | 1.228 | 0.428 | 0.307 | 2.307 | 2.047 | 1.781 | 0.179 | 0.179 | 0.143 |
| 0.1 μg/ml CDDP | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 0.01 ng/ml A | 1.161 | 1.323 | 2.002 | 2.647 | 2.675 | 2.844 | 0.234 | 0.473 | 0.61 |
| 1 μg/ml CDDP | 0.942 | 0.23 | 0.395 | 2.327 | 2.098 | 1.804 | 0.023 | 0.028 | 0.032 |
| 0.01 ng/ml A + 1 | 0.908 | 0.313 | 0.201 | 2.414 | 1.856 | 1.907 | 0.02 | 0.021 | 0.019 |
| μg/ml CDDP | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 0.01 ng/ml A | 1.161 | 1.323 | 2.002 | 2.647 | 2.675 | 2.844 | 0.234 | 0.473 | 0.61 |
| 10 μg/ml CDDP | 1.075 | 0.281 | 0.326 | 2.387 | 1.814 | 1.74 | 0.053 | 0.031 | 0.028 |
| 0.01 ng/ml A + 10 | 0.817 | 0.463 | 0.278 | 2.395 | 2.017 | 1.953 | 0.144 | 0.041 | 0.021 |
| μg/ml CDDP | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 0.01 ng/ml A | 1.161 | 1.323 | 2.002 | 2.647 | 2.675 | 2.844 | 0.234 | 0.473 | 0.61 |
| 100 μg/ml CDDP | 0.758 | 0.286 | 0.244 | 2.766 | 1.508 | 2.019 | 0.018 | 0.025 | 0.021 |
| 0.01 ng/ml A + | 1.384 | 0.553 | 0.492 | 2.614 | 2.292 | 2.339 | 0.016 | 0.018 | 0.016 |
| 100 μg CDDP | | | | | | | | | |
| Average | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 0.1 ng/ml A | 1.492 | 1.545 | 2.133 | 2.525 | 2.731 | 2.839 | 0.315 | 0.511 | 0.51 |
| 0.1 μg/ml CDDP | 1.745 | 0.793 | 0.937 | 2.445 | 2.408 | 2.564 | 0.215 | 0.381 | 0.488 |
| 0.1 ng/ml A + 0.1 | 1.474 | 0.697 | 0.595 | 2.519 | 2.112 | 2.142 | 0.239 | 0.309 | 0.207 |
| μg/ml CDDP | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 0.1 ng/ml A | 1.492 | 1.545 | 2.133 | 2.525 | 2.731 | 2.839 | 0.315 | 0.511 | 0.51 |
| 1 μg/ml CDDP | 0.942 | 0.23 | 0.395 | 2.327 | 2.098 | 1.804 | 0.023 | 0.028 | 0.032 |
| 0.1 ng/ml A + 1 | 0.907 | 0.426 | 0.318 | 2.507 | 2.011 | 2.065 | 0.017 | 0.022 | 0.019 |
| μg/ml CDDP | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 0.1 ng/ml A | 1.492 | 1.545 | 2.133 | 2.525 | 2.731 | 2.839 | 0.315 | 0.511 | 0.51 |
| 10 μg/ml CDDP | 1.075 | 0.281 | 0.326 | 2.387 | 1.814 | 1.74 | 0.053 | 0.031 | 0.028 |
| 0.1 ng/ml A + 10 | 1.533 | 0.545 | 0.335 | 2.348 | 2.214 | 2.24 | 0.099 | 0.031 | 0.027 |
| μg/ml CDDP | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 0.1 ng/ml A | 1.492 | 1.545 | 2.133 | 2.525 | 2.731 | 2.839 | 0.315 | 0.511 | 0.51 |
| 100 μg/ml CDDP | 0.758 | 0.286 | 0.244 | 2.766 | 1.508 | 2.019 | 0.018 | 0.025 | 0.021 |
| 0.1 ng/ml A + 100 | 0.661 | 0.326 | 0.225 | 2.726 | 2.226 | 2.495 | 0.017 | 0.018 | 0.018 |
| μg/ml CDDP | | | | | | | | | |
| Average | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 1 ng/ml A | 1.484 | 1.632 | 2.63 | 2.748 | 2.536 | 2.861 | 0.242 | 0.412 | 0.457 |
| 0.1 μg/ml CDDP | 1.745 | 0.793 | 0.937 | 2.445 | 2.408 | 2.564 | 0.215 | 0.381 | 0.488 |
| 1 ng/ml A + 0.1 | 1.802 | 0.901 | 0.696 | 2.686 | 2.299 | 2.362 | 0.167 | 0.27 | 0.219 |
| μg/ml CDDP | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 1 ng/ml A | 1.484 | 1.632 | 2.63 | 2.748 | 2.536 | 2.861 | 0.242 | 0.412 | 0.457 |
| 1 μg/ml CDDP | 0.942 | 0.23 | 0.395 | 2.327 | 2.098 | 1.804 | 0.023 | 0.028 | 0.032 |
| 1 ng/ml A + 1 | 1.092 | 0.626 | 0.279 | 2.803 | 2.209 | 2.144 | 0.019 | 0.024 | 0.019 |
| μg/ml CDDP | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 1 ng/ml A | 1.484 | 1.632 | 2.63 | 2.748 | 2.536 | 2.861 | 0.242 | 0.412 | 0.457 |
| 10 μg/ml CDDP | 1.075 | 0.281 | 0.326 | 2.387 | 1.814 | 1.74 | 0.053 | 0.031 | 0.028 |
| 1 ng/ml A + 10 | 1.173 | 0.738 | 0.361 | 2.746 | 2.323 | 2.158 | 0.151 | 0.054 | 0.029 |
| μg/ml CDDP | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 1 ng/ml A | 1.484 | 1.632 | 2.63 | 2.748 | 2.536 | 2.861 | 0.242 | 0.412 | 0.457 |
| 100 μg/ml CDDP | 0.758 | 0.286 | 0.244 | 2.766 | 1.508 | 2.019 | 0.018 | 0.025 | 0.021 |
| 1 ng/ml A + 100 | 0.892 | 0.841 | 0.35 | 2.732 | 2.546 | 2.397 | 0.021 | 0.019 | 0.021 |
| μg/ml CDDP | | | | | | | | | |
| Average | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 10 ng/ml A | 1.437 | 1.599 | 2.513 | 2.735 | 2.636 | 2.854 | 0.25 | 0.477 | 0.519 |
| 0.1 μg/ml CDDP | 1.745 | 0.793 | 0.937 | 2.445 | 2.408 | 2.564 | 0.215 | 0.381 | 0.488 |
| 10 ng/ml A + 0.1 | 1.427 | 0.892 | 0.668 | 2.705 | 2.519 | 2.343 | 0.231 | 0.324 | 0.213 |
| μg/ml CDDP | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 10 ng/ml A | 1.437 | 1.599 | 2.513 | 2.735 | 2.636 | 2.854 | 0.25 | 0.477 | 0.519 |
| 1 μg/ml CDDP | 0.942 | 0.23 | 0.395 | 2.327 | 2.098 | 1.804 | 0.023 | 0.028 | 0.032 |
| 10 ng/ml A + 1 | 1.098 | 0.303 | 0.293 | 2.752 | 1.9 | 2.051 | 0.02 | 0.022 | 0.02 |
| μg/ml CDDP | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 10 ng/ml A | 1.437 | 1.599 | 2.513 | 2.735 | 2.636 | 2.854 | 0.25 | 0.477 | 0.519 |
| 10 μg/ml CDDP | 1.075 | 0.281 | 0.326 | 2.387 | 1.814 | 1.74 | 0.053 | 0.031 | 0.028 |
| 10 ng/ml A + 10 | 1.723 | 0.838 | 0.255 | 2.706 | 1.879 | 2.131 | 0.112 | 0.039 | 0.028 |
| μg/ml CDDP | | | | | | | | | |
| Unstimulated | 1.041 | 1.25 | 1.915 | 2.662 | 2.621 | 2.809 | 0.224 | 0.469 | 0.619 |
| cells | | | | | | | | | |
| 10 ng/ml A | 1.437 | 1.599 | 2.513 | 2.735 | 2.636 | 2.854 | 0.25 | 0.477 | 0.519 |
| 100 μg/ml CDDP | 0.758 | 0.286 | 0.244 | 2.766 | 1.508 | 2.019 | 0.018 | 0.025 | 0.021 |
| 10 ng/ml A + 100 | 1.368 | 1.058 | 0.424 | 2.821 | 2.044 | 1.993 | 0.019 | 0.021 | 0.02 |
| μg/ml CDDP | | | | | | | | | |

TABLE 28

| | HT55 | Run 3 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.766 | 1.22 | 2.264 | 2.169 | 2.742 | 2.692 | 0.209 | 0.427 | 0.639 |
| cells | | | | | | | | | |
| 0.01 ng/ml A | 0.767 | 1.66 | 2.304 | 2.316 | 2.714 | 2.719 | 0.21 | 0.377 | 0.533 |
| 0.1 μg/ml CDDP | 0.591 | 1.099 | 1.228 | 2.101 | 2.648 | 2.177 | 0.205 | 0.331 | 0.359 |
| 0.01 ng/ml A + | 0.581 | 0.832 | 0.896 | 2.117 | 2.536 | 1.872 | 0.176 | 0.265 | 0.196 |
| 0.1 μg/ml CDDP | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 0.01 ng/ml A | 0.767 | 1.66 | 2.304 | 2.316 | 2.714 | 2.719 | 0.21 | 0.377 | 0.533 |
| 1 μg/ml CDDP | 0.201 | 0.561 | 0.577 | 1.801 | 2.071 | 1.424 | 0.035 | 0.04 | 0.032 |
| 0.01 ng/ml A + 1 | 0.098 | 0.295 | 0.457 | 1.956 | 1.873 | 1.693 | 0.025 | 0.035 | 0.017 |
| μg/ml CDDP | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 0.01 ng/ml A | 0.767 | 1.66 | 2.304 | 2.316 | 2.714 | 2.719 | 0.21 | 0.377 | 0.533 |
| 10 μg/ml CDDP | 0.812 | 1.712 | 2.329 | 2.557 | 2.649 | 2.784 | 0.206 | 0.361 | 0.353 |
| 0.01 ng/ml A + 10 | 0.365 | 0.573 | 0.777 | 2.26 | 1.939 | 1.805 | 0.13 | 0.076 | 0.032 |
| μg/ml CDDP | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 0.01 ng/ml A | 0.767 | 1.66 | 2.304 | 2.316 | 2.714 | 2.719 | 0.21 | 0.377 | 0.533 |
| 100 μg/ml CDDP | 0.15 | 0.285 | 0.431 | 2.038 | 1.727 | 1.831 | 0.028 | 0.038 | 0.031 |
| 0.01 ng/ml A + | 0.266 | 0.792 | 0.705 | 2.257 | 1.825 | 1.994 | 0.02 | 0.028 | 0.016 |
| 100 μg/ml CDDP | | | | | | | | | |
| Average | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 0.1 ng/ml A | 0.837 | 1.639 | 2.102 | 2.343 | 2.724 | 2.737 | 0.215 | 0.391 | 0.426 |
| 0.1 μg/ml CDDP | 0.591 | 1.099 | 1.228 | 2.101 | 2.648 | 2.177 | 0.205 | 0.331 | 0.359 |
| 0.1 ng/ml A + 0.1 | 0.581 | 1.123 | 1.162 | 2.292 | 2.216 | 2.018 | 0.201 | 0.31 | 0.229 |
| μg/ml CDDP | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 0.1 ng/ml A | 0.837 | 1.639 | 2.102 | 2.343 | 2.724 | 2.737 | 0.215 | 0.391 | 0.426 |
| 1 μg/ml CDDP | 0.201 | 0.561 | 0.577 | 1.801 | 2.071 | 1.424 | 0.035 | 0.04 | 0.032 |
| 0.1 ng/ml A + 1 | 0.163 | 0.463 | 0.876 | 2.241 | 1.684 | 1.757 | 0.024 | 0.03 | 0.015 |
| μg/ml CDDP | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 0.1 ng/ml A | 0.837 | 1.639 | 2.102 | 2.343 | 2.724 | 2.737 | 0.215 | 0.391 | 0.426 |
| 10 μg/ml CDDP | 0.812 | 1.712 | 2.329 | 2.557 | 2.649 | 2.784 | 0.206 | 0.361 | 0.353 |
| 0.1 ng/ml A + 10 | 0.352 | 0.609 | 0.923 | 2.15 | 2.193 | 1.89 | 0.152 | 0.076 | 0.031 |
| μg/ml CDDP | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 0.1 ng/ml A | 0.837 | 1.639 | 2.102 | 2.343 | 2.724 | 2.737 | 0.215 | 0.391 | 0.426 |
| 100 μg/ml CDDP | 0.15 | 0.285 | 0.431 | 2.038 | 1.727 | 1.831 | 0.028 | 0.038 | 0.031 |
| 0.1 ng/ml A + 100 | 0.284 | 0.824 | 1.5 | 2.367 | 2.107 | 2.346 | 0.022 | 0.03 | 0.016 |
| μg/ml CDDP | | | | | | | | | |
| Average | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 1 ng/ml A | 0.756 | 1.962 | 1.603 | 2.534 | 2.721 | 2.756 | 0.23 | 0.334 | 0.361 |
| 0.1 μg/ml CDDP | 0.591 | 1.099 | 1.228 | 2.101 | 2.648 | 2.177 | 0.205 | 0.331 | 0.359 |
| 1 ng/ml A + 0.1 | 0.673 | 1.297 | 1.399 | 2.56 | 2.434 | 2.102 | 0.204 | 0.285 | 0.187 |
| μg/ml CDDP | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 1 ng/ml A | 0.756 | 1.962 | 1.603 | 2.534 | 2.721 | 2.756 | 0.23 | 0.334 | 0.361 |
| 1 μg/ml CDDP | 0.201 | 0.561 | 0.577 | 1.801 | 2.071 | 1.424 | 0.035 | 0.04 | 0.032 |
| 1 ng/ml A + 1 | 0.197 | 0.773 | 0.789 | 2.211 | 2.017 | 2.021 | 0.03 | 0.034 | 0.02 |
| μg/ml CDDP | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 1 ng/ml A | 0.756 | 1.962 | 1.603 | 2.534 | 2.721 | 2.756 | 0.23 | 0.334 | 0.361 |
| 10 μg/ml CDDP | 0.812 | 1.712 | 2.329 | 2.557 | 2.649 | 2.784 | 0.206 | 0.361 | 0.353 |
| 1 ng/ml A + 10 | 0.445 | 0.688 | 1.822 | 2.477 | 1.849 | 2.491 | 0.165 | 0.074 | 0.031 |
| μg/ml CDDP | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 1 ng/ml A | 0.756 | 1.962 | 1.603 | 2.534 | 2.721 | 2.756 | 0.23 | 0.334 | 0.361 |
| 100 μg/ml CDDP | 0.15 | 0.285 | 0.431 | 2.038 | 1.727 | 1.831 | 0.028 | 0.038 | 0.031 |
| 1 ng/ml A + 100 | 0.241 | 0.807 | 1.881 | 2.309 | 2.109 | 2.412 | 0.029 | 0.029 | 0.018 |
| μg/ml CDDP | | | | | | | | | |
| Average | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 10 ng/ml A | 0.713 | 1.687 | 2.442 | 2.294 | 2.606 | 2.748 | 0.193 | 0.403 | 0.513 |
| 0.1 μg/ml CDDP | 0.591 | 1.099 | 1.228 | 2.101 | 2.648 | 2.177 | 0.205 | 0.331 | 0.359 |
| 10 ng/ml A + 0.1 | 0.542 | 1.355 | 1.408 | 2.486 | 2.303 | 1.975 | 0.184 | 0.286 | 0.246 |
| μg/ml CDDP | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 10 ng/ml A | 0.713 | 1.687 | 2.442 | 2.294 | 2.606 | 2.748 | 0.193 | 0.403 | 0.513 |
| 1 μg/ml CDDP | 0.201 | 0.561 | 0.577 | 1.801 | 2.071 | 1.424 | 0.035 | 0.04 | 0.032 |
| 10 ng/ml A + 1 | 0.205 | 1.103 | 0.524 | 2.268 | 1.691 | 1.672 | 0.027 | 0.031 | 0.017 |
| μg/ml CDDP | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 10 ng/ml A | 0.713 | 1.687 | 2.442 | 2.294 | 2.606 | 2.748 | 0.193 | 0.403 | 0.513 |
| 10 μg/ml CDDP | 0.812 | 1.712 | 2.329 | 2.557 | 2.649 | 2.784 | 0.206 | 0.361 | 0.353 |
| 10 ng/ml A + 10 | 0.369 | 0.953 | 0.676 | 2.345 | 1.721 | 1.804 | 0.104 | 0.071 | 0.028 |
| μg/ml CDDP | | | | | | | | | |
| Unstimulated | 0.766 | 1.22 | 2.264 | 2.169 | 2.742 | 2.692 | 0.209 | 0.427 | 0.639 |
| cells | | | | | | | | | |
| 10 ng/ml A | 0.713 | 1.687 | 2.442 | 2.294 | 2.606 | 2.748 | 0.193 | 0.403 | 0.513 |
| 100 μg/ml CDDP | 0.15 | 0.285 | 0.431 | 2.038 | 1.727 | 1.831 | 0.028 | 0.038 | 0.031 |
| 10 ng/ml A + 100 | 0.197 | 0.985 | 1.166 | 2.284 | 1.991 | 2.052 | 0.021 | 0.029 | 0.017 |
| μg/ml CDDP | | | | | | | | | |

TABLE 29

| | LNCaP | Run 1 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.507 | 1.362 | 2.768 | 1.437 | 2.873 | 2.952 | 0.096 | 0.511 | 0.487 |
| cells | | | | | | | | | |
| 0.01 ng/ml A | 0.501 | 1.309 | 2.804 | 1.55 | 2.928 | 2.912 | 0.058 | 0.314 | 0.422 |
| 0.1 μg/ml CDDP | 0.418 | 0.541 | 0.684 | 1.611 | 2.798 | 2.532 | 0.061 | 0.116 | 0.132 |
| 0.01 ng/ml A + | 0.382 | 0.383 | 0.478 | 1.443 | 2.752 | 2.242 | 0.052 | 0.087 | 0.103 |
| 0.1 μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 0.01 ng/ml A | 0.501 | 1.309 | 2.804 | 1.55 | 2.928 | 2.912 | 0.058 | 0.314 | 0.422 |
| 1 μg/ml CDDP | 0.327 | 0.252 | 0.558 | 1.294 | 2.888 | 2.483 | 0.012 | 0.015 | 0.016 |
| 0.01 ng/ml A + 1 | 0.106 | 0.2 | 0.197 | 1.364 | 2.63 | 2.2 | 0.009 | 0.002 | 0.008 |
| μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 0.01 ng/ml A | 0.501 | 1.309 | 2.804 | 1.55 | 2.928 | 2.912 | 0.058 | 0.314 | 0.422 |
| 10 μg/ml CDDP | 0.289 | 0.467 | 0.423 | 1.205 | 2.893 | 2.648 | 0.02 | 0.011 | 0.01 |
| 0.01 ng/ml A + 10 | 0.17 | 0.213 | 0.191 | 1.247 | 2.719 | 1.992 | 0.018 | 0.005 | 0.008 |
| μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 0.01 ng/ml A | 0.501 | 1.309 | 2.804 | 1.55 | 2.928 | 2.912 | 0.058 | 0.314 | 0.422 |
| 100 μg/ml CDDP | 0.359 | 0.306 | 0.398 | 1.2 | 2.555 | 2.649 | 0.011 | 0.017 | 0.01 |
| 0.01 ng/ml A + | 0.163 | 0.161 | 0.237 | 1.744 | 2.763 | 1.88 | 0.01 | 0.006 | 0.012 |
| 100 μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 0.1 ng/ml A | 0.829 | 1.366 | 2.318 | 1.468 | 2.961 | 2.993 | 0.089 | 0.229 | 0.455 |
| 0.1 μg/ml CDDP | 0.418 | 0.541 | 0.684 | 1.611 | 2.798 | 2.532 | 0.061 | 0.116 | 0.132 |
| 0.1 ng/ml A + 0.1 | 0.435 | 0.483 | 0.513 | 1.62 | 2.891 | 2.368 | 0.044 | 0.11 | 0.082 |
| μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 0.1 ng/ml A | 0.829 | 1.366 | 2.318 | 1.468 | 2.961 | 2.993 | 0.089 | 0.229 | 0.455 |
| 1 μg/ml CDDP | 0.327 | 0.252 | 0.558 | 1.294 | 2.888 | 2.483 | 0.012 | 0.015 | 0.016 |
| 0.1 ng/ml A + 1 | 0.25 | 0.341 | 0.369 | 1.462 | 2.824 | 2.494 | 0.009 | 0.007 | 0.011 |
| μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 0.1 ng/ml A | 0.829 | 1.366 | 2.318 | 1.468 | 2.961 | 2.993 | 0.089 | 0.229 | 0.455 |
| 10 μg/ml CDDP | 0.289 | 0.467 | 0.423 | 1.205 | 2.893 | 2.648 | 0.02 | 0.011 | 0.01 |
| 0.1 ng/ml A + 10 | 0.366 | 0.286 | 0.282 | 1.511 | 2.842 | 2.449 | 0.022 | 0.004 | 0.011 |
| μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 0.1 ng/ml A | 0.829 | 1.366 | 2.318 | 1.468 | 2.961 | 2.993 | 0.089 | 0.229 | 0.455 |
| 100 μg/ml CDDP | 0.359 | 0.306 | 0.398 | 1.2 | 2.555 | 2.649 | 0.011 | 0.017 | 0.01 |
| 0.1 ng/ml A + 100 | 0.374 | 0.183 | 0.172 | 1.96 | 2.6 | 1.864 | 0.009 | 0.007 | 0.012 |
| μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 1 ng/ml A | 0.685 | 1.792 | 2.829 | 1.486 | 2.933 | 2.987 | 0.093 | 0.235 | 0.416 |
| 0.1 μg/ml CDDP | 0.418 | 0.541 | 0.684 | 1.611 | 2.798 | 2.532 | 0.061 | 0.116 | 0.132 |
| 1 ng/ml A + 0.1 | 0.565 | 0.694 | 0.561 | 1.404 | 2.942 | 2.684 | 0.04 | 0.096 | 0.092 |
| μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 1 ng/ml A | 0.685 | 1.792 | 2.829 | 1.486 | 2.933 | 2.987 | 0.093 | 0.235 | 0.416 |
| 1 μg/ml CDDP | 0.327 | 0.252 | 0.558 | 1.294 | 2.888 | 2.483 | 0.012 | 0.015 | 0.016 |
| 1 ng/ml A + 1 | 0.209 | 0.238 | 0.285 | 1.535 | 2.887 | 2.684 | 0.008 | 0.011 | 0.009 |
| μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 1 ng/ml A | 0.685 | 1.792 | 2.829 | 1.486 | 2.933 | 2.987 | 0.093 | 0.235 | 0.416 |
| 10 μg/ml CDDP | 0.289 | 0.467 | 0.423 | 1.205 | 2.893 | 2.648 | 0.02 | 0.011 | 0.01 |
| 1 ng/ml A + 10 | 0.334 | 0.264 | 0.271 | 1.595 | 2.793 | 2.505 | 0.023 | 0.009 | 0.012 |
| μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 1 ng/ml A | 0.685 | 1.792 | 2.829 | 1.486 | 2.933 | 2.987 | 0.093 | 0.235 | 0.416 |
| 100 μg/ml CDDP | 0.359 | 0.306 | 0.398 | 1.2 | 2.555 | 2.649 | 0.011 | 0.017 | 0.01 |
| 1 ng/ml A + 100 | 0.274 | 0.135 | 0.213 | 1.716 | 2.535 | 1.878 | 0.013 | 0.009 | 0.014 |
| μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 10 ng/ml A | 0.836 | 1.406 | 2.657 | 1.376 | 2.959 | 2.973 | 0.089 | 0.232 | 0.491 |
| 0.1 μg/ml CDDP | 0.418 | 0.541 | 0.684 | 1.611 | 2.798 | 2.532 | 0.061 | 0.116 | 0.132 |
| 10 ng/ml A + 0.1 | 0.711 | 0.629 | 0.495 | 1.497 | 2.81 | 2.754 | 0.072 | 0.105 | 0.14 |
| μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 10 ng/ml A | 0.836 | 1.406 | 2.657 | 1.376 | 2.959 | 2.973 | 0.089 | 0.232 | 0.491 |
| 1 μg/ml CDDP | 0.327 | 0.252 | 0.558 | 1.294 | 2.888 | 2.483 | 0.012 | 0.015 | 0.016 |
| 10 ng/ml A + 1 | 0.176 | 0.232 | 0.205 | 1.268 | 2.63 | 2.808 | 0.009 | 0.009 | 0.008 |
| μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 10 ng/ml A | 0.836 | 1.406 | 2.657 | 1.376 | 2.959 | 2.973 | 0.089 | 0.232 | 0.491 |
| 10 μg/ml CDDP | 0.289 | 0.467 | 0.423 | 1.205 | 2.893 | 2.648 | 0.02 | 0.011 | 0.01 |
| 10 ng/ml A + 10 | 0.339 | 0.188 | 0.249 | 1.371 | 2.192 | 2.136 | 0.028 | 0.012 | 0.019 |
| μg/ml CDDP | | | | | | | | | |
| Unstimulated | 0.507 | 1.362 | 2.768 | 1.437 | 2.873 | 2.952 | 0.096 | 0.511 | 0.487 |
| cells | | | | | | | | | |
| 10 ng/ml A | 0.836 | 1.406 | 2.657 | 1.376 | 2.959 | 2.973 | 0.089 | 0.232 | 0.491 |
| 100 μg/ml CDDP | 0.359 | 0.306 | 0.398 | 1.2 | 2.555 | 2.649 | 0.011 | 0.017 | 0.01 |
| 10 ng/ml A + 100 | 0.333 | 0.139 | 0.185 | 1.449 | 2.233 | 1.882 | 0.011 | 0.008 | 0.014 |
| μg/ml CDDP | | | | | | | | | |

TABLE 30

| | LNCaP | Run 2 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.675 | 1.307 | 2.08 | 1.668 | 2.616 | 2.906 | 0.169 | 0.429 | 0.553 |
| cells | | | | | | | | | |
| 0.01 ng/ml A | 0.561 | 1.249 | 2.416 | 1.751 | 2.667 | 2.927 | 0.125 | 0.292 | 0.373 |
| 0.1 μg/ml CDDP | 0.463 | 0.408 | 0.448 | 1.78 | 1.681 | 1.766 | 0.136 | 0.161 | 0.17 |
| 0.01 ng/ml A + | 0.235 | 0.178 | 0.187 | 1.116 | 1.033 | 1.095 | 0.049 | 0.033 | 0.035 |
| 0.1 μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 0.01 ng/ml A | 0.561 | 1.249 | 2.416 | 1.751 | 2.667 | 2.927 | 0.125 | 0.292 | 0.373 |
| 1 μg/ml CDDP | 0.227 | 0.29 | 0.522 | 1.495 | 1.14 | 1.396 | 0.01 | 0.016 | 0.026 |
| 0.01 ng/ml A + 1 | 0.152 | 0.248 | 0.348 | 1.018 | 0.949 | 1.161 | 0.008 | 0.011 | 0.009 |
| μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 0.01 ng/ml A | 0.561 | 1.249 | 2.416 | 1.751 | 2.667 | 2.927 | 0.125 | 0.292 | 0.373 |
| 10 μg/ml CDDP | 0.278 | 0.207 | 0.299 | 0.982 | 1.077 | 1.28 | 0.014 | 0.015 | 0.012 |
| 0.01 ng/ml A + 10 | 0.222 | 0.22 | 0.318 | 1.192 | 1.13 | 1.135 | 0.012 | 0.016 | 0.011 |
| μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 0.01 ng/ml A | 0.561 | 1.249 | 2.416 | 1.751 | 2.667 | 2.927 | 0.125 | 0.292 | 0.373 |
| 100 μg/ml CDDP | 0.276 | 0.237 | 0.214 | 0.985 | 1.138 | 1.25 | 0.017 | 0.015 | 0.012 |
| 0.01 ng/ml A + | 0.189 | 0.253 | 0.257 | 1.687 | 1.305 | 1.497 | 0.009 | 0.018 | 0.014 |
| 100 μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 0.1 ng/ml A | 0.641 | 1.061 | 2.112 | 1.707 | 2.51 | 2.9 | 0.15 | 0.273 | 0.38 |
| 0.1 μg/ml CDDP | 0.463 | 0.408 | 0.448 | 1.78 | 1.681 | 1.766 | 0.136 | 0.161 | 0.17 |
| 0.1 ng/ml A + 0.1 | 0.512 | 0.391 | 0.53 | 1.375 | 1.24 | 1.304 | 0.065 | 0.091 | 0.072 |
| μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 0.1 ng/ml A | 0.641 | 1.061 | 2.112 | 1.707 | 2.51 | 2.9 | 0.15 | 0.273 | 0.38 |
| 1 μg/ml CDDP | 0.227 | 0.29 | 0.522 | 1.495 | 1.14 | 1.396 | 0.01 | 0.016 | 0.026 |
| 0.1 ng/ml A + 1 | 0.306 | 0.278 | 0.196 | 1.184 | 1.16 | 1.487 | 0.018 | 0.018 | 0.008 |
| μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 0.1 ng/ml A | 0.641 | 1.061 | 2.112 | 1.707 | 2.51 | 2.9 | 0.15 | 0.273 | 0.38 |
| 10 μg/ml CDDP | 0.278 | 0.207 | 0.299 | 0.982 | 1.077 | 1.28 | 0.014 | 0.015 | 0.012 |
| 0.1 ng/ml A + 10 | 1.514 | 0.317 | 0.505 | 1.521 | 1.471 | 1.664 | 0.018 | 0.013 | 0.01 |
| μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 0.1 ng/ml A | 0.641 | 1.061 | 2.112 | 1.707 | 2.51 | 2.9 | 0.15 | 0.273 | 0.38 |
| 100 μg/ml CDDP | 0.276 | 0.237 | 0.214 | 0.985 | 1.138 | 1.25 | 0.017 | 0.015 | 0.012 |
| 0.1 ng/ml A + 100 | 0.389 | 0.302 | 0.38 | 1.846 | 1.571 | 1.916 | 0.015 | 0.018 | 0.014 |
| μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 1 ng/ml A | 0.797 | 1.477 | 2.878 | 1.482 | 2.273 | 3.013 | 0.125 | 0.209 | 0.337 |
| 0.1 μg/ml CDDP | 0.463 | 0.408 | 0.448 | 1.78 | 1.681 | 1.766 | 0.136 | 0.161 | 0.17 |
| 1 ng/ml A + 0.1 | 0.691 | 0.582 | 0.436 | 1.51 | 1.414 | 1.566 | 0.071 | 0.085 | 0.083 |
| μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 1 ng/ml A | 0.797 | 1.477 | 2.878 | 1.482 | 2.273 | 3.013 | 0.125 | 0.209 | 0.337 |
| 1 μg/ml CDDP | 0.227 | 0.29 | 0.522 | 1.495 | 1.14 | 1.396 | 0.01 | 0.016 | 0.026 |
| 1 ng/ml A + 1 | 0.309 | 0.206 | 0.293 | 1.482 | 1.29 | 1.75 | 0.014 | 0.016 | 0.013 |
| μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 1 ng/ml A | 0.797 | 1.477 | 2.878 | 1.482 | 2.273 | 3.013 | 0.125 | 0.209 | 0.337 |
| 10 μg/ml CDDP | 0.278 | 0.207 | 0.299 | 0.982 | 1.077 | 1.28 | 0.014 | 0.015 | 0.012 |
| 1 ng/ml A + 10 | 0.718 | 0.809 | 0.546 | 2.592 | 2.677 | 2.39 | 0.163 | 0.264 | 0.33 |
| μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 1 ng/ml A | 0.797 | 1.477 | 2.878 | 1.482 | 2.273 | 3.013 | 0.125 | 0.209 | 0.337 |
| 100 μg/ml CDDP | 0.276 | 0.237 | 0.214 | 0.985 | 1.138 | 1.25 | 0.017 | 0.015 | 0.012 |
| 1 ng/ml A + 100 | 0.57 | 0.396 | 0.258 | 1.99 | 1.814 | 2.194 | 0.022 | 0.024 | 0.019 |
| μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 10 ng/ml A | 0.742 | 1.584 | 2.671 | 1.406 | 2.559 | 2.878 | 0.127 | 0.231 | 0.385 |
| 0.1 μg/ml CDDP | 0.463 | 0.408 | 0.448 | 1.78 | 1.681 | 1.766 | 0.136 | 0.161 | 0.17 |
| 10 ng/ml A + 0.1 | 0.831 | 0.497 | 0.521 | 1.426 | 1.301 | 1.465 | 0.273 | 0.085 | 0.068 |
| μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 10 ng/ml A | 0.742 | 1.584 | 2.671 | 1.406 | 2.559 | 2.878 | 0.127 | 0.231 | 0.385 |
| 1 μg/ml CDDP | 0.227 | 0.29 | 0.522 | 1.495 | 1.14 | 1.396 | 0.01 | 0.016 | 0.026 |
| 10 ng/ml A + 1 | 0.327 | 0.206 | 0.315 | 1.48 | 1.092 | 1.616 | 0.025 | 0.023 | 0.011 |
| μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 10 ng/ml A | 0.742 | 1.584 | 2.671 | 1.406 | 2.559 | 2.878 | 0.127 | 0.231 | 0.385 |
| 10 μg/ml CDDP | 0.278 | 0.207 | 0.299 | 0.982 | 1.077 | 1.28 | 0.014 | 0.015 | 0.012 |
| 10 ng/ml A + 10 | 1.251 | 0.571 | 1.022 | 2.542 | 2.537 | 2.551 | 0.285 | 0.307 | 0.794 |
| μg/ml CDDP | | | | | | | | | |
| Unstimulated | 0.675 | 1.307 | 2.08 | 1.668 | 2.616 | 2.906 | 0.169 | 0.429 | 0.553 |
| cells | | | | | | | | | |
| 10 ng/ml A | 0.742 | 1.584 | 2.671 | 1.406 | 2.559 | 2.878 | 0.127 | 0.231 | 0.385 |
| 100 μg/ml CDDP | 0.276 | 0.237 | 0.214 | 0.985 | 1.138 | 1.25 | 0.017 | 0.015 | 0.012 |
| 10 ng/ml A + 100 | 0.481 | 0.203 | 0.328 | 1.611 | 1.426 | 1.944 | 0.012 | 0.016 | 0.031 |
| μg/ml CDDP | | | | | | | | | |

TABLE 31

| | LNCaP | Run 3 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.526 | 1.008 | 2.252 | 1.66 | 2.716 | 2.881 | 0.191 | 0.443 | 0.514 |
| cells | | | | | | | | | |
| 0.01 ng/ml A | 0.521 | 1.118 | 2.533 | 2.043 | 2.657 | 2.894 | 0.155 | 0.39 | 0.435 |
| 0.1 μg/ml CDDP | 0.425 | 0.387 | 0.619 | 1.975 | 1.761 | 2.009 | 0.11 | 0.157 | 0.171 |
| 0.01 ng/ml A + | 0.345 | 0.301 | 0.281 | 1.45 | 1.636 | 1.725 | 0.097 | 0.121 | 0.07 |
| 0.1 μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 0.01 ng/ml A | 0.521 | 1.118 | 2.533 | 2.043 | 2.657 | 2.894 | 0.155 | 0.39 | 0.435 |
| 1 μg/ml CDDP | 0.152 | 0.24 | 0.312 | 1.541 | 1.56 | 1.584 | 0.008 | 0.011 | 0.01 |
| 0.01 ng/ml A + 1 | 0.13 | 0.194 | 0.146 | 1.103 | 1.696 | 1.467 | 0.002 | 0.007 | 0.006 |
| μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 0.01 ng/ml A | 0.521 | 1.118 | 2.533 | 2.043 | 2.657 | 2.894 | 0.155 | 0.39 | 0.435 |
| 10 μg/ml CDDP | 0.171 | 0.267 | 0.303 | 1.242 | 1.46 | 1.561 | 0.017 | 0.009 | 0.009 |
| 0.01 ng/ml A + 10 | 0.229 | 0.285 | 0.198 | 1.134 | 1.306 | 1.345 | 0.013 | 0.006 | 0.005 |
| μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 0.01 ng/ml A | 0.521 | 1.118 | 2.533 | 2.043 | 2.657 | 2.894 | 0.155 | 0.39 | 0.435 |
| 100 μg/ml CDDP | 0.133 | 0.3 | 0.289 | 1.2 | 1.284 | 1.421 | 0.006 | 0.009 | 0.008 |
| 0.01 ng/ml A + | 0.217 | 0.327 | 0.417 | 1.393 | 1.411 | 1.633 | 0.005 | 0.007 | 0.006 |
| 100 μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 0.1 ng/ml A | 0.538 | 1.086 | 2.306 | 2.013 | 2.49 | 2.887 | 0.149 | 0.307 | 0.315 |
| 0.1 μg/ml CDDP | 0.425 | 0.387 | 0.619 | 1.975 | 1.761 | 2.009 | 0.11 | 0.157 | 0.171 |
| 0.1 ng/ml A + 0.1 | 0.348 | 0.501 | 0.386 | 1.592 | 2.255 | 1.811 | 0.07 | 0.109 | 0.093 |
| μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 0.1 ng/ml A | 0.538 | 1.086 | 2.306 | 2.013 | 2.49 | 2.887 | 0.149 | 0.307 | 0.315 |
| 1 μg/ml CDDP | 0.152 | 0.24 | 0.312 | 1.541 | 1.56 | 1.584 | 0.008 | 0.011 | 0.01 |
| 0.1 ng/ml A + 1 | 0.163 | 0.216 | 0.438 | 1.539 | 1.933 | 1.537 | 0.003 | 0.007 | 0.008 |
| μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 0.1 ng/ml A | 0.538 | 1.086 | 2.306 | 2.013 | 2.49 | 2.887 | 0.149 | 0.307 | 0.315 |
| 10 μg/ml CDDP | 0.171 | 0.267 | 0.303 | 1.242 | 1.46 | 1.561 | 0.017 | 0.009 | 0.009 |
| 0.1 ng/ml A + 10 | 0.248 | 0.324 | 0.356 | 1.618 | 1.596 | 1.297 | 0.009 | 0.008 | 0.005 |
| μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 0.1 ng/ml A | 0.538 | 1.086 | 2.306 | 2.013 | 2.49 | 2.887 | 0.149 | 0.307 | 0.315 |
| 100 μg/ml CDDP | 0.133 | 0.3 | 0.289 | 1.2 | 1.284 | 1.421 | 0.006 | 0.009 | 0.008 |
| 0.1 ng/ml A + 100 | 0.202 | 0.312 | 1.896 | 1.441 | 1.625 | 1.826 | 0.006 | 0.006 | 0.007 |
| μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 1 ng/ml A | 0.599 | 1.327 | 2.743 | 1.897 | 2.611 | 2.884 | 0.089 | 0.241 | 0.322 |
| 0.1 μg/ml CDDP | 0.425 | 0.387 | 0.619 | 1.975 | 1.761 | 2.009 | 0.11 | 0.157 | 0.171 |
| 1 ng/ml A + 0.1 | 0.388 | 0.717 | 0.625 | 1.503 | 1.923 | 1.729 | 0.073 | 0.085 | 0.08 |
| μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 1 ng/ml A | 0.599 | 1.327 | 2.743 | 1.897 | 2.611 | 2.884 | 0.089 | 0.241 | 0.322 |
| 1 μg/ml CDDP | 0.152 | 0.24 | 0.312 | 1.541 | 1.56 | 1.584 | 0.008 | 0.011 | 0.01 |
| 1 ng/ml A + 1 | 0.137 | 0.554 | 0.322 | 1.875 | 1.654 | 1.772 | 0.006 | 0.007 | 0.004 |
| μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 1 ng/ml A | 0.599 | 1.327 | 2.743 | 1.897 | 2.611 | 2.884 | 0.089 | 0.241 | 0.322 |
| 10 μg/ml CDDP | 0.171 | 0.267 | 0.303 | 1.242 | 1.46 | 1.561 | 0.017 | 0.009 | 0.009 |
| 1 ng/ml A + 10 | 0.264 | 0.654 | 0.601 | 1.383 | 1.399 | 1.866 | 0.02 | 0.008 | 0.007 |
| μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 1 ng/ml A | 0.599 | 1.327 | 2.743 | 1.897 | 2.611 | 2.884 | 0.089 | 0.241 | 0.322 |
| 100 μg/ml CDDP | 0.133 | 0.3 | 0.289 | 1.2 | 1.284 | 1.421 | 0.006 | 0.009 | 0.008 |
| 1 ng/ml A + 100 | 0.501 | 1.064 | 0.87 | 1.412 | 1.644 | 1.739 | 0.005 | 0.01 | 0.007 |
| μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 10 ng/ml A | 0.568 | 1.343 | 2.616 | 1.544 | 2.667 | 2.857 | 0.14 | 0.305 | 0.333 |
| 0.1 μg/ml CDDP | 0.425 | 0.387 | 0.619 | 1.975 | 1.761 | 2.009 | 0.11 | 0.157 | 0.171 |
| 10 ng/ml A + 0.1 | 0.367 | 0.541 | 0.583 | 1.648 | 1.638 | 1.651 | 0.081 | 0.094 | 0.107 |
| μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 10 ng/ml A | 0.568 | 1.343 | 2.616 | 1.544 | 2.667 | 2.857 | 0.14 | 0.305 | 0.333 |
| 1 μg/ml CDDP | 0.152 | 0.24 | 0.312 | 1.541 | 1.56 | 1.584 | 0.008 | 0.011 | 0.01 |
| 10 ng/ml A + 1 | 0.17 | 0.445 | 0.172 | 1.651 | 1.607 | 1.546 | 0.003 | 0.008 | 0.006 |
| μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 10 ng/ml A | 0.568 | 1.343 | 2.616 | 1.544 | 2.667 | 2.857 | 0.14 | 0.305 | 0.333 |
| 10 μg/ml CDDP | 0.171 | 0.267 | 0.303 | 1.242 | 1.46 | 1.561 | 0.017 | 0.009 | 0.009 |
| 10 ng/ml A + 10 | 0.221 | 0.388 | 0.549 | 1.197 | 1.656 | 1.611 | 0.013 | 0.009 | 0.006 |
| μg/ml CDDP | | | | | | | | | |
| Unstimulated | 0.526 | 1.008 | 2.252 | 1.66 | 2.716 | 2.881 | 0.191 | 0.443 | 0.514 |
| cells | | | | | | | | | |
| 10 ng/ml A | 0.568 | 1.343 | 2.616 | 1.544 | 2.667 | 2.857 | 0.14 | 0.305 | 0.333 |
| 100 μg/ml CDDP | 0.133 | 0.3 | 0.289 | 1.2 | 1.284 | 1.421 | 0.006 | 0.009 | 0.008 |
| 10 ng/ml A + 100 | 0.47 | 0.712 | 0.729 | 1.437 | 1.62 | 1.502 | 0.003 | 0.007 | 0.006 |
| μg/ml CDDP | | | | | | | | | |

TABLE 32

| | MCF-7 | Run 1 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.876 | 1.611 | 2.413 | 2.264 | 2.854 | 2.855 | 0.763 | 1.023 | 0.904 |
| cells | | | | | | | | | |
| 0.01 ng/ml A | 0.884 | 1.82 | 2.432 | 2.167 | 2.903 | 2.865 | 0.775 | 0.859 | 0.772 |
| 0.1 μg/ml CDDP | 0.978 | 1.112 | 1.632 | 2.066 | 2.67 | 2.779 | 0.574 | 0.719 | 0.788 |
| 0.01 ng/ml A + | 0.539 | 0.413 | 0.465 | 1.777 | 2.103 | 1.629 | 0.411 | 0.349 | 0.267 |
| 0.1 μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 0.01 ng/ml A | 0.884 | 1.82 | 2.432 | 2.167 | 2.903 | 2.865 | 0.775 | 0.859 | 0.772 |
| 1 μg/ml CDDP | 0.376 | 0.158 | 0.325 | 1.404 | 1.873 | 1.762 | 0.066 | 0.051 | 0.048 |
| 0.01 ng/ml A + 1 | 0.157 | 0.239 | 0.327 | 1.215 | 1.379 | 1.459 | 0.041 | 0.033 | 0.026 |
| μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 0.01 ng/ml A | 0.884 | 1.82 | 2.432 | 2.167 | 2.903 | 2.865 | 0.775 | 0.859 | 0.772 |
| 10 μg/ml CDDP | 0.468 | 0.303 | 0.237 | 1.825 | 1.731 | 1.702 | 0.194 | 0.069 | 0.047 |
| 0.01 ng/ml A + 10 | 0.499 | 0.57 | 0.62 | 1.746 | 1.349 | 1.186 | 0.19 | 0.04 | 0.031 |
| μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 0.01 ng/ml A | 0.884 | 1.82 | 2.432 | 2.167 | 2.903 | 2.865 | 0.775 | 0.859 | 0.772 |
| 100 μg/ml CDDP | 0.285 | 0.153 | 0.284 | 1.579 | 1.452 | 1.024 | 0.034 | 0.025 | 0.023 |
| 0.01 ng/ml A + | 0.361 | 0.54 | 0.557 | 1.635 | 1.649 | 1.205 | 0.025 | 0.018 | 0.023 |
| 100 μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 0.1 ng/ml A | 1.001 | 2.084 | 2.388 | 2.103 | 2.916 | 2.86 | 0.586 | 1.058 | 0.883 |
| 0.1 μg/ml CDDP | 0.978 | 1.112 | 1.632 | 2.066 | 2.67 | 2.779 | 0.574 | 0.719 | 0.788 |
| 0.1 ng/ml A + 0.1 | 0.688 | 0.594 | 0.722 | 2.138 | 2.188 | 2.04 | 0.406 | 0.428 | 0.356 |
| μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 0.1 ng/ml A | 1.001 | 2.084 | 2.388 | 2.103 | 2.916 | 2.86 | 0.586 | 1.058 | 0.883 |
| 1 μg/ml CDDP | 0.376 | 0.158 | 0.325 | 1.404 | 1.873 | 1.762 | 0.066 | 0.051 | 0.048 |
| 0.1 ng/ml A + 1 | 0.346 | 0.18 | 0.315 | 1.5 | 1.578 | 1.276 | 0.027 | 0.023 | 0.021 |
| μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 0.1 ng/ml A | 1.001 | 2.084 | 2.388 | 2.103 | 2.916 | 2.86 | 0.586 | 1.058 | 0.883 |
| 10 μg/ml CDDP | 0.468 | 0.303 | 0.237 | 1.825 | 1.731 | 1.702 | 0.194 | 0.069 | 0.047 |
| 0.1 ng/ml A + 10 | 0.569 | 0.449 | 0.948 | 1.972 | 1.595 | 1.411 | 0.192 | 0.048 | 0.032 |
| μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 0.1 ng/ml A | 1.001 | 2.084 | 2.388 | 2.103 | 2.916 | 2.86 | 0.586 | 1.058 | 0.883 |
| 100 μg/ml CDDP | 0.285 | 0.153 | 0.284 | 1.579 | 1.452 | 1.024 | 0.034 | 0.025 | 0.023 |
| 0.1 ng/ml A + 100 | 0.523 | 0.629 | 0.514 | 2.15 | 1.915 | 1.523 | 0.02 | 0.021 | 0.022 |
| μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 1 ng/ml A | 1.046 | 2.372 | 2.998 | 2.298 | 2.921 | 2.973 | 0.52 | 0.863 | 0.843 |
| 0.1 μg/ml CDDP | 0.978 | 1.112 | 1.632 | 2.066 | 2.67 | 2.779 | 0.574 | 0.719 | 0.788 |
| 1 ng/ml A + 0.1 | 0.902 | 0.943 | 0.885 | 2.154 | 2.159 | 2.231 | 0.353 | 0.4 | 0.367 |
| μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 1 ng/ml A | 1.046 | 2.372 | 2.998 | 2.298 | 2.921 | 2.973 | 0.52 | 0.863 | 0.843 |
| 1 μg/ml CDDP | 0.376 | 0.158 | 0.325 | 1.404 | 1.873 | 1.762 | 0.066 | 0.051 | 0.048 |
| 1 ng/ml A + 1 | 0.355 | 0.202 | 0.388 | 1.538 | 1.634 | 1.438 | 0.03 | 0.038 | 0.029 |
| μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 1 ng/ml A | 1.046 | 2.372 | 2.998 | 2.298 | 2.921 | 2.973 | 0.52 | 0.863 | 0.843 |
| 10 μg/ml CDDP | 0.468 | 0.303 | 0.237 | 1.825 | 1.731 | 1.702 | 0.194 | 0.069 | 0.047 |
| 1 ng/ml A + 10 | 0.806 | 0.338 | 0.837 | 2.274 | 1.849 | 1.54 | 0.192 | 0.038 | 0.029 |
| μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 1 ng/ml A | 1.046 | 2.372 | 2.998 | 2.298 | 2.921 | 2.973 | 0.52 | 0.863 | 0.843 |
| 100 μg/ml CDDP | 0.285 | 0.153 | 0.284 | 1.579 | 1.452 | 1.024 | 0.034 | 0.025 | 0.023 |
| 1 ng/ml A + 100 | 0.673 | 0.734 | 0.421 | 2.387 | 2.339 | 2.249 | 0.026 | 0.022 | 0.024 |
| μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 10 ng/ml A | 1.068 | 2.148 | 3.025 | 2.368 | 2.777 | 2.995 | 0.527 | 0.988 | 0.927 |
| 0.1 μg/ml CDDP | 0.978 | 1.112 | 1.632 | 2.066 | 2.67 | 2.779 | 0.574 | 0.719 | 0.788 |
| 10 ng/ml A + 0.1 | 0.954 | 0.787 | 0.781 | 2.204 | 2.221 | 1.915 | 0.518 | 0.373 | 0.344 |
| μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 10 ng/ml A | 1.068 | 2.148 | 3.025 | 2.368 | 2.777 | 2.995 | 0.527 | 0.988 | 0.927 |
| 1 μg/ml CDDP | 0.376 | 0.158 | 0.325 | 1.404 | 1.873 | 1.762 | 0.066 | 0.051 | 0.048 |
| 10 ng/ml A + 1 | 0.412 | 0.221 | 0.292 | 1.596 | 1.789 | 1.628 | 0.034 | 0.022 | 0.021 |
| μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 10 ng/ml A | 1.068 | 2.148 | 3.025 | 2.368 | 2.777 | 2.995 | 0.527 | 0.988 | 0.927 |
| 10 μg/ml CDDP | 0.468 | 0.303 | 0.237 | 1.825 | 1.731 | 1.702 | 0.194 | 0.069 | 0.047 |
| 10 ng/ml A + 10 | 0.968 | 0.408 | 0.352 | 2.429 | 1.862 | 1.582 | 0.12 | 0.033 | 0.03 |
| μg/ml CDDP | | | | | | | | | |
| Unstimulated | 0.876 | 1.611 | 2.413 | 2.264 | 2.854 | 2.855 | 0.763 | 1.023 | 0.904 |
| cells | | | | | | | | | |
| 10 ng/ml A | 1.068 | 2.148 | 3.025 | 2.368 | 2.777 | 2.995 | 0.527 | 0.988 | 0.927 |
| 100 μg/ml CDDP | 0.285 | 0.153 | 0.284 | 1.579 | 1.452 | 1.024 | 0.034 | 0.025 | 0.023 |
| 10 ng/ml A + 100 | 0.465 | 0.393 | 0.501 | 2.064 | 1.976 | 1.889 | 0.022 | 0.019 | 0.02 |
| μg/ml CDDP | | | | | | | | | |

TABLE 33

| | MCF-7 | Run 2 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.898 | 1.634 | 1.672 | 1.675 | 2.724 | 2.854 | 0.307 | 0.748 | 0.969 |
| cells | | | | | | | | | |
| 0.01 ng/ml A | 0.903 | 1.428 | 1.925 | 1.832 | 2.721 | 2.902 | 0.275 | 0.671 | 0.72 |
| 0.1 μg/ml CDDP | 0.71 | 0.664 | 0.678 | 1.754 | 2.037 | 2.289 | 0.263 | 0.37 | 0.39 |
| 0.01 ng/ml A + | 0.568 | 0.326 | 0.27 | 1.535 | 1.428 | 1.58 | 0.231 | 0.239 | 0.199 |
| 0.1 μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 0.01 ng/ml A | 0.903 | 1.428 | 1.925 | 1.832 | 2.721 | 2.902 | 0.275 | 0.671 | 0.72 |
| 1 μg/ml CDDP | 0.192 | 0.239 | 0.151 | 1.225 | 0.874 | 1.409 | 0.027 | 0.025 | 0.038 |
| 0.01 ng/ml A + 1 | 0.154 | 0.126 | 0.335 | 1.228 | 0.946 | 1.363 | 0.017 | 0.028 | 0.028 |
| μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 0.01 ng/ml A | 0.903 | 1.428 | 1.925 | 1.832 | 2.721 | 2.902 | 0.275 | 0.671 | 0.72 |
| 10 μg/ml CDDP | 0.612 | 0.24 | 0.158 | 1.717 | 0.88 | 1.179 | 0.124 | 0.045 | 0.028 |
| 0.01 ng/ml A + 10 | 0.414 | 0.308 | 0.115 | 1.642 | 1.062 | 1.105 | 0.144 | 0.036 | 0.024 |
| μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 0.01 ng/ml A | 0.903 | 1.428 | 1.925 | 1.832 | 2.721 | 2.902 | 0.275 | 0.671 | 0.72 |
| 100 μg/ml CDDP | 0.288 | 0.221 | 0.147 | 1.056 | 0.985 | 0.897 | 0.029 | 0.029 | 0.013 |
| 0.01 ng/ml A + | 0.235 | 0.343 | 0.1 | 1.471 | 1.193 | 1.288 | 0.022 | 0.025 | 0.02 |
| 100 μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 0.1 ng/ml A | 1.126 | 1.431 | 1.709 | 2.039 | 2.724 | 2.918 | 0.37 | 0.734 | 0.728 |
| 0.1 μg/ml CDDP | 0.71 | 0.664 | 0.678 | 1.754 | 2.037 | 2.289 | 0.263 | 0.37 | 0.39 |
| 0.1 ng/ml A + 0.1 | 0.761 | 0.384 | 0.352 | 1.715 | 1.326 | 1.917 | 0.215 | 0.277 | 0.234 |
| μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 0.1 ng/ml A | 1.126 | 1.431 | 1.709 | 2.039 | 2.724 | 2.918 | 0.37 | 0.734 | 0.728 |
| 1 μg/ml CDDP | 0.192 | 0.239 | 0.151 | 1.225 | 0.874 | 1.409 | 0.027 | 0.025 | 0.038 |
| 0.1 ng/ml A + 1 | 0.248 | 0.244 | 0.228 | 1.28 | 0.999 | 1.609 | 0.019 | 0.027 | 0.037 |
| μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 0.1 ng/ml A | 1.126 | 1.431 | 1.709 | 2.039 | 2.724 | 2.918 | 0.37 | 0.734 | 0.728 |
| 10 μg/ml CDDP | 0.612 | 0.24 | 0.158 | 1.717 | 0.88 | 1.179 | 0.124 | 0.045 | 0.028 |
| 0.1 ng/ml A + 10 | 0.568 | 0.241 | 0.152 | 1.874 | 1.109 | 1.375 | 0.127 | 0.037 | 0.021 |
| μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 0.1 ng/ml A | 1.126 | 1.431 | 1.709 | 2.039 | 2.724 | 2.918 | 0.37 | 0.734 | 0.728 |
| 100 μg/ml CDDP | 0.288 | 0.221 | 0.147 | 1.056 | 0.985 | 0.897 | 0.029 | 0.029 | 0.013 |
| 0.1 ng/ml A + 100 | 0.345 | 0.5 | 0.139 | 1.785 | 1.288 | 1.681 | 0.02 | 0.023 | 0.021 |
| μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 1 ng/ml A | 1.442 | 2.206 | 2.175 | 2.039 | 2.712 | 2.981 | 0.273 | 0.677 | 0.653 |
| 0.1 μg/ml CDDP | 0.71 | 0.664 | 0.678 | 1.754 | 2.037 | 2.289 | 0.263 | 0.37 | 0.39 |
| 1 ng/ml A + 0.1 | 1.016 | 0.595 | 0.468 | 1.665 | 1.408 | 1.89 | 0.165 | 0.255 | 0.19 |
| μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 1 ng/ml A | 1.442 | 2.206 | 2.175 | 2.039 | 2.712 | 2.981 | 0.273 | 0.677 | 0.653 |
| 1 μg/ml CDDP | 0.192 | 0.239 | 0.151 | 1.225 | 0.874 | 1.409 | 0.027 | 0.025 | 0.038 |
| 1 ng/ml A + 1 | 0.3 | 0.393 | 0.163 | 1.679 | 1.072 | 1.478 | 0.02 | 0.03 | 0.031 |
| μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 1 ng/ml A | 1.442 | 2.206 | 2.175 | 2.039 | 2.712 | 2.981 | 0.273 | 0.677 | 0.653 |
| 10 μg/ml CDDP | 0.612 | 0.24 | 0.158 | 1.717 | 0.88 | 1.179 | 0.124 | 0.045 | 0.028 |
| 1 ng/ml A + 10 | 0.739 | 0.3 | 0.143 | 1.952 | 1.4 | 1.705 | 0.124 | 0.037 | 0.025 |
| μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 1 ng/ml A | 1.442 | 2.206 | 2.175 | 2.039 | 2.712 | 2.981 | 0.273 | 0.677 | 0.653 |
| 100 μg/ml CDDP | 0.288 | 0.221 | 0.147 | 1.056 | 0.985 | 0.897 | 0.029 | 0.029 | 0.013 |
| 1 ng/ml A + 100 | 0.582 | 0.433 | 0.344 | 1.836 | 1.544 | 1.862 | 0.03 | 0.028 | 0.022 |
| μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 10 ng/ml A | 1.435 | 2.165 | 1.831 | 1.885 | 2.752 | 2.932 | 0.326 | 0.76 | 0.575 |
| 0.1 μg/ml CDDP | 0.71 | 0.664 | 0.678 | 1.754 | 2.037 | 2.289 | 0.263 | 0.37 | 0.39 |
| 10 ng/ml A + 0.1 | 0.958 | 0.569 | 0.37 | 1.506 | 1.727 | 1.523 | 0.162 | 0.276 | 0.129 |
| μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 10 ng/ml A | 1.435 | 2.165 | 1.831 | 1.885 | 2.752 | 2.932 | 0.326 | 0.76 | 0.575 |
| 1 μg/ml CDDP | 0.192 | 0.239 | 0.151 | 1.225 | 0.874 | 1.409 | 0.027 | 0.025 | 0.038 |
| 10 ng/ml A + 1 | 0.306 | 0.227 | 0.122 | 1.176 | 1.731 | 1.165 | 0.024 | 0.027 | 0.022 |
| μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 10 ng/ml A | 1.435 | 2.165 | 1.831 | 1.885 | 2.752 | 2.932 | 0.326 | 0.76 | 0.575 |
| 10 μg/ml CDDP | 0.612 | 0.24 | 0.158 | 1.717 | 0.88 | 1.179 | 0.124 | 0.045 | 0.028 |
| 10 ng/ml A + 10 | 0.65 | 0.367 | 0.267 | 1.441 | 1.531 | 1.562 | 0.091 | 0.036 | 0.027 |
| μg/ml CDDP | | | | | | | | | |
| Unstimulated | 0.898 | 1.634 | 1.672 | 1.675 | 2.724 | 2.854 | 0.307 | 0.748 | 0.969 |
| cells | | | | | | | | | |
| 10 ng/ml A | 1.435 | 2.165 | 1.831 | 1.885 | 2.752 | 2.932 | 0.326 | 0.76 | 0.575 |
| 100 μg/ml CDDP | 0.288 | 0.221 | 0.147 | 1.056 | 0.985 | 0.897 | 0.029 | 0.029 | 0.013 |
| 10 ng/ml A + 100 | 0.434 | 0.514 | 0.319 | 1.816 | 1.742 | 1.763 | 0.027 | 0.025 | 0.02 |
| μg/ml CDDP | | | | | | | | | |

TABLE 34

| | MCF-7 | Run 3 | | | | | | | |
|---|---|---|---|---|---|---|---|---|---|
| | CVE | CVE | CVE | SRB | SRB | SRB | MTT | MTT | MTT |
| Average | 24 h | 48 h | 72 h | 24 h | 48 h | 72 h | 24 h | 48 h | 72 h |
| Unstimulated | 0.573 | 1.5 | 2.932 | 2.85 | 2.42 | 2.846 | 0.792 | 1.295 | 1.118 |
| cells | | | | | | | | | |
| 0.01 ng/ml A | 0.734 | 1.653 | 2.626 | 2.889 | 2.627 | 2.869 | 0.753 | 1.191 | 1.03 |
| 0.1 μg/ml CDDP | 0.454 | 0.811 | 1.312 | 2.843 | 2.371 | 2.522 | 0.516 | 0.525 | 0.546 |
| 0.01 ng/ml A + | 0.344 | 0.299 | 0.399 | 2.533 | 1.619 | 1.68 | 0.309 | 0.164 | 0.092 |
| 0.1 μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 0.01 ng/ml A | 0.734 | 1.653 | 2.626 | 2.889 | 2.627 | 2.869 | 0.753 | 1.191 | 1.03 |
| 1 μg/ml CDDP | 0.183 | 0.871 | 0.51 | 1.805 | 1.787 | 1.885 | 0.039 | 0.041 | 0.035 |
| 0.01 ng/ml A + 1 | 0.124 | 0.138 | 0.218 | 1.241 | 1.47 | 1.503 | 0.024 | 0.015 | 0.02 |
| μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 0.01 ng/ml A | 0.734 | 1.653 | 2.626 | 2.889 | 2.627 | 2.869 | 0.753 | 1.191 | 1.03 |
| 10 μg/ml CDDP | 0.307 | 0.769 | 0.449 | 2.092 | 1.338 | 1.622 | 0.135 | 0.03 | 0.032 |
| 0.01 ng/ml A + 10 | 0.289 | 0.156 | 0.276 | 1.991 | 1.353 | 1.397 | 0.09 | 0.022 | 0.476 |
| μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 0.01 ng/ml A | 0.734 | 1.653 | 2.626 | 2.889 | 2.627 | 2.869 | 0.753 | 1.191 | 1.03 |
| 100 μg/ml CDDP | 0.129 | 0.439 | 0.253 | 1.077 | 1.673 | 1.181 | 0.026 | 0.032 | 0.018 |
| 0.01 ng/ml A + | 0.217 | 0.255 | 0.291 | 1.82 | 1.843 | 1.62 | 0.02 | 0.014 | 0.019 |
| 100 μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 0.1 ng/ml A | 0.881 | 1.463 | 2.003 | 2.869 | 2.513 | 2.866 | 0.772 | 1.065 | 1.069 |
| 0.1 μg/ml CDDP | 0.454 | 0.811 | 1.312 | 2.843 | 2.371 | 2.522 | 0.516 | 0.525 | 0.546 |
| 0.1 ng/ml A + 0.1 | 0.788 | 0.613 | 0.61 | 2.676 | 1.97 | 2.194 | 0.507 | 0.315 | 0.296 |
| μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 0.1 ng/ml A | 0.881 | 1.463 | 2.003 | 2.869 | 2.513 | 2.866 | 0.772 | 1.065 | 1.069 |
| 1 μg/ml CDDP | 0.183 | 0.871 | 0.51 | 1.805 | 1.787 | 1.885 | 0.039 | 0.041 | 0.035 |
| 0.1 ng/ml A + 1 | 0.273 | 0.215 | 0.209 | 1.517 | 1.372 | 1.695 | 0.021 | 0.013 | 0.016 |
| μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 0.1 ng/ml A | 0.881 | 1.463 | 2.003 | 2.869 | 2.513 | 2.866 | 0.772 | 1.065 | 1.069 |
| 10 μg/ml CDDP | 0.307 | 0.769 | 0.449 | 2.092 | 1.338 | 1.622 | 0.135 | 0.03 | 0.032 |
| 0.1 ng/ml A + 10 | 0.45 | 0.537 | 0.325 | 2.219 | 1.801 | 1.816 | 0.101 | 0.025 | 0.069 |
| μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 0.1 ng/ml A | 0.881 | 1.463 | 2.003 | 2.869 | 2.513 | 2.866 | 0.772 | 1.065 | 1.069 |
| 100 μg/ml CDDP | 0.129 | 0.439 | 0.253 | 1.077 | 1.673 | 1.181 | 0.026 | 0.032 | 0.018 |
| 0.1 ng/ml A + 100 | 0.276 | 0.335 | 0.52 | 1.749 | 1.48 | 2.189 | 0.019 | 0.016 | 0.02 |
| μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 1 ng/ml A | 0.87 | 2.35 | 2.621 | 2.843 | 2.82 | 2.965 | 0.512 | 0.989 | 0.985 |
| 0.1 μg/ml CDDP | 0.454 | 0.811 | 1.312 | 2.843 | 2.371 | 2.522 | 0.516 | 0.525 | 0.546 |
| 1 ng/ml A + 0.1 | 0.651 | 0.908 | 0.912 | 2.677 | 2.073 | 2.038 | 0.375 | 0.288 | 0.298 |
| μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 1 ng/ml A | 0.87 | 2.35 | 2.621 | 2.843 | 2.82 | 2.965 | 0.512 | 0.989 | 0.985 |
| 1 μg/ml CDDP | 0.183 | 0.871 | 0.51 | 1.805 | 1.787 | 1.885 | 0.039 | 0.041 | 0.035 |
| 1 ng/ml A + 1 | 0.309 | 0.51 | 0.318 | 1.789 | 1.79 | 1.854 | 0.027 | 0.022 | 0.021 |
| μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 1 ng/ml A | 0.87 | 2.35 | 2.621 | 2.843 | 2.82 | 2.965 | 0.512 | 0.989 | 0.985 |
| 10 μg/ml CDDP | 0.307 | 0.769 | 0.449 | 2.092 | 1.338 | 1.622 | 0.135 | 0.03 | 0.032 |
| 1 ng/ml A + 10 | 0.353 | 0.808 | 0.351 | 2.46 | 2.026 | 2.25 | 0.121 | 0.022 | 0.025 |
| μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 1 ng/ml A | 0.87 | 2.35 | 2.621 | 2.843 | 2.82 | 2.965 | 0.512 | 0.989 | 0.985 |
| 100 μg/ml CDDP | 0.129 | 0.439 | 0.253 | 1.077 | 1.673 | 1.181 | 0.026 | 0.032 | 0.018 |
| 1 ng/ml A + 100 | 0.311 | 0.879 | 0.449 | 2.193 | 2.004 | 2.234 | 0.024 | 0.017 | 0.018 |
| μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 10 ng/ml A | 0.861 | 2.15 | 2.792 | 2.591 | 2.821 | 2.859 | 0.545 | 1.31 | 1.066 |
| 0.1 μg/ml CDDP | 0.454 | 0.811 | 1.312 | 2.843 | 2.371 | 2.522 | 0.516 | 0.525 | 0.546 |
| 10 ng/ml A + 0.1 | 0.596 | 0.935 | 0.676 | 2.087 | 2.175 | 1.846 | 0.501 | 0.511 | 0.28 |
| μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 10 ng/ml A | 0.861 | 2.15 | 2.792 | 2.591 | 2.821 | 2.859 | 0.545 | 1.31 | 1.066 |
| 1 μg/ml CDDP | 0.183 | 0.871 | 0.51 | 1.805 | 1.787 | 1.885 | 0.039 | 0.041 | 0.035 |
| 10 ng/ml A + 1 | 0.158 | 0.595 | 0.241 | 1.163 | 1.741 | 1.703 | 0.023 | 0.027 | 0.018 |
| μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 10 ng/ml A | 0.861 | 2.15 | 2.792 | 2.591 | 2.821 | 2.859 | 0.545 | 1.31 | 1.066 |
| 10 μg/ml CDDP | 0.307 | 0.769 | 0.449 | 2.092 | 1.338 | 1.622 | 0.135 | 0.03 | 0.032 |
| 10 ng/ml A + 10 | 0.336 | 0.895 | 0.382 | 1.605 | 1.828 | 1.558 | 0.09 | 0.029 | 0.023 |
| μg/ml CDDP | | | | | | | | | |
| Unstimulated | 0.573 | 1.5 | 2.932 | 2.85 | 2.42 | 2.846 | 0.792 | 1.295 | 1.118 |
| cells | | | | | | | | | |
| 10 ng/ml A | 0.861 | 2.15 | 2.792 | 2.591 | 2.821 | 2.859 | 0.545 | 1.31 | 1.066 |
| 100 μg/ml CDDP | 0.129 | 0.439 | 0.253 | 1.077 | 1.673 | 1.181 | 0.026 | 0.032 | 0.018 |
| 10 ng/ml A + 100 | 0.246 | 0.787 | 0.373 | 1.465 | 2.205 | 1.809 | 0.021 | 0.015 | 0.016 |
| μg/ml CDDP | | | | | | | | | |

TABLE 35

| | MDA-MB | Run 1 | | | | | | | |
|---|---|---|---|---|---|---|---|---|---|
| | 231 | CVE | CVE | SRB | SRB | SRB | MTT | MTT | MTT |
| Average | CVE 24 h | 48 h | 72 h | 24 h | 48 h | 72 h | 24 h | 48 h | 72 h |
| Unstimulated | 0.27 | 1.355 | 1.343 | 1.897 | 2.86 | 2.91 | 0.042 | 0.003 | 0.049 |
| cells | | | | | | | | | |
| 0.01 ng/ml A | 0.268 | 1.132 | 1.268 | 2.011 | 2.766 | 2.932 | 0.024 | 0.002 | 0.084 |
| 0.1 μg/ml CDDP | 0.161 | 0.939 | 0.768 | 1.792 | 2.678 | 2.873 | 0.019 | 0.002 | 0.04 |
| 0.01 ng/ml A + | 0.159 | 0.699 | 0.552 | 1.756 | 2.632 | 2.787 | 0.018 | 0.002 | 0.05 |
| 0.1 μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 0.01 ng/ml A | 0.268 | 1.132 | 1.268 | 2.011 | 2.766 | 2.932 | 0.024 | 0.002 | 0.084 |
| 1 μg/ml CDDP | 0.124 | 0.549 | 0.375 | 1.395 | 2.254 | 2.928 | 0.007 | 0.004 | 0.006 |
| 0.01 ng/ml A + 1 | 0.042 | 0.318 | 0.409 | 1.636 | 2.296 | 2.749 | 0.006 | 0.001 | 0.006 |
| μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 0.01 ng/ml A | 0.268 | 1.132 | 1.268 | 2.011 | 2.766 | 2.932 | 0.024 | 0.002 | 0.084 |
| 10 μg/ml CDDP | 0.149 | 0.586 | 0.416 | 1.785 | 2.489 | 2.962 | 0.015 | 0.007 | 0.015 |
| 0.01 ng/ml A + 10 | 0.072 | 0.217 | 0.388 | 2.027 | 2.003 | 2.414 | 0.011 | 0.004 | 0.01 |
| μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 0.01 ng/ml A | 0.268 | 1.132 | 1.268 | 2.011 | 2.766 | 2.932 | 0.024 | 0.002 | 0.084 |
| 100 μg/ml CDDP | 0.098 | 0.504 | 0.538 | 1.752 | 2.457 | 2.852 | 0.008 | 0.008 | 0.006 |
| 0.01 ng/ml A + | 0.111 | 0.099 | 0.145 | 1.952 | 1.842 | 2.362 | 0.004 | 0.001 | 0.005 |
| 100 μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 0.1 ng/ml A | 0.209 | 1.663 | 1.105 | 1.86 | 2.912 | 3.005 | 0.016 | 0.002 | 0.047 |
| 0.1 μg/ml CDDP | 0.161 | 0.939 | 0.768 | 1.792 | 2.678 | 2.873 | 0.019 | 0.002 | 0.04 |
| 0.1 ng/ml A + 0.1 | 0.179 | 0.736 | 0.609 | 1.814 | 2.618 | 2.924 | 0.016 | 0.006 | 0.06 |
| μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 0.1 ng/ml A | 0.209 | 1.663 | 1.105 | 1.86 | 2.912 | 3.005 | 0.016 | 0.002 | 0.047 |
| 1 μg/ml CDDP | 0.124 | 0.549 | 0.375 | 1.395 | 2.254 | 2.928 | 0.007 | 0.004 | 0.006 |
| 0.1 ng/ml A + 1 | 0.061 | 0.357 | 0.459 | 1.641 | 2.376 | 2.903 | 0.008 | 0.002 | 0.005 |
| μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 0.1 ng/ml A | 0.209 | 1.663 | 1.105 | 1.86 | 2.912 | 3.005 | 0.016 | 0.002 | 0.047 |
| 10 μg/ml CDDP | 0.149 | 0.586 | 0.416 | 1.785 | 2.489 | 2.962 | 0.015 | 0.007 | 0.015 |
| 0.1 ng/ml A + 10 | 0.194 | 0.455 | 0.674 | 2.079 | 2.357 | 2.808 | 0.015 | 0.004 | 0.02 |
| μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 0.1 ng/ml A | 0.209 | 1.663 | 1.105 | 1.86 | 2.912 | 3.005 | 0.016 | 0.002 | 0.047 |
| 100 μg/ml CDDP | 0.098 | 0.504 | 0.538 | 1.752 | 2.457 | 2.852 | 0.008 | 0.008 | 0.006 |
| 0.1 ng/ml A + 100 | 0.084 | 0.129 | 0.183 | 2.181 | 1.873 | 2.542 | 0.006 | 0.003 | 0.005 |
| μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 1 ng/ml A | 0.315 | 1.138 | 1.424 | 2.025 | 2.873 | 3.069 | 0.026 | 0.005 | 0.087 |
| 0.1 μg/ml CDDP | 0.161 | 0.939 | 0.768 | 1.792 | 2.678 | 2.873 | 0.019 | 0.002 | 0.04 |
| 1 ng/ml A + 0.1 | 0.301 | 0.653 | 0.696 | 2.286 | 2.668 | 2.875 | 0.02 | 0.007 | 0.035 |
| μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 1 ng/ml A | 0.315 | 1.138 | 1.424 | 2.025 | 2.873 | 3.069 | 0.026 | 0.005 | 0.087 |
| 1 μg/ml CDDP | 0.124 | 0.549 | 0.375 | 1.395 | 2.254 | 2.928 | 0.007 | 0.004 | 0.006 |
| 1 ng/ml A + 1 | 0.044 | 0.41 | 0.325 | 1.798 | 2.578 | 2.913 | 0.023 | 0.008 | 0.007 |
| μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 1 ng/ml A | 0.315 | 1.138 | 1.424 | 2.025 | 2.873 | 3.069 | 0.026 | 0.005 | 0.087 |
| 10 μg/ml CDDP | 0.149 | 0.586 | 0.416 | 1.785 | 2.489 | 2.962 | 0.015 | 0.007 | 0.015 |
| 1 ng/ml A + 10 | 0.237 | 0.579 | 0.405 | 2.312 | 2.438 | 2.865 | 0.018 | 0.007 | 0.037 |
| μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 1 ng/ml A | 0.315 | 1.138 | 1.424 | 2.025 | 2.873 | 3.069 | 0.026 | 0.005 | 0.087 |
| 100 μg/ml CDDP | 0.098 | 0.504 | 0.538 | 1.752 | 2.457 | 2.852 | 0.008 | 0.008 | 0.006 |
| 1 ng/ml A + 100 | 0.128 | 0.109 | 0.212 | 2.374 | 2.112 | 2.482 | 0.01 | 0.005 | 0.004 |
| μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 10 ng/ml A | 0.326 | 1.271 | 1.191 | 2.109 | 2.835 | 2.941 | 0.021 | 0.004 | 0.045 |
| 0.1 μg/ml CDDP | 0.161 | 0.939 | 0.768 | 1.792 | 2.678 | 2.873 | 0.019 | 0.002 | 0.04 |
| 10 ng/ml A + 0.1 | 0.268 | 0.772 | 0.726 | 2.283 | 2.7 | 2.889 | 0.034 | 0.008 | 0.048 |
| μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 10 ng/ml A | 0.326 | 1.271 | 1.191 | 2.109 | 2.835 | 2.941 | 0.021 | 0.004 | 0.045 |
| 1 μg/ml CDDP | 0.124 | 0.549 | 0.375 | 1.395 | 2.254 | 2.928 | 0.007 | 0.004 | 0.006 |
| 10 ng/ml A + 1 | 0.049 | 0.406 | 0.41 | 1.775 | 2.537 | 2.807 | 0.009 | 0.004 | 0.005 |
| μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 10 ng/ml A | 0.326 | 1.271 | 1.191 | 2.109 | 2.835 | 2.941 | 0.021 | 0.004 | 0.045 |
| 10 μg/ml CDDP | 0.149 | 0.586 | 0.416 | 1.785 | 2.489 | 2.962 | 0.015 | 0.007 | 0.015 |
| 10 ng/ml A + 10 | 0.262 | 0.317 | 0.278 | 2.368 | 2.535 | 2.749 | 0.026 | 0.01 | 0.029 |
| μg/ml CDDP | | | | | | | | | |
| Unstimulated | 0.27 | 1.355 | 1.343 | 1.897 | 2.86 | 2.91 | 0.042 | 0.003 | 0.049 |
| cells | | | | | | | | | |
| 10 ng/ml A | 0.326 | 1.271 | 1.191 | 2.109 | 2.835 | 2.941 | 0.021 | 0.004 | 0.045 |
| 100 μg/ml CDDP | 0.098 | 0.504 | 0.538 | 1.752 | 2.457 | 2.852 | 0.008 | 0.008 | 0.006 |
| 10 ng/ml A + 100 | 0.142 | 0.1 | 0.182 | 2.211 | 1.766 | 2.275 | 0.01 | 0.004 | 0.004 |
| μg/ml CDDP | | | | | | | | | |

TABLE 36

| | MDA- | Run 2 | | | | | | | |
|---|---|---|---|---|---|---|---|---|---|
| | MB 231 | CVE | CVE | SRB | SRB | SRB | MTT | MTT | MTT |
| Average | CVE 24 h | 48 h | 72 h | 24 h | 48 h | 72 h | 24 h | 48 h | 72 h |
| Unstimulated | 0.385 | 0.725 | 0.955 | 1.672 | 2.379 | 2.565 | 0.052 | 0.071 | 0.123 |
| cells | | | | | | | | | |
| 0.01 ng/ml A | 0.262 | 0.526 | 0.897 | 1.703 | 2.279 | 2.605 | 0.053 | 0.094 | 0.141 |
| 0.1 μg/ml CDDP | 0.118 | 0.305 | 0.485 | 1.576 | 1.83 | 2.1 | 0.044 | 0.063 | 0.106 |
| 0.01 ng/ml A + | 0.096 | 0.255 | 0.309 | 1.691 | 1.811 | 1.807 | 0.043 | 0.049 | 0.085 |
| 0.1 μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 0.01 ng/ml A | 0.262 | 0.526 | 0.897 | 1.703 | 2.279 | 2.605 | 0.053 | 0.094 | 0.141 |
| 1 μg/ml CDDP | 0.042 | 0.047 | 0.036 | 1.27 | 1.162 | 0.883 | 0.009 | 0.007 | 0.005 |
| 0.01 ng/ml A + 1 | 0.031 | 0.076 | 0.033 | 1.195 | 1.068 | 0.93 | 0.003 | 0.006 | 0.005 |
| μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 0.01 ng/ml A | 0.262 | 0.526 | 0.897 | 1.703 | 2.279 | 2.605 | 0.053 | 0.094 | 0.141 |
| 10 μg/ml CDDP | 0.125 | 0.27 | 0.301 | 1.598 | 1.867 | 1.755 | 0.039 | 0.049 | 0.07 |
| 0.01 ng/ml A + 10 | 0.137 | 0.297 | 0.22 | 1.641 | 1.763 | 1.535 | 0.035 | 0.048 | 0.061 |
| μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 0.01 ng/ml A | 0.262 | 0.526 | 0.897 | 1.703 | 2.279 | 2.605 | 0.053 | 0.094 | 0.141 |
| 100 μg/ml CDDP | 0.074 | 0.062 | 0.11 | 1.509 | 1.048 | 1.004 | 0.008 | 0.006 | 0.002 |
| 0.01 ng/ml A + | 0.049 | 0.055 | 0.07 | 1.467 | 1.166 | 1.75 | 0.007 | 0.006 | 0.004 |
| 100 μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 0.1 ng/ml A | 0.343 | 0.502 | 0.635 | 1.801 | 2.218 | 2.378 | 0.047 | 0.071 | 0.094 |
| 0.1 μg/ml CDDP | 0.118 | 0.305 | 0.485 | 1.576 | 1.83 | 2.1 | 0.044 | 0.063 | 0.106 |
| 0.1 ng/ml A + 0.1 | 0.147 | 0.262 | 0.255 | 1.643 | 1.956 | 1.641 | 0.041 | 0.062 | 0.086 |
| μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 0.1 ng/ml A | 0.343 | 0.502 | 0.635 | 1.801 | 2.218 | 2.378 | 0.047 | 0.071 | 0.094 |
| 1 μg/ml CDDP | 0.042 | 0.047 | 0.036 | 1.27 | 1.162 | 0.883 | 0.009 | 0.007 | 0.005 |
| 0.1 ng/ml A + 1 | 0.117 | 0.033 | 0.045 | 1.283 | 1.228 | 1.026 | 0.008 | 0.007 | 0.007 |
| μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 0.1 ng/ml A | 0.343 | 0.502 | 0.635 | 1.801 | 2.218 | 2.378 | 0.047 | 0.071 | 0.094 |
| 10 μg/ml CDDP | 0.125 | 0.27 | 0.301 | 1.598 | 1.867 | 1.755 | 0.039 | 0.049 | 0.07 |
| 0.1 ng/ml A + 10 | 0.124 | 0.241 | 0.24 | 1.637 | 1.978 | 1.653 | 0.035 | 0.031 | 0.007 |
| μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 0.1 ng/ml A | 0.343 | 0.502 | 0.635 | 1.801 | 2.218 | 2.378 | 0.047 | 0.071 | 0.094 |
| 100 μg/ml CDDP | 0.074 | 0.062 | 0.11 | 1.509 | 1.048 | 1.004 | 0.008 | 0.006 | 0.002 |
| 0.1 ng/ml A + 100 | 0.083 | 0.083 | 0.11 | 1.515 | 1.381 | 1.58 | 0.007 | 0.006 | 0.004 |
| μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 1 ng/ml A | 0.276 | 0.554 | 1.209 | 1.658 | 2.307 | 2.559 | 0.054 | 0.079 | 0.137 |
| 0.1 μg/ml CDDP | 0.118 | 0.305 | 0.485 | 1.576 | 1.83 | 2.1 | 0.044 | 0.063 | 0.106 |
| 1 ng/ml A + 0.1 | 0.089 | 0.26 | 0.319 | 1.62 | 1.901 | 1.865 | 0.043 | 0.053 | 0.067 |
| μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 1 ng/ml A | 0.276 | 0.554 | 1.209 | 1.658 | 2.307 | 2.559 | 0.054 | 0.079 | 0.137 |
| 1 μg/ml CDDP | 0.042 | 0.047 | 0.036 | 1.27 | 1.162 | 0.883 | 0.009 | 0.007 | 0.005 |
| 1 ng/ml A + 1 | 0.039 | 0.03 | 0.048 | 1.304 | 1.291 | 0.93 | 0.006 | 0.006 | 0.005 |
| μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 1 ng/ml A | 0.276 | 0.554 | 1.209 | 1.658 | 2.307 | 2.559 | 0.054 | 0.079 | 0.137 |
| 10 μg/ml CDDP | 0.125 | 0.27 | 0.301 | 1.598 | 1.867 | 1.755 | 0.039 | 0.049 | 0.07 |
| 1 ng/ml A + 10 | 0.16 | 0.249 | 0.223 | 1.707 | 2.006 | 1.783 | 0.031 | 0.049 | 0.05 |
| μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 1 ng/ml A | 0.276 | 0.554 | 1.209 | 1.658 | 2.307 | 2.559 | 0.054 | 0.079 | 0.137 |
| 100 μg/ml CDDP | 0.074 | 0.062 | 0.11 | 1.509 | 1.048 | 1.004 | 0.008 | 0.006 | 0.002 |
| 1 ng/ml A + 100 | 0.133 | 0.099 | 0.181 | 1.658 | 1.459 | 1.48 | 0.006 | 0.005 | 0.005 |
| μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 10 ng/ml A | 0.354 | 0.562 | 0.965 | 2.175 | 2.33 | 2.555 | 0.058 | 0.088 | 0.114 |
| 0.1 μg/ml CDDP | 0.118 | 0.305 | 0.485 | 1.576 | 1.83 | 2.1 | 0.044 | 0.063 | 0.106 |
| 10 ng/ml A + 0.1 | 0.117 | 0.257 | 0.398 | 1.825 | 1.749 | 1.877 | 0.042 | 0.046 | 0.053 |
| μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 10 ng/ml A | 0.354 | 0.562 | 0.965 | 2.175 | 2.33 | 2.555 | 0.058 | 0.088 | 0.114 |
| 1 μg/ml CDDP | 0.042 | 0.047 | 0.036 | 1.27 | 1.162 | 0.883 | 0.009 | 0.007 | 0.005 |
| 10 ng/ml A + 1 | 0.053 | 0.058 | 0.096 | 1.514 | 1.299 | 1.144 | 0.008 | 0.006 | 0.003 |
| μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 10 ng/ml A | 0.354 | 0.562 | 0.965 | 2.175 | 2.33 | 2.555 | 0.058 | 0.088 | 0.114 |
| 10 μg/ml CDDP | 0.125 | 0.27 | 0.301 | 1.598 | 1.867 | 1.755 | 0.039 | 0.049 | 0.07 |
| 10 ng/ml A + 10 | 0.296 | 0.264 | 0.358 | 1.959 | 1.825 | 1.794 | 0.041 | 0.027 | 0.031 |
| μg/ml CDDP | | | | | | | | | |
| Unstimulated | 0.385 | 0.725 | 0.955 | 1.672 | 2.379 | 2.565 | 0.052 | 0.071 | 0.123 |
| cells | | | | | | | | | |
| 10 ng/ml A | 0.354 | 0.562 | 0.965 | 2.175 | 2.33 | 2.555 | 0.058 | 0.088 | 0.114 |
| 100 μg/ml CDDP | 0.074 | 0.062 | 0.11 | 1.509 | 1.048 | 1.004 | 0.008 | 0.006 | 0.002 |
| 10 ng/ml A + 100 | 0.158 | 0.077 | 0.81 | 1.882 | 1.362 | 1.321 | 0.007 | 0.005 | 0.004 |
| μg/ml CDDP | | | | | | | | | |

TABLE 37

| | MDA-MB | Run 3 | | | | | | | |
|---|---|---|---|---|---|---|---|---|---|
| | 231 | CVE | CVE | SRB | SRB | SRB | MTT | MTT | MTT |
| Average | CVE 24 h | 48 h | 72 h | 24 h | 48 h | 72 h | 24 h | 48 h | 72 h |
| Unstimulated | 1.204 | 1.786 | 2.727 | 2.675 | 2.805 | 2.952 | 0.056 | 0.068 | 0.051 |
| cells | | | | | | | | | |
| 0.01 ng/ml A | 1.235 | 1.342 | 2.611 | 2.723 | 2.804 | 2.956 | 0.065 | 0.058 | 0.058 |
| 0.1 μg/ml CDDP | 1.198 | 1.239 | 1.573 | 2.643 | 2.742 | 2.87 | 0.083 | 0.035 | 0.046 |
| 0.01 ng/ml A + | 0.779 | 1.129 | 1.343 | 2.617 | 2.671 | 2.849 | 0.093 | 0.041 | 0.036 |
| 0.1 μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 0.01 ng/ml A | 1.235 | 1.342 | 2.611 | 2.723 | 2.804 | 2.956 | 0.065 | 0.058 | 0.058 |
| 1 μg/ml CDDP | 0.528 | 0.231 | 0.144 | 1.382 | 1.537 | 2.07 | 0.013 | 0.008 | 0.008 |
| 0.01 ng/ml A + 1 | 0.06 | 0.526 | 0.226 | 1.278 | 1.158 | 2.029 | 0.011 | 0.005 | 0.004 |
| μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 0.01 ng/ml A | 1.235 | 1.342 | 2.611 | 2.723 | 2.804 | 2.956 | 0.065 | 0.058 | 0.058 |
| 10 μg/ml CDDP | 0.693 | 0.79 | 0.472 | 2.134 | 2.364 | 2.593 | 0.046 | 0.057 | 0.068 |
| 0.01 ng/ml A + 10 | 0.725 | 0.754 | 1.294 | 2.363 | 2.553 | 2.85 | 0.081 | 0.064 | 0.084 |
| μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 0.01 ng/ml A | 1.235 | 1.342 | 2.611 | 2.723 | 2.804 | 2.956 | 0.065 | 0.058 | 0.058 |
| 100 μg/ml CDDP | 0.168 | 0.154 | 0.089 | 1.141 | 1.464 | 1.493 | 0.012 | 0.012 | 0.01 |
| 0.01 ng/ml A + | 0.316 | 0.637 | 0.277 | 1.624 | 1.564 | 1.959 | 0.009 | 0.006 | 0.112 |
| 100 μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 0.1 ng/ml A | 1.236 | 1.928 | 2.695 | 2.64 | 2.814 | 2.977 | 0.078 | 0.048 | 0.052 |
| 0.1 μg/ml CDDP | 1.198 | 1.239 | 1.573 | 2.643 | 2.742 | 2.87 | 0.083 | 0.035 | 0.046 |
| 0.1 ng/ml A + 0.1 | 0.755 | 1.231 | 1.898 | 2.579 | 2.669 | 2.876 | 0.075 | 0.031 | 0.036 |
| μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 0.1 ng/ml A | 1.236 | 1.928 | 2.695 | 2.64 | 2.814 | 2.977 | 0.078 | 0.048 | 0.052 |
| 1 μg/ml CDDP | 0.528 | 0.231 | 0.144 | 1.382 | 1.537 | 2.07 | 0.013 | 0.008 | 0.008 |
| 0.1 ng/ml A + 1 | 0.095 | 0.214 | 0.56 | 1.385 | 1.276 | 1.967 | 0.015 | 0.008 | 0.008 |
| μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 0.1 ng/ml A | 1.236 | 1.928 | 2.695 | 2.64 | 2.814 | 2.977 | 0.078 | 0.048 | 0.052 |
| 10 μg/ml CDDP | 0.693 | 0.79 | 0.472 | 2.134 | 2.364 | 2.593 | 0.046 | 0.057 | 0.068 |
| 0.1 ng/ml A + 10 | 0.761 | 0.993 | 1.04 | 2.65 | 2.692 | 2.832 | 0.079 | 0.078 | 0.093 |
| μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 0.1 ng/ml A | 1.236 | 1.928 | 2.695 | 2.64 | 2.814 | 2.977 | 0.078 | 0.048 | 0.052 |
| 100 μg/ml CDDP | 0.168 | 0.154 | 0.089 | 1.141 | 1.464 | 1.493 | 0.012 | 0.012 | 0.01 |
| 0.1 ng/ml A + 100 | 0.325 | 0.433 | 0.228 | 1.789 | 1.677 | 2.448 | 0.007 | 0.007 | 0.008 |
| μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 1 ng/ml A | 1.386 | 2.213 | 3.002 | 2.731 | 2.826 | 3.081 | 0.065 | 0.064 | 0.057 |
| 0.1 μg/ml CDDP | 1.198 | 1.239 | 1.573 | 2.643 | 2.742 | 2.87 | 0.083 | 0.035 | 0.046 |
| 1 ng/ml A + 0.1 | 1.023 | 1.379 | 1.638 | 2.638 | 2.67 | 2.873 | 0.06 | 0.033 | 0.038 |
| μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 1 ng/ml A | 1.386 | 2.213 | 3.002 | 2.731 | 2.826 | 3.081 | 0.065 | 0.064 | 0.057 |
| 1 μg/ml CDDP | 0.528 | 0.231 | 0.144 | 1.382 | 1.537 | 2.07 | 0.013 | 0.008 | 0.008 |
| 1 ng/ml A + 1 | 0.075 | 0.261 | 0.409 | 1.413 | 1.679 | 1.97 | 0.009 | 0.008 | 0.009 |
| μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 1 ng/ml A | 1.386 | 2.213 | 3.002 | 2.731 | 2.826 | 3.081 | 0.065 | 0.064 | 0.057 |
| 10 μg/ml CDDP | 0.693 | 0.79 | 0.472 | 2.134 | 2.364 | 2.593 | 0.046 | 0.057 | 0.068 |
| 1 ng/ml A + 10 | 1.149 | 1.417 | 1.151 | 2.712 | 2.714 | 2.832 | 0.058 | 0.064 | 0.08 |
| μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 1 ng/ml A | 1.386 | 2.213 | 3.002 | 2.731 | 2.826 | 3.081 | 0.065 | 0.064 | 0.057 |
| 100 μg/ml CDDP | 0.168 | 0.154 | 0.089 | 1.141 | 1.464 | 1.493 | 0.012 | 0.012 | 0.01 |
| 1 ng/ml A + 100 | 0.853 | 0.332 | 0.268 | 2.112 | 1.707 | 2.612 | 0.01 | 0.007 | 0.008 |
| μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 10 ng/ml A | 1.709 | 2.105 | 2.664 | 2.718 | 2.821 | 3.139 | 0.062 | 0.065 | 0.066 |
| 0.1 μg/ml CDDP | 1.198 | 1.239 | 1.573 | 2.643 | 2.742 | 2.87 | 0.083 | 0.035 | 0.046 |
| 10 ng/ml A + 0.1 | 1.624 | 1.438 | 1.353 | 2.532 | 2.697 | 2.901 | 0.059 | 0.036 | 0.042 |
| μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 10 ng/ml A | 1.709 | 2.105 | 2.664 | 2.718 | 2.821 | 3.139 | 0.062 | 0.065 | 0.066 |
| 1 μg/ml CDDP | 0.528 | 0.231 | 0.144 | 1.382 | 1.537 | 2.07 | 0.013 | 0.008 | 0.008 |
| 10 ng/ml A + 1 | 0.249 | 0.246 | 0.202 | 1.731 | 1.494 | 2.089 | 0.01 | 0.006 | 0.007 |
| μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 10 ng/ml A | 1.709 | 2.105 | 2.664 | 2.718 | 2.821 | 3.139 | 0.062 | 0.065 | 0.066 |
| 10 μg/ml CDDP | 0.693 | 0.79 | 0.472 | 2.134 | 2.364 | 2.593 | 0.046 | 0.057 | 0.068 |
| 10 ng/ml A + 10 | 1.454 | 1.155 | 1.31 | 2.699 | 2.604 | 2.76 | 0.054 | 0.074 | 0.118 |
| μg/ml CDDP | | | | | | | | | |
| Unstimulated | 1.204 | 1.786 | 2.727 | 2.675 | 2.805 | 2.952 | 0.056 | 0.068 | 0.051 |
| cells | | | | | | | | | |
| 10 ng/ml A | 1.709 | 2.105 | 2.664 | 2.718 | 2.821 | 3.139 | 0.062 | 0.065 | 0.066 |
| 100 μg/ml CDDP | 0.168 | 0.154 | 0.089 | 1.141 | 1.464 | 1.493 | 0.012 | 0.012 | 0.01 |
| 10 ng/ml A + 100 | 0.63 | 0.42 | 0.37 | 1.8 | 1.979 | 1.925 | 0.008 | 0.016 | 0.007 |
| μg/ml CDDP | | | | | | | | | |

TABLE 38

| | PANC-1 | Run 1 | | | | | | | |
|---|---|---|---|---|---|---|---|---|---|
| | CVE | CVE | CVE | SRB | SRB | SRB | MTT | MTT | MTT |
| | 24 h | 48 h | 72 h | 24 h | 48 h | 72 h | 24 h | 48 h | 72 h |
| Average | | | | | | | | | |
| Unstimulated cells | 0.406 | 1.307 | 2.769 | 1.912 | 2.636 | 2.906 | 0.222 | 0.748 | 1.119 |
| 0.01 ng/ml A | 0.415 | 1.265 | 2.541 | 1.881 | 2.72 | 2.896 | 0.226 | 0.626 | 1.075 |
| 0.1 μg/ml CDDP | 0.324 | 0.717 | 0.676 | 1.369 | 2.223 | 2.191 | 0.169 | 0.24 | 0.132 |
| 0.01 ng/ml A + | 0.239 | 0.56 | 0.277 | 1.448 | 1.7 | 1.633 | 0.132 | 0.133 | 0.116 |
| 0.1 μg/ml CDDP | | | | | | | | | |
| Unstimulated cells | 0.406 | 1.307 | 2.769 | 1.912 | 2.636 | 2.906 | 0.222 | 0.748 | 1.119 |
| 0.01 ng/ml A | 0.415 | 1.265 | 2.541 | 1.881 | 2.72 | 2.896 | 0.226 | 0.626 | 1.075 |
| 1 μg/ml CDDP | 0.125 | 0.429 | 0.262 | 1.748 | 1.748 | 1.47 | 0.035 | 0.035 | 0.03 |
| 0.01 ng/ml A + | 0.105 | 0.444 | 0.163 | 1.681 | 1.668 | 1.617 | 0.025 | 0.03 | 0.022 |
| 1 μg/ml CDDP | | | | | | | | | |
| Unstimulated cells | 0.406 | 1.307 | 2.769 | 1.912 | 2.636 | 2.906 | 0.222 | 0.748 | 1.119 |
| 0.01 ng/ml A | 0.415 | 1.265 | 2.541 | 1.881 | 2.72 | 2.896 | 0.226 | 0.626 | 1.075 |
| 10 μg/ml CDDP | 0.191 | 0.466 | 0.337 | 1.597 | 1.339 | 1.435 | 0.048 | 0.038 | 0.035 |
| 0.01 ng/ml A + | 0.149 | 0.325 | 0.266 | 1.558 | 1.305 | 1.454 | 0.05 | 0.039 | 0.031 |
| 10 μg/ml CDDP | | | | | | | | | |
| Unstimulated cells | 0.406 | 1.307 | 2.769 | 1.912 | 2.636 | 2.906 | 0.222 | 0.748 | 1.119 |
| 0.01 ng/ml A | 0.415 | 1.265 | 2.541 | 1.881 | 2.72 | 2.896 | 0.226 | 0.626 | 1.075 |
| 100 μg/ml CDDP | 0.153 | 0.381 | 0.28 | 1.993 | 1.53 | 1.433 | 0.034 | 0.039 | 0.032 |
| 0.01 ng/ml A + | 0.14 | 0.236 | 0.394 | 1.91 | 1.419 | 1.305 | 0.027 | 0.028 | 0.024 |
| 100 μg/ml CDDP | | | | | | | | | |
| Average | | | | | | | | | |
| Unstimulated cells | 0.406 | 1.307 | 2.769 | 1.912 | 2.636 | 2.906 | 0.222 | 0.748 | 1.119 |
| 0.1 ng/ml A | 0.464 | 1.558 | 2.639 | 2.342 | 2.787 | 2.967 | 0.217 | 0.763 | 1.05 |
| 0.1 μg/ml CDDP | 0.324 | 0.717 | 0.676 | 1.369 | 2.223 | 2.191 | 0.169 | 0.24 | 0.132 |
| 0.1 ng/ml A + | 0.303 | 0.893 | 0.411 | 1.959 | 1.917 | 1.894 | 0.144 | 0.169 | 0.131 |
| 0.1 μg/ml CDDP | | | | | | | | | |
| Unstimulated cells | 0.406 | 1.307 | 2.769 | 1.912 | 2.636 | 2.906 | 0.222 | 0.748 | 1.119 |
| 0.1 ng/ml A | 0.464 | 1.558 | 2.639 | 2.342 | 2.787 | 2.967 | 0.217 | 0.763 | 1.05 |
| 1 μg/ml CDDP | 0.125 | 0.429 | 0.262 | 1.748 | 1.748 | 1.47 | 0.035 | 0.035 | 0.03 |
| 0.1 ng/ml A + | 0.123 | 0.382 | 0.23 | 1.81 | 1.662 | 1.593 | 0.027 | 0.029 | 0.022 |
| 1 μg/ml CDDP | | | | | | | | | |
| Unstimulated cells | 0.406 | 1.307 | 2.769 | 1.912 | 2.636 | 2.906 | 0.222 | 0.748 | 1.119 |
| 0.1 ng/ml A | 0.464 | 1.558 | 2.639 | 2.342 | 2.787 | 2.967 | 0.217 | 0.763 | 1.05 |
| 10 μg/ml CDDP | 0.191 | 0.466 | 0.337 | 1.597 | 1.339 | 1.435 | 0.048 | 0.038 | 0.035 |
| 0.1 ng/ml A + | 0.166 | 0.269 | 0.214 | 1.825 | 1.429 | 1.202 | 0.051 | 0.034 | 0.029 |
| 10 μg/ml CDDP | | | | | | | | | |
| Unstimulated cells | 0.406 | 1.307 | 2.769 | 1.912 | 2.636 | 2.906 | 0.222 | 0.748 | 1.119 |
| 0.1 ng/ml A | 0.464 | 1.558 | 2.639 | 2.342 | 2.787 | 2.967 | 0.217 | 0.763 | 1.05 |
| 100 μg/ml CDDP | 0.153 | 0.381 | 0.28 | 1.993 | 1.53 | 1.433 | 0.034 | 0.039 | 0.032 |
| 0.1 ng/ml A + | 0.164 | 0.479 | 0.265 | 2.291 | 1.486 | 1.541 | 0.026 | 0.024 | 0.026 |
| 100 μg/ml CDDP | | | | | | | | | |
| Average | | | | | | | | | |
| Unstimulated cells | 0.406 | 1.307 | 2.769 | 1.912 | 2.636 | 2.906 | 0.222 | 0.748 | 1.119 |
| 1 ng/ml A | 0.558 | 1.825 | 3.341 | 2.131 | 2.781 | 2.975 | 0.191 | 0.523 | 1.108 |
| 0.1 μg/ml CDDP | 0.324 | 0.717 | 0.676 | 1.369 | 2.223 | 2.191 | 0.169 | 0.24 | 0.132 |
| 1 ng/ml A + | 0.4 | 1.084 | 0.734 | 2.269 | 2.026 | 2.094 | 0.131 | 0.174 | 0.203 |
| 0.1 μg/ml CDDP | | | | | | | | | |
| Unstimulated cells | 0.406 | 1.307 | 2.769 | 1.912 | 2.636 | 2.906 | 0.222 | 0.748 | 1.119 |
| 1 ng/ml A | 0.558 | 1.825 | 3.341 | 2.131 | 2.781 | 2.975 | 0.191 | 0.523 | 1.108 |
| 1 μg/ml CDDP | 0.125 | 0.429 | 0.262 | 1.748 | 1.748 | 1.47 | 0.035 | 0.035 | 0.03 |
| 1 ng/ml A + | 0.147 | 0.377 | 0.281 | 1.99 | 1.639 | 1.71 | 0.029 | 0.027 | 0.028 |
| 1 μg/ml CDDP | | | | | | | | | |
| Unstimulated cells | 0.406 | 1.307 | 2.769 | 1.912 | 2.636 | 2.906 | 0.222 | 0.748 | 1.119 |
| 1 ng/ml A | 0.558 | 1.825 | 3.341 | 2.131 | 2.781 | 2.975 | 0.191 | 0.523 | 1.108 |
| 10 μg/ml CDDP | 0.191 | 0.466 | 0.337 | 1.597 | 1.339 | 1.435 | 0.048 | 0.038 | 0.035 |
| 1 ng/ml A + | 0.197 | 0.483 | 0.403 | 1.986 | 1.522 | 1.44 | 0.054 | 0.04 | 0.037 |
| 10 μg/ml CDDP | | | | | | | | | |
| Unstimulated cells | 0.406 | 1.307 | 2.769 | 1.912 | 2.636 | 2.906 | 0.222 | 0.748 | 1.119 |
| 1 ng/ml A | 0.558 | 1.825 | 3.341 | 2.131 | 2.781 | 2.975 | 0.191 | 0.523 | 1.108 |
| 100 μg/ml CDDP | 0.153 | 0.381 | 0.28 | 1.993 | 1.53 | 1.433 | 0.034 | 0.039 | 0.032 |
| 1 ng/ml A + | 0.209 | 0.56 | 0.407 | 2.416 | 2.149 | 2.126 | 0.032 | 0.034 | 0.03 |
| 100 μg/ml CDDP | | | | | | | | | |
| Average | | | | | | | | | |
| Unstimulated cells | 0.406 | 1.307 | 2.769 | 1.912 | 2.636 | 2.906 | 0.222 | 0.748 | 1.119 |
| 10 ng/ml A | 0.65 | 1.884 | 3.161 | 1.995 | 2.754 | 2.961 | 0.218 | 0.623 | 1.028 |
| 0.1 μg/ml CDDP | 0.324 | 0.717 | 0.676 | 1.369 | 2.223 | 2.191 | 0.169 | 0.24 | 0.132 |
| 10 ng/ml A + | 0.425 | 0.771 | 0.628 | 2.028 | 2.081 | 1.864 | 0.172 | 0.235 | 0.269 |
| 0.1 μg/ml CDDP | | | | | | | | | |
| Unstimulated cells | 0.406 | 1.307 | 2.769 | 1.912 | 2.636 | 2.906 | 0.222 | 0.748 | 1.119 |
| 10 ng/ml A | 0.65 | 1.884 | 3.161 | 1.995 | 2.754 | 2.961 | 0.218 | 0.623 | 1.028 |
| 1 μg/ml CDDP | 0.125 | 0.429 | 0.262 | 1.748 | 1.748 | 1.47 | 0.035 | 0.035 | 0.03 |
| 10 ng/ml A + | 0.129 | 0.458 | 0.438 | 2.159 | 1.872 | 1.723 | 0.033 | 0.032 | 0.027 |
| 1 μg/ml CDDP | | | | | | | | | |
| Unstimulated cells | 0.406 | 1.307 | 2.769 | 1.912 | 2.636 | 2.906 | 0.222 | 0.748 | 1.119 |
| 10 ng/ml A | 0.65 | 1.884 | 3.161 | 1.995 | 2.754 | 2.961 | 0.218 | 0.623 | 1.028 |
| 10 μg/ml CDDP | 0.191 | 0.466 | 0.337 | 1.597 | 1.339 | 1.435 | 0.048 | 0.038 | 0.035 |
| 10 ng/ml A + | 0.19 | 0.857 | 0.424 | 1.874 | 1.577 | 1.717 | 0.059 | 0.038 | 0.032 |
| 10 μg/ml CDDP | | | | | | | | | |
| Unstimulated cells | 0.406 | 1.307 | 2.769 | 1.912 | 2.636 | 2.906 | 0.222 | 0.748 | 1.119 |
| 10 ng/ml A | 0.65 | 1.884 | 3.161 | 1.995 | 2.754 | 2.961 | 0.218 | 0.623 | 1.028 |
| 100 μg/ml CDDP | 0.153 | 0.381 | 0.28 | 1.993 | 1.53 | 1.433 | 0.034 | 0.039 | 0.032 |
| 10 ng/ml A + | 0.185 | 0.646 | 0.428 | 2.368 | 1.801 | 1.827 | 0.032 | 0.032 | 0.027 |
| 100 μg/ml CDDP | | | | | | | | | |

TABLE 39

| | PANC-1 | Run 2 | | | | | | | |
|---|---|---|---|---|---|---|---|---|---|
| | CVE | CVE | CVE | SRB | SRB | SRB | MTT | MTT | MTT |
| | 24 h | 48 h | 72 h | 24 h | 48 h | 72 h | 24 h | 48 h | 72 h |
| Average | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 0.01 ng/ml A | 0.757 | 0.998 | 2.607 | 2.448 | 2.47 | 2.898 | 0.248 | 0.412 | 0.767 |
| 0.1 μg/ml CDDP | 0.793 | 0.575 | 0.839 | 1.924 | 1.505 | 2.471 | 0.096 | 0.178 | 0.241 |
| 0.01 ng/ml A + | 0.529 | 0.432 | 0.463 | 1.97 | 1.4 | 1.884 | 0.078 | 0.134 | 0.111 |
| 0.1 μg/ml CDDP | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 0.01 ng/ml A | 0.757 | 0.998 | 2.607 | 2.448 | 2.47 | 2.898 | 0.248 | 0.412 | 0.767 |
| 1 μg/ml CDDP | 0.456 | 0.249 | 0.514 | 2.391 | 1.549 | 2.171 | 0.029 | 0.037 | 0.033 |
| 0.01 ng/ml A + | 0.326 | 0.25 | 0.518 | 2.253 | 0.812 | 1.407 | 0.024 | 0.028 | 0.025 |
| 1 μg/ml CDDP | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 0.01 ng/ml A | 0.757 | 0.998 | 2.607 | 2.448 | 2.47 | 2.898 | 0.248 | 0.412 | 0.767 |
| 10 μg/ml CDDP | 0.602 | 0.33 | 0.49 | 2.075 | 1.106 | 1.815 | 0.058 | 0.041 | 0.04 |
| 0.01 ng/ml A + | 0.33 | 0.195 | 0.284 | 1.695 | 0.996 | 1.206 | 0.036 | 0.037 | 0.031 |
| 10 μg/ml CDDP | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 0.01 ng/ml A | 0.757 | 0.998 | 2.607 | 2.448 | 2.47 | 2.898 | 0.248 | 0.412 | 0.767 |
| 100 μg/ml CDDP | 0.345 | 0.323 | 0.385 | 1.821 | 0.752 | 1.374 | 0.029 | 0.034 | 0.033 |
| 0.01 ng/ml A + | 0.407 | 0.219 | 0.29 | 1.805 | 1.5 | 1.793 | 0.025 | 0.031 | 0.027 |
| 100 μg/ml CDDP | | | | | | | | | |
| Average | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 0.1 ng/ml A | 0.525 | 1.11 | 2.781 | 1.762 | 2.475 | 2.903 | 0.191 | 0.291 | 0.71 |
| 0.1 μg/ml CDDP | 0.793 | 0.575 | 0.839 | 1.924 | 1.505 | 2.471 | 0.096 | 0.178 | 0.241 |
| 0.1 ng/ml A + | 0.597 | 0.495 | 0.759 | 2.304 | 1.761 | 1.98 | 0.156 | 0.118 | 0.167 |
| 0.1 μg/ml CDDP | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 0.1 ng/ml A | 0.525 | 1.11 | 2.781 | 1.762 | 2.475 | 2.903 | 0.191 | 0.291 | 0.71 |
| 1 μg/ml CDDP | 0.456 | 0.249 | 0.514 | 2.391 | 1.549 | 2.171 | 0.029 | 0.037 | 0.033 |
| 0.1 ng/ml A + | 0.367 | 0.645 | 0.455 | 2.094 | 0.987 | 1.774 | 0.027 | 0.03 | 0.025 |
| 1 μg/ml CDDP | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 0.1 ng/ml A | 0.525 | 1.11 | 2.781 | 1.762 | 2.475 | 2.903 | 0.191 | 0.291 | 0.71 |
| 10 μg/ml CDDP | 0.602 | 0.33 | 0.49 | 2.075 | 1.106 | 1.815 | 0.058 | 0.041 | 0.04 |
| 0.1 ng/ml A + | 0.526 | 0.297 | 0.432 | 1.299 | 1.309 | 1.512 | 0.042 | 0.034 | 0.031 |
| 10 μg/ml CDDP | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 0.1 ng/ml A | 0.525 | 1.11 | 2.781 | 1.762 | 2.475 | 2.903 | 0.191 | 0.291 | 0.71 |
| 100 μg/ml CDDP | 0.345 | 0.323 | 0.385 | 1.821 | 0.752 | 1.374 | 0.029 | 0.034 | 0.033 |
| 0.1 ng/ml A + | 0.417 | 0.284 | 0.391 | 1.945 | 2.159 | 2.274 | 0.025 | 0.032 | 0.025 |
| 100 μg/ml CDDP | | | | | | | | | |
| Average | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 1 ng/ml A | 0.65 | 1.57 | 3.291 | 2.18 | 2.262 | 2.916 | 0.145 | 0.302 | 0.707 |
| 0.1 μg/ml CDDP | 0.793 | 0.575 | 0.839 | 1.924 | 1.505 | 2.471 | 0.096 | 0.178 | 0.241 |
| 1 ng/ml A + | 0.748 | 0.623 | 1.056 | 2.303 | 1.52 | 2.075 | 0.099 | 0.134 | 0.16 |
| 0.1 μg/ml CDDP | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 1 ng/ml A | 0.65 | 1.57 | 3.291 | 2.18 | 2.262 | 2.916 | 0.145 | 0.302 | 0.707 |
| 1 μg/ml CDDP | 0.456 | 0.249 | 0.514 | 2.391 | 1.549 | 2.171 | 0.029 | 0.037 | 0.033 |
| 1 ng/ml A + | 0.556 | 0.44 | 0.525 | 2.692 | 1.598 | 2.073 | 0.029 | 0.035 | 0.03 |
| 1 μg/ml CDDP | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 1 ng/ml A | 0.65 | 1.57 | 3.291 | 2.18 | 2.262 | 2.916 | 0.145 | 0.302 | 0.707 |
| 10 μg/ml CDDP | 0.602 | 0.33 | 0.49 | 2.075 | 1.106 | 1.815 | 0.058 | 0.041 | 0.04 |
| 1 ng/ml A + | 0.673 | 0.317 | 0.422 | 1.738 | 1.212 | 1.882 | 0.05 | 0.042 | 0.039 |
| 10 μg/ml CDDP | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 1 ng/ml A | 0.65 | 1.57 | 3.291 | 2.18 | 2.262 | 2.916 | 0.145 | 0.302 | 0.707 |
| 100 μg/ml CDDP | 0.345 | 0.323 | 0.385 | 1.821 | 0.752 | 1.374 | 0.029 | 0.034 | 0.033 |
| 1 ng/ml A + | 0.545 | 0.383 | 0.529 | 1.832 | 1.394 | 2.188 | 0.03 | 0.037 | 0.034 |
| 100 μg/ml CDDP | | | | | | | | | |
| Average | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 10 ng/ml A | 0.718 | 1.648 | 3.033 | 1.188 | 2.04 | 2.879 | 0.145 | 0.312 | 0.848 |
| 0.1 μg/ml CDDP | 0.793 | 0.575 | 0.839 | 1.924 | 1.505 | 2.471 | 0.096 | 0.178 | 0.241 |
| 10 ng/ml A + | 0.645 | 0.677 | 0.867 | 1.978 | 1.543 | 2.116 | 0.091 | 0.147 | 0.183 |
| 0.1 μg/ml CDDP | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 10 ng/ml A | 0.718 | 1.648 | 3.033 | 1.188 | 2.04 | 2.879 | 0.145 | 0.312 | 0.848 |
| 1 μg/ml CDDP | 0.456 | 0.249 | 0.514 | 2.391 | 1.549 | 2.171 | 0.029 | 0.037 | 0.033 |
| 10 ng/ml A + | 0.527 | 0.308 | 0.385 | 1.72 | 1.169 | 1.833 | 0.03 | 0.033 | 0.03 |
| 1 μg/ml CDDP | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 10 ng/ml A | 0.718 | 1.648 | 3.033 | 1.188 | 2.04 | 2.879 | 0.145 | 0.312 | 0.848 |
| 10 μg/ml CDDP | 0.602 | 0.33 | 0.49 | 2.075 | 1.106 | 1.815 | 0.058 | 0.041 | 0.04 |
| 10 ng/ml A + | 0.453 | 0.391 | 0.588 | 1.617 | 1.346 | 2.11 | 0.043 | 0.041 | 0.036 |
| 10 μg/ml CDDP | | | | | | | | | |
| Unstimulated | 0.643 | 1.172 | 2.707 | 1.271 | 2.353 | 2.893 | 0.199 | 0.474 | 0.735 |
| cells | | | | | | | | | |
| 10 ng/ml A | 0.718 | 1.648 | 3.033 | 1.188 | 2.04 | 2.879 | 0.145 | 0.312 | 0.848 |
| 100 μg/ml CDDP | 0.345 | 0.323 | 0.385 | 1.821 | 0.752 | 1.374 | 0.029 | 0.034 | 0.033 |
| 10 ng/ml A + | 0.346 | 0.786 | 0.45 | 1.577 | 1.511 | 1.412 | 0.031 | 0.035 | 0.034 |
| 100 μg/ml CDDP | | | | | | | | | |

TABLE 40

| | PANC-1 | Run 3 | | | | | | | |
|---|---|---|---|---|---|---|---|---|---|
| | CVE | CVE | CVE | SRB | SRB | SRB | MTT | MTT | MTT |
| | 24 h | 48 h | 72 h | 24 h | 48 h | 72 h | 24 h | 48 h | 72 h |
| Average | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 0.01 ng/ml A | 0.409 | 1.04 | 2.711 | 1.793 | 2.328 | 2.882 | 0.091 | 0.518 | 0.954 |
| 0.1 μg/ml CDDP | 0.476 | 0.753 | 0.872 | 1.881 | 1.861 | 2.536 | 0.076 | 0.286 | 0.344 |
| 0.01 ng/ml A + | 0.295 | 0.283 | 0.336 | 1.672 | 1.25 | 1.837 | 0.055 | 0.143 | 0.115 |
| 0.1 μg/ml CDDP | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 0.01 ng/ml A | 0.409 | 1.04 | 2.711 | 1.793 | 2.328 | 2.882 | 0.091 | 0.518 | 0.954 |
| 1 μg/ml CDDP | 0.457 | 0.395 | 0.358 | 2.008 | 1.165 | 2.281 | 0.02 | 0.043 | 0.049 |
| 0.01 ng/ml A + | 0.159 | 0.135 | 0.25 | 1.82 | 0.994 | 1.583 | 0.021 | 0.036 | 0.038 |
| 1 μg/ml CDDP | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 0.01 ng/ml A | 0.409 | 1.04 | 2.711 | 1.793 | 2.328 | 2.882 | 0.091 | 0.518 | 0.954 |
| 10 μg/ml CDDP | 0.698 | 0.218 | 0.421 | 1.74 | 0.954 | 2.07 | 0.04 | 0.045 | 0.05 |
| 0.01 ng/ml A + | 0.186 | 0.253 | 0.554 | 1.755 | 1.19 | 1.237 | 0.028 | 0.042 | 0.042 |
| 10 μg/ml CDDP | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 0.01 ng/ml A | 0.409 | 1.04 | 2.711 | 1.793 | 2.328 | 2.882 | 0.091 | 0.518 | 0.954 |
| 100 μg/ml CDDP | 0.428 | 0.18 | 0.253 | 2.087 | 0.858 | 1.584 | 0.022 | 0.044 | 0.049 |
| 0.01 ng/ml A + | 0.506 | 0.297 | 0.456 | 1.799 | 1.24 | 1.571 | 0.02 | 0.037 | 0.041 |
| 100 μg/ml CDDP | | | | | | | | | |
| Average | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 0.1 ng/ml A | 0.801 | 1.036 | 2.713 | 1.831 | 2.212 | 2.98 | 0.163 | 0.368 | 0.909 |
| 0.1 μg/ml CDDP | 0.476 | 0.753 | 0.872 | 1.881 | 1.861 | 2.536 | 0.076 | 0.286 | 0.344 |
| 0.1 ng/ml A + | 0.59 | 0.45 | 0.496 | 1.623 | 1.387 | 2.045 | 0.104 | 0.119 | 0.143 |
| 0.1 μg/ml CDDP | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 0.1 ng/ml A | 0.801 | 1.036 | 2.713 | 1.831 | 2.212 | 2.98 | 0.163 | 0.368 | 0.909 |
| 1 μg/ml CDDP | 0.457 | 0.395 | 0.358 | 2.008 | 1.165 | 2.281 | 0.02 | 0.043 | 0.049 |
| 0.1 ng/ml A + | 0.287 | 0.202 | 0.289 | 1.923 | 0.911 | 1.698 | 0.021 | 0.034 | 0.037 |
| 1 μg/ml CDDP | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 0.1 ng/ml A | 0.801 | 1.036 | 2.713 | 1.831 | 2.212 | 2.98 | 0.163 | 0.368 | 0.909 |
| 10 μg/ml CDDP | 0.698 | 0.218 | 0.421 | 1.74 | 0.954 | 2.07 | 0.04 | 0.045 | 0.05 |
| 0.1 ng/ml A + | 0.345 | 0.435 | 0.834 | 1.686 | 0.899 | 1.291 | 0.041 | 0.041 | 0.044 |
| 10 μg/ml CDDP | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 0.1 ng/ml A | 0.801 | 1.036 | 2.713 | 1.831 | 2.212 | 2.98 | 0.163 | 0.368 | 0.909 |
| 100 μg/ml CDDP | 0.428 | 0.18 | 0.253 | 2.087 | 0.858 | 1.584 | 0.022 | 0.044 | 0.049 |
| 0.1 ng/ml A + | 0.462 | 0.329 | 0.688 | 1.688 | 1.129 | 1.979 | 0.02 | 0.037 | 0.039 |
| 100 μg/ml CDDP | | | | | | | | | |
| Average | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 1 ng/ml A | 0.934 | 0.995 | 3.17 | 1.757 | 1.477 | 2.209 | 0.11 | 0.298 | 0.402 |
| 0.1 μg/ml CDDP | 0.476 | 0.753 | 0.872 | 1.881 | 1.861 | 2.536 | 0.076 | 0.286 | 0.344 |
| 1 ng/ml A + | 0.88 | 0.629 | 0.757 | 2.149 | 1.504 | 2.054 | 0.081 | 0.168 | 0.195 |
| 0.1 μg/ml CDDP | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 1 ng/ml A | 0.934 | 0.995 | 3.17 | 1.757 | 1.477 | 2.209 | 0.11 | 0.298 | 0.402 |
| 1 μg/ml CDDP | 0.457 | 0.395 | 0.358 | 2.008 | 1.165 | 2.281 | 0.02 | 0.043 | 0.049 |
| 1 ng/ml A + | 0.563 | 0.259 | 0.305 | 2.321 | 0.992 | 1.797 | 0.021 | 0.038 | 0.039 |
| 1 μg/ml CDDP | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 1 ng/ml A | 0.934 | 0.995 | 3.17 | 1.757 | 1.477 | 2.209 | 0.11 | 0.298 | 0.402 |
| 10 μg/ml CDDP | 0.698 | 0.218 | 0.421 | 1.74 | 0.954 | 2.07 | 0.04 | 0.045 | 0.05 |
| 1 ng/ml A + | 0.59 | 0.49 | 0.594 | 2.007 | 1.246 | 2.125 | 0.04 | 0.052 | 0.051 |
| 10 μg/ml CDDP | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 1 ng/ml A | 0.934 | 0.995 | 3.17 | 1.757 | 1.477 | 2.209 | 0.11 | 0.298 | 0.402 |
| 100 μg/ml CDDP | 0.428 | 0.18 | 0.253 | 2.087 | 0.858 | 1.584 | 0.022 | 0.044 | 0.049 |
| 1 ng/ml A + | 0.501 | 0.481 | 0.589 | 2.392 | 1.451 | 2.299 | 0.022 | 0.04 | 0.046 |
| 100 μg/ml CDDP | | | | | | | | | |
| Average | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 10 ng/ml A | 0.964 | 1.63 | 3.289 | 2.113 | 2.209 | 2.903 | 0.19 | 0.59 | 0.884 |
| 0.1 μg/ml CDDP | 0.476 | 0.753 | 0.872 | 1.881 | 1.861 | 2.536 | 0.076 | 0.286 | 0.344 |
| 10 ng/ml A + | 0.804 | 0.608 | 0.638 | 1.937 | 1.303 | 2.068 | 0.092 | 0.234 | 0.226 |
| 0.1 μg/ml CDDP | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 10 ng/ml A | 0.964 | 1.63 | 3.289 | 2.113 | 2.209 | 2.903 | 0.19 | 0.59 | 0.884 |
| 1 μg/ml CDDP | 0.457 | 0.395 | 0.358 | 2.008 | 1.165 | 2.281 | 0.02 | 0.043 | 0.049 |
| 10 ng/ml A + | 0.547 | 0.289 | 0.314 | 1.998 | 1.002 | 2.261 | 0.021 | 0.04 | 0.042 |
| 1 μg/ml CDDP | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 10 ng/ml A | 0.964 | 1.63 | 3.289 | 2.113 | 2.209 | 2.903 | 0.19 | 0.59 | 0.884 |
| 10 μg/ml CDDP | 0.698 | 0.218 | 0.421 | 1.74 | 0.954 | 2.07 | 0.04 | 0.045 | 0.05 |
| 10 ng/ml A + | 0.544 | 0.723 | 0.369 | 1.823 | 0.889 | 2.101 | 0.035 | 0.047 | 0.048 |
| 10 μg/ml CDDP | | | | | | | | | |
| Unstimulated | 0.418 | 0.964 | 2.862 | 1.929 | 2.332 | 2.924 | 0.114 | 0.546 | 1.012 |
| cells | | | | | | | | | |
| 10 ng/ml A | 0.964 | 1.63 | 3.289 | 2.113 | 2.209 | 2.903 | 0.19 | 0.59 | 0.884 |
| 100 μg/ml CDDP | 0.428 | 0.18 | 0.253 | 2.087 | 0.858 | 1.584 | 0.022 | 0.044 | 0.049 |
| 10 ng/ml A + | 0.618 | 0.742 | 0.332 | 2.294 | 1.068 | 2.144 | 0.02 | 0.04 | 0.045 |
| 100 μg/ml CDDP | | | | | | | | | |

TABLE 41

| | PC3 | Run 1 | | | | | | | |
|---|---|---|---|---|---|---|---|---|---|
| | CVE | CVE | CVE | SRB | SRB | SRB | MTT | MTT | MTT |
| | 24 h | 48 h | 72 h | 24 h | 48 h | 72 h | 24 h | 48 h | 72 h |
| Average | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 0.01 μg/ml A | 0.733 | 1.562 | 1.876 | 1.91 | 2.402 | 2.706 | 0.136 | 0.235 | 0.443 |
| 0.1 μg/ml CDDP | 0.73 | 0.938 | 1.091 | 2.058 | 2.343 | 2.256 | 0.137 | 0.162 | 0.188 |
| 0.01 ng/ml A + | 0.647 | 0.766 | 0.687 | 1.546 | 2.052 | 1.997 | 0.113 | 0.144 | 0.103 |
| 0.1 μg/ml CDDP | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 0.01 ng/ml A | 0.733 | 1.562 | 1.876 | 1.91 | 2.402 | 2.706 | 0.136 | 0.235 | 0.443 |
| 1 μg/ml CDDP | 0.368 | 0.483 | 0.415 | 1.352 | 1.677 | 2.227 | 0.049 | 0.027 | 0.034 |
| 0.01 ng/ml A + | 0.266 | 0.339 | 0.402 | 1.247 | 1.759 | 1.247 | 0.043 | 0.022 | 0.026 |
| 1 μg/ml CDDP | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 0.01 ng/ml A | 0.733 | 1.562 | 1.876 | 1.91 | 2.402 | 2.706 | 0.136 | 0.235 | 0.443 |
| 10 μg/ml CDDP | 0.548 | 0.706 | 0.64 | 1.604 | 2.159 | 2.554 | 0.091 | 0.064 | 0.044 |
| 0.01 ng/ml A + | 0.646 | 0.837 | 0.657 | 1.808 | 1.697 | 1.778 | 0.093 | 0.072 | 0.054 |
| 10 μg/ml CDDP | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 0.01 ng/ml A | 0.733 | 1.562 | 1.876 | 1.91 | 2.402 | 2.706 | 0.136 | 0.235 | 0.443 |
| 100 μg/ml CDDP | 0.337 | 0.619 | 0.421 | 1.605 | 1.851 | 1.543 | 0.053 | 0.029 | 0.029 |
| 0.01 ng/ml A + | 0.743 | 0.51 | 0.517 | 1.67 | 1.589 | 1.523 | 0.042 | 0.02 | 0.029 |
| 100 μg/ml CDDP | | | | | | | | | |
| Average | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 0.1 ng/ml A | 0.851 | 1.497 | 1.992 | 1.919 | 2.655 | 2.744 | 0.161 | 0.281 | 0.419 |
| 0.1 μg/ml CDDP | 0.73 | 0.938 | 1.091 | 2.058 | 2.343 | 2.256 | 0.137 | 0.162 | 0.188 |
| 0.1 ng/ml A + | 0.691 | 0.873 | 0.718 | 1.82 | 2.439 | 2.057 | 0.121 | 0.128 | 0.114 |
| 0.1 μg/ml CDDP | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 0.1 ng/ml A | 0.851 | 1.497 | 1.992 | 1.919 | 2.655 | 2.744 | 0.161 | 0.281 | 0.419 |
| 1 μg/ml CDDP | 0.368 | 0.483 | 0.415 | 1.352 | 1.677 | 2.227 | 0.049 | 0.027 | 0.034 |
| 0.1 ng/ml A + | 0.381 | 0.417 | 0.497 | 1.559 | 2.145 | 1.483 | 0.042 | 0.018 | 0.027 |
| 1 μg/ml CDDP | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 0.1 ng/ml A | 0.851 | 1.497 | 1.992 | 1.919 | 2.655 | 2.744 | 0.161 | 0.281 | 0.419 |
| 10 μg/ml CDDP | 0.548 | 0.706 | 0.64 | 1.604 | 2.159 | 2.554 | 0.091 | 0.064 | 0.044 |
| 0.1 ng/ml A + | 0.558 | 0.787 | 0.681 | 2.09 | 2.29 | 1.862 | 0.104 | 0.066 | 0.063 |
| 10 μg/ml CDDP | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 0.1 ng/ml A | 0.851 | 1.497 | 1.992 | 1.919 | 2.655 | 2.744 | 0.161 | 0.281 | 0.419 |
| 100 μg/ml CDDP | 0.337 | 0.619 | 0.421 | 1.605 | 1.851 | 1.543 | 0.053 | 0.029 | 0.029 |
| 0.1 ng/ml A + | 0.64 | 0.774 | 0.493 | 1.921 | 2.156 | 1.835 | 0.046 | 0.021 | 0.03 |
| 100 μg/ml CDDP | | | | | | | | | |
| Average | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 1 ng/ml A | 1.004 | 1.772 | 2.797 | 1.947 | 2.607 | 2.749 | 0.161 | 0.289 | 0.352 |
| 0.1 μg/ml CDDP | 0.73 | 0.938 | 1.091 | 2.058 | 2.343 | 2.256 | 0.137 | 0.162 | 0.188 |
| 1 ng/ml A + | 0.81 | 1.076 | 0.949 | 2.008 | 2.687 | 2.291 | 0.102 | 0.136 | 0.117 |
| 0.1 μg/ml CDDP | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 1 ng/ml A | 1.004 | 1.772 | 2.797 | 1.947 | 2.607 | 2.749 | 0.161 | 0.289 | 0.352 |
| 1 μg/ml CDDP | 0.368 | 0.483 | 0.415 | 1.352 | 1.677 | 2.227 | 0.049 | 0.027 | 0.034 |
| 1 ng/ml A + | 0.385 | 0.524 | 0.519 | 1.706 | 2.433 | 2.226 | 0.054 | 0.025 | 0.033 |
| 1 μg/ml CDDP | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 1 ng/ml A | 1.004 | 1.772 | 2.797 | 1.947 | 2.607 | 2.749 | 0.161 | 0.289 | 0.352 |
| 10 μg/ml CDDP | 0.548 | 0.706 | 0.64 | 1.604 | 2.159 | 2.554 | 0.091 | 0.064 | 0.044 |
| 1 ng/ml A + | 0.861 | 0.995 | 0.878 | 2.525 | 2.343 | 2.085 | 0.129 | 0.08 | 0.059 |
| 10 μg/ml CDDP | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 1 ng/ml A | 1.004 | 1.772 | 2.797 | 1.947 | 2.607 | 2.749 | 0.161 | 0.289 | 0.352 |
| 100 μg/ml CDDP | 0.337 | 0.619 | 0.421 | 1.605 | 1.851 | 1.543 | 0.053 | 0.029 | 0.029 |
| 1 ng/ml A + | 0.381 | 0.647 | 0.931 | 2.129 | 2.058 | 2.701 | 0.057 | 0.026 | 0.03 |
| 100 μg/ml CDDP | | | | | | | | | |
| Average | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 10 ng/ml A | 1.066 | 1.801 | 2.598 | 1.951 | 2.382 | 2.61 | 0.192 | 0.351 | 0.487 |
| 0.1 μg/ml CDDP | 0.73 | 0.938 | 1.091 | 2.058 | 2.343 | 2.256 | 0.137 | 0.162 | 0.188 |
| 10 ng/ml A + | 1.062 | 1.124 | 0.916 | 2.143 | 2.267 | 2.321 | 0.159 | 0.144 | 0.133 |
| 0.1 μg/ml CDDP | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 10 ng/ml A | 1.066 | 1.801 | 2.598 | 1.951 | 2.382 | 2.61 | 0.192 | 0.351 | 0.487 |
| 1 μg/ml CDDP | 0.368 | 0.483 | 0.415 | 1.352 | 1.677 | 2.227 | 0.049 | 0.027 | 0.034 |
| 10 ng/ml A + | 0.477 | 0.718 | 0.665 | 1.872 | 1.812 | 2.505 | 0.05 | 0.028 | 0.029 |
| 1 μg/ml CDDP | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 10 ng/ml A | 1.066 | 1.801 | 2.598 | 1.951 | 2.382 | 2.61 | 0.192 | 0.351 | 0.487 |
| 10 μg/ml CDDP | 0.548 | 0.706 | 0.64 | 1.604 | 2.159 | 2.554 | 0.091 | 0.064 | 0.044 |
| 10 ng/ml A + | 0.955 | 1.117 | 1.166 | 2.277 | 2.24 | 2.15 | 0.2 | 0.101 | 0.077 |
| 10 μg/ml CDDP | | | | | | | | | |
| Unstimulated | 0.693 | 1.508 | 1.742 | 1.797 | 2.322 | 2.531 | 0.168 | 0.298 | 0.39 |
| cells | | | | | | | | | |
| 10 ng/ml A | 1.066 | 1.801 | 2.598 | 1.951 | 2.382 | 2.61 | 0.192 | 0.351 | 0.487 |
| 100 μg/ml CDDP | 0.337 | 0.619 | 0.421 | 1.605 | 1.851 | 1.543 | 0.053 | 0.029 | 0.029 |
| 10 ng/ml A + | 0.98 | 0.721 | 1.069 | 2.398 | 1.696 | 2.162 | 0.053 | 0.026 | 0.03 |
| 100 μg/ml CDDP | | | | | | | | | |

TABLE 42

| | PC3 | Run 2 | | | | | | | |
|---|---|---|---|---|---|---|---|---|---|
| | CVE | CVE | CVE | SRB | SRB | SRB | MTT | MTT | MTT |
| | 24 h | 48 h | 72 h | 24 h | 48 h | 72 h | 24 h | 48 h | 72 h |
| Average | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 0.0 ng/ml A | 0.5 | 1.048 | 1.121 | 1.452 | 2.438 | 2.407 | 0.066 | 0.267 | 0.332 |
| 0.1 μg/ml CDDP | 0.489 | 0.714 | 0.76 | 1.578 | 2.279 | 2.069 | 0.072 | 0.195 | 0.223 |
| 0.01 ng/ml A + | 0.422 | 0.59 | 0.48 | 1.664 | 1.893 | 1.62 | 0.071 | 0.146 | 0.113 |
| 0.1 μg/ml CDDP | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 0.01 ng/ml A | 0.5 | 1.048 | 1.121 | 1.452 | 2.438 | 2.407 | 0.066 | 0.267 | 0.332 |
| 1 μg/ml CDDP | 0.238 | 0.393 | 0.284 | 1.469 | 2.066 | 1.64 | 0.04 | 0.051 | 0.039 |
| 0.01 ng/ml A + | 0.195 | 0.365 | 0.254 | 1.678 | 1.549 | 1.255 | 0.032 | 0.041 | 0.032 |
| 1 μg/ml CDDP | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 0.01 ng/ml A | 0.5 | 1.048 | 1.121 | 1.452 | 2.438 | 2.407 | 0.066 | 0.267 | 0.332 |
| 10 μg/ml CDDP | 0.498 | 0.694 | 0.526 | 1.403 | 2.144 | 1.63 | 0.079 | 0.074 | 0.068 |
| 0.01 ng/ml A + | 0.386 | 0.542 | 0.419 | 1.525 | 2.057 | 1.452 | 0.068 | 0.116 | 0.07 |
| 10 μg/ml CDDP | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 0.01 ng/ml A | 0.5 | 1.048 | 1.121 | 1.452 | 2.438 | 2.407 | 0.066 | 0.267 | 0.332 |
| 100 μg/ml CDDP | 0.309 | 0.419 | 0.316 | 1.5 | 1.77 | 1.271 | 0.052 | 0.047 | 0.037 |
| 0.01 ng/ml A + | 0.479 | 0.328 | 0.706 | 1.795 | 2.246 | 1.399 | 0.031 | 0.04 | 0.031 |
| 100 μg/ml CDDP | | | | | | | | | |
| Average | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 0.1 ng/ml A | 0.581 | 1.104 | 1.305 | 1.542 | 2.302 | 2.245 | 0.092 | 0.149 | 0.343 |
| 0.1 μg/ml CDDP | 0.489 | 0.714 | 0.76 | 1.578 | 2.279 | 2.069 | 0.072 | 0.195 | 0.223 |
| 0.1 ng/ml A + | 0.44 | 0.727 | 0.516 | 1.442 | 2.018 | 1.838 | 0.072 | 0.091 | 0.125 |
| 0.1 μg/ml CDDP | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 0.1 ng/ml A | 0.581 | 1.104 | 1.305 | 1.542 | 2.302 | 2.245 | 0.092 | 0.149 | 0.343 |
| 1 μg/ml CDDP | 0.238 | 0.393 | 0.284 | 1.469 | 2.066 | 1.64 | 0.04 | 0.051 | 0.039 |
| 0.1 ng/ml A + | 0.257 | 0.379 | 0.359 | 1.518 | 1.702 | 1.401 | 0.04 | 0.046 | 0.035 |
| 1 μg/ml CDDP | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 0.1 ng/ml A | 0.581 | 1.104 | 1.305 | 1.542 | 2.302 | 2.245 | 0.092 | 0.149 | 0.343 |
| 10 μg/ml CDDP | 0.498 | 0.694 | 0.526 | 1.403 | 2.144 | 1.63 | 0.079 | 0.074 | 0.068 |
| 0.1 ng/ml A + | 0.442 | 0.661 | 0.534 | 1.61 | 1.964 | 1.504 | 0.083 | 0.083 | 0.072 |
| 10 μg/ml CDDP | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 0.1 ng/ml A | 0.581 | 1.104 | 1.305 | 1.542 | 2.302 | 2.245 | 0.092 | 0.149 | 0.343 |
| 100 μg/ml CDDP | 0.309 | 0.419 | 0.316 | 1.5 | 1.77 | 1.271 | 0.052 | 0.047 | 0.037 |
| 0.1 ng/ml A + | 0.482 | 0.524 | 0.646 | 1.554 | 2.129 | 1.55 | 0.036 | 0.044 | 0.033 |
| 100 μg/ml CDDP | | | | | | | | | |
| Average | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 1 ng/ml A | 0.668 | 1.197 | 1.58 | 1.514 | 2.258 | 2.411 | 0.095 | 0.119 | 0.29 |
| 0.1 μg/ml CDDP | 0.489 | 0.714 | 0.76 | 1.578 | 2.279 | 2.069 | 0.072 | 0.195 | 0.223 |
| 1 ng/ml A + | 0.611 | 0.881 | 0.773 | 1.593 | 2.001 | 2.028 | 0.075 | 0.094 | 0.146 |
| 0.1 μg/ml CDDP | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 1 ng/ml A | 0.668 | 1.197 | 1.58 | 1.514 | 2.258 | 2.411 | 0.095 | 0.119 | 0.29 |
| 1 μg/ml CDDP | 0.238 | 0.393 | 0.284 | 1.469 | 2.066 | 1.64 | 0.04 | 0.051 | 0.039 |
| 1 ng/ml A + | 0.308 | 0.381 | 0.362 | 2.122 | 1.963 | 1.717 | 0.044 | 0.047 | 0.039 |
| 1 μg/ml CDDP | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 1 ng/ml A | 0.668 | 1.197 | 1.58 | 1.514 | 2.258 | 2.411 | 0.095 | 0.119 | 0.29 |
| 10 μg/ml CDDP | 0.498 | 0.694 | 0.526 | 1.403 | 2.144 | 1.63 | 0.079 | 0.074 | 0.068 |
| 1 ng/ml A + | 0.556 | 0.705 | 0.587 | 1.685 | 2.099 | 1.955 | 0.074 | 0.074 | 0.069 |
| 10 μg/ml CDDP | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 1 ng/ml A | 0.668 | 1.197 | 1.58 | 1.514 | 2.258 | 2.411 | 0.095 | 0.119 | 0.29 |
| 100 μg/ml CDDP | 0.309 | 0.419 | 0.316 | 1.5 | 1.77 | 1.271 | 0.052 | 0.047 | 0.037 |
| 1 ng/ml A + 0.43 | 0.542 | 0.519 | 2.031 | 2.242 | 1.858 | 0.043 | 0.049 | 0.036 | |
| 100 μg/ml CDDP | | | | | | | | | |
| Average | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 10 ng/ml A | 0.824 | 1.201 | 1.651 | 1.408 | 2.242 | 2.323 | 0.113 | 0.14 | 0.296 |
| 0.1 μg/ml CDDP | 0.489 | 0.714 | 0.76 | 1.578 | 2.279 | 2.069 | 0.072 | 0.195 | 0.223 |
| 10 ng/ml A + | 0.657 | 1.057 | 0.821 | 1.455 | 1.863 | 1.701 | 0.125 | 0.112 | 0.119 |
| 0.1 μg/ml CDDP | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 10 ng/ml A | 0.824 | 1.201 | 1.651 | 1.408 | 2.242 | 2.323 | 0.113 | 0.14 | 0.296 |
| 1 μg/ml CDDP | 0.238 | 0.393 | 0.284 | 1.469 | 2.066 | 1.64 | 0.04 | 0.051 | 0.039 |
| 10 ng/ml A + | 0.282 | 0.39 | 0.41 | 1.587 | 1.985 | 1.512 | 0.058 | 0.045 | 0.036 |
| 1 μg/ml CDDP | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 10 ng/ml A | 0.824 | 1.201 | 1.651 | 1.408 | 2.242 | 2.323 | 0.113 | 0.14 | 0.296 |
| 10 μg/ml CDDP | 0.498 | 0.694 | 0.526 | 1.403 | 2.144 | 1.63 | 0.079 | 0.074 | 0.068 |
| 10 ng/ml A + | 0.554 | 0.608 | 0.736 | 1.68 | 2.14 | 1.852 | 0.107 | 0.082 | 0.065 |
| 10 μg/ml CDDP | | | | | | | | | |
| Unstimulated | 0.529 | 1.228 | 1.132 | 1.356 | 2.096 | 2.333 | 0.074 | 0.249 | 0.341 |
| cells | | | | | | | | | |
| 10 ng/ml A | 0.824 | 1.201 | 1.651 | 1.408 | 2.242 | 2.323 | 0.113 | 0.14 | 0.296 |
| 100 μg/ml CDDP | 0.309 | 0.419 | 0.316 | 1.5 | 1.77 | 1.271 | 0.052 | 0.047 | 0.037 |
| 10 ng/ml A + | 0.45 | 0.744 | 0.589 | 2.222 | 2.26 | 1.635 | 0.053 | 0.039 | 0.036 |
| 100 μg/ml CDDP | | | | | | | | | |

TABLE 43

| | PC3 | Run 3 | | | | | | | |
|---|---|---|---|---|---|---|---|---|---|
| | CVE | CVE | CVE | SRB | SRB | SRB | MTT | MTT | MTT |
| | 24 h | 48 h | 72 h | 24 h | 48 h | 72 h | 24 h | 48 h | 72 h |
| Average | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 0.01 ng/ml A | 0.568 | 0.773 | 1.338 | 1.453 | 2.069 | 2.274 | 0.085 | 0.15 | 0.26 |
| 0.1 μg/ml CDDP | 0.653 | 0.599 | 0.927 | 1.595 | 1.52 | 1.643 | 0.088 | 0.129 | 0.151 |
| 0.01 ng/ml A + | 0.721 | 0.497 | 0.62 | 1.281 | 1.369 | 1.177 | 0.086 | 0.094 | 0.095 |
| 0.1 μg/ml CDDP | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 0.01 ng/ml A | 0.568 | 0.773 | 1.338 | 1.453 | 2.069 | 2.274 | 0.085 | 0.15 | 0.26 |
| 1 μg/ml CDDP | 0.551 | 0.4 | 1.001 | 1.326 | 1.1 | 1.069 | 0.041 | 0.033 | 0.027 |
| 0.01 ng/ml A + | 0.307 | 0.243 | 0.21 | 1.069 | 0.978 | 0.899 | 0.036 | 0.026 | 0.026 |
| 1 μg/ml CDDP | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 0.01 ng/ml A | 0.568 | 0.773 | 1.338 | 1.453 | 2.069 | 2.274 | 0.085 | 0.15 | 0.26 |
| 10 μg/ml CDDP | 0.638 | 0.599 | 0.666 | 1.24 | 1.474 | 1.122 | 0.096 | 0.062 | 0.057 |
| 0.01 ng/ml A + | 0.418 | 0.39 | 0.339 | 1.237 | 1.197 | 1.153 | 0.079 | 0.08 | 0.068 |
| 10 μg/ml CDDP | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 0.01 ng/ml A | 0.568 | 0.773 | 1.338 | 1.453 | 2.069 | 2.274 | 0.085 | 0.15 | 0.26 |
| 100 μg/ml CDDP | 0.301 | 0.367 | 0.282 | 1.125 | 1.153 | 0.839 | 0.053 | 0.03 | 0.028 |
| 0.01 ng/ml A + | 0.298 | 0.289 | 0.394 | 1.45 | 1.499 | 1.392 | 0.038 | 0.026 | 0.03 |
| 100 μg/ml CDDP | | | | | | | | | |
| Average | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 0.1 ng/ml A | 0.697 | 0.828 | 1.365 | 1.398 | 2.09 | 2.247 | 0.12 | 0.173 | 0.329 |
| 0.1 μg/ml CDDP | 0.653 | 0.599 | 0.927 | 1.595 | 1.52 | 1.643 | 0.088 | 0.129 | 0.151 |
| 0.1 ng/ml A + | 0.625 | 0.569 | 0.526 | 1.272 | 1.419 | 1.087 | 0.107 | 0.11 | 0.119 |
| 0.1 μg/ml CDDP | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 0.1 ng/ml A | 0.697 | 0.828 | 1.365 | 1.398 | 2.09 | 2.247 | 0.12 | 0.173 | 0.329 |
| 1 μg/ml CDDP | 0.551 | 0.4 | 1.001 | 1.326 | 1.1 | 1.069 | 0.041 | 0.033 | 0.027 |
| 0.1 ng/ml A + | 0.348 | 0.291 | 0.41 | 1.162 | 1.076 | 0.815 | 0.039 | 0.026 | 0.024 |
| 1 μg/ml CDDP | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 0.1 ng/ml A | 0.697 | 0.828 | 1.365 | 1.398 | 2.09 | 2.247 | 0.12 | 0.173 | 0.329 |
| 10 μg/ml CDDP | 0.638 | 0.599 | 0.666 | 1.24 | 1.474 | 1.122 | 0.096 | 0.062 | 0.057 |
| 0.1 ng/ml A + | 0.634 | 0.4 | 0.454 | 1.466 | 1.622 | 1.099 | 0.09 | 0.085 | 0.063 |
| 10 μg/ml CDDP | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 0.1 ng/ml A | 0.697 | 0.828 | 1.365 | 1.398 | 2.09 | 2.247 | 0.12 | 0.173 | 0.329 |
| 100 μg/ml CDDP | 0.301 | 0.367 | 0.282 | 1.125 | 1.153 | 0.839 | 0.053 | 0.03 | 0.028 |
| 0.1 ng/ml A + | 0.511 | 0.35 | 0.509 | 2.118 | 1.46 | 1.012 | 0.039 | 0.026 | 0.029 |
| 100 μg/ml CDDP | | | | | | | | | |
| Average | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 1 ng/ml A | 0.775 | 1.076 | 1.649 | 1.319 | 2.092 | 2.274 | 0.125 | 0.137 | 0.268 |
| 0.1 μg/ml CDDP | 0.653 | 0.599 | 0.927 | 1.595 | 1.52 | 1.643 | 0.088 | 0.129 | 0.151 |
| 1 ng/ml A + | 0.658 | 0.782 | 0.682 | 1.35 | 1.703 | 1.195 | 0.096 | 0.096 | 0.093 |
| 0.1 μg/ml CDDP | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 1 ng/ml A | 0.775 | 1.076 | 1.649 | 1.319 | 2.092 | 2.274 | 0.125 | 0.137 | 0.268 |
| 1 μg/ml CDDP | 0.551 | 0.4 | 1.001 | 1.326 | 1.1 | 1.069 | 0.041 | 0.033 | 0.027 |
| 1 ng/ml A + | 0.373 | 0.288 | 0.344 | 1.353 | 1.59 | 1.093 | 0.044 | 0.031 | 0.03 |
| 1 μg/ml CDDP | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 1 ng/ml A | 0.775 | 1.076 | 1.649 | 1.319 | 2.092 | 2.274 | 0.125 | 0.137 | 0.268 |
| 10 μg/ml CDDP | 0.638 | 0.599 | 0.666 | 1.24 | 1.474 | 1.122 | 0.096 | 0.062 | 0.057 |
| 1 ng/ml A + | 0.621 | 0.556 | 1.119 | 1.771 | 1.877 | 1.307 | 0.097 | 0.077 | 0.066 |
| 10 μg/ml CDDP | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 1 ng/ml A | 0.775 | 1.076 | 1.649 | 1.319 | 2.092 | 2.274 | 0.125 | 0.137 | 0.268 |
| 100 μg/ml CDDP | 0.301 | 0.367 | 0.282 | 1.125 | 1.153 | 0.839 | 0.053 | 0.03 | 0.028 |
| 1 ng/ml A + | 0.417 | 0.58 | 0.67 | 1.629 | 1.638 | 1.328 | 0.044 | 0.031 | 0.032 |
| 100 μg/ml CDDP | | | | | | | | | |
| Average | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 10 ng/ml A | 0.826 | 1.128 | 1.546 | 1.22 | 1.98 | 2.225 | 0.108 | 0.167 | 0.262 |
| 0.1 μg/ml CDDP | 0.653 | 0.599 | 0.927 | 1.595 | 1.52 | 1.643 | 0.088 | 0.129 | 0.151 |
| 10 ng/ml A + | 0.717 | 0.792 | 0.725 | 1.473 | 1.601 | 1.224 | 0.12 | 0.131 | 0.124 |
| 0.1 μg/ml CDDP | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 10 ng/ml A | 0.826 | 1.128 | 1.546 | 1.22 | 1.98 | 2.225 | 0.108 | 0.167 | 0.262 |
| 1 μg/ml CDDP | 0.551 | 0.4 | 1.001 | 1.326 | 1.1 | 1.069 | 0.041 | 0.033 | 0.027 |
| 10 ng/ml A + 1 | 0.328 | 0.415 | 0.315 | 1.272 | 1.422 | 1.174 | 0.048 | 0.031 | 0.024 |
| 1 μg/ml CDDP | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 10 ng/ml A | 0.826 | 1.128 | 1.546 | 1.22 | 1.98 | 2.225 | 0.108 | 0.167 | 0.262 |
| 10 μg/ml CDDP | 0.638 | 0.599 | 0.666 | 1.24 | 1.474 | 1.122 | 0.096 | 0.062 | 0.057 |
| 10 ng/ml A + 10 | 0.624 | 0.669 | 0.555 | 1.685 | 1.626 | 1.472 | 0.113 | 0.088 | 0.083 |
| 10 μg/ml CDDP | | | | | | | | | |
| Unstimulated | 0.581 | 0.762 | 1.134 | 1.377 | 1.795 | 2.29 | 0.105 | 0.166 | 0.288 |
| cells | | | | | | | | | |
| 10 ng/ml A | 0.826 | 1.128 | 1.546 | 1.22 | 1.98 | 2.225 | 0.108 | 0.167 | 0.262 |
| 100 μg/ml CDDP | 0.301 | 0.367 | 0.282 | 1.125 | 1.153 | 0.839 | 0.053 | 0.03 | 0.028 |
| 10 ng/ml A + | 0.438 | 0.448 | 0.58 | 1.432 | 1.62 | 1.463 | 0.046 | 0.028 | 0.027 |
| 100 μg/ml CDDP | | | | | | | | | |

TABLE 44

| | T47D | Run 1 | | | | | | | |
|---|---|---|---|---|---|---|---|---|---|
| | CVE | CVE | CVE | SRB | SRB | SRB | MTT | MTT | MTT |
| | 24 h | 48 h | 72 h | 24 h | 48 h | 72 h | 24 h | 48 h | 72 h |
| Average | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 0.01 ng/ml A | 0.642 | 0.799 | 2.015 | 1.235 | 2.413 | 2.854 | 0.186 | 0.409 | 0.675 |
| 0.1 μg/ml CDDP | 0.64 | 0.67 | 0.654 | 1.24 | 2.029 | 2.34 | 0.166 | 0.214 | 0.336 |
| 0.01 ng/ml A + | 0.558 | 0.694 | 0.535 | 1.258 | 2.08 | 2.209 | 0.168 | 0.175 | 0.158 |
| 0.1 μg/ml CDDP | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 0.01 ng/ml A | 0.642 | 0.799 | 2.015 | 1.235 | 2.413 | 2.854 | 0.186 | 0.409 | 0.675 |
| 1 μg/ml CDDP | 0.367 | 0.369 | 0.347 | 1.786 | 1.913 | 1.863 | 0.033 | 0.039 | 0.04 |
| 0.01 ng/ml A + | 0.382 | 0.145 | 0.254 | 1.013 | 1.582 | 1.307 | 0.031 | 0.029 | 0.034 |
| 1 μg/ml CDDP | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 0.01 ng/ml A | 0.642 | 0.799 | 2.015 | 1.235 | 2.413 | 2.854 | 0.186 | 0.409 | 0.675 |
| 10 μg/ml CDDP | 0.417 | 0.518 | 0.34 | 1.586 | 1.768 | 1.428 | 0.046 | 0.044 | 0.05 |
| 0.01 ng/ml A + | 0.791 | 0.22 | 0.283 | 1.193 | 1.552 | 1.577 | 0.066 | 0.07 | 0.068 |
| 10 μg/ml CDDP | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 0.01 ng/ml A | 0.642 | 0.799 | 2.015 | 1.235 | 2.413 | 2.854 | 0.186 | 0.409 | 0.675 |
| 100 μg/ml CDDP | 0.457 | 0.319 | 0.293 | 1.494 | 1.717 | 1.271 | 0.045 | 0.038 | 0.049 |
| 0.01 ng/ml A + | 0.491 | 0.543 | 0.318 | 1.546 | 1.727 | 2.019 | 0.028 | 0.03 | 0.038 |
| 100 μg/ml CDDP | | | | | | | | | |
| Average | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 0.1 ng/ml A | 0.941 | 0.936 | 1.902 | 1.429 | 2.523 | 2.818 | 0.17 | 0.415 | 0.6 |
| 0.1 μg/ml CDDP | 0.64 | 0.67 | 0.654 | 1.24 | 2.029 | 2.34 | 0.166 | 0.214 | 0.336 |
| 0.1 ng/ml A + | 0.62 | 0.7 | 0.636 | 1.681 | 1.943 | 2.136 | 0.142 | 0.228 | 0.213 |
| 0.1 μg/ml CDDP | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 0.1 ng/ml A | 0.941 | 0.936 | 1.902 | 1.429 | 2.523 | 2.818 | 0.17 | 0.415 | 0.6 |
| 1 μg/ml CDDP | 0.367 | 0.369 | 0.347 | 1.786 | 1.913 | 1.863 | 0.033 | 0.039 | 0.04 |
| 0.1 ng/ml A + | 0.584 | 0.235 | 0.428 | 1.458 | 1.701 | 1.77 | 0.029 | 0.028 | 0.03 |
| 1 μg/ml CDDP | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 0.1 ng/ml A | 0.941 | 0.936 | 1.902 | 1.429 | 2.523 | 2.818 | 0.17 | 0.415 | 0.6 |
| 10 μg/ml CDDP | 0.417 | 0.518 | 0.34 | 1.586 | 1.768 | 1.428 | 0.046 | 0.044 | 0.05 |
| 0.1 ng/ml A + | 0.575 | 0.244 | 0.292 | 1.593 | 1.875 | 1.708 | 0.066 | 0.048 | 0.04 |
| 10 μg/ml CDDP | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 0.1 ng/ml A | 0.941 | 0.936 | 1.902 | 1.429 | 2.523 | 2.818 | 0.17 | 0.415 | 0.6 |
| 100 μg/ml CDDP | 0.457 | 0.319 | 0.293 | 1.494 | 1.717 | 1.271 | 0.045 | 0.038 | 0.049 |
| 0.1 ng/ml A + | 0.62 | 0.562 | 0.441 | 2.022 | 2.406 | 1.953 | 0.03 | 0.033 | 0.037 |
| 100 μg/ml CDDP | | | | | | | | | |
| Average | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 1 ng/ml A | 1.115 | 1.143 | 2.31 | 1.562 | 2.621 | 2.825 | 0.141 | 0.432 | 0.863 |
| 0.1 μg/ml CDDP | 0.64 | 0.67 | 0.654 | 1.24 | 2.029 | 2.34 | 0.166 | 0.214 | 0.336 |
| 1 ng/ml A + | 0.809 | 0.857 | 0.877 | 1.74 | 2.119 | 2.111 | 0.114 | 0.232 | 0.217 |
| 0.1 μg/ml CDDP | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 1 ng/ml A | 1.115 | 1.143 | 2.31 | 1.562 | 2.621 | 2.825 | 0.141 | 0.432 | 0.863 |
| 1 μg/ml CDDP | 0.367 | 0.369 | 0.347 | 1.786 | 1.913 | 1.863 | 0.033 | 0.039 | 0.04 |
| 1 ng/ml A + | 0.683 | 0.352 | 0.287 | 1.452 | 2.387 | 1.615 | 0.035 | 0.036 | 0.038 |
| 1 μg/ml CDDP | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 1 ng/ml A | 1.115 | 1.143 | 2.31 | 1.562 | 2.621 | 2.825 | 0.141 | 0.432 | 0.863 |
| 10 μg/ml CDDP | 0.417 | 0.518 | 0.34 | 1.586 | 1.768 | 1.428 | 0.046 | 0.044 | 0.05 |
| 1 ng/ml A + | 1.227 | 0.369 | 0.36 | 1.496 | 2.184 | 1.604 | 0.081 | 0.082 | 0.074 |
| 10 μg/ml CDDP | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 1 ng/ml A | 1.115 | 1.143 | 2.31 | 1.562 | 2.621 | 2.825 | 0.141 | 0.432 | 0.863 |
| 100 μg/ml CDDP | 0.457 | 0.319 | 0.293 | 1.494 | 1.717 | 1.271 | 0.045 | 0.038 | 0.049 |
| 1 ng/ml A + | 0.556 | 0.568 | 0.543 | 1.884 | 2.255 | 1.608 | 0.028 | 0.033 | 0.044 |
| 100 μg/ml CDDP | | | | | | | | | |
| Average | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 10 ng/ml A | 0.932 | 1.34 | 2.05 | 1.402 | 2.459 | 2.794 | 0.436 | 0.268 | 0.902 |
| 0.1 μg/ml CDDP | 0.64 | 0.67 | 0.654 | 1.24 | 2.029 | 2.34 | 0.166 | 0.214 | 0.336 |
| 10 ng/ml A + | 0.773 | 0.706 | 0.815 | 1.349 | 2.18 | 1.675 | 0.278 | 0.163 | 0.219 |
| 0.1 μg/ml CDDP | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 10 ng/ml A | 0.932 | 1.34 | 2.05 | 1.402 | 2.459 | 2.794 | 0.436 | 0.268 | 0.902 |
| 1 μg/ml CDDP | 0.367 | 0.369 | 0.347 | 1.786 | 1.913 | 1.863 | 0.033 | 0.039 | 0.04 |
| 10 ng/ml A + | 0.45 | 0.192 | 0.32 | 1.491 | 1.893 | 1.242 | 0.03 | 0.033 | 0.039 |
| 1 μg/ml CDDP | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 10 ng/ml A | 0.932 | 1.34 | 2.05 | 1.402 | 2.459 | 2.794 | 0.436 | 0.268 | 0.902 |
| 10 μg/ml CDDP | 0.417 | 0.518 | 0.34 | 1.586 | 1.768 | 1.428 | 0.046 | 0.044 | 0.05 |
| 10 ng/ml A + | 0.728 | 0.252 | 0.43 | 1.794 | 1.852 | 1.197 | 0.088 | 0.044 | 0.052 |
| 10 μg/ml CDDP | | | | | | | | | |
| Unstimulated | 0.778 | 0.938 | 1.942 | 1.232 | 2.264 | 2.821 | 0.258 | 0.412 | 0.859 |
| cells | | | | | | | | | |
| 10 ng/ml A | 0.932 | 1.34 | 2.05 | 1.402 | 2.459 | 2.794 | 0.436 | 0.268 | 0.902 |
| 100 μg/ml CDDP | 0.457 | 0.319 | 0.293 | 1.494 | 1.717 | 1.271 | 0.045 | 0.038 | 0.049 |
| 10 ng/ml A + | 0.841 | 0.43 | 0.649 | 2.115 | 2.072 | 1.197 | 0.029 | 0.034 | 0.042 |
| 100 μg/ml CDDP | | | | | | | | | |

TABLE 45

| | T47D | Run 2 | | | | | | | |
|---|---|---|---|---|---|---|---|---|---|
| | CVE | CVE | CVE | SRB | SRB | SRB | MTT | MTT | MTT |
| | 24 h | 48 h | 72 h | 24 h | 48 h | 72 h | 24 h | 48 h | 72 h |
| Average | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 0.01 ng/ml A | 0.604 | 0.864 | 2.93 | 2.586 | 2.511 | 2.714 | 0.197 | 0.33 | 0.643 |
| 0.1 μg/ml CDDP | 0.454 | 0.45 | 1.124 | 2.474 | 2.112 | 1.969 | 0.181 | 0.262 | 0.397 |
| 0.01 ng/ml A + | 0.436 | 0.643 | 0.662 | 2.487 | 1.739 | 1.59 | 0.134 | 0.225 | 0.244 |
| 0.1 μg/ml CDDP | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 0.01 ng/ml A | 0.604 | 0.864 | 2.93 | 2.586 | 2.511 | 2.714 | 0.197 | 0.33 | 0.643 |
| 1 μg/ml CDDP | 0.385 | 0.223 | 0.33 | 1.601 | 1.969 | 1.201 | 0.051 | 0.052 | 0.046 |
| 0.01 ng/ml A + | 0.39 | 0.256 | 0.447 | 1.561 | 1.658 | 1.336 | 0.046 | 0.044 | 0.034 |
| 1 μg/ml CDDP | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 0.01 ng/ml A | 0.604 | 0.864 | 2.93 | 2.586 | 2.511 | 2.714 | 0.197 | 0.33 | 0.643 |
| 10 μg/ml CDDP | 0.597 | 0.315 | 0.334 | 1.417 | 1.988 | 1.567 | 0.07 | 0.066 | 0.041 |
| 0.01 ng/ml A + | 0.503 | 0.229 | 0.441 | 1.75 | 1.632 | 1.386 | 0.102 | 0.085 | 0.06 |
| 10 μg/ml CDDP | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 0.01 ng/ml A | 0.604 | 0.864 | 2.93 | 2.586 | 2.511 | 2.714 | 0.197 | 0.33 | 0.643 |
| 100 μg/ml CDDP | 0.317 | 0.398 | 0.381 | 1.102 | 1.708 | 1.513 | 0.051 | 0.054 | 0.037 |
| 0.01 ng/ml A + | 0.732 | 0.512 | 1.101 | 1.559 | 1.978 | 1.799 | 0.043 | 0.045 | 0.035 |
| 100 μg/ml CDDP | | | | | | | | | |
| Average | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 0.1 ng/ml A | 0.755 | 1.052 | 2.911 | 2.489 | 2.545 | 2.68 | 0.197 | 0.362 | 0.644 |
| 0.1 μg/ml CDDP | 0.454 | 0.45 | 1.124 | 2.474 | 2.112 | 1.969 | 0.181 | 0.262 | 0.397 |
| 0.1 ng/ml A + | 0.61 | 0.63 | 0.75 | 2.253 | 1.932 | 1.969 | 0.148 | 0.214 | 0.226 |
| 0.1 μg/ml CDDP | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 0.1 ng/ml A | 0.755 | 1.052 | 2.911 | 2.489 | 2.545 | 2.68 | 0.197 | 0.362 | 0.644 |
| 1 μg/ml CDDP | 0.385 | 0.223 | 0.33 | 1.601 | 1.969 | 1.201 | 0.051 | 0.052 | 0.046 |
| 0.1 ng/ml A + | 0.495 | 0.288 | 0.349 | 1.825 | 1.999 | 1.551 | 0.044 | 0.041 | 0.035 |
| 1 μg/ml CDDP | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 0.1 ng/ml A | 0.755 | 1.052 | 2.911 | 2.489 | 2.545 | 2.68 | 0.197 | 0.362 | 0.644 |
| 10 μg/ml CDDP | 0.597 | 0.315 | 0.334 | 1.417 | 1.988 | 1.567 | 0.07 | 0.066 | 0.041 |
| 0.1 ng/ml A + | 0.851 | 0.309 | 0.5 | 1.755 | 2.004 | 1.383 | 0.079 | 0.06 | 0.041 |
| 10 μg/ml CDDP | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 0.1 ng/ml A | 0.755 | 1.052 | 2.911 | 2.489 | 2.545 | 2.68 | 0.197 | 0.362 | 0.644 |
| 100 μg/ml CDDP | 0.317 | 0.398 | 0.381 | 1.102 | 1.708 | 1.513 | 0.051 | 0.054 | 0.037 |
| 0.1 ng/ml A + | 0.694 | 0.718 | 0.481 | 1.964 | 2.426 | 1.781 | 0.044 | 0.046 | 0.035 |
| 100 μg/ml CDDP | | | | | | | | | |
| Average | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 1 ng/ml A | 0.807 | 1.31 | 3.061 | 2.394 | 2.606 | 2.686 | 0.249 | 0.411 | 0.584 |
| 0.1 μg/ml CDDP | 0.454 | 0.45 | 1.124 | 2.474 | 2.112 | 1.969 | 0.181 | 0.262 | 0.397 |
| 1 ng/ml A + | 0.631 | 0.825 | 0.975 | 2.412 | 2.253 | 2.402 | 0.227 | 0.173 | 0.205 |
| 0.1 μg/ml CDDP | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 1 ng/ml A | 0.807 | 1.31 | 3.061 | 2.394 | 2.606 | 2.686 | 0.249 | 0.411 | 0.584 |
| 1 μg/ml CDDP | 0.385 | 0.223 | 0.33 | 1.601 | 1.969 | 1.201 | 0.051 | 0.052 | 0.046 |
| 1 ng/ml A + | 0.444 | 0.279 | 0.377 | 1.73 | 2.016 | 1.579 | 0.051 | 0.053 | 0.035 |
| 1 μg/ml CDDP | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 1 ng/ml A | 0.807 | 1.31 | 3.061 | 2.394 | 2.606 | 2.686 | 0.249 | 0.411 | 0.584 |
| 10 μg/ml CDDP | 0.597 | 0.315 | 0.334 | 1.417 | 1.988 | 1.567 | 0.07 | 0.066 | 0.041 |
| 1 ng/ml A + | 0.553 | 0.538 | 0.445 | 2.126 | 2.211 | 1.979 | 0.156 | 0.096 | 0.054 |
| 10 μg/ml CDDP | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 1 ng/ml A | 0.807 | 1.31 | 3.061 | 2.394 | 2.606 | 2.686 | 0.249 | 0.411 | 0.584 |
| 100 μg/ml CDDP | 0.317 | 0.398 | 0.381 | 1.102 | 1.708 | 1.513 | 0.051 | 0.054 | 0.037 |
| 1 ng/ml A + | 0.379 | 0.77 | 0.476 | 1.838 | 2.645 | 2.1 | 0.052 | 0.056 | 0.037 |
| 100 μg/ml CDDP | | | | | | | | | |
| Average | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 10 ng/ml A | 0.763 | 1.249 | 2.448 | 2.178 | 2.541 | 2.569 | 0.279 | 0.476 | 0.584 |
| 0.1 μg/ml CDDP | 0.454 | 0.45 | 1.124 | 2.474 | 2.112 | 1.969 | 0.181 | 0.262 | 0.397 |
| 10 ng/ml A + | 0.66 | 0.705 | 0.744 | 2.085 | 1.992 | 1.853 | 0.202 | 0.255 | 0.198 |
| 0.1 μg/ml CDDP | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 10 ng/ml A | 0.763 | 1.249 | 2.448 | 2.178 | 2.541 | 2.569 | 0.279 | 0.476 | 0.584 |
| 1 μg/ml CDDP | 0.385 | 0.223 | 0.33 | 1.601 | 1.969 | 1.201 | 0.051 | 0.052 | 0.046 |
| 10 ng/ml A + | 0.42 | 0.251 | 0.433 | 1.45 | 1.716 | 1.236 | 0.043 | 0.047 | 0.033 |
| 1 μg/ml CDDP | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 10 ng/ml A | 0.763 | 1.249 | 2.448 | 2.178 | 2.541 | 2.569 | 0.279 | 0.476 | 0.584 |
| 10 μg/ml CDDP | 0.597 | 0.315 | 0.334 | 1.417 | 1.988 | 1.567 | 0.07 | 0.066 | 0.041 |
| 10 ng/ml A + | 0.473 | 0.406 | 0.462 | 1.927 | 1.692 | 1.257 | 0.096 | 0.07 | 0.04 |
| 10 μg/ml CDDP | | | | | | | | | |
| Unstimulated | 0.54 | 0.912 | 2.719 | 2.588 | 2.481 | 2.593 | 0.237 | 0.404 | 0.512 |
| cells | | | | | | | | | |
| 10 ng/ml A | 0.763 | 1.249 | 2.448 | 2.178 | 2.541 | 2.569 | 0.279 | 0.476 | 0.584 |
| 100 μg/ml CDDP | 0.317 | 0.398 | 0.381 | 1.102 | 1.708 | 1.513 | 0.051 | 0.054 | 0.037 |
| 10 ng/ml A + | 0.45 | 0.394 | 0.729 | 1.487 | 2.32 | 1.771 | 0.045 | 0.056 | 0.033 |
| 100 μg/ml CDDP | | | | | | | | | |

TABLE 46

| | T47D | Run 3 | | | | | | | |
|---|---|---|---|---|---|---|---|---|---|
| | CVE | CVE | CVE | SRB | SRB | SRB | MTT | MTT | MTT |
| | 24 h | 48 h | 72 h | 24 h | 48 h | 72 h | 24 h | 48 h | 72 h |
| Average | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 0.01 ng/ml A | 0.549 | 1.377 | 1.736 | 1.436 | 2.549 | 2.878 | 0.162 | 0.45 | 0.906 |
| 0.1 μg/ml CDDP | 0.561 | 0.778 | 0.722 | 1.615 | 2.223 | 2.044 | 0.148 | 0.261 | 0.371 |
| 0.01 ng/ml A + | 0.416 | 0.562 | 0.445 | 1.691 | 1.822 | 1.622 | 0.135 | 0.222 | 0.179 |
| 0.1 μg/ml CDDP | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 0.01 ng/ml A | 0.549 | 1.377 | 1.736 | 1.436 | 2.549 | 2.878 | 0.162 | 0.45 | 0.906 |
| 1 μg/ml CDDP | 0.4 | 0.435 | 0.223 | 1.846 | 2.03 | 1.344 | 0.033 | 0.047 | 0.035 |
| 0.01 ng/ml A + | 0.207 | 0.383 | 0.17 | 1.804 | 2.067 | 1.156 | 0.029 | 0.039 | 0.025 |
| 1 μg/ml CDDP | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 0.01 ng/ml A | 0.549 | 1.377 | 1.736 | 1.436 | 2.549 | 2.878 | 0.162 | 0.45 | 0.906 |
| 10 μg/ml CDDP | 0.488 | 0.619 | 0.31 | 1.368 | 1.974 | 1.461 | 0.074 | 0.077 | 0.057 |
| 0.01 ng/ml A + | 0.364 | 0.598 | 0.232 | 1.558 | 1.391 | 1.054 | 0.079 | 0.079 | 0.044 |
| 10 μg/ml CDDP | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 0.01 ng/ml A | 0.549 | 1.377 | 1.736 | 1.436 | 2.549 | 2.878 | 0.162 | 0.45 | 0.906 |
| 100 μg/ml CDDP | 0.385 | 0.544 | 0.22 | 1.34 | 1.758 | 1.056 | 0.047 | 0.051 | 0.035 |
| 0.01 ng/ml A + | 0.7 | 0.522 | 0.394 | 1.666 | 1.654 | 1.082 | 0.029 | 0.045 | 0.025 |
| 100 μg/ml CDDP | | | | | | | | | |
| Average | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 0.1 ng/ml A | 0.597 | 1.51 | 1.823 | 1.429 | 2.602 | 2.842 | 0.154 | 0.319 | 0.79 |
| 0.1 μg/ml CDDP | 0.561 | 0.778 | 0.722 | 1.615 | 2.223 | 2.044 | 0.148 | 0.261 | 0.371 |
| 0.1 ng/ml A + | 0.533 | 0.654 | 0.438 | 1.949 | 2.19 | 1.844 | 0.115 | 0.15 | 0.261 |
| 0.1 μg/ml CDDP | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 0.1 ng/ml A | 0.597 | 1.51 | 1.823 | 1.429 | 2.602 | 2.842 | 0.154 | 0.319 | 0.79 |
| 1 μg/ml CDDP | 0.4 | 0.435 | 0.223 | 1.846 | 2.03 | 1.344 | 0.033 | 0.047 | 0.035 |
| 0.1 ng/ml A + | 0.305 | 0.425 | 0.17 | 2.218 | 2.049 | 1.478 | 0.029 | 0.042 | 0.026 |
| 1 μg/ml CDDP | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 0.1 ng/ml A | 0.597 | 1.51 | 1.823 | 1.429 | 2.602 | 2.842 | 0.154 | 0.319 | 0.79 |
| 10 μg/ml CDDP | 0.488 | 0.619 | 0.31 | 1.368 | 1.974 | 1.461 | 0.074 | 0.077 | 0.057 |
| 0.1 ng/ml A + | 0.329 | 0.602 | 0.478 | 1.475 | 1.814 | 1.42 | 0.052 | 0.051 | 0.031 |
| 10 μg/ml CDDP | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 0.1 ng/ml A | 0.597 | 1.51 | 1.823 | 1.429 | 2.602 | 2.842 | 0.154 | 0.319 | 0.79 |
| 100 μg/ml CDDP | 0.385 | 0.544 | 0.22 | 1.34 | 1.758 | 1.056 | 0.047 | 0.051 | 0.035 |
| 0.1 ng/ml A + | 0.467 | 0.71 | 0.606 | 1.568 | 1.972 | 1.052 | 0.029 | 0.045 | 0.026 |
| 100 μg/ml CDDP | | | | | | | | | |
| Average | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 1 ng/ml A | 0.727 | 1.715 | 2.619 | 1.54 | 2.666 | 2.874 | 0.141 | 0.294 | 0.669 |
| 0.1 μg/ml CDDP | 0.561 | 0.778 | 0.722 | 1.615 | 2.223 | 2.044 | 0.148 | 0.261 | 0.371 |
| 1 ng/ml A + | 0.768 | 1.124 | 0.629 | 1.684 | 2.245 | 1.921 | 0.098 | 0.165 | 0.224 |
| 0.1 μg/ml CDDP | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 1 ng/ml A | 0.727 | 1.715 | 2.619 | 1.54 | 2.666 | 2.874 | 0.141 | 0.294 | 0.669 |
| 1 μg/ml CDDP | 0.4 | 0.435 | 0.223 | 1.846 | 2.03 | 1.344 | 0.033 | 0.047 | 0.035 |
| 1 ng/ml A + | 0.357 | 0.497 | 0.18 | 1.759 | 2.162 | 1.237 | 0.032 | 0.047 | 0.028 |
| 1 μg/ml CDDP | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 1 ng/ml A | 0.727 | 1.715 | 2.619 | 1.54 | 2.666 | 2.874 | 0.141 | 0.294 | 0.669 |
| 10 μg/ml CDDP | 0.488 | 0.619 | 0.31 | 1.368 | 1.974 | 1.461 | 0.074 | 0.077 | 0.057 |
| 1 ng/ml A + | 0.532 | 0.793 | 0.303 | 1.607 | 1.944 | 1.293 | 0.083 | 0.091 | 0.05 |
| 10 μg/ml CDDP | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 1 ng/ml A | 0.727 | 1.715 | 2.619 | 1.54 | 2.666 | 2.874 | 0.141 | 0.294 | 0.669 |
| 100 μg/ml CDDP | 0.385 | 0.544 | 0.22 | 1.34 | 1.758 | 1.056 | 0.047 | 0.051 | 0.035 |
| 1 ng/ml A + | 0.413 | 0.5 | 0.656 | 1.49 | 2.289 | 1.367 | 0.04 | 0.049 | 0.031 |
| 100 μg/ml CDDP | | | | | | | | | |
| Average | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 10 ng/ml A | 0.818 | 1.786 | 2.344 | 1.443 | 2.555 | 2.831 | 0.152 | 0.326 | 0.67 |
| 0.1 μg/ml CDDP | 0.561 | 0.778 | 0.722 | 1.615 | 2.223 | 2.044 | 0.148 | 0.261 | 0.371 |
| 10 ng/ml A + | 0.827 | 1.201 | 0.707 | 1.729 | 1.974 | 1.581 | 0.105 | 0.192 | 0.231 |
| 0.1 μg/ml CDDP | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 10 ng/ml A | 0.818 | 1.786 | 2.344 | 1.443 | 2.555 | 2.831 | 0.152 | 0.326 | 0.67 |
| 1 μg/ml CDDP | 0.4 | 0.435 | 0.223 | 1.846 | 2.03 | 1.344 | 0.033 | 0.047 | 0.035 |
| 10 ng/ml A + | 0.349 | 0.571 | 0.238 | 1.658 | 1.936 | 1.07 | 0.043 | 0.037 | 0.029 |
| 1 μg/ml CDDP | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 10 ng/ml A | 0.818 | 1.786 | 2.344 | 1.443 | 2.555 | 2.831 | 0.152 | 0.326 | 0.67 |
| 10 μg/ml CDDP | 0.488 | 0.619 | 0.31 | 1.368 | 1.974 | 1.461 | 0.074 | 0.077 | 0.057 |
| 10 ng/ml A + | 0.517 | 0.655 | 0.382 | 1.412 | 1.472 | 1.239 | 0.081 | 0.057 | 0.038 |
| 10 μg/ml CDDP | | | | | | | | | |
| Unstimulated | 0.526 | 1.299 | 1.748 | 1.403 | 2.508 | 2.872 | 0.183 | 0.477 | 0.851 |
| cells | | | | | | | | | |
| 10 ng/ml A | 0.818 | 1.786 | 2.344 | 1.443 | 2.555 | 2.831 | 0.152 | 0.326 | 0.67 |
| 100 μg/ml CDDP | 0.385 | 0.544 | 0.22 | 1.34 | 1.758 | 1.056 | 0.047 | 0.051 | 0.035 |
| 10 ng/ml A + | 0.623 | 0.615 | 0.604 | 1.504 | 1.712 | 1.345 | 0.046 | 0.044 | 0.032 |
| 100 μg/ml CDDP | | | | | | | | | |

1. A therapeutic combination comprising (I) an anti-neoplastic agent comprising platinum and (II) a hot water-based extract from a species of the genus Nerium.

2. The therapeutic combination of claim 1, wherein (I) is present in an amount that, in the absence of (II), is effective to produce an increase in anti-neoplastic activity.

3. The therapeutic combination of claim 1, wherein (I) is present in an amount that, in the absence of (II), is ineffective to produce an increase in anti-neoplastic activity.

4. The therapeutic combination of claim 1, wherein (I) is selected from the group consisting of cisplatin, carboplatin, oxaliplatin, satraplatin, picoplatin, nedaplatin, triplatin, and phosphaplatins.

5. The therapeutic combination of claim 1, wherein (I) is cisplatin.

6. The therapeutic combination of claim 1, wherein (II) is an extract from a species selected from the group consisting of Nerium indicum, Nerium oleander, and Nerium odorum.

7. The therapeutic combination of claim 1, wherein (II) is an extract from the species Nerium oleander.

8. (canceled)

9. (canceled)

10. The therapeutic combination of claim 1, wherein (I) is cisplatin and (II) is an extract from the species Nerium oleander.

11. The therapeutic combination of claim 1, wherein (I) and (II) are present as separate formulations.

12. A method for treating cancer comprising administering to a patient in need thereof a therapeutically effective amount of the therapeutic combination of claim 1.

13. The method of claim 12, wherein (I) is present in an amount that, in the absence of (II), is effective to produce an increase in anti-neoplastic activity.

14. The method of claim 12, wherein (I) is present in an amount that, in the absence of (II), is ineffective to produce an increase in anti-neoplastic activity.

15. The method of claim 12, wherein (I) is selected from the group consisting of cisplatin, carboplatin, oxaliplatin, satraplatin, picoplatin, nedaplatin, triplatin, and phosphaplatins.

16. The method of claim 12, wherein (I) is cisplatin.

17. The method of claim 12, wherein (II) is an extract from a species selected from the group consisting of Nerium indicum, Nerium oleander, and Nerium odorum.

18. The method of claim 12, wherein (II) is an extract from the species Nerium oleander.

19. (canceled)

20. (canceled)

21. The method of claim 12, wherein (I) is cisplatin and (II) is an extract from the species Nerium oleander.

22. The method of claim 12, wherein (I) and (II) of said therapeutic combination are administered to the patient simultaneously, separately, or sequentially.

23. The method of claim 12, wherein (II) is administered to the patient intramuscularly, sublingually, or intramuscularly and sublingually.

24. The method of claim 12, wherein (II) is administered to the patient sublingually in two or more doses.

25. (canceled)
